(19) United States
(12) Patent Application Publication
(10) Pub. No.: US 2024/0117351 A1
(43) Pub. Date: Apr. 11, 2024

(54) COMPOSITIONS AND METHODS FOR INHIBITING GENE EXPRESSION IN THE CENTRAL NERVOUS SYSTEM

(71) Applicant: Dicerna Pharmaceuticals, Inc., Lexington, MA (US)

(72) Inventors: Maire JUNG, Lincoln, MA (US); Travis GRIM, Maynard, MA (US); Weimin WANG, Winchester, MA (US); Bob Dale BROWN, Littleton, MA (US)

(21) Appl. No.: 18/274,826

(22) PCT Filed: Jan. 28, 2022
(86) PCT No.: PCT/US2022/014346

§ 371 Date: Jul. 28, 2023

Related U.S. Application Data
(60) Provisional application No. 63/142,877, filed on Jan. 28, 2021.

(51) Int. Cl. C12N 15/113; A61K 47/54

(52) U.S. Cl. CPC C12N 15/113; A61K 47/543; C12N 2310/3515; C12N 2310/531; C12N 2310/315; C12N 2310/321; C12N 2310/322; C12N 2320/32

(57) ABSTRACT

Oligonucleotide conjugates are provided herein that inhibit or reduce expression of target genes in the CNS. Also provided are compositions including the same and uses thereof, particularly uses relating to treating diseases, disorders and/or conditions associated with target gene expression in the CNS.

### CROSS-REFERENCE TO RELATED APPLICATIONS

This application claims priority to U.S. Provisional Patent Application No. 63/142,877, filed Jan. 28, 2021, the contents of which are incorporated herein by reference.

### TECHNICAL FIELD OF THE DISCLOSURE

The present disclosure relates to nucleic acid-hydrophobic ligand conjugates and oligonucleotide-hydrophobic ligand conjugates. Specifically, the present disclosure relates to nucleic acid-lipid conjugates and oligonucleotide-lipid conjugates, methods to prepare them, their chemical configuration, and methods to modulate (e.g., inhibit or reduce) the expression of a target gene in the central nervous system (abbreviated “CNS” herein) (e.g., in a cell, tissue, or region of the CNS) using the conjugated nucleic acids and oligonucleotides according to the description provided herein. The disclosure also provides pharmaceutically acceptable compositions comprising the conjugates of the present description and methods of using said compositions in the treatment of various diseases or disorders.

### BACKGROUND OF THE DISCLOSURE

Regulation of gene expression by modified nucleic acids shows great potential as both a research tool in the laboratory and a therapeutic approach in the clinic. Several classes of oligonucleotide or nucleic acid-based therapeutics have been under clinical investigation, including antisense oligonucleotides (ASO), short interfering RNA (siRNA), double-stranded nucleic acids (dsNA), aptamers, ribozymes, exon-skipping and splice-altering oligonucleotides, immunomodulatory oligonucleotides, mRNAs, and CRISPR. Chemical modifications in the relevant molecules play a key role in overcoming challenges of oligonucleotide therapeutics, including improving nuclease stability, RNA-binding affinity, and pharmacokinetics. Various chemical modification strategies for oligonucleotides have been developed in the past three decades including modification of the sugars, nucleobases, and phosphodiester backbone to improve and optimize performance and therapeutic efficacy (Deleavey and Darma, CHEM. BIOL. 2012, 19(8):937-54; Wan and Seth, J. MED. CHEM. 2016, 59(21):9645-67; and Egli and Manoharan, ACC. CHEM. RES. 2019, 54(4):1036-47).

Therapeutic gene silencing mediated by RNAi oligonucleotide-based therapeutics comprising siRNAs or double-stranded nucleic acids (dsNAs) offer the potential for considerable expansion of the druggable target space and the possibility for treating orphan diseases that may be therapeutically unapproachable by other drug modalities (e.g., antibodies and/or small molecules). RNAi oligonucleotide-based therapeutics that inhibit or reduce expression of specific target genes in the liver have been developed and are currently in clinical use (Sehgal et al., (2013) JOURNAL OF HEPATOLOGY 59:1354-1359). Technological hurdles remain for the development and clinical use of RNAi oligonucleotides in extrahepatic cells, tissues, and organs (e.g., the central nervous system or CNS). Therapeutic gene silencing mediated by RNAi oligonucleotide-based therapeutics in the CNS is of particular interest to treat neurological diseases (Boudreau & Davidson (2010) BRAIN RESEARCH 1338:112-21). Thus, an ongoing need exists in the art for the successful development of new and effective RNAi oligonucleotides to modulate the expression of a target genes in extrahepatic cells, tissues, and/or organs (e.g, the CNS).

### SUMMARY

The mammalian CNS is a complex system of tissues, including cells, fluids and chemicals that interact in concert to enable a wide variety of functions, including movement, navigation, cognition, speech, vision, and emotion. Unfortunately, a variety of diseases and disorders of the CNS are known (e.g., neurological disorders) and affect or disrupt some or all of these functions. Typically, treatments for diseases and disorders of the CNS have been limited to small molecule drugs, antibodies and/or to adaptive or behavioral therapies. There exists an ongoing need to develop treatment of diseases and disorders of the CNS associated with inappropriate gene expression. Accordingly, the present disclosure is directed to compositions comprising oligonucleotide-ligand conjugates (e.g., RNAi oligonucleotide conjugates) comprising one or more hydrophobic moieties that modulate (e.g., reduce or inhibit) target gene expression in the CNS. The present disclosure is also directed to methods of preparation and methods of use and treatment of said oligonucleotide conjugates.

The present application relates to novel nucleic acids, oligonucleotides or analogues thereof comprising hydrophobic ligands, including but not limited to adamantyl and lipid conjugates. The present disclosure relates to nucleic acid-lipid conjugates and oligonucleotide-lipid conjugates, which function to modulate the expression of a target gene in a cell, and methods of preparation and uses thereof. Without being bound by theory, lipophilic/hydrophobic moieties, such as fatty acids and adamantyl group when attached to these highly hydrophilic nucleic acids/oligonucleotides substantially enhance plasma protein binding and consequently circulation half-life. The conjugated nucleic acids, oligonucleotides, and analogues thereof provided herein are stable and bind to RNA targets to elicit broad extrahepatic RNase H activity and are also useful in splice switching and RNAi. Without wishing to be bound by theory, incorporation of the hydrophobic moiety, such as a lipid, facilitates systemic delivery of the novel nucleic acids, oligonucleotides, or analogues thereof into several tissues, including but not limited to, the CNS, muscle, adipose, and adrenal gland.

Suitable nucleic acid-hydrophobic ligand conjugates and oligonucleotide-hydrophobic ligand conjugates include nucleic acid inhibitor molecules, such as dsRNA inhibitor molecules, dsRNAi inhibitor molecules, antisense oligonucleotides, miRNA, ribozymes, antagomirs, aptamers, and single-stranded RNAi inhibitor molecules. In particular, the present disclosure provides nucleic acid-lipid conjugates, oligonucleotide-lipid conjugates, and analogues thereof, which find utility as modulators of intracellular RNA levels. Nucleic acid inhibitor molecules of the disclosure modulate RNA expression through a diverse set of mechanisms, for example by RNA interference (RNAi). An advantage of the nucleic acid-hydrophobic ligand conjugates, oligonucleotide-hydrophobic ligand conjugates and analogues thereof provided herein is that a broad range of pharmacological activities is possible, consistent with the modulation of intracellular RNA levels. In addition, the disclosure provides methods of using an effective amount of the conjugates described herein for the treatment or amelioration of a disease condition by modulating the intracellular RNA levels.

The present disclosure relates to RNAi oligonucleotide conjugates comprising one or more nucleic acid-ligand conjugate units that modulate target gene expression in the CNS via RNA interference (RNAi). In particular, the present disclosure relates to novel RNAi oligonucleotide conjugates comprising one or more hydrophobic moiety ligand(s), including, but not limited to, lipid moieties, that modulate (e.g., reduce or inhibit) target gene expression in the CNS, compositions of said RNAi oligonucleotide conjugates, and methods of preparation and uses thereof.

The present disclosure is based, at least in part, on the discovery of RNAi oligonucleotide-lipid conjugates that effectively reduce target gene expression in the CNS for a prolonged period. Exemplary RNAi oligonucleotide-lipid conjugates provided herein have demonstrated sustained, multi-month reduction of target gene expression in the CNS following a single administration. Further, exemplary RNAi oligonucleotide-lipid conjugates provided herein have demonstrated pharmacological activity in multiple regions throughout the CNS. Without being bound by theory, the hydrophobic moiety (e.g., lipid) facilitates delivery and distribution of the RNAi oligonucleotide-lipid conjugate into the CNS, thereby increasing efficacy and durability of gene knockdown. Furthermore, the present disclosure provides RNAi oligonucleotide-lipid conjugates that effectively reduce target gene expression in the CNS, without reducing target gene expression in the liver. Accordingly, the disclosure provides methods of treating a disease or disorder by modulating target gene expression in the CNS using the RNAi oligonucleotide-lipid conjugates, and pharmaceutically acceptable compositions thereof, described herein. The disclosure further provides methods of using the RNAi oligonucleotide-lipid conjugates in the manufacture of a medicament for treating a disease or disorder by modulating target gene expression in the CNS.

RNAi has evolved rapidly as a tool for directed gene silencing. The RNAi machinery present in cells can be co-opted in many ways to achieve gene expression knockdown of a select target, even in the CNS. According to the present disclosure and without being bound by theory, the synthetic siRNAs are introduced into cells, which are loaded directly into RISC or, in the case of longer dsRNAs (25-27 nucleotides), first processed by Dicer and then loaded into the RISC to achieve gene silencing. The RNAi therapy provided by the various embodiments of the present disclosure are well suited for diseases and disorders where the disease-causing gene acquires a negative or disruptive ‘gain of function’ effect. The identification of such disease-causing genes in the literature along with the current disclosure will allow the design of RNAi oligonucleotide molecules to target the disease-causing alleles and provide therapeutic relief. The types of diseases/disorders targeted by the disclosure provided herein include, without limitation: Progressive Supranuclear Palsy (PSP), Corticobasal degeneration (CBD), Argyrophilic grain disease (AGD), Globular glial tauopathy (GGT), Aging-related tau astrogliopathy (ARTAG), Familial Frontotemporal Dementia 17 (FTD-17), Tauopathy with Respiratory Failure, Dementia with Seizures, Pick's disease, Myotonic dystrophy 1 or 2 (MD1 or MD2), Down's syndrome, Spastic Paraplegia (SP), Niemann-Pick disease type C, Dementia with Lewy bodies (DLB), Lewy body dysphagia, Lewy body disease, Olivopontocerebellar atrophy, Striatonigral degeneration, Shy-Drager syndrome, Spinal muscular atrophy V (SMAV), Huntington's Disease (HD), Alzheimer's Disease, SCA1, SCA2, SCA3, SCA7, SCA10 (spinocerebellar ataxia type 1, 2, 3, 7 or 10), Multiple System Atrophy (MSA), Spinal and Bulbar Muscular Atrophy (SBMA, Kennedy disease), Friedrich Ataxia, Fragile X-associated tremor/ataxia syndrome (FXTAS), Fragile X syndrome (FRAXA), X-Linked Mental Retardation (XLMR), Parkinson's Disease, Dystonia, SBMA (spinobulbar muscular atrophy), neuropathic pain disorders, spinal cord injury, Dentatorubral-pallidoluysian atrophy (DRPLA), recessive CNS disorders, ALS (amyotrophic lateral sclerosis), M2DS (MECP2 duplication syndrome), FTD (frontotemporal dementia), Prion disease, Adult Onset Leukodystrophy, Alexander's Disease, Krabbe Disease, Chronic Traumatic Encephalopathy, Pelizaeus-Merzbacher disease (PMD), Lafora disease, stroke, Cerebral Amyloid Angiopathy (CAA), and Metachromatic Leukodystrophy (MLD). In some embodiments, the disease or disorder is PMD, spinal cord injury, stroke, Krabbe Disease, MLD, SCA3, prion disease, Alzheimer's Disease, Alexander's Disease, Adult-onset leukodystrophy, MECP2 duplication syndrome, Charcot-Marie-Tooth, Multiple Sclerosis, Kennedy's Disease, Huntington's Disease, X-linked adrenoleukodystrophy, or SCA1. In some embodiments the disease or disorder is PMD. In some embodiments the disease or disorder is spinal cord injury. In some embodiments the disease or disorder is stroke. In some embodiments the disease or disorder is Krabbe Disease. In some embodiments the disease or disorder is MLD. In some embodiments the disease or disorder is SCA3. In some embodiments the disease or disorder is prion disease. In some embodiments the disease or disorder is Alzheimer's Disease. In some embodiments the disease or disorder is Alexander's Disease. In some embodiments the disease or disorder is Adult-onset leukodystrophy. In some embodiments the disease or disorder is MECP2 duplication syndrome. In some embodiments the disease or disorder is Charcot-Marie-Tooth. In some embodiments the disease or disorder is Multiple Sclerosis. In some embodiments the disease or disorder is Kennedy's Disease. In some embodiments the disease or disorder is Huntington's Disease. In some embodiments the disease or disorder is X-linked adrenoleukodystrophy. In some embodiments the disease or disorder is SCA1.

In some embodiments, the nucleic acid-hydrophobic ligand conjugates of the present disclosure, and pharmaceutically acceptable compositions thereof, are effective as modulators of intracellular RNA levels and/or RNA function. Such nucleic acid-lipid conjugates thereof comprising one or more lipid conjugates are represented by formula I-a:

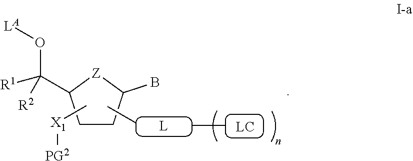

or a pharmaceutically acceptable salt thereof, wherein each variable is as defined and described herein.

In certain embodiments, the nucleic acid-ligand conjugates are represented by formula I-b, I-c, I-Ib or I-Ic:

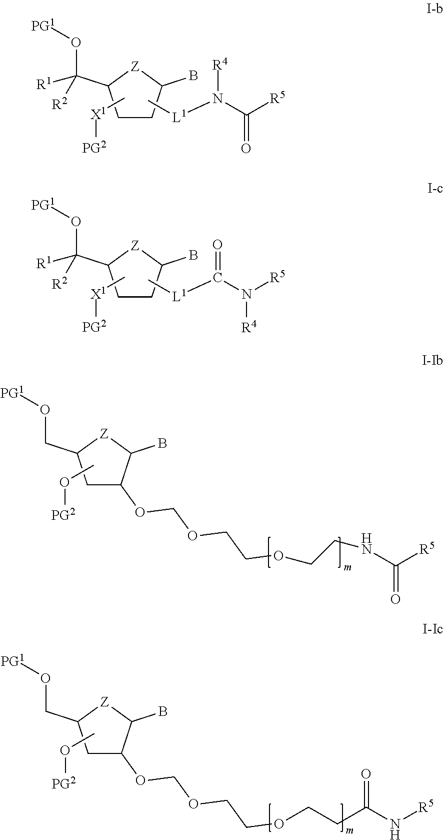

or a pharmaceutically acceptable salt thereof, wherein each variable is as defined and described herein.

In another aspect, the present disclosure presents oligonucleotide-ligand conjugates represented by formula II-a:

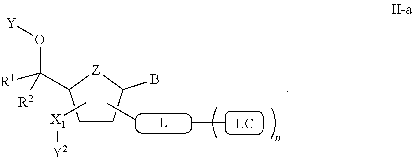

or a pharmaceutically acceptable salt thereof, wherein each variable is as defined and described herein.

In certain embodiments, the oligonucleotide-lipid conjugates are represented by formula II-b, II-c, II-Ib or II-Ic:

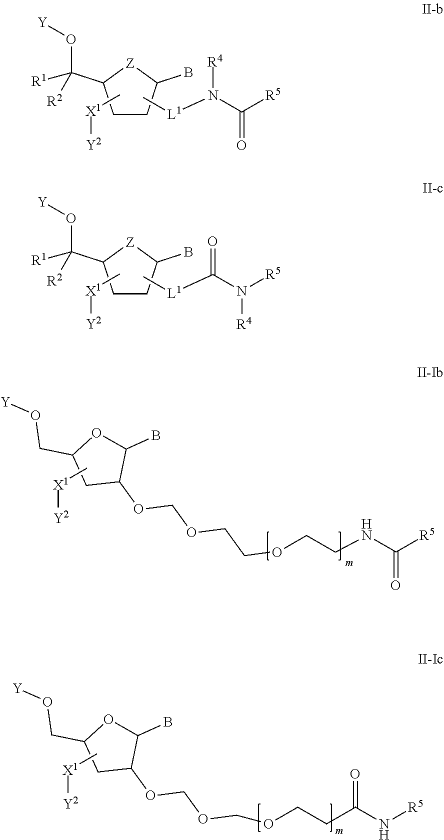

or a pharmaceutically acceptable salt thereof.

In certain embodiments, the oligonucleotide-ligand conjugate of any of the above disclosed embodiments comprises 1-10 nucleic acid-ligand or nucleic acid analog-ligand conjugate units. In some embodiments, the oligonucleotide-ligand conjugate comprises 1, 2 or 3 nucleic acid-ligand conjugate units.

In some embodiments, the oligonucleotide-ligand conjugate comprises a sense strand of 10-53 nucleotides in length and an antisense strand of 15-53 nucleotides in length, wherein the antisense oligonucleotide strand has sequence complementary to at least 15 consecutive nucleotides of a target gene sequence and reduces expression of the target gene when the oligonucleotide-conjugate is introduced into a mammalian cell. In some embodiments, the oligonucleotide-ligand conjugate comprises a nucleotide strand of 9 to 30 nucleotides in length. In some embodiments, the oligonucleotide-ligand conjugate is single-stranded. In some embodiments, the oligonucleotide-ligand conjugate is double-stranded.

In certain embodiments of the oligonucleotide-ligand conjugate, the antisense strand is 19 to 27 nucleotides in length. In certain embodiments of the oligonucleotide-ligand conjugate, the sense strand is 12 to 40 nucleotides in length. In certain embodiments of the oligonucleotide-ligand conjugate, the sense strand forms a duplex region with the antisense strand. In certain embodiments of the oligonucleotide-ligand conjugate, the region of complementarity is fully complementary to the target sequence.

In certain embodiments of the oligonucleotide-ligand conjugate, the sense strand comprises at its 3′-end a stem-loop set forth as: S₁-L-S₂, wherein S₁ is complementary to S₂, and wherein L forms a loop between S₁ and S₂ of 3 to 5 nucleotides in length. In certain embodiments of the oligonucleotide-ligand conjugate, L is a tetraloop, and L comprises a sequence set forth as GAAA. In some embodiments, L is a tetraloop having a nucleotide sequence set forth as 5′-GAAA-3′.

In certain embodiments, the oligonucleotide-ligand conjugate further comprises a 3′-overhang sequence on the antisense strand of two nucleotides in length. In certain embodiments, the oligonucleotide-ligand conjugate further comprises a 3′-overhang sequence of one or more nucleotides in length, wherein the 3′-overhang sequence is present on the antisense strand, the sense strand, or the antisense strand and sense strand.

In certain embodiments, the oligonucleotide-ligand conjugate comprises at least one modified nucleotide, and wherein the modified nucleotide comprises a 2′-modification. In some embodiments, the 2′-modification is a modification selected from: 2′-aminoethyl, 2′-fluoro, 2′-O-methyl, 2′-O-methoxyethyl, 2′-deoxy-2′-fluoro, and 2′-deoxy-2′-fluoro-β-d-arabino.

In certain embodiments of the oligonucleotide-ligand conjugate, all the nucleotides of the oligonucleotide are modified.

In certain embodiments of the oligonucleotide-ligand conjugate, the oligonucleotide comprises at least one modified internucleotide linkage. In some embodiments of the oligonucleotide-ligand conjugate, the at least one modified internucleotide linkage is a phosphorothioate linkage.

In some embodiments of the oligonucleotide-ligand conjugate, the 4′-carbon of the sugar of the 5′-nucleotide of the antisense strand comprises a phosphate analog. The phosphate analog is oxymethylphosphonate, vinylphosphonate, or malonylphosphonate.

In some aspects, the disclosure provides a nucleic acid-ligand conjugate represented by formula I-a:

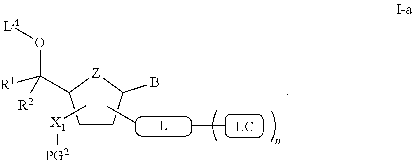

or a pharmaceutically acceptable salt thereof, wherein:

B is a nucleobase or hydrogen;

R¹ and R² are independently hydrogen, halogen, R^{A}, —CN, —S(O)R, —S(O)₂R, —Si(OR)₂R, —Si(OR)R₂, or —SiR₃, or

R¹ and R² on the same carbon are taken together with their intervening atoms to form a 3-membered saturated or partially unsaturated ring having 0-3 heteroatoms, independently selected from nitrogen, oxygen, and sulfur;

each R^{A} is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;

each R is independently hydrogen, a suitable protecting group, or an optionally substituted group selected from C1-6 aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:

two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur;

L^{A} is independently PG¹, or -L-ligand;

PG¹ is hydrogen or a suitable hydroxyl protecting group;

each ligand is independently -(LC)n, and/or an adamantyl group;

each LC is independently a lipid conjugate moiety comprising a saturated or unsaturated, straight, or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, or —P(S)OR—;

each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, adamantanenyl, a 4-7 membered saturated or partially unsaturated heterocyclylenyl having 1-3 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;

n is 1-10;

L is a covalent bond or a bivalent saturated or unsaturated, straight or branched C₁₋₅₀ hydrocarbon chain, wherein 0-10 methylene units of the hydrocarbon chain are independently replaced by -Cy-, —O—, —NR—, —N(R)—C(O)—, —S—, —C(O)—, —S(O)—, —S(O)₂—, —P(O)OR—, —P(S)OR—, —V¹CR²W¹— or

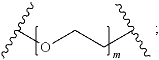

m is 1-50;

X¹, V¹ and W¹ are independently —C(R)₂—, —OR, —O—, —S—, —Se—, or —NR—;

Z is —O—, —S—, —NR—, or —CR₂—; and

PG² is hydrogen, a phosphoramidite analogue, or a suitable protecting group.

In some aspects, the conjugate is represented by formula I-b or I-c:

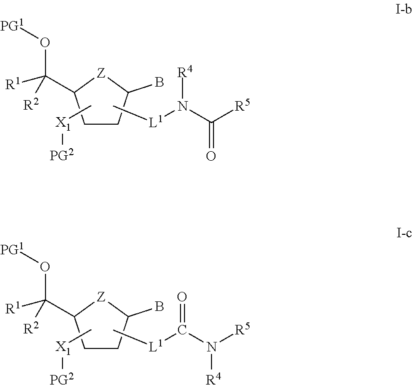

or a pharmaceutically acceptable salt thereof; wherein

L¹ is a covalent bond or a bivalent saturated or unsaturated, straight or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, —P(S)OR—, or

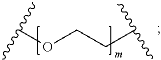

R⁴ is hydrogen, R^{A}, or a suitable amine protection group; and

R⁵ is adamantyl, or a saturated or unsaturated, straight, or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, or —P(S)OR—.

In some aspects, the nucleic acid-ligand conjugate is represented by formula I-Ib or I-Ic:

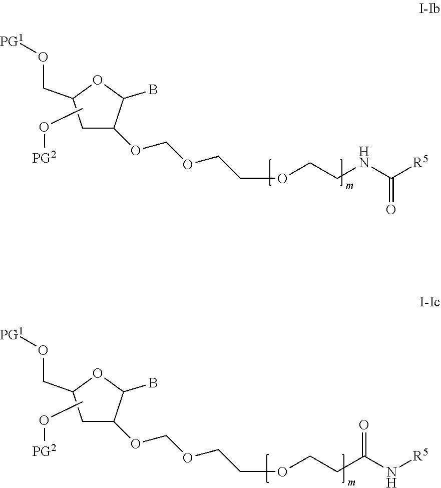

or a pharmaceutically acceptable salt thereof; wherein

B is a nucleobase or hydrogen;

m is 1-50;

PG¹ and PG² are independently a hydrogen, a phosphoramidite analogue, or a suitable protecting group; and

R⁵ is adamantyl, or a saturated or unsaturated, straight, or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, or —P(S)OR—.

In any of the foregoing or related aspects, R⁵ is selected from

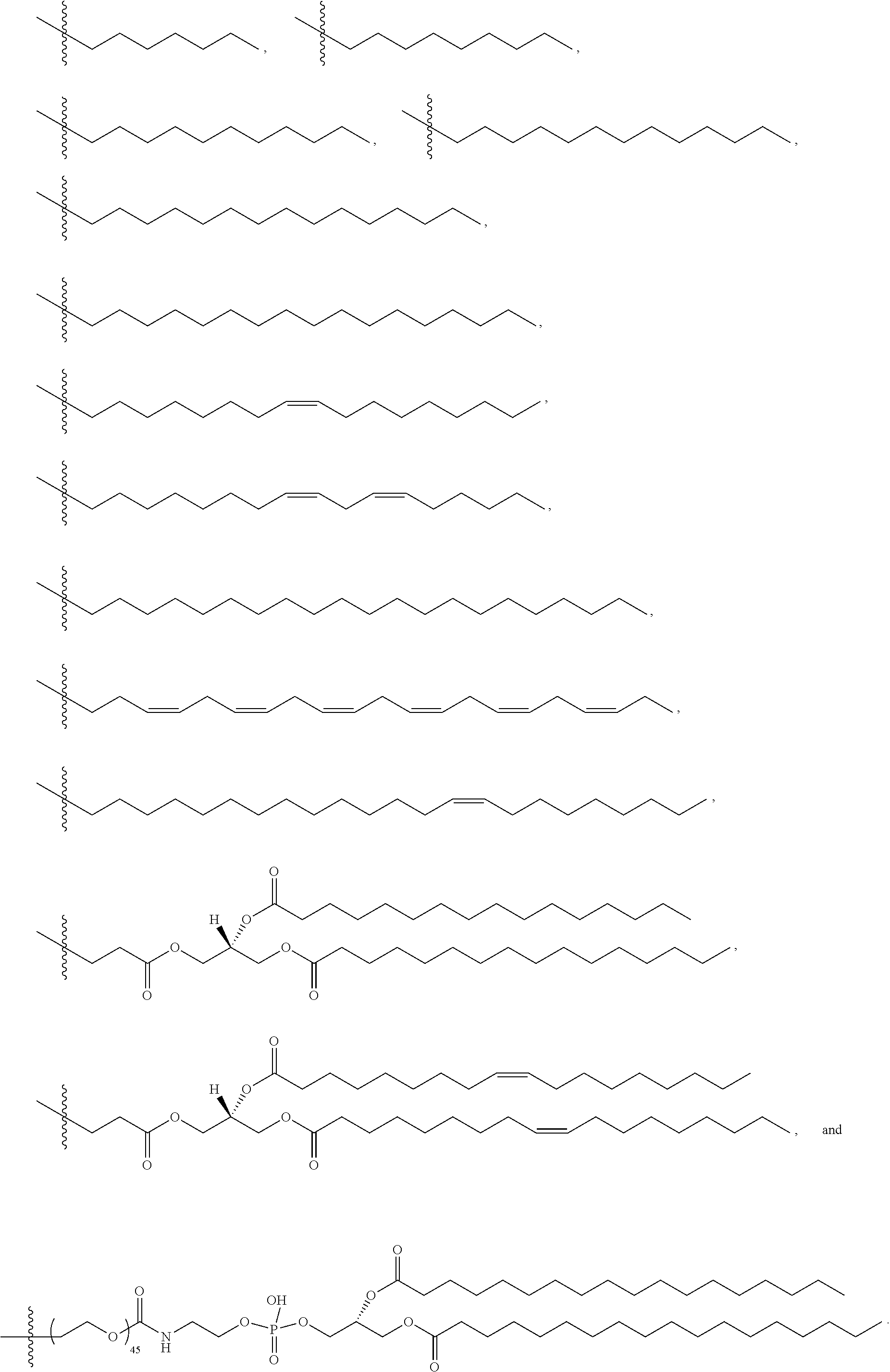

In any of the foregoing or related aspects, R⁵ is selected from

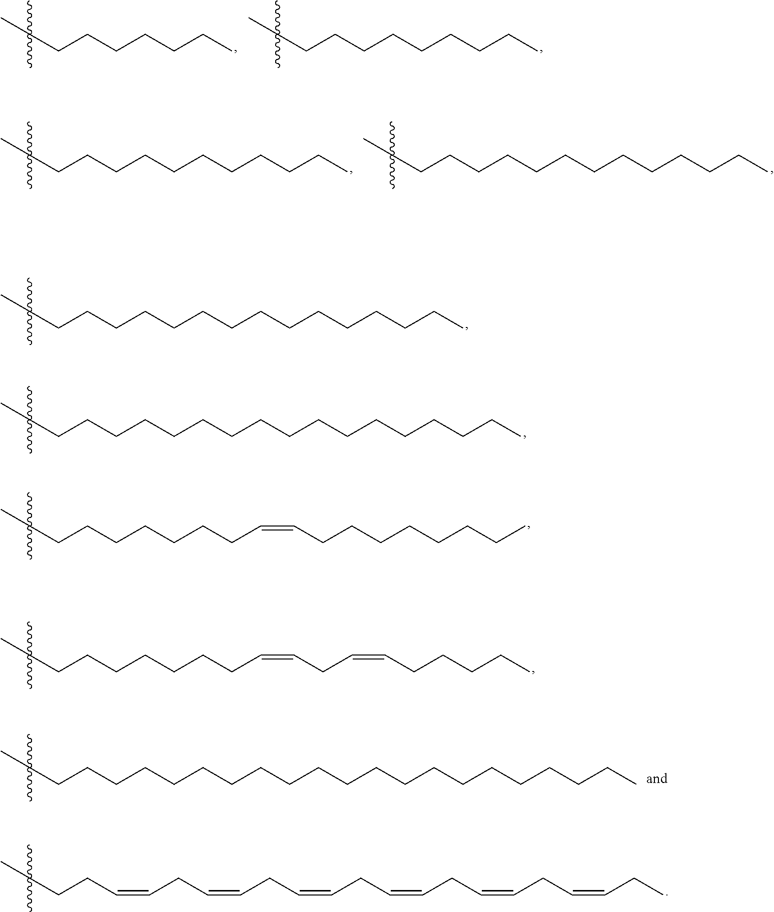

In some aspects, the disclosure provides an oligonucleotide-ligand conjugate comprising one or more nucleic acid-ligand conjugate described herein. In some aspects, the oligonucleotide-ligand conjugate comprises 1, 2, 3, 4, 5, 6, 7, 8, 9 or 10 nucleic acid-ligand conjugates.

In some aspects, the disclosure provides an oligonucleotide-ligand conjugate comprising one or more nucleic acid-ligand conjugates represented by formula II-a:

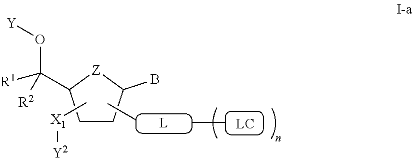

or a pharmaceutically acceptable salt thereof, wherein:

B is a nucleobase or hydrogen;

R¹ and R² are independently hydrogen, halogen, R^{A}, —CN, —S(O)R, —S(O)₂R, —Si(OR)₂R, —Si(OR)R₂, or —SiR₃; or

R¹ and R² on the same carbon are taken together with their intervening atoms to form a 3-7 membered saturated or partially unsaturated ring having 0-3 heteroatoms, independently selected from nitrogen, oxygen, and sulfur;

each R^{A} is independently an optionally substituted group selected from C1-6 aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;

each R is independently hydrogen, a suitable protecting group, or an optionally substituted group selected from C1-6 aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur; or

two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur;

each LC is independently a lipid conjugate moiety comprising a saturated or unsaturated, straight, or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, —P(S)OR—;

each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-3 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;

n is 1-10;

L is a covalent bond or a bivalent saturated or unsaturated, straight or branched C₁₋₅₀ hydrocarbon chain, wherein 0-10 methylene units of the hydrocarbon chain are independently replaced by -Cy-, —O—, —C(O) NR—, —NR—, —S—, —C(O)—, —C(O)O—, —S(O)—, —S(O)₂—, —P(O)OR—, —P(S)OR—, —V¹CR²W¹—, or

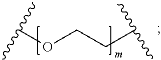

m is 1-50;

X¹, V¹ and W¹ are independently —C(R)₂—, —OR, —O—, —S—, —Se—, or —NR—;

Y is hydrogen, a suitable hydroxyl protecting group,

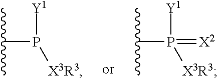

R³ is hydrogen, a suitable protecting group, a suitable prodrug, or an optionally substituted group selected from C1-6 aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur;

X² is O, S, or NR;

X³ is —O—, —S—, —BH₂—, or a covalent bond;

Y¹ is a linking group attaching to the 2′- or 3′-terminal of a nucleoside, a nucleotide, or an oligonucleotide;

Y² is hydrogen, a suitable protecting group, a phosphoramidite analogue, an internucleotide linking group attaching to the 5′-terminal of a nucleoside, a nucleotide, or an oligonucleotide, or a linking group attaching to a solid support; and

Z is —O—, —S—, —NR—, or —CR₂—.

In any of the foregoing or related aspects, the oligonucleotide-ligand conjugate is represented by formula II-b or II-c:

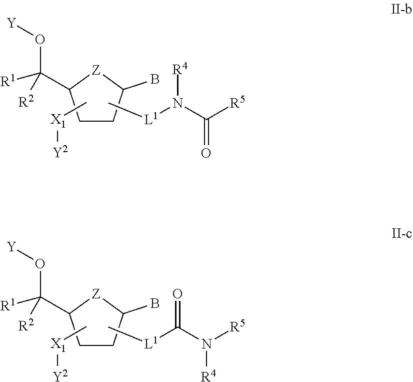

or a pharmaceutically acceptable salt thereof, wherein:

L¹ is a covalent bond, a monovalent or a bivalent saturated or unsaturated, straight or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, —P(S)OR—, or

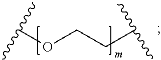

R⁴ is hydrogen, R^{A}, or a suitable amine protection group; and

R⁵ is adamantyl, or a saturated or unsaturated, straight, or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, or —P(S)OR.

In any of the foregoing or related aspects, R⁵ is selected from

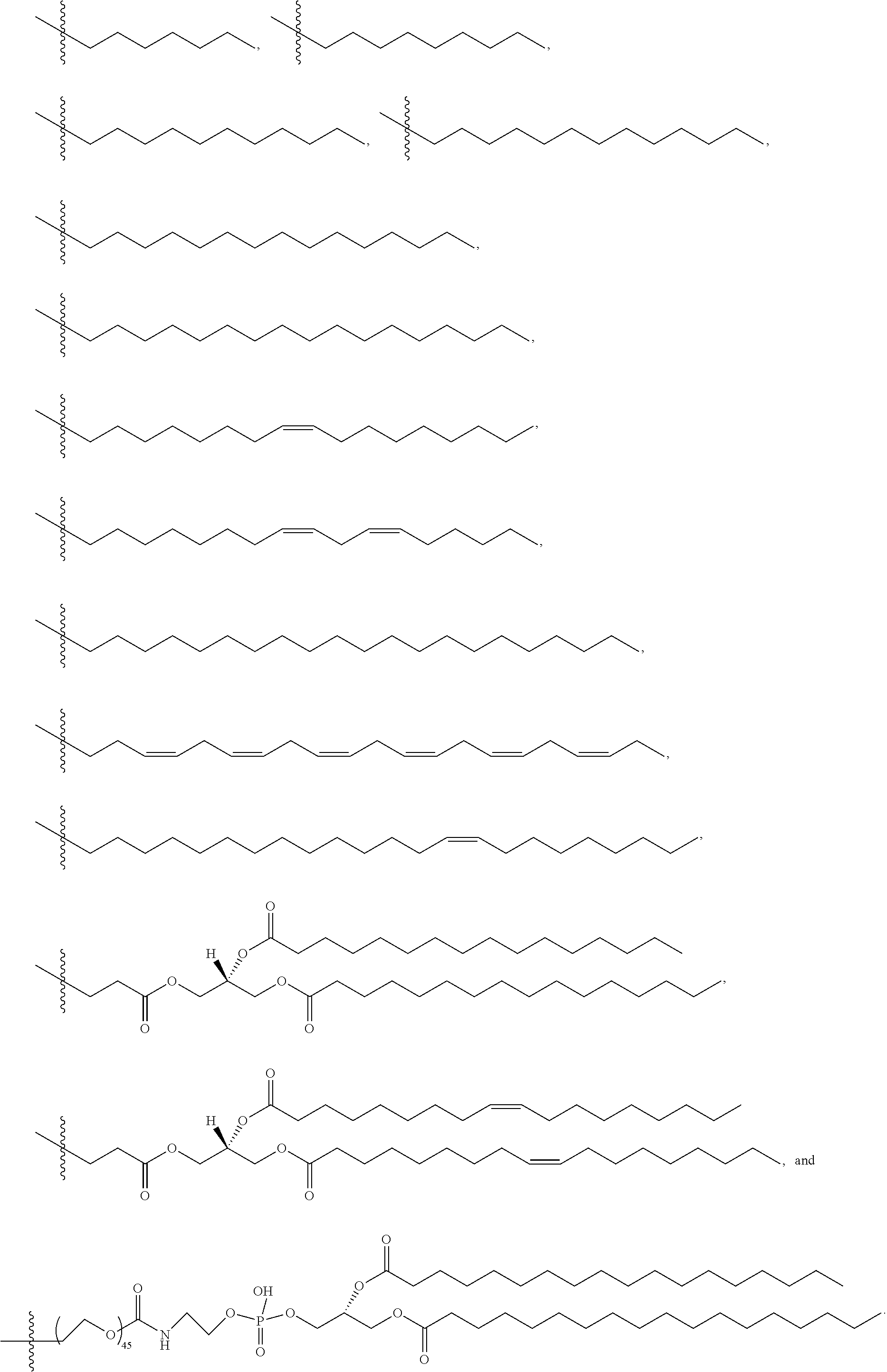

In some aspects, R⁵ is selected from

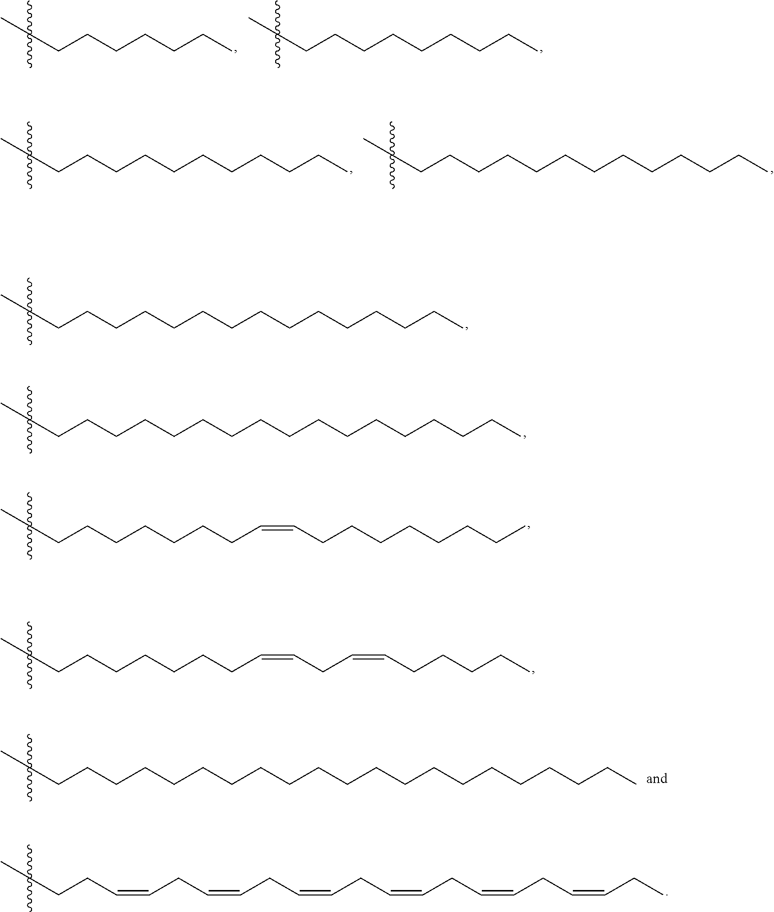

In some aspects, the disclosure provides an oligonucleotide-ligand conjugate represented by formula II-Ib or II-Ic:

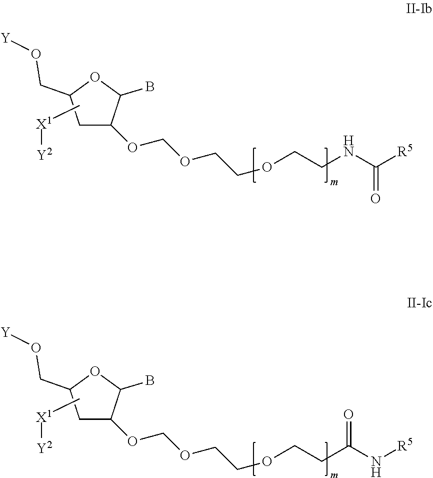

or a pharmaceutically acceptable salt thereof; wherein

B is a nucleobase or hydrogen;

m is 1-50;

X¹ is —O—, or —S—;

Y is hydrogen,

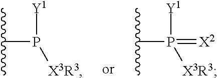

R³ is hydrogen, or a suitable protecting group;
X² is O, or S;
X³ is —O—, —S—, or a covalent bond;

Y¹ is a linking group attaching to the 2′- or 3′-terminal of a nucleoside, a nucleotide, or an oligonucleotide;

Y² is hydrogen, a phosphoramidite analogue, an internucleotide linking group attaching to the 5′-terminal of a nucleoside, a nucleotide, or an oligonucleotide, or a linking group attaching to a solid support;

R⁵ is adamantyl, or a saturated or unsaturated, straight, or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, or —P(S)OR—; and

R is hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur.

In any of the foregoing or related aspects, R⁵ is selected from

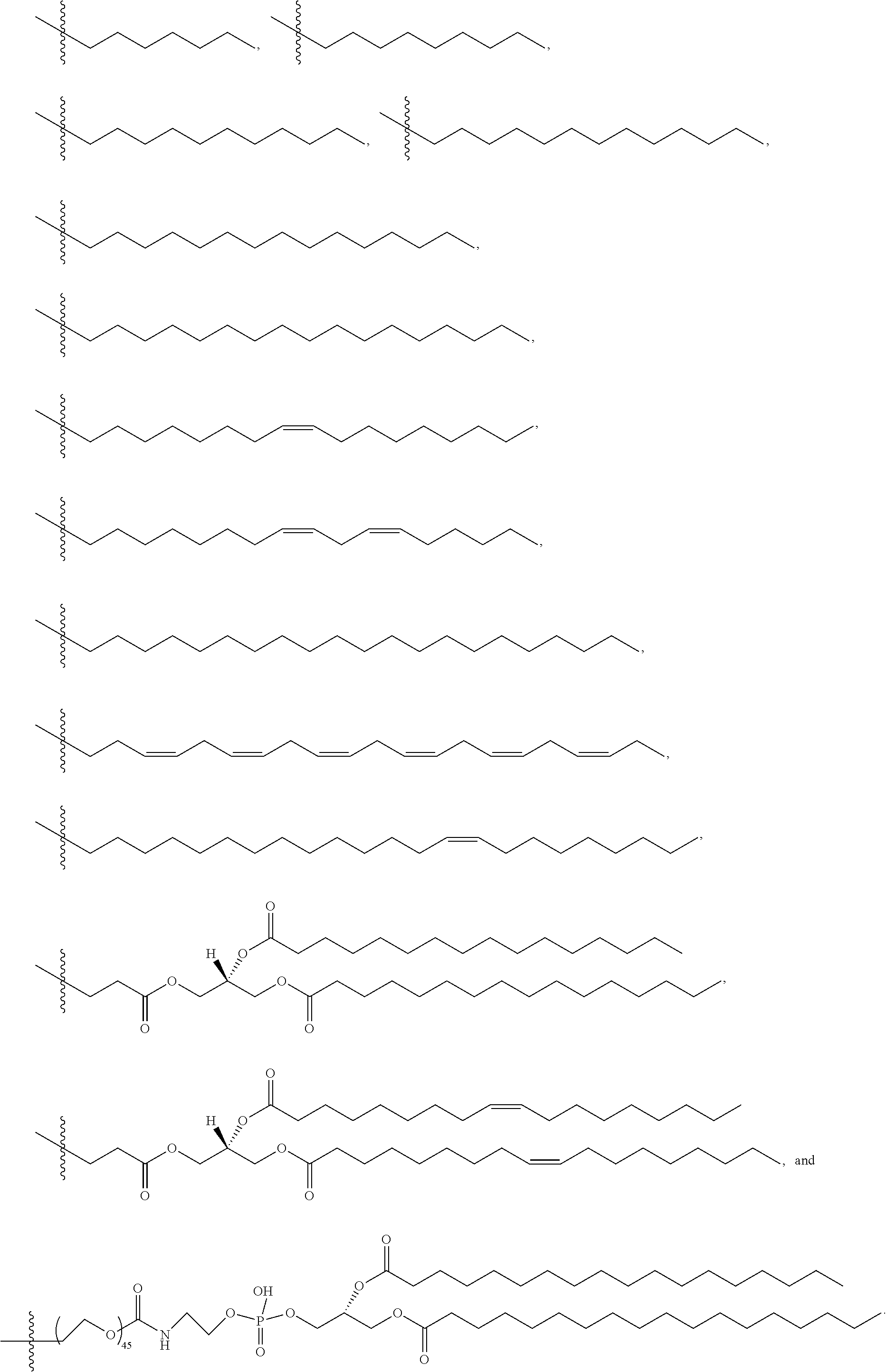

In any of the foregoing or related aspects, R⁵ is

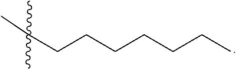 [SMILES: *CCCCCCC]

In any of the foregoing or related aspects, R⁵ is

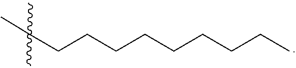 [SMILES: *CCCCCCCCC]

In any of the foregoing or related aspects, R⁵ is

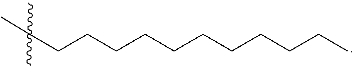 [SMILES: *CCCCCCCCCCC]

In any of the foregoing or related aspects, R⁵ is

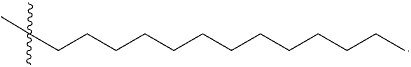 [SMILES: *CCCCCCCCCCCCC]

In any of the foregoing or related aspects, R⁵ is

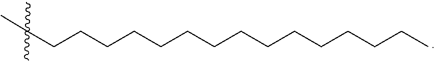 [SMILES: *CCCCCCCCCCCCCCC]

In any of the foregoing or related aspects, R⁵ is

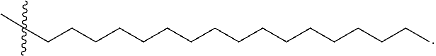 [SMILES: *CCCCCCCCCCCCCCCCC]

In any of the foregoing or related aspects, R⁵ is

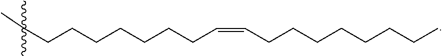 [SMILES: *CCCCCCC/C=C\CCCCCCCC]

In any of the forego or related aspects, R⁵ is

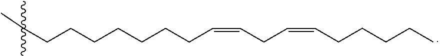 [SMILES: *CCCCCCC/C=C\C/C=C\CCCCC]

In any of the foregoing or related aspects, R⁵ is

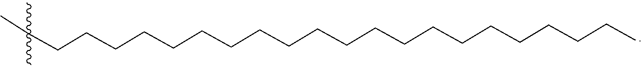 [SMILES: *CCCCCCCCCCCCCCCCCCCCC]

In any of the foregoing or related aspects, R⁵ is

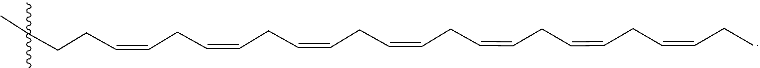 [SMILES: *CC/C=C\C/C=C\C/C=C\C/C=C\C/C=C\C/C=C\C/C=C\CC]

In any of the foregoing or related aspects, the oligonucleotide-ligand conjugate comprises 1-10 nucleic acid-ligand conjugate units. In some aspects, the conjugate comprises 1, 2 or 3 nucleic acid-ligand conjugate units.

In any of the foregoing or related aspects, the oligonucleotide comprises a sense strand of 10-53 nucleotides in length and an antisense strand of 15-53 nucleotides in length, wherein the antisense oligonucleotide strand has sequence complementary to at least 15 consecutive nucleotides of a target gene sequence and reduces the target gene expression when the oligonucleotide-conjugate is introduced into a mammalian cell.

In any of the foregoing or related aspects, the nucleic acid-ligand conjugate units are present in the sense strand. In some aspects, the antisense strand is 19 to 27 nucleotides in length. In some aspects, the sense strand is 12 to 40 nucleotides in length.

In any of the foregoing or related aspects, the sense strand forms a duplex region with the antisense strand.

In any of the foregoing or related aspects, the region of complementarity is fully complementary to the target sequence.

In any of the foregoing or related aspects, the sense strand comprises at its 3′-end a stem-loop set forth as: S₁-L-S₂, wherein S₁ is complementary to S₂, and wherein L forms a loop between S₁ and S₂ of 3 to 5 nucleotides in length. In some aspects, L is a tetraloop. In some aspects, L comprises a sequence set forth as GAAA. In some aspects, L comprises the nucleotide sequence set forth as 5′-GAAA-3′. In some aspects, L consists of the nucleotide sequence set forth as 5′-GAAA-3′.

In some aspects, the disclosure provides an oligonucleotide-ligand conjugate for reducing expression of a target mRNA in the central nervous system (CNS) comprising (i) a double-stranded oligonucleotide comprising an antisense strand of 15 to 30 nucleotides in length and a sense strand of 15 to 40 nucleotides in length, wherein the antisense strand and the sense strand each comprise a 5′ end and a 3′ end, wherein the antisense strand and the sense strand form a duplex region, wherein the antisense strand has a region of complementarity to a target sequence in the target mRNA in the CNS, wherein the region of complementarity is at least 15 contiguous nucleotides in length, wherein the oligonucleotide comprises a stem-loop; and (ii) one or more lipid moieties conjugated to the stem-loop.

In any of the foregoing or related aspects, the lipid moiety comprises a saturated C16, C17, C18, C19, C20, C21, or C22 hydrocarbon chain. In some aspects, the lipid moiety comprises an unsaturated C16, C17, C18, C19, C20, C21, or C22 hydrocarbon chain, optionally wherein the lipid moiety comprises C22:6. In some embodiments, an unsaturated hydrocarbon chain comprises at least one double bound. For example, a C18 hydrocarbon chain having one double bond is referred to as C18:1, whereas a C18 hydrocarbon chain having two double bonds is referred to as C18:2. In some aspects, the lipid moiety comprises an unsaturated C18 hydrocarbon chain (e.g., C18:1). In some aspects, the lipid moiety comprises an unsaturated C22 hydrocarbon chain (e.g., C22:6).

In any of the foregoing or related aspects, the one or more lipid moieties comprise a saturated or unsaturated C1-C50 hydrocarbon chain. In some aspects, the one or more lipid moieties comprise a saturated or unsaturated C5-C25 hydrocarbon chain. In some aspects, the one or more lipid moieties comprise a saturated C8, C9, C10, C11, C12, C13, or C14 hydrocarbon chain. In some aspects, the oligonucleotide-ligand conjugate reduces expression of the target mRNA in the CNS without reducing expression of the target mRNA outside the CNS, optionally without reducing expression of the target mRNA in the liver. In some aspects, the target mRNA is expressed in liver cells, wherein the oligonucleotide-ligand conjugate does not substantially reduce expression of the target mRNA in liver cells relative to expression of the target mRNA in the cells of the CNS. In some aspects, the target mRNA is expressed in liver cells, wherein the oligonucleotide-ligand conjugate does not reduce expression of the target mRNA in liver cells to the same level as in the cells of the CNS.

In some aspects, the disclosure provides an oligonucleotide-ligand conjugate for reducing expression of a target mRNA in the CNS comprising (i) a double-stranded oligonucleotide comprising an antisense strand of 15 to 30 nucleotides in length and a sense strand of 15 to 40 nucleotides in length, wherein the antisense strand and the sense strand each comprise a 5′ end and a 3′ end, wherein the antisense strand and the sense strand form a duplex region, wherein the antisense strand has a region of complementarity to a target sequence in the target mRNA in the CNS, wherein the region of complementarity is at least 15 contiguous nucleotides in length, wherein the oligonucleotide comprises a stem-loop; and (ii) one or more lipid moieties conjugated to the stem-loop, wherein the one or more lipid moieties are selected from saturated or unsaturated C8-C14 hydrocarbon chains, wherein expression of the target mRNA in the liver is not reduced to the same or similar level as in the CNS. In some aspects, the one or more lipid moieties is a saturated or unsaturated C8 hydrocarbon chain. In some aspects, the one or more lipid moieties is a saturated or unsaturated C9 hydrocarbon chain. In some aspects, the one or more lipid moieties is a saturated or unsaturated C10 hydrocarbon chain. In some aspects, the one or more lipid moieties is a saturated or unsaturated C11 hydrocarbon chain. In some aspects, the one or more lipid moieties is a saturated or unsaturated C12 hydrocarbon chain. In some aspects, the one or more lipid moieties is a saturated or unsaturated C13 hydrocarbon chain. In some aspects, the one or more lipid moieties is a saturated or unsaturated C14 hydrocarbon chain.

In some aspects, the sense strand comprises the stem-loop at its 3′ end. In some aspects, the stem-loop comprises a nucleotide sequence represented by the formula: 5′-S₁-L-S₂-3′, wherein S1 is complementary to S2, and wherein L forms a loop between S1 and S2. In some aspects, S1 and S2 are 1 to 8 nucleotides in length. In some aspects, the one or more lipid moieties is conjugated to a nucleotide of S1. In some aspects, the one or more lipid moieties is conjugated to a nucleotide of S2.

In any of the foregoing or related aspects, L is 3 to 5 nucleotides in length. In some aspects, L is a tetraloop, optionally wherein L is 4 nucleotides in length. In some aspects, L comprises the nucleotide sequence set forth as 5′-GAAA-3′. In some aspects, L consists of the nucleotide sequence set forth as 5′-GAAA-3′. In some aspects, the one or more lipid moieties is conjugated to a nucleotide of L.

In any of the foregoing or related aspects, L is 3 nucleotides in length and has 5′ to 3′ a first, second, and third nucleotide, wherein the oligonucleotide-ligand conjugate comprises one lipid moiety, and wherein the lipid moiety is conjugated to the first, second, or third nucleotide of L. In some aspects, L is 4 nucleotides in length and has 5′ to 3′ a first, second, third, and fourth nucleotide, wherein the oligonucleotide-ligand conjugate comprises one lipid moiety, and wherein the lipid moiety is conjugated to the first, second, third, or fourth nucleotide of L. In some aspects, the lipid moiety is conjugated to the second nucleotide of L. In some aspects, L consists of 5′-GAAA-3′. In some aspects, the lipid moiety is conjugated to the second nucleotide of L. In some aspects, L is 5 nucleotides in length and has from 5′ to 3′ a first, second, third, fourth, and fifth nucleotide, wherein the oligonucleotide-ligand conjugate comprises one lipid moiety, and wherein the lipid moiety is conjugated to the first, second, third, fourth, or fifth nucleotide of L.

In any of the foregoing or related aspects, the antisense strand is 19 to 27 nucleotides in length. In some aspects, the antisense strand is 21 to 27 nucleotides in length. In some aspects, the antisense strand is 22 nucleotides in length.

In any of the foregoing or related aspects, the sense strand is 19 to 40 nucleotides in length. In some aspects, the sense strand is 36 nucleotides in length.

In any of the foregoing or related aspects, the duplex region is at least 19 nucleotides in length. In some aspects, the duplex region is at least 20 nucleotides in length. In some aspects, the duplex region is 21 nucleotides in length.

In any of the foregoing or related aspects, the oligonucleotide-ligand conjugate comprises a 3′-overhang sequence on the antisense strand of two nucleotides in length. In some aspects, the oligonucleotide comprises a 3′-overhang sequence of one or more nucleotides in length, wherein the 3′-overhang sequence is present on the antisense strand, the sense strand, or the antisense strand and sense strand. In some aspects, the 3′-overhang sequence is on the antisense strand. In some aspects, the 3′-overhang sequence is two nucleotides in length.

In any of the foregoing or related aspects, the oligonucleotide comprises at least one modified nucleotide. In some aspects, the modified nucleotide comprises a 2′-modification. In some aspects, the 2′-modification is a modification selected from: 2′-aminoethyl, 2′-fluoro, 2′-O-methyl, 2′-O-methoxyethyl, 2′-deoxy-2′-fluoro, and 2′-deoxy-2′-fluoro-β-d-arabino. In some aspects, all the nucleotides of the oligonucleotide are modified.

In any of the foregoing or related aspects, the oligonucleotide comprises at least one modified internucleotide linkage. In some aspects, the at least one modified internucleotide linkage is a phosphorothioate linkage. In any of the foregoing or related aspects, the oligonucleotide comprises at least one modified internucleoside linkage. In some aspects, the at least one modified internucleoside linkage is a phosphorothioate linkage.

In any of the foregoing or related aspects, the 4′-carbon of the sugar of the 5′-nucleotide of the antisense strand comprises a phosphate analog. In some aspects, the phosphate analog is oxymethylphosphonate, vinylphosphonate, or malonylphosphonate.

In some aspects, the disclosure provides a composition comprising an oligonucleotide-ligand conjugate described herein and an excipient.

In some aspects, the disclosure provides a pharmaceutical composition comprising an oligonucleotide-ligand conjugate described herein, and a pharmaceutically acceptable carrier.

In other aspects, the disclosure provides a method of delivering an oligonucleotide-ligand conjugate to a subject, the method comprising administering a composition described herein to the subject.

In some aspects, the disclosure provides method of reducing or inhibiting expression of a target mRNA in a subject expressed by a population of cells associated with the CNS in a subject, comprising administering an oligonucleotide-ligand conjugate described herein, a composition described herein, or a pharmaceutical composition described herein to the subject. In some aspects, the level of expression of the target mRNA is reduced in the population of cells associated with the CNS compared to a control population of cells. In some aspects, the level of expression of the target mRNA is not reduced in population of cells residing outside the CNS compared to a control population of cells. In some aspects, the population of cells residing outside the CNS are in the liver.

In further aspects, the disclosure provides an oligonucleotide-ligand conjugate for reducing expression of a target gene. In some aspects, the target gene is expressed in the CNS. In some aspects, the target gene is associated with a disease or disorder, optionally a neurological disease or disorder. In some aspects, the disease or disorder is selected from Progressive Supranuclear Palsy (PSP), Corticobasal degeneration (CBD), Argyrophilic grain disease (AGD), Globular glial tauopathy (GGT), Aging-related tau astrogliopathy (ARTAG), Familial Frontotemporal Dementia 17 (FTD-17), Tauopathy with Respiratory Failure, Dementia with Seizures, Pick's disease, Myotonic dystrophy 1 or 2 (MD1 or MD2), Down's syndrome, Spastic Paraplegia (SP), Niemann-Pick disease type C, Dementia with Lewy bodies (DLB), Lewy body dysphagia, Lewy body disease, Olivopontocerebellar atrophy, Striatonigral degeneration, Shy-Drager syndrome, Spinal muscular atrophy V (SMAV), Huntington's Disease (HD), Alzheimer's Disease, SCA1, SCA2, SCA3, SCA7, SCA10 (spinocerebellar ataxia type 1, 2, 3, 7 or 103), Multiple System Atrophy (MSA), Spinal and Bulbar Muscular Atrophy (SBMA, Kennedy disease), Friedrich Ataxia, Fragile X-associated tremor/ataxia syndrome (FXTAS), Fragile X syndrome (FRAXA), X-Linked Mental Retardation (XLMR), Parkinson's Disease, Dystonia, SBMA (spinobulbar muscular atrophy), neuropathic pain disorders, spinal cord injury, Dentatorubral-pallidoluysian atrophy (DRPLA), recessive CNS disorders, and ALS (amyotrophic lateral sclerosis), M2DS (MECP2 duplication syndrome), FTD (frontotemporal dementia), Prion disease, Adult Onset Leukodystrophy, Alexander's Disease, Krabbe Disease, Chronic Traumatic Encephalopathy, Pelizaeus-Merzbacher disease (PMD), Lafora disease, stroke, Cerebral Amyloid Angiopathy (CAA), and Metachromatic Leukodystrophy (MLD).

### BRIEF DESCRIPTION OF FIGURES

FIG. 1 provides a graph depicting the efficacy of an RNAi oligonucleotide designed to inhibit murine Aldh2mRNA expression following administration into the CNS of mice (GalXC-ALDH2 RNAi oligonucleotide). The percent (%) of Aldh2 mRNA remaining in samples from various CNS tissues, as indicated, was measured in female CD-1 mice 5 days following intracerebroventricular (i.c.v.) administration with 100 μg of the GalXC-ALDH2 RNAi oligonucleotide formulated in PBS relative to the % of Aldh2mRNA in PBS treated mice.

FIG. 2 provides graphs depicting the dose response of the GalXC-ALDH2 RNAi oligonucleotide following i.c.v. administration into the CNS of mice. The percent (%) of murine Aldh2 mRNA remaining in various CNS tissues, as indicated, was measured in mice at various timepoints, as indicated, following i.c.v. administration with 250 μg or 500 μg of the GalXC-ALDH2 RNAi oligonucleotide formulated in PBS relative to the % of Aldh2 mRNA in PBS treated mice. Percent (%) Aldh2 mRNA was determined in the frontal cortex, hippocampus, hypothalamus, striatum, somatosensory cortex, and cerebellum, as indicated.

FIG. 3 provides a depiction of the mouse brain and spinal cord regions, along with graphs depicting the dose response of the GalXC-ALDH2 RNAi oligonucleotide following i.c.v. administration into the CNS of mice. The percent (%) of murine Aldh2 mRNA remaining in various CNS tissues, as indicated, was measured in mice at various timepoints, as indicated, following i.c.v. administration with 250 μg or 500 μg of the GalXC-ALDH2 RNAi oligonucleotide formulated in PBS relative to the % of Aldh2mRNA in PBS treated mice. Percent (%) Aldh2 mRNA was determined in the cervical spinal cord, thoracic spinal cord, and lumbar spinal cord, as indicated.

FIG. 4 provides a graph depicting the efficacy of an RNAi oligonucleotide designed to inhibit murine Aldh2mRNA expression following administration into the CNS of mice (GalXC-ALDH2 RNAi oligonucleotide). The percent (%) of Aldh2 mRNA remaining in samples from various CNS tissues, as indicated, was measured in mice 7 days following intrathecal (i.t.) administration with 500 μg of the GalXC-ALDH2 RNAi oligonucleotide formulated in PBS relative to the % of Aldh2mRNA in PBS treated mice.

FIG. 5 provides graphs depicting the dose response of the GalXC-ALDH2 RNAi oligonucleotide following administration into the CNS of rats. The percent (%) of rat Aldh2 mRNA remaining in various CNS tissues, as indicated, was measured in rat at 7 days and at 28 days, as indicated, following lumbar i.t. administration with 1000 μg or 6000 μg of the GalXC-ALDH2 RNAi oligonucleotide formulated in PBS relative to the % of Aldh2 mRNA in PBS treated rats. Percent (%) Aldh2 mRNA was determined in the frontal cortex, striatum, hippocampus, brain stem, spinal cord regions SC1-SC8, C1-C7 dorsal root ganglion (DRG), T1-T12 DRG, L1-L6 DRG, and liver.

FIG. 6 provides a graph depicting the efficacy of an RNAi oligonucleotide designed to inhibit human/monkey ALDH2 mRNA expression (GalXC-ALDH2 RNAi oligonucleotide) following administration into the CNS of non-human primates (NHPs). The percent (%) of ALDH2 mRNA remaining in samples from various CNS tissues, as indicated, was measured in cynomolgus macaques 28 days following i.t. administration with 75 mg of the GalXC-ALDH2 RNAi oligonucleotide formulated in PBS relative to the % of ALDH2 mRNA in PBS treated NHPs.

FIG. 7 provides a graph depicting the dose response of the GalXC-ALDH2 RNAi oligonucleotide following administration into the CNS of NHPs. The percent (%) of human/monkey ALDH2mRNA remaining in various CNS tissues, as indicated, was measured in rhesus macaques at 28 days following i.t. administration with 75 mg, 150 mg, or 300 mg of the GalXC-ALDH2 RNAi oligonucleotide formulated in PBS relative to the % of ALDH2mRNA in PBS treated NHPs. Percent (%) ALDH2 mRNA was determined in the frontal cortex, hippocampus, temporal cortex, cerebellum, brainstem, cervical spinal cord, thoracic spinal cord, lumbar spinal cord, dorsal root ganglion (DRG), and liver.

FIG. 8 provides a graph depicting the efficacy of the GalXC-ALDH2 RNAi oligonucleotide following administration (infusion) into the CNS of NHPs through a surgically implanted lumbar port. The percent (%) of human/monkey ALDH2mRNA remaining in various CNS tissues, as indicated, was measured in cynomolgus macaques at 28 days following i.t. administration with 75 mg of the GalXC-ALDH2 RNAi oligonucleotide formulated in PBS relative to the % of ALDH2 mRNA in PBS treated NHPs. Percent (%) ALDH2 mRNA was determined in the frontal cortex, caudate nucleus, hippocampus, midbrain, parietal cortex, occipital cortex, thalamus, temporal cortex, cerebellum, brain stem, cervical spinal cord, thoracic spinal cord, lumbar spinal cord, dorsal root ganglion (DRG), and liver.

FIG. 9 provides a graph depicting the efficacy of the GalXC-ALDH2 RNAi oligonucleotide following administration into the CNS of NHPs. The percent (%) of human/monkey ALDH2mRNA remaining in various CNS tissues, as indicated, was measured in cynomolgus macaques at 28 days following intra-cisterna magna (i.c.m.) administration with 50 mg of the GalXC-ALDH2 RNAi oligonucleotide formulated in PBS relative to the % of ALDH2 mRNA in PBS treated NHPs. The percent (%) of ALDH2 mRNA was determined in the frontal cortex, caudate nucleus, hippocampus, midbrain, parietal cortex, occipital cortex, thalamus, temporal cortex, cerebellum, brain stem, cervical spinal cord, thoracic spinal cord, lumbar spinal cord, dorsal root ganglion (DRG), and liver.

FIG. 10A provides a graph depicting the efficacy of a series of GalXC-ALDH2 RNAi oligonucleotide-lipid conjugates (C8:0-C22:6) following administration into the CNS of mice. The percent (%) of murine Aldh2 mRNA remaining in various CNS tissues, as indicated, was measured in female CD-1 mice 7 days following i.c.v. administration with 250 μg of GalXC-ALDH2 RNAi oligonucleotide-lipid conjugates formulated in PBS relative to the % of Aldh2 mRNA in PBS treated mice. Percent (%) Aldh2 mRNA was determined in the somatosensory cortex (SS), hippocampus (HP), hypothalamus (HY), cervical spinal cord (CSC), thoracic spinal cord (TSC), and lumbar spinal cord (LSC).

FIG. 10B provides a graph depicting percent (%) expression of Aldh2 mRNA in the liver for the same mice described in FIG. 10A that received GalXC-ALDH2 RNAi oligonucleotide-lipid conjugates (C8:0-C18:0) by i.c.v. administration. Shown is the percent (%) of murine Aldh2 mRNA remaining in liver tissue harvested at 7 days following i.c.v. administration relative to the percent (%) of Aldh2 mRNA in liver tissue harvested from mice administered PBS only.

FIG. 11 provides graphs depicting the efficacy of a series of GalXC-ALDH2 RNAi oligonucleotide-lipid conjugates (C10:0-C18:2) following administration into the CNS of mice. The percent (%) of murine Aldh2 mRNA remaining in various CNS tissues, as indicated, was measured in female C57BL/6 mice 7 days following i.t. administration with 250 μg of GalXC-ALDH2 RNAi oligonucleotide-lipid conjugates formulated in PBS relative to the % of Aldh2 mRNA in PBS treated mice. Percent (%) Aldh2 mRNA was determined in the frontal cortex, hippocampus (HP), brainstem (BS), cervical spinal cord (CSC), thoracic spinal cord (TSC), and lumbar spinal cord (LSC).

FIG. 12 provides a schematic of a modified GalXC oligonucleotide.

FIG. 13 provides a schematic of a modified lipid conjugated-GalXC oligonucleotide.

### DETAILED DESCRIPTION

In some aspects, the disclosure provides RNAi oligonucleotide conjugates (e.g., RNAi oligonucleotide-lipid conjugates) that reduce expression of a target gene in the CNS. In other aspects, the disclosure provides methods of treating a disease or disorder (e.g., a neurological disease and/or by inappropriate gene expression) using the RNAi oligonucleotide conjugates, or pharmaceutically acceptable compositions thereof, described herein. In other aspects, the disclosure provides methods of using the RNAi oligonucleotide conjugates described herein in the manufacture of a medicament for treating a disease or disorder. In other aspects, the RNAi oligonucleotide conjugates provided herein are used to treat a neurological disease or disorder by modulating (e.g., inhibiting or reducing) expression of a target gene associated with the neurological disease or disorder in the CNS. In some aspects, the disclosure provides methods of treating a neurological disease or disorder by reducing expression of a target gene associated with the neurological disease or disorder in the CNS (e.g., in cells, tissues or regions of the CNS).

### RNAi Oligonucleotide Conjugates

The disclosure provides, inter alia, RNAi oligonucleotide conjugates (e.g., RNAi oligonucleotide-lipid conjugates) that reduce target gene expression in the CNS. In some embodiments, an RNAi oligonucleotide conjugate provided by the disclosure is targeted to an mRNA encoding the target gene. Messenger RNA (mRNA) that encodes a target gene and is targeted by an RNAi oligonucleotide conjugate of the disclosure is referred to herein as “target mRNA”. In some embodiments, the RNAi oligonucleotide conjugate reduces target gene expression in the CNS (e.g., in the somatosensory cortex (SS cortex), hippocampus (HP), striatum, frontal cortex, cerebellum, hypothalamus (HY), cervical spinal cord (CSC), thoracic spinal cord (TSC), and/or lumbar spinal cord (LSC)). In some embodiments, the RNAi oligonucleotide conjugate reduces target gene expression in the CNS (e.g., in the SS cortex, HP, HY, CSC, TSC, and/or LSC), without reducing expression of the target mRNA outside the CNS. In some embodiments, the RNAi oligonucleotide conjugate reduces target gene expression in the CNS (e.g., in the SS cortex, HP, HY, CSC, TSC, and/or LSC), without reducing expression of the target mRNA in the liver. In some embodiments, the RNAi oligonucleotide conjugate does not result in a reduction in the expression of the target mRNA in the liver to the same or similar level as in the CNS.

mRNA Target Sequences

In some embodiments, the RNAi oligonucleotide conjugate is targeted to a target sequence comprising a target mRNA. In some embodiments, the RNAi oligonucleotide conjugate is targeted to a target sequence within a target mRNA. In some embodiments, the target mRNA is expressed in the CNS. In some embodiments, a target mRNA expressed in the CNS is referred to herein as “a CNS target mRNA.” In some embodiments, the RNAi oligonucleotide conjugate, or a portion, fragment, or strand thereof (e.g., an antisense strand or a guide strand of a double-stranded oligonucleotide) binds or anneals to a target sequence comprising a target mRNA, thereby reducing target gene expression. In some embodiments, the RNAi oligonucleotide conjugate is targeted to a target sequence comprising a target mRNA for the purpose of reducing target gene expression in vivo. In some embodiments, the amount or extent of reduction of target gene expression by an RNAi oligonucleotide conjugate targeted to a specific target sequence correlates with the potency of the RNAi oligonucleotide conjugate. In some embodiments, the amount or extent of reduction of target gene expression by an RNAi oligonucleotide conjugate targeted to a specific target sequence correlates with the amount or extent of therapeutic benefit in a subject or patient having a disease, disorder or condition associated with target gene expression treated with the RNAi oligonucleotide conjugate.

Through examination of the nucleotide sequence of mRNAs encoding target genes, including mRNAs of multiple different species (e.g., human, cynomolgus monkey, mouse, and rat) and as a result of in vitro and in vivo testing, it has been discovered that certain nucleotide sequences comprising target mRNA are more amenable than others to RNAi oligonucleotide-mediated reduction and are thus useful as target sequences for the RNAi oligonucleotides conjugates herein. In some embodiments, a sense strand of an RNAi oligonucleotide conjugate (e.g., RNAi oligonucleotide-lipid conjugate), or a portion or fragment thereof, described herein, comprises a nucleotide sequence that is similar (e.g., having no more than 4 mismatches) or is identical to a target sequence comprising a target mRNA. In some embodiments, a portion or region of the sense strand of a double-stranded oligonucleotide described herein comprises a target sequence comprising a target mRNA.

### RNAi Oligonucleotide Targeting Sequences

In some embodiments, the RNAi oligonucleotide conjugates provided by the disclosure comprise a targeting sequence. As used herein, the term “targeting sequence” refers to a nucleotide sequence having a region of complementarity to a nucleotide sequence comprising an mRNA (e.g., a target mRNA). In some embodiments, the RNAi oligonucleotide conjugates provided by the disclosure comprise a targeting sequence having a region of complementarity to a nucleotide sequence comprising a target sequence of a target mRNA. In some embodiments, the RNAi oligonucleotide conjugates provided by the disclosure comprise a targeting sequence having a region of complementarity to a nucleotide sequence comprising a target sequence of a CNS target mRNA. The targeting sequence imparts the RNAi oligonucleotide conjugate with the ability to specifically target an mRNA by binding or annealing to a target sequence comprising a target mRNA by complementary (Watson-Crick) base pairing. In some embodiments, the RNAi oligonucleotide conjugates herein (or a strand thereof, e.g., an antisense strand or a guide strand of a double-stranded oligonucleotide) comprise targeting sequence having a region of complementarity that binds or anneals to a target sequence comprising a target mRNA by complementary (Watson-Crick) base pairing. The targeting sequence is generally of suitable length and base content to enable binding or annealing of the RNAi oligonucleotide conjugate (or a strand thereof) to a specific target mRNA for purposes of inhibiting target gene expression. In some embodiments, the targeting sequence is at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29 or at least about 30 nucleotides in length. In some embodiments, the targeting sequence is at least 12, at least 13, at least 14, at least 15, at least 16, at least 17, at least 18, at least 19 or at least 20 nucleotides. In some embodiments, the targeting sequence is about 12 to about 30 (e.g., 12 to 30, 12 to 22, 15 to 25, 17 to 21, 18 to 27, 19 to 27, or 15 to 30) nucleotides in length. In some embodiments, the targeting sequence is about 12, 13, 14, 15, 16, 17, 18, 19, 20, 21, 22, 23, 24, 25, 26, 27, 28, 29 or 30 nucleotides in length. In some embodiments, the targeting sequence is 18 nucleotides in length. In some embodiments, the targeting sequence is 19 nucleotides in length. In some embodiments, the targeting sequence is 20 nucleotides in length. In some embodiments, the targeting sequence is 21 nucleotides in length. In some embodiments, the targeting sequence is 22 nucleotides in length. In some embodiments, the targeting sequence is 23 nucleotides in length. In some embodiments, the targeting sequence is 24 nucleotides in length.

In some embodiments, the RNAi oligonucleotide conjugates herein comprise a targeting sequence that is fully complementary to a target sequence comprising a target mRNA. In some embodiments, the targeting sequence is partially complementary to a target sequence comprising a target mRNA. In some embodiments, the targeting sequence comprises a region of contiguous nucleotides comprising the antisense strand.

In some embodiments, the RNAi oligonucleotide conjugates herein comprise a targeting sequence that is complementary to a contiguous sequence of nucleotides comprising a target mRNA, wherein the contiguous sequence of nucleotides is about 12 to about 30 nucleotides in length (e.g., 12 to 30, 12 to 28, 12 to 26, 12 to 24, 12 to 20, 12 to 18, 12 to 16, 14 to 22, 16 to 20, 18 to 20 or 18 to 19 nucleotides in length). In some embodiments, the RNAi oligonucleotide conjugates comprise a targeting sequence that is complementary to a contiguous sequence of nucleotides comprising a target mRNA, wherein the contiguous sequence of nucleotides is 10, 11, 12, 13, 14, 15, 16, 17, 18, 19 or 20 nucleotides in length. In some embodiments, the RNAi oligonucleotide conjugates comprise a targeting sequence that is complementary to a contiguous sequence of nucleotides comprising a target mRNA, wherein the contiguous sequence of nucleotides is 19 nucleotides in length. In some embodiments, the RNAi oligonucleotide conjugates comprise a targeting sequence that is complementary to a contiguous sequence of nucleotides comprising a target mRNA, wherein the contiguous sequence of nucleotides is 20 nucleotides in length.

In some embodiments, the RNAi oligonucleotide conjugated comprises a targeting sequence that is complementary to a contiguous sequence of nucleotides comprising a target mRNA (e.g., a CNS target mRNA), wherein the contiguous sequence of nucleotides is 15 nucleotides in length. In some embodiments, the RNAi oligonucleotide conjugate comprises a targeting sequence that is complementary to a contiguous sequence of nucleotides comprising a target mRNA (e.g., a CNS target mRNA), wherein the contiguous sequence of nucleotides is 19 nucleotides in length.

In some embodiments, a targeting sequence of an RNAi oligonucleotide conjugate herein is fully complementary (e.g., having no mismatches) to a target sequence comprising a target mRNA and comprises the entire length of an antisense strand. In some embodiments, a targeting sequence of an RNAi oligonucleotide conjugate herein is fully complementary (e.g., having no mismatches) to a target sequence comprising a target mRNA and comprises a portion of the entire length of an antisense strand. In some embodiments, a targeting sequence of an RNAi oligonucleotide conjugate herein is fully complementary (e.g., having no mismatches) to a target sequence comprising a target mRNA and comprises 10 to 20 nucleotides of the antisense strand. In some embodiments, a targeting sequence of an RNAi oligonucleotide conjugate herein is fully complementary (e.g., having no mismatches) to a target sequence comprising a target mRNA and comprises 15 to 19 nucleotides of the antisense strand. In some embodiments, a targeting sequence of an RNAi oligonucleotide conjugate herein is fully complementary (e.g., having no mismatches) to a target sequence comprising a target mRNA and comprises 15 nucleotides, 16 nucleotides, 17 nucleotides, 18 nucleotides, 19 nucleotides, 20 nucleotides, 21 nucleotides, or 22 nucleotides of the antisense strand. In some embodiments, a targeting sequence of an RNAi oligonucleotide conjugate herein is fully complementary (e.g., having no mismatches) to a target sequence comprising a target mRNA and comprises 19 nucleotides of the antisense strand. In some embodiments, a targeting sequence of an RNAi oligonucleotide conjugate herein is fully complementary (e.g., having no mismatches) to a target sequence comprising a target mRNA and comprises 20 nucleotides of the antisense strand.

In some embodiments, a targeting sequence of an RNAi oligonucleotide conjugate herein is partially complementary (e.g., having no more than 4 mismatches) to a target sequence comprising a target mRNA and comprises the entire length of an antisense strand. In some embodiments, a targeting sequence of an RNAi oligonucleotide conjugate herein is partially complementary (e.g., having no more than 4 mismatches) to a target sequence comprising a target mRNA and comprises a portion of the entire length of an antisense strand. In some embodiments, a targeting sequence of an RNAi oligonucleotide conjugate herein is partially complementary (e.g., having no more than 4 mismatches) to a target sequence comprising a target mRNA and comprises 10 to 20 nucleotides of the antisense strand. In some embodiments, a targeting sequence of an RNAi oligonucleotide conjugate herein is partially complementary (e.g., having no more than 4 mismatches) to a target sequence comprising a target mRNA and comprises 15 to 19 nucleotides of the antisense strand. In some embodiments, a targeting sequence of an RNAi oligonucleotide conjugate herein is partially complementary (e.g., having no more than 4 mismatches) to a target sequence comprising a target mRNA and comprises 15 nucleotides, 16 nucleotides, 17 nucleotides, 18 nucleotides, 19 nucleotides, 20 nucleotides, 21 nucleotides, or 22 nucleotides of the antisense strand. In some embodiments, a targeting sequence of an RNAi oligonucleotide conjugate herein is partially complementary (e.g., having no more than 4 mismatches) to a target sequence comprising a target mRNA and comprises 19 nucleotides of the antisense strand. In some embodiments, a targeting sequence of an RNAi oligonucleotide conjugate herein is partially complementary (e.g., having no more than 4 mismatches) to a target sequence comprising a target mRNA and comprises 20 nucleotides of the antisense strand.

In some embodiments, an RNAi oligonucleotide conjugate herein comprises a targeting sequence having one or more base pair (bp) mismatches with the corresponding target sequence comprising a target mRNA. In some embodiments, the targeting sequence has a 1 bp mismatch, a 2 bp mismatch, a 3 bp mismatch, a 4 bp mismatch, or a 5 bp mismatch with the corresponding target sequence comprising a target mRNA provided that the ability of the targeting sequence to bind or anneal to the target sequence under appropriate hybridization conditions and/or the ability of the RNAi oligonucleotide conjugate to inhibit or reduce target gene expression is maintained (e.g., under physiological conditions). Alternatively, in some embodiments, the targeting sequence comprises no more than 1, no more than 2, no more than 3, no more than 4, or no more than 5 bp mismatches with the corresponding target sequence comprising a target mRNA provided that the ability of the targeting sequence to bind or anneal to the target sequence under appropriate hybridization conditions and/or the ability of the RNAi oligonucleotide conjugate to inhibit or reduce target gene expression is maintained. In some embodiments, the RNAi oligonucleotide conjugate comprises a targeting sequence having 1 mismatch with the corresponding target sequence. In some embodiments, the RNAi oligonucleotide conjugate comprises a targeting sequence having 2 mismatches with the corresponding target sequence. In some embodiments, the RNAi oligonucleotide conjugate comprises a targeting sequence having 3 mismatches with the corresponding target sequence. In some embodiments, the RNAi oligonucleotide conjugate comprises a targeting sequence having 4 mismatches with the corresponding target sequence. In some embodiments, the RNAi oligonucleotide conjugate comprises a targeting sequence having 5 mismatches with the corresponding target sequence. In some embodiments, the RNAi oligonucleotide conjugate comprises a targeting sequence having more than one mismatch (e.g., 2, 3, 4, 5 or more mismatches) with the corresponding target sequence, wherein at least 2 (e.g., all) of the mismatches are positioned consecutively (e.g., 2, 3, 4, 5 or more mismatches in a row), or wherein the mismatches are interspersed in any position throughout the targeting sequence. In some embodiments, the RNAi oligonucleotide conjugate comprises a targeting sequence having more than one mismatch (e.g., 2, 3, 4, 5 or more mismatches) with the corresponding target sequence, wherein at least 2 (e.g., all) of the mismatches are positioned consecutively (e.g., 2, 3, 4, 5 or more mismatches in a row), or wherein at least one or more non-mismatched base pair is located between the mismatches, or a combination thereof.

### Types of Oligonucleotides

A variety of RNAi oligonucleotide types and/or structures are useful for reducing target gene expression in the methods herein. Any of the RNAi oligonucleotide types described herein or elsewhere are contemplated for use as a framework to incorporate a targeting sequence herein for the purposes of inhibiting or reducing corresponding target gene expression in the CNS.

In some embodiments, the RNAi oligonucleotide conjugates herein inhibit target gene expression by engaging with RNA interference (RNAi) pathways upstream or downstream of Dicer involvement. For example, RNAi oligonucleotides have been developed with each strand having sizes of about 19-25 nucleotides with at least one 3′ overhang of 1 to 5 nucleotides (see, e.g., U.S. Pat. No. 8,372,968). Longer oligonucleotides also have been developed that are processed by Dicer to generate active RNAi products (see, e.g., U.S. Pat. No. 8,883,996). Further work produced extended double-stranded oligonucleotides where at least one end of at least one strand is extended beyond a duplex targeting region, including structures where one of the strands includes a thermodynamically-stabilizing tetraloop structure (see, e.g., U.S. Pat. Nos. 8,513,207 and 8,927,705, as well as Intl. Patent Application Publication No. WO 2010/033225). Such structures may include single-stranded extensions (on one or both sides of the molecule) as well as double-stranded extensions.

In some embodiments, the RNAi oligonucleotide conjugates herein engage with the RNAi pathway downstream of the involvement of Dicer (e.g., Dicer cleavage). In some embodiments, the RNAi oligonucleotide conjugates described herein are Dicer substrates. In some embodiments, upon endogenous Dicer processing, double-stranded nucleic acids of 19-23 nucleotides in length capable of reducing expression of a target mRNA (e.g., a CNS target mRNA) are produced. In some embodiments, the RNAi oligonucleotide conjugate has an overhang (e.g., of 1, 2, or 3 nucleotides in length) in the 3′ end of the sense strand. In some embodiments, the RNAi oligonucleotide conjugate (e.g., siRNA conjugate) comprises a 21-nucleotide guide strand that is antisense to a target mRNA and a complementary passenger strand, in which both strands anneal to form a 19-bp duplex and 2 nucleotide overhangs at either or both 3′ ends. Longer oligonucleotide designs also are contemplated including oligonucleotides having a guide strand of 23 nucleotides and a passenger strand of 21 nucleotides, where there is a blunt end on the right side of the molecule (3′ end of passenger strand/5′ end of guide strand) and a two nucleotide 3′-guide strand overhang on the left side of the molecule (5′ end of the passenger strand/3′ end of the guide strand). In such molecules, there is a 21 bp duplex region. See, e.g., U.S. Pat. Nos. 9,012,138; 9,012,621 and 9,193,753.

In some embodiments, the RNAi oligonucleotide conjugates disclosed herein comprise sense and antisense strands that are both in the range of about 17 to 26 (e.g., 17 to 26, 20 to 25 or 21-23) nucleotides in length. In some embodiments, the RNAi oligonucleotide conjugates disclosed herein comprise a sense and antisense strand that are both in the range of about 19-22 nucleotides in length. In some embodiments, the sense and antisense strands are of equal length. In some embodiments, the RNAi oligonucleotide conjugates disclosed herein comprise sense and antisense strands, such that there is a 3′-overhang on either the sense strand or the antisense strand, or both the sense and antisense strand. In some embodiments, for RNAi oligonucleotide conjugates that have sense and antisense strands that are both in the range of about 21-23 nucleotides in length, a 3′ overhang on the sense, antisense, or both sense and antisense strands is 1 or 2 nucleotides in length. In some embodiments, an RNAi oligonucleotide conjugate has a guide strand of 22 nucleotides and a passenger strand of 20 nucleotides, where there is a blunt end on the right side of the molecule (3′ end of passenger strand/5′ end of guide strand) and a 2 nucleotide 3′-guide strand overhang on the left side of the molecule (5′ end of the passenger strand/3′ end of the guide strand). In such molecules, there is a 20 bp duplex region.

Other RNAi oligonucleotide designs for use with the compositions and methods herein include: 16-mer siRNAs (see, e.g., Nucleic Acids in Chemistry and Biology, Blackburn (ed.), ROYAL SOCIETY OF CHEMISTRY, 2006), shRNAs (e.g., having 19 bp or shorter stems; see, e.g., Moore et al. (2010) METHODS MOL. BIOL. 629:141-58), blunt siRNAs (e.g., of 19 bps in length; see, e.g., Kraynack & Baker (2006) RNA 12:163-76), asymmetrical siRNAs (aiRNA; see, e.g., Sun et al. (2008) NAT. BIOTECHNOL. 26:1379-82), asymmetric shorter-duplex siRNA (see, e.g., Chang et al. (2009) Mol. Ther. 17:725-732), fork siRNAs (see, e.g., Hohjoh (2004) FEBS Lett. 557:193-98), single-stranded siRNAs (Elsner (2012) NAT. BIOTECHNOL. 30:1063), dumbbell-shaped circular siRNAs (see, e.g., Abe et al. (2007) J. AM. CHEM. SOC. 129:15108-09), and small internally segmented interfering RNA (siRNA; see, e.g., Bramsen et al. (2007) Nucleic Acids Res. 35:5886-97). Further non-limiting examples of an oligonucleotide structure that may be used in some embodiments to reduce or inhibit the expression of a target gene are microRNA (miRNA), short hairpin RNA (shRNA) and short siRNA (see, e.g., Hamilton et al. (2002) EMBO J. 21:4671-79; see also, US Patent Application Publication No. 2009/0099115).

Still, in some embodiments, a RNAi oligonucleotide conjugate herein for reducing or inhibiting target gene expression is single-stranded (ss). Such structures may include but are not limited to single-stranded RNAi molecules. Recent efforts have demonstrated the activity of single-stranded RNAi molecules (see, e.g., Matsui et al. (2016) MOL. THER. 24:946-955). However, in some embodiments, the oligonucleotide-ligand conjugates described herein comprise an antisense oligonucleotide (ASO). An antisense oligonucleotide is a single-stranded oligonucleotide that has a nucleobase sequence which, when written or depicted in the 5′ to 3′ direction, comprises the reverse complement of a targeted segment of a particular nucleic acid and is suitably modified (e.g., as a gapmer) so as to induce RNaseH-mediated cleavage of its target RNA in cells or (e.g., as a mixmer) so as to inhibit translation of the target mRNA in cells. ASOs for use herein may be modified in any suitable manner known in the art including, for example, as shown in U.S. Pat. No. 9,567,587 (including, e.g., length, sugar moieties of the nucleobase (pyrimidine, purine), and alterations of the heterocyclic portion of the nucleobase). Further, ASOs have been used for decades to reduce expression of specific target genes (see, e.g., Bennett et al. (2017) ANNU. REV. PHARMACOL. 57:81-105).

### Structure of Nucleic-Acid Ligand Conjugates

In certain aspects, the disclosure provides a nucleic acid-ligand conjugate represented by formula I-a:

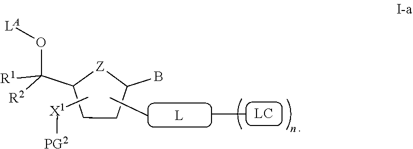

or a pharmaceutically acceptable salt thereof, wherein:
B is a nucleobase or hydrogen;
R¹ and R² are independently hydrogen, halogen, R^{A}, —CN, —S(O)R, —S(O)₂R, —Si(OR)₂R, —Si(OR)R₂, or —SiR₃, or
   R¹ and R² on the same carbon are taken together with their intervening atoms to form a 3-membered saturated or partially unsaturated ring having 0-3 heteroatoms, independently selected from nitrogen, oxygen, and sulfur;
each R^{A} is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, a suitable protecting group, or an optionally substituted group selected from C1-6 aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur;
   L^{A} is independently PG¹, or -L-ligand;
   PG¹ is hydrogen or a suitable hydroxyl protecting group;
   each ligand is independently -(LC)n, and/or an adamantyl group;
each LC is independently a lipid conjugate moiety comprising a saturated or unsaturated, straight, or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, or —P(S)OR—;
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, adamantanenyl, a 4-7 membered saturated or partially unsaturated heterocyclylenyl having 1-3 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
n is 1-10;
L is a covalent bond or a bivalent saturated or unsaturated, straight or branched C₁₋₅₀ hydrocarbon chain, wherein 0-10 methylene units of the hydrocarbon chain are independently replaced by -Cy-, —O—, —NR—, —N(R)—C(O)—, —S—, —C(O)—, —S(O)—, —S(O)₂—, —P(O)OR—, —P(S)OR—, —V¹CR²W¹— or

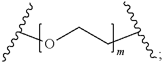

m is 1-50;
X¹, V¹ and W¹ are independently —C(R)₂—, —OR, —O—, —S—, —Se—, or —NR—;
Z is —O—, —S—, —NR—, or —CR₂—; and
PG² is hydrogen, a phosphoramidite analogue, or a suitable protecting group.

In certain embodiments, the nucleic acid-ligand conjugate is represented by formula I-b or I-c:

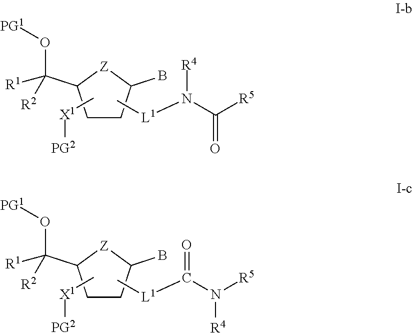

or a pharmaceutically acceptable salt thereof; wherein
L¹ is a covalent bond or a bivalent saturated or unsaturated, straight or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, —P(S)OR—, or

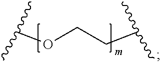

R⁴ is hydrogen, R^{A}, or a suitable amine protection group; and
R⁵ is adamantyl, or a saturated or unsaturated, straight, or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, or —P(S)OR—.

In some embodiments, a nucleic acid-ligand conjugate is represented by formula I-Ib or I-Ic:

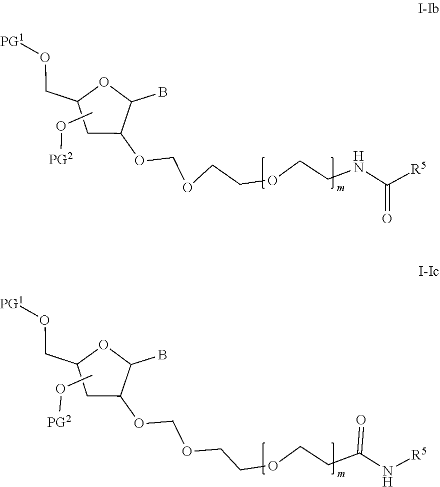

or a pharmaceutically acceptable salt thereof; wherein
B is a nucleobase or hydrogen;
m is 1-50;
PG¹ and PG² are independently a hydrogen, a phosphoramidite analogue, or a suitable protecting group; and
R⁵ is adamantyl, or a saturated or unsaturated, straight, or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, or —P(S)OR—.

In certain embodiments of the nucleic acid-ligand conjugate, R⁵ is selected from

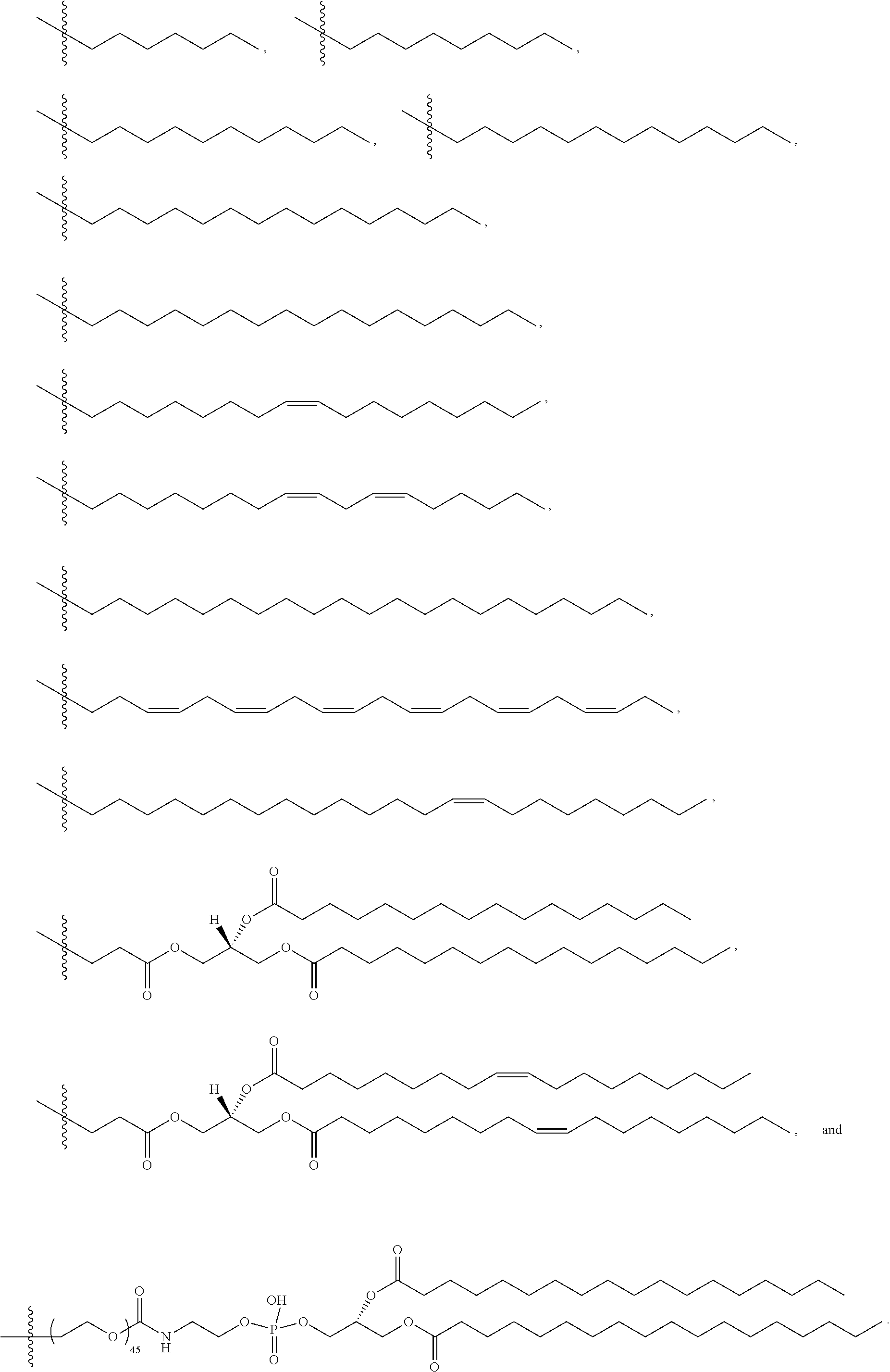

In some embodiments of the nucleic acid-ligand conjugate, R⁵ is selected from:

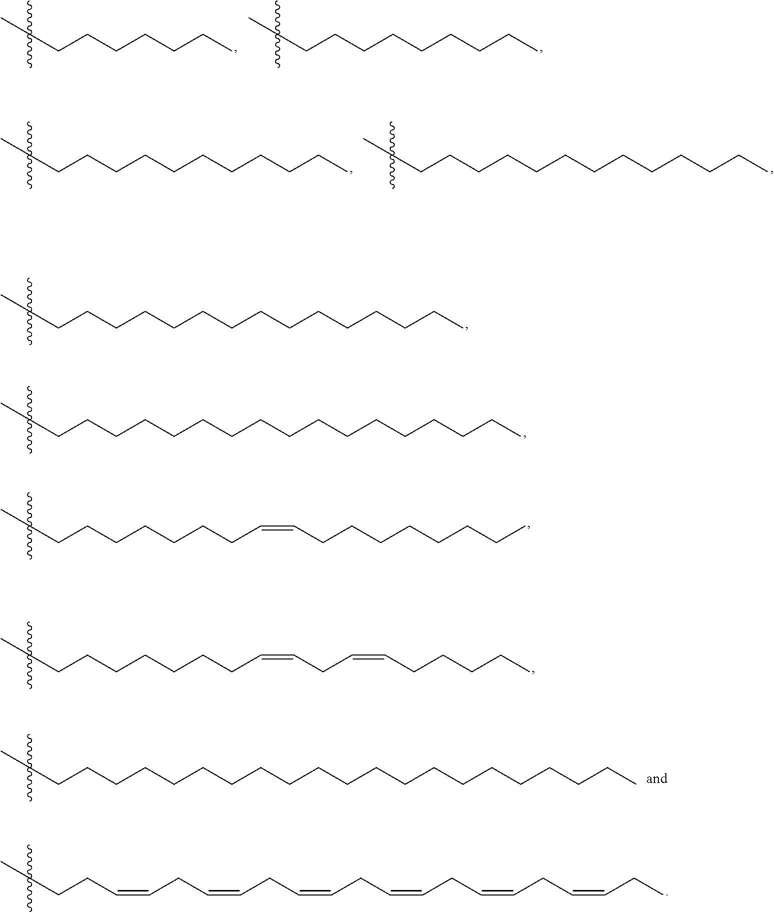

### Double-Stranded RNAi Oligonucleotide Conjugates

### Structure

In some aspects, the disclosure provides an oligonucleotide-ligand conjugate or an oligonucleotide conjugate comprising one or more nucleic acid-ligand conjugates represented by formula II-a:

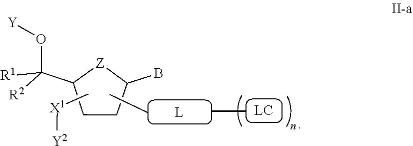

or a pharmaceutically acceptable salt thereof, wherein:
B is a nucleobase or hydrogen;
R¹ and R² are independently hydrogen, halogen, R^{A}, —CN, —S(O)R, —S(O)₂R, —Si(OR)₂R, —Si(OR)R₂, or —SiR₃; or
   R¹ and R² on the same carbon are taken together with their intervening atoms to form a 3-7 membered saturated or partially unsaturated ring having 0-3 heteroatoms, independently selected from nitrogen, oxygen, and sulfur;
each R^{A} is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur; or
   two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur;
each LC is independently a lipid conjugate moiety comprising a saturated or unsaturated, straight, or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, —P(S)OR—;
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-3 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur; n is 1-10; L is a covalent bond or a bivalent saturated or unsaturated, straight or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, —P(S)OR—, —V¹CR²W¹—, or

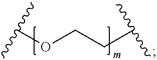

m is 1-50;
X¹, V¹ and W¹ are independently —C(R)₂—, —OR, —O—, —S—, —Se—, or —NR—;
Y is hydrogen, a suitable hydroxyl protecting group

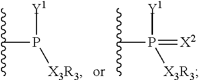

R³ is hydrogen, a suitable protecting group, a suitable prodrug, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur;
X² is O, S, or NR;
X³ is —O—, —S—, —BH₂—, or a covalent bond;
Y¹ is a linking group attaching to the 2′- or 3′-terminal of a nucleoside, a nucleotide, or an oligonucleotide;
Y² is hydrogen, a suitable protecting group, a phosphoramidite analogue, an internucleotide linking group attaching to the 5′-terminal of a nucleoside, a nucleotide, or an oligonucleotide, or a linking group attaching to a solid support; and
Z is —O—, —S—, —NR—, or —CR₂—.

In certain embodiments, the oligonucleotide-ligand conjugate (oligonucleotide conjugate) is represented by formula II-b or II-c:

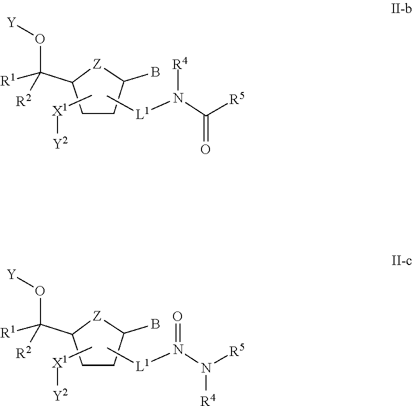

or a pharmaceutically acceptable salt thereof, wherein:
L¹ is a covalent bond, a monovalent or a bivalent saturated or unsaturated, straight or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, —P(S)OR—, or

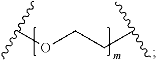

R⁴ is hydrogen, R^{A}, or a suitable amine protection group; and
R⁵ is adamantyl, or a saturated or unsaturated, straight, or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, or —P(S)OR.

In some embodiments of the oligonucleotide-ligand conjugate, R⁵ is selected from:

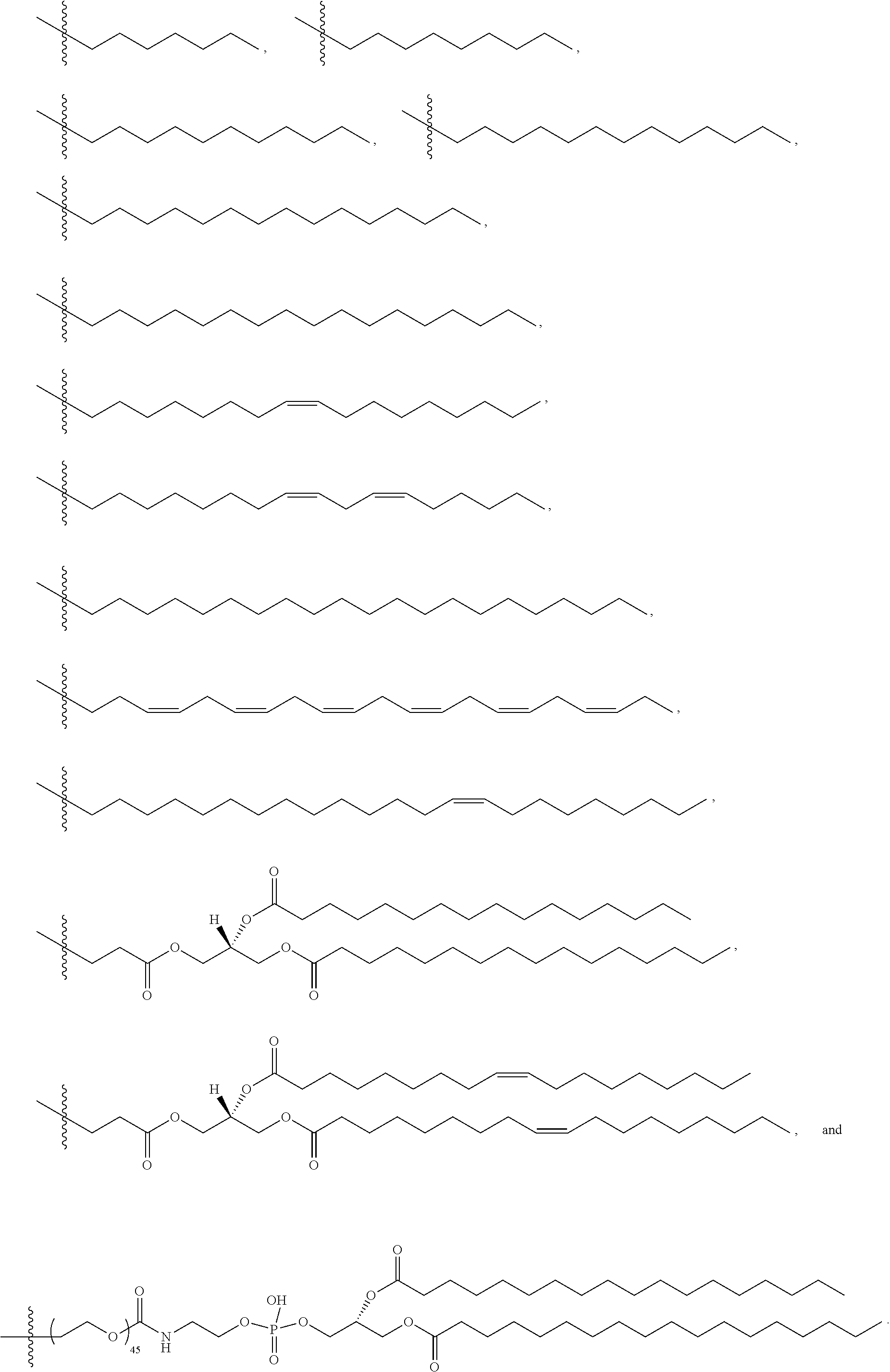

In certain embodiments of the oligonucleotide-ligand conjugate, R⁵ is selected from:

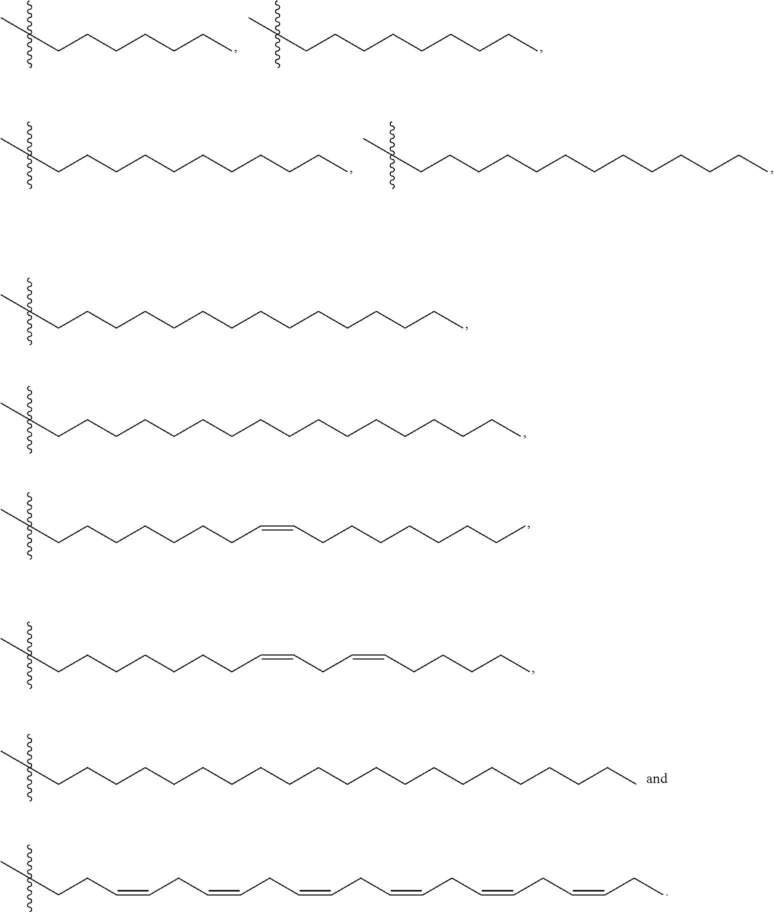

In some embodiments, R⁵ is

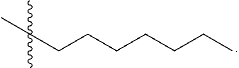 [SMILES: *CCCCCCC]

In some embodiments, R⁵ is

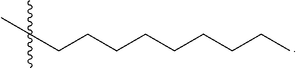 [SMILES: *CCCCCCCCC]

In some embodiments, R⁵ is

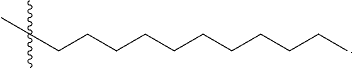 [SMILES: *CCCCCCCCCCC]

In some embodiments, R⁵ is

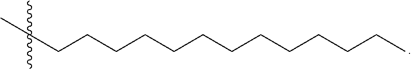 [SMILES: *CCCCCCCCCCCCC]

In some embodiments, R⁵ is

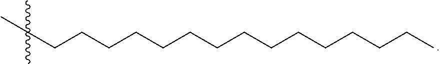 [SMILES: *CCCCCCCCCCCCCCC]

In some embodiments, R⁵ is

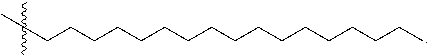 [SMILES: *CCCCCCCCCCCCCCCCC]

In some embodiments, R⁵ is

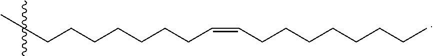 [SMILES: *CCCCCCC/C=C\CCCCCCCC]

In some embodiments, R is

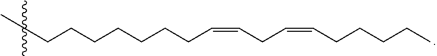 [SMILES: *CCCCCCC/C=C\C/C=C\CCCCC]

In some embodiments, R⁵ is

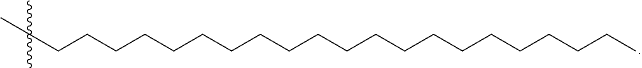 [SMILES: *CCCCCCCCCCCCCCCCCCCCC]

In some embodiments, R⁵ is

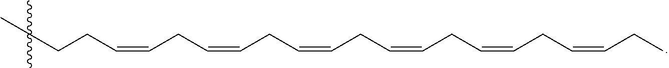 [SMILES: *CC/C=C\C/C=C\C/C=C\C/C=C\C/C=C\C/C=C\CC]

In some embodiments, R⁵ is

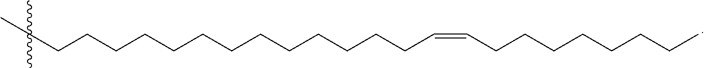 [SMILES: *CCCCCCCCCCCCC/C=C\CCCCCCCC]

In some embodiments, R⁵ is

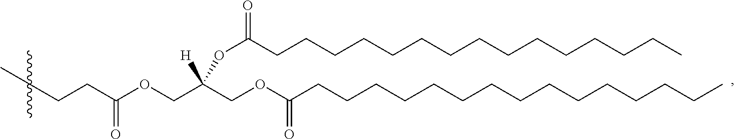 [SMILES: *CCC(=O)OC[C@@H](COC(=O)CCCCCCCCCCCCCCC)OC(=O)CCCCCCCCCCCCCCC]

In some embodiments, R⁵ is

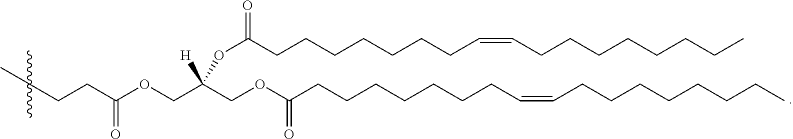 [SMILES: *CCC(=O)OC[C@@H](COC(=O)CCCCCCC/C=C\CCCCCCCC)OC(=O)CCCCCCC/C=C\CCCCCCCC]

In some embodiments, R⁵ is

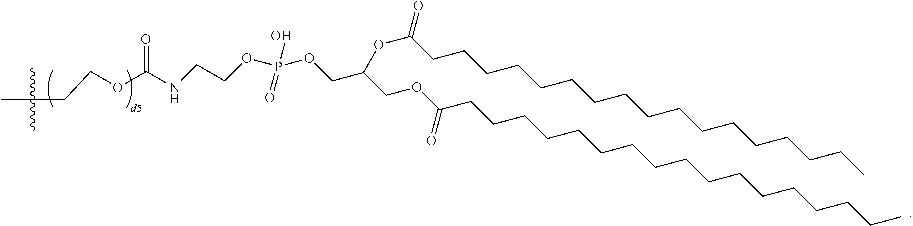

In some embodiments, an oligonucleotide-ligand conjugate is represented by formula II-Ib or II-Ic:

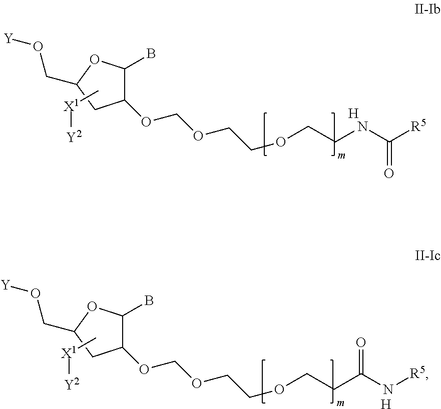

or a pharmaceutically acceptable salt thereof; wherein
B is a nucleobase or hydrogen;
m is 1-50;
X¹ is —O—, or —S—;
Y is hydrogen,

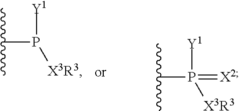

R³ is hydrogen, or a suitable protecting group;
X² is O, or S;
X³ is —O—, —S—, or a covalent bond;
Y¹ is a linking group attaching to the 2′- or 3′-terminal of a nucleoside, a nucleotide, or an oligonucleotide;
Y² is hydrogen, a phosphoramidite analogue, an internucleotide linking group attaching to the 5′-terminal of a nucleoside, a nucleotide, or an oligonucleotide, or a linking group attaching to a solid support;
R⁵ is adamantyl, or a saturated or unsaturated, straight, or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, or —P(S)OR—; and
R is hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur.

In certain embodiments of the oligonucleotide-ligand conjugate, R⁵ is selected from:

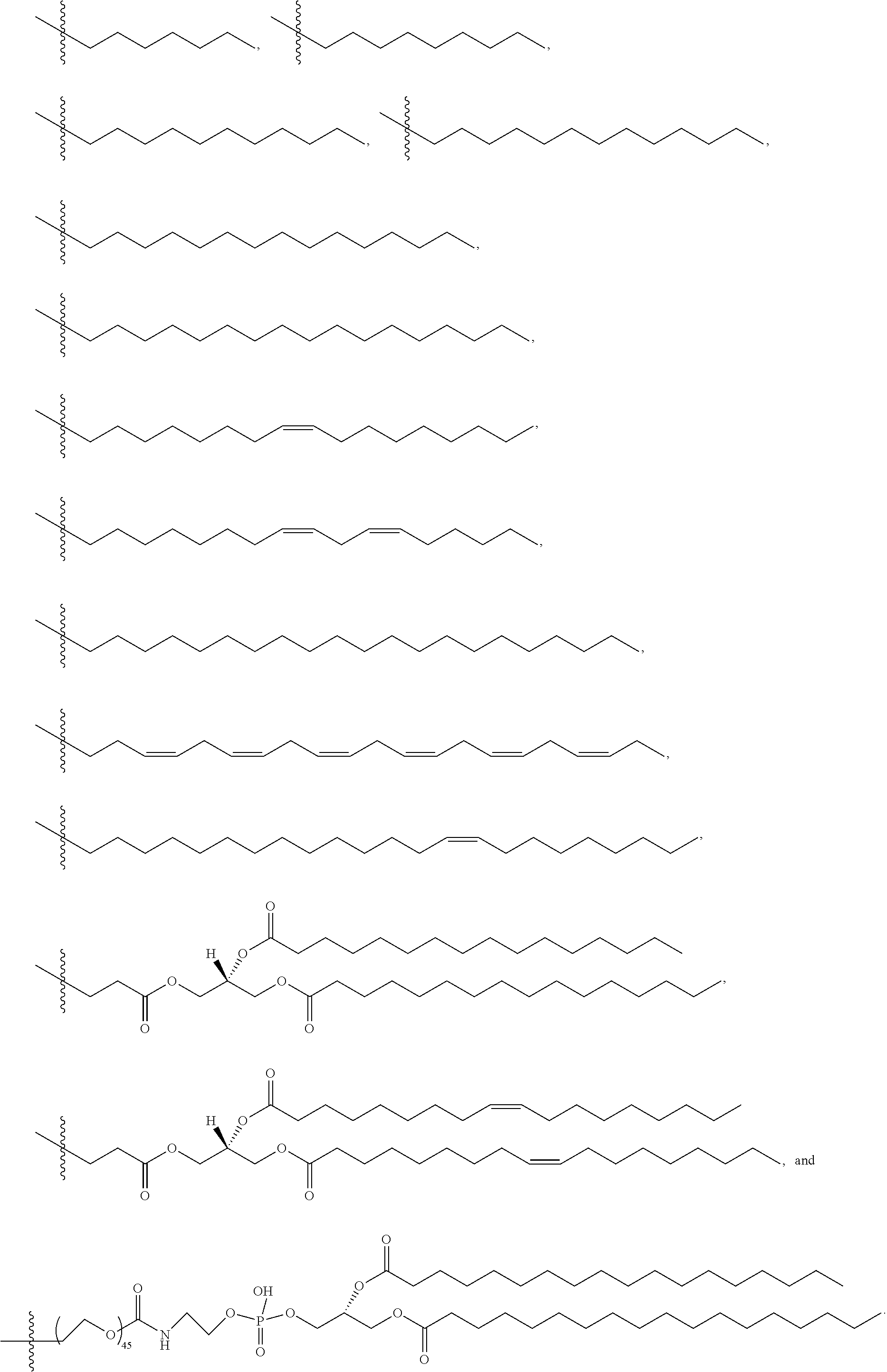

In certain embodiments, the oligonucleotide-ligand conjugate comprises one or more nucleic acid-ligand conjugate configurations of any one of the above disclosed embodiments.

In certain embodiments, the oligonucleotide-ligand conjugate comprises 1, 2, 3, 4, 5, 6, 7, 8, 9 or 10 nucleic acid-ligand conjugate units.

In some aspects, the disclosure provides RNAi oligonucleotide conjugates for targeting a target mRNA (e.g., a target mRNA expressed in the CNS) and inhibiting or reducing target gene expression (e.g., via the RNAi pathway), wherein the RNAi oligonucleotide conjugate is a double-stranded (ds) nucleic acid molecule comprising a sense strand (also referred to herein as a passenger strand) and an antisense strand (also referred to herein as a guide strand). In some embodiments, the sense strand and antisense strand are separate strands and are not covalently linked. In some embodiments, the sense strand and antisense strand are covalently linked. In some embodiments, the sense strand and antisense strand form a duplex region, wherein the sense strand and antisense strand, or a portion thereof, binds or anneals to one another in a complementary manner (e.g., by Watson-Crick base pairing).

In some embodiments, the sense strand has a first region (R1) and a second region (R2), wherein R2 comprises a first subregion (S1), a tetraloop (L) or triloop (triL), and a second subregion (S2), wherein L or triL is located between S1 and S2, and wherein S1 and S2 form a second duplex (D2). D2 may have various lengths. In some embodiments, D2 is about 1-6 bp in length. In some embodiments, D2 is 2-6, 3-6, 4-6, 5-6, 1-5, 2-5, 3-5 or 4-5 bp in length. In some embodiments, D2 is 1, 2, 3, 4, 5 or 6 bp in length. In some embodiments, D2 is 6 bp in length.

In some embodiments, R1 of the sense strand and the antisense strand form a first duplex (D1). In some embodiments, D1 is at least about 15 (e.g., at least 15, at least 16, at least 17, at least 18, at least 19, at least 20 or at least 21) nucleotides in length. In some embodiments, D1 is in the range of about 12 to 30 nucleotides in length (e.g., 12 to 30, 12 to 27, 15 to 22, 18 to 22, 18 to 25, 18 to 27, 18 to 30 or 21 to 30 nucleotides in length). In some embodiments, D1 is at least 12 nucleotides in length (e.g., at least 12, at least 15, at least 20, at least 25, or at least 30 nucleotides in length). In some embodiments, D1 is 12, 13, 14, 15, 16, 17, 18, 19, 20, 21, 22, 23, 24, 25, 26, 27, 28, 29 or 30 nucleotides in length. In some embodiments, D1 is 19 nucleotides in length. In some embodiments, D1 is 20 nucleotides in length. In some embodiments, D1 comprising the sense strand and antisense strand does not span the entire length of the sense strand and/or antisense strand. In some embodiments, D1 comprising the sense strand and antisense strand spans the entire length of either the sense strand or antisense strand or both. In certain embodiments, D1 comprising the sense strand and antisense strand spans the entire length of both the sense strand and the antisense strand.

It should be appreciated that, in some embodiments, sequences presented in the Sequence Listing may be referred to in describing the structure of an oligonucleotide (e.g., a RNAi oligonucleotide conjugate) or other nucleic acid. In such embodiments, the actual oligonucleotide or other nucleic acid may have one or more alternative nucleotides (e.g., an RNA counterpart of a DNA nucleotide or a DNA counterpart of an RNA nucleotide) and/or one or more modified nucleotides and/or one or more modified internucleotide linkages and/or one or more other modification when compared with the specified sequence while retaining essentially same or similar complementary properties as the specified sequence.

In some embodiments, an RNAi oligonucleotide conjugate herein comprises a 25-nucleotide sense strand and a 27-nucleotide antisense strand that when acted upon by a Dicer enzyme results in an antisense strand that is incorporated into the mature RISC. In some embodiments, the sense strand of the RNAi oligonucleotide conjugate is longer than 27 nucleotides (e.g., 28, 29, 30, 31, 32, 33, 34, 35, 36, 37, 38, 39, 40, 41, 42, 43, 44, 45, 46, 47, 48, 49 or 50 nucleotides). In some embodiments, the sense strand of the RNAi oligonucleotide conjugate is longer than 25 nucleotides (e.g., 26, 27, 28, 29 or 30 nucleotides).

In some embodiments, the RNAi oligonucleotide conjugates herein have one 5′ end that is thermodynamically less stable when compared to the other 5′ end. In some embodiments, an asymmetric RNAi oligonucleotide conjugate is provided that comprises a blunt end at the 3′ end of a sense strand and a 3′-overhang at the 3′ end of an antisense strand. In some embodiments, the 3′-overhang on the antisense strand is about 1-8 nucleotides in length (e.g., 1, 2, 3, 4, 5, 6, 7 or 8 nucleotides in length). Typically, an RNAi oligonucleotide conjugate has a two-nucleotide overhang on the 3′ end of the antisense (guide) strand. However, other overhangs are possible. In some embodiments, an overhang is a 3′-overhang comprising a length of between 1 and 6 nucleotides, optionally 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, 5 to 6 nucleotides, or 1, 2, 3, 4, 5 or 6 nucleotides. However, in some embodiments, the overhang is a 5′-overhang comprising a length of between 1 and 6 nucleotides, optionally 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, 5 to 6 nucleotides, or 1, 2, 3, 4, 5 or 6 nucleotides.

In some embodiments, two terminal nucleotides on the 3′ end of an antisense strand are modified. In some embodiments, the two terminal nucleotides on the 3′ end of the antisense strand are complementary with the target mRNA (e.g., a target mRNA expressed in the CNS). In some embodiments, the two terminal nucleotides on the 3′ end of the antisense strand are not complementary with the target mRNA. In some embodiments, the two terminal nucleotides on the 3′ end of the antisense strand of an RNAi oligonucleotide conjugate herein are unpaired. In some embodiments, the two terminal nucleotides on the 3′ end of the antisense strand of an RNAi oligonucleotide conjugate herein comprise an unpaired GG. In some embodiments, the two terminal nucleotides on the 3′ end of the antisense strand of an RNAi oligonucleotide conjugate herein are not complementary to the target mRNA. In some embodiments, two terminal nucleotides on each 3′ end of an RNAi oligonucleotide conjugate are GG. Typically, one or both of the two terminal GG nucleotides on each 3′ end of a double-stranded oligonucleotide (e.g., an RNAi oligonucleotide conjugate) is not complementary with the target mRNA.

In some embodiments, there is one or more (e.g., 1, 2, 3, 4 or 5) mismatch(s) between a sense and antisense strand. If there is more than one mismatch between a sense and antisense strand, they may be positioned consecutively (e.g., 2, 3 or more in a row), or interspersed throughout the region of complementarity. In some embodiments, the 3′ end of the sense strand contains one or more mismatches. In one embodiment, two mismatches are incorporated at the 3′ end of the sense strand. In some embodiments, base mismatches, or destabilization of segments at the 3′ end of the sense strand of an RNAi oligonucleotide conjugate herein improves or increases the potency and/or efficacy of the RNAi oligonucleotide conjugate.

In some embodiments, the disclosure provides an RNAi oligonucleotide conjugate comprising an RNAi oligonucleotide and a lipid moiety. In some embodiments, the lipid moiety is conjugated to the sense strand of the RNAi oligonucleotide. In some embodiments, the RNAi oligonucleotide comprises a stem loop. In some embodiments, the ligand is conjugated to any of the nucleotides in the stem loop. In some embodiments, the ligand is conjugated to the first nucleotide from 5′ to 3′, in the stem loop. In some embodiments, the ligand is conjugated to the second nucleotide from 5′ to 3′ in the stem loop. In some embodiments, the ligand is conjugated to the third nucleotide from 5′ to 3′ in the stem loop. In some embodiments, the ligand is conjugated to the fourth nucleotide from 5′ to 3′ in the stem loop. In some embodiments, the ligand is conjugated to one, two, three, or four of the nucleotides in the stem loop. In some embodiments, the ligand is conjugated to three of the nucleotides in the stem loop.

In some embodiments, the oligonucleotide-ligand conjugate comprises a sense strand of 36 nucleotides with positions numbered 1-36 from 5′ to 3′. In some embodiments, the oligonucleotide-ligand conjugate comprises a lipid conjugated to position 27 of a 36-nucleotide sense strand. In some embodiments, the oligonucleotide-ligand conjugate comprises a lipid conjugated to position 28 of a 36-nucleotide sense strand. In some embodiments, the oligonucleotide conjugate comprises a lipid conjugated to position 29 of a 36-nucleotide sense strand. In some embodiments, the oligonucleotide conjugate comprises a lipid conjugated to position 30 of a 36-nucleotide sense strand.

In some embodiments, an oligonucleotide-ligand conjugate comprises an antisense strand of 15 to 30 nucleotides and a sense strand of 15 to 40 nucleotide, wherein the sense and antisense strands form a duplex region, wherein the antisense strand comprises a region of complementarity to a target sequence expressed in a tissue or cell of the CNS, wherein the sense strand comprises at its 3′ end a stem-loop comprising a tetraloop comprising 4 nucleosides, wherein one or more of the 4 nucleosides is represented by formula II-Ib:

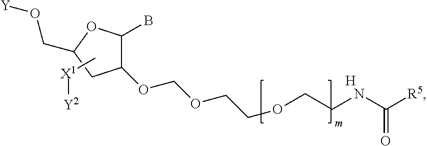

wherein B is selected from an adenine and a guanine nucleobase, and wherein R⁵ is a hydrocarbon chain. In some embodiments, m is 1, X1 is O, Y2 is an internucleotide linking group attaching to the 5′ terminal of a nucleoside,
Y is represented by

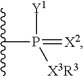

Y1 is a linking group attaching to the 2′ or 3′ terminal of a nucleotide, X2 is O, X3 is O, and R3 is H. In some embodiments, the hydrocarbon chain is a C8-C30 hydrocarbon chain. In some embodiments, the hydrocarbon chain is a C16 hydrocarbon chain. In some embodiments, the C16 hydrocarbon chain is represented by

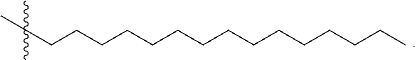 [SMILES: *CCCCCCCCCCCCCCC]

In some embodiments, the 4 nucleosides of the tetraloop are numbered 1-4 from 5′ to 3′ and position 1 is represented by formula II-Ib. In some embodiments, position 2 is represented by formula II-Ib. In some embodiments, position 3 is represented by formula II-Ib. In some embodiments, position 4 is represented by formula II-Ib. In some embodiments, the sense strand is 36 nucleotides with positions numbered 1-36 from 5′ to 3′, wherein the stem-loop comprises nucleotides at positions 21-36, and wherein one or more nucleosides at positions 27-30 are represented by formula II-Ib. In some embodiments, the antisense strand is 22 nucleotides.

### Antisense Strands

In some embodiments, an antisense strand of a RNAi oligonucleotide conjugate is referred to as a “guide strand.” For example, an antisense strand that engages with RNA-induced silencing complex (RISC) and binds to an Argonaute protein such as Ago2, or engages with or binds to one or more similar factors, and directs silencing of a target gene, the antisense strand is referred to as a guide strand. In some embodiments, a sense strand complementary to a guide strand is referred to as a “passenger strand.”

In some embodiments, an RNAi oligonucleotide conjugate herein comprises an antisense strand of up to about 50 nucleotides in length (e.g., up to 50, up to 40, up to 35, up to 30, up to 27, up to 25, up to 21, up to 19, up to 17, up to 15, or up to 12 nucleotides in length). In some embodiments, an RNAi oligonucleotide conjugate herein comprises an antisense strand of at least about 12 nucleotides in length (e.g., at least 12, at least 15, at least 19, at least 21, at least 22, at least 25, at least 27, at least 30, at least 35 or at least 38 nucleotides in length). In some embodiments, an RNAi oligonucleotide conjugate herein comprises an antisense strand in a range of about 12 to about 40 (e.g., 12 to 40, 12 to 36, 12 to 32, 12 to 28, 15 to 40, 15 to 36, 15 to 32, 15 to 30, 15 to 28, 17 to 22, 17 to 25, 19 to 27, 19 to 30, 20 to 40, 22 to 40, 25 to 40 or 32 to 40) nucleotides in length. In some embodiments, an RNAi oligonucleotide conjugate herein comprises an antisense of 15 to 30 nucleotides in length. In some embodiments, an antisense strand of any one of the RNAi oligonucleotide conjugates disclosed herein is of 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length. In some embodiments, an RNAi oligonucleotide conjugate comprises an antisense strand of 22 nucleotides in length.

### Sense Strands

In some embodiments, an RNAi oligonucleotide conjugate disclosed herein comprises a sense strand (or passenger strand) of up to about 50 nucleotides in length (e.g., up to 50, up to 40, up to 36, up to 30, up to 27, up to 25, up to 21, up to 19, up to 17 or up to 12 nucleotides in length). In some embodiments, an RNAi oligonucleotide conjugate herein comprises a sense strand of at least about 12 nucleotides in length (e.g., at least 12, at least 15, at least 19, at least 21, at least 25, at least 27, at least 30, at least 36 or at least 38 nucleotides in length). In some embodiments, an RNAi oligonucleotide conjugate herein comprises a sense strand in a range of about 12 to about 50 (e.g., 12 to 50, 12 to 40, 12 to 36, 12 to 32, 12 to 28, 15 to 40, 15 to 36, 15 to 32, 15 to 28, 17 to 21, 17 to 25, 19 to 27, 19 to 30, 20 to 40, 22 to 40, 25 to 40 or 32 to 40) nucleotides in length. In some embodiments, an RNAi oligonucleotide conjugate herein comprises a sense strand 15 to 50 nucleotides in length. In some embodiments, an RNAi oligonucleotide conjugate herein comprises a sense strand 18 to 36 nucleotides in length. In some embodiments, an RNAi oligonucleotide conjugate herein comprises a sense strand of 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, an RNAi oligonucleotide conjugate herein comprises a sense strand of 36 nucleotides in length.

In some embodiments, a sense strand comprises a stem-loop structure at its 3′ end. In some embodiments, the stem-loop is formed by intrastrand base pairing. In some embodiments, a sense strand comprises a stem-loop structure at its 5′ end. In some embodiments, a stem is a duplex of 2, 3, 4, 5, 6, 7, 8, 9, 10, 11, 12, 13 or 14 nucleotides in length. In some embodiments, a stem-loop provides the RNAi oligonucleotide conjugate protection against degradation (e.g., enzymatic degradation), facilitates or improves targeting and/or delivery to a target cell, tissue, or organ (e.g., the liver), or both. For example, in some embodiments, the loop of a stem-loop provides nucleotides comprising one or more modifications that facilitate, improve, or increase targeting to a target mRNA (e.g., a target mRNA expressed in the CNS), inhibition of target gene expression, and/or delivery to a target cell, tissue, or organ (e.g., the CNS), or a combination thereof. In some embodiments, the stem-loop itself or modification(s) to the stem-loop do not substantially affect the inherent gene expression inhibition activity of the RNAi oligonucleotide conjugate, but facilitates, improves, or increases stability (e.g., provides protection against degradation) and/or delivery of the RNAi oligonucleotide conjugate to a target cell, tissue, or organ (e.g., the CNS). In certain embodiments, an RNAi oligonucleotide conjugate herein comprises a sense strand comprising (e.g., at its 3′ end) a stem-loop set forth as: S1-L-S2, in which S1 is complementary to S2, and in which L forms a single-stranded loop between S1 and S2 of up to about 10 nucleotides in length (e.g., 3, 4, 5, 6, 7, 8, 9 or 10 nucleotides in length). In some embodiments, the loop (L) is 3 nucleotides in length. In some embodiments, the loop (L) is 4 nucleotides in length.

In some embodiments, the tetraloop comprises the sequence 5′-GAAA-3′. In some embodiments, the stem loop comprises the sequence 5′-GCAGCCGAAAGGCUGC-3′ (SEQ ID NO: 15).

In some embodiments, a loop (L) of a stem-loop having the structure S1-L-S2 as described above is a triloop. In some embodiments, the triloop comprises ribonucleotides, deoxyribonucleotides, modified nucleotides, delivery ligands, and combinations thereof.

In some embodiments, a loop (L) of a stem-loop having the structure S1-L-S2 as described above is a tetraloop (e.g., within a nicked tetraloop structure). In some embodiments, the tetraloop comprises ribonucleotides, deoxyribonucleotides, modified nucleotides, delivery ligands, and combinations thereof.

In some embodiments, a loop (L) of a stem-loop having the structure S1-L-S2 as described above is a tetraloop as described in U.S. Pat. No. 10,131,912, incorporated herein by reference (e.g., within a nicked tetraloop structure).

### Duplex Length

In some embodiments, a duplex formed between a sense and antisense strand is at least 12 (e.g., at least 15, at least 16, at least 17, at least 18, at least 19, at least 20, or at least 21) nucleotides in length. In some embodiments, a duplex formed between a sense and antisense strand is in the range of 12-30 nucleotides in length (e.g., 12 to 30, 12 to 27, 12 to 22, 15 to 25, 18 to 30, 18 to 22, 18 to 25, 18 to 27, 18 to 30, 19 to 30 or 21 to 30 nucleotides in length). In some embodiments, a duplex formed between a sense and antisense strand is 12, 13, 14, 15, 16, 17, 18, 19, 29, 21, 22, 23, 24, 25, 26, 27, 28, 29 or 30 nucleotides in length. In some embodiments, a duplex formed between a sense and antisense strand does not span the entire length of the sense strand and/or antisense strand. In some embodiments, a duplex between a sense and antisense strand spans the entire length of either the sense or antisense strands. In some embodiments, a duplex between a sense and antisense strand spans the entire length of both the sense strand and the antisense strand.

### Oligonucleotide Ends

In some embodiments, an RNAi oligonucleotide conjugate disclosed herein comprises sense and antisense strands, such that there is a 3′-overhang on either the sense strand or the antisense strand, or both the sense and antisense strand. In some embodiments, an RNAi oligonucleotide conjugate herein has one 5′end that is thermodynamically less stable compared to the other 5′ end. In some embodiments, an asymmetric RNAi oligonucleotide conjugate is provided that includes a blunt end at the 3′end of a sense strand and overhang at the 3′ end of the antisense strand. In some embodiments, a 3′ overhang on an antisense strand is 1-8 nucleotides in length (e.g., 1, 2, 3, 4, 5, 6, 7 or 8 nucleotides in length).

Typically, an oligonucleotide for RNAi has a two (2) nucleotide overhand on the 3′ end of the antisense (guide) strand. However, other overhangs are possible. In some embodiments, an overhang is a 3′ overhang comprising a length of between one and six nucleotides, optionally one to five, one to four, one to three, one to two, two to six, two to five, two to four, two to three, three to six, three to five, three to four, four to six, four to five, five to six nucleotides or one, two, three, four, five or six nucleotides. In some embodiments, the overhang is a 5′ overhang comprising a length of between one and six nucleotides, optionally one to five, one to four, one to three, one to two, two to six, two to five, two to four, two to three, three to six, three to five, three to four, four to six, four to five, five to six nucleotides or one, two, three, four, five or six nucleotides.

In some embodiments, one or more (e.g., 2, 3, or 4) terminal nucleotides of the 3′ end or 5′ end of a sense and/or antisense strand are modified. For example, in some embodiments, one or two terminal nucleotides of the 3′ end of the antisense strand are modified. In some embodiments, the last nucleotide at the 3′ end of an antisense strand is modified, e.g., comprises 2′ modification, e.g., a 2′-O-methoxyethyl. In some embodiments, the last one or two terminal nucleotides at the 3′ end of an antisense strand are complementary with the target. In some embodiments, the last one or two nucleotides at the 3′ end of the antisense strand are not complementary with the target.

In some embodiments, an RNAi oligonucleotide conjugate disclosed herein comprises a stem-loop structure at the 3′ end of the sense strand and comprises two terminal overhang nucleotides at the 3′ end of the antisense strand. In some embodiments, an RNAi oligonucleotide conjugate herein comprises a nicked tetraloop structure, wherein the 3′ end of the sense strand comprises a stem-tetraloop structure and comprises two terminal overhang nucleotides at the 3′ end of the antisense strand. In some embodiments, the two terminal overhang nucleotides are GG. Typically, one or both of the two terminal GG nucleotides of the antisense strand are not complementary with the target.

In some embodiments, the 5′ end and/or the 3′end of a sense or antisense strand has an inverted cap nucleotide.

In some embodiments, one or more (e.g., 2, 3, 4, 5, 6) modified internucleotide linkages are provided between terminal nucleotides of the 3′ end or 5′ end of a sense and/or antisense strand. In some embodiments, modified internucleotide linkages are provided between overhang nucleotides at the 3′ end or 5′ end of a sense and/or antisense strand.

### Oligonucleotide Modifications

In some embodiments, an RNAi oligonucleotide conjugate disclosed herein comprises one or more modifications. Oligonucleotides (e.g., RNAi oligonucleotides) may be modified in various ways to improve or control specificity, stability, delivery, bioavailability, resistance from nuclease degradation, immunogenicity, base-pairing properties, RNA distribution and cellular uptake and other features relevant to therapeutic research use.

In some embodiments, the modification is a modified sugar. In some embodiments, the modification is a 5′-terminal phosphate group. In some embodiments, the modification is a modified internucleoside linkage. In some embodiments, the modification is a modified base. In some embodiments, the modification is a reversible modification. In some embodiments, an oligonucleotide described herein can comprise any one of the modifications described herein or any combination thereof. For example, in some embodiments, an oligonucleotide described herein comprises at least one modified sugar, a 5′-terminal phosphate group, at least one modified internucleoside linkage, at least one modified base, and at least one reversible modification.

The number of modifications on an oligonucleotide (e.g., an RNAi oligonucleotide) and the position of those nucleotide modifications may influence the properties of an oligonucleotide. For example, oligonucleotides may be delivered in vivo by conjugating them to or encompassing them in a lipid nanoparticle (LNP) or similar carrier. However, when an oligonucleotide is not protected by an LNP or similar carrier, it may be advantageous for at least some of the nucleotides to be modified. Accordingly, in some embodiments, all or substantially all of the nucleotides of an oligonucleotides are modified. In some embodiments, more than half of the nucleotides are modified. In some embodiments, less than half of the nucleotides are modified. In some embodiments, the sugar moiety of all nucleotides comprising the oligonucleotide is modified at the 2′ position. The modifications may be reversible or irreversible. In some embodiments, an oligonucleotide as disclosed herein has a number and type of modified nucleotides sufficient to cause the desired characteristics (e.g., protection from enzymatic degradation, capacity to target a desired cell after in vivo administration, and/or thermodynamic stability).

### Sugar Modifications

In some embodiments, an RNAi oligonucleotide conjugate described herein comprises a modified sugar. In some embodiments, a modified sugar (also referred herein to a sugar analog) includes a modified deoxyribose or ribose moiety in which, for example, one or more modifications occur at the 2′, 3′, 4′ and/or 5′ carbon position of the sugar. In some embodiments, a modified sugar may also include non-natural alternative carbon structures such as those present in locked nucleic acids (“LNA”; see, e.g., Koshkin et al. (1998) TETRAHEDON 54:3607-30), unlocked nucleic acids (“UNA”; see, e.g., Snead et al. (2013) MOL. THER-NUCL. ACIDS 2:e103) and bridged nucleic acids (“BNA”; see, e.g., Imanishi & Obika (2002) CHEM COMMUN. (CAMB) 21:1653-59).

In some embodiments, a nucleotide modification in a sugar comprises a 2′-modification. In some embodiments, a 2′-modification may be 2′-O-propargyl, 2′-O-propylamin, 2′-amino, 2′-ethyl, 2′-fluoro (2′-F), 2′-aminoethyl (EA), 2′-O-methyl (2′-OMe), 2′-O-methoxyethyl (2′-MOE), 2′-O-[2-(methylamino)-2-oxoethyl] (2′-O-NMA) or 2′-deoxy-2′-fluoro-β-d-arabinonucleic acid (2′-FANA). In some embodiments, the modification is 2′-F, 2′-OMe or 2′-MOE. In some embodiments, a modification in a sugar comprises a modification of the sugar ring, which may comprise modification of one or more carbons of the sugar ring. For example, a modification of a sugar of a nucleotide may comprise a 2′-oxygen of a sugar is linked to a 1′-carbon or 4′-carbon of the sugar, or a 2′-oxygen is linked to the 1′-carbon or 4′-carbon via an ethylene or methylene bridge. In some embodiments, a modified nucleotide has an acyclic sugar that lacks a 2′-carbon to 3′-carbon bond. In some embodiments, a modified nucleotide has a thiol group, e.g., in the 4′ position of the sugar.

In some embodiments, an RNAi oligonucleotide conjugate described herein comprises at least about 1 modified nucleotide (e.g., at least 1, at least 5, at least 10, at least 15, at least 20, at least 25, at least 30, at least 35, at least 40, at least 45, at least 50, at least 55, at least 60, or more). In some embodiments, the sense strand of the RNAi oligonucleotide conjugate comprises at least about 1 modified nucleotide (e.g., at least 1, at least 5, at least 10, at least 15, at least 20, at least 25, at least 30, at least 35, or more). In some embodiments, the antisense strand of the RNAi oligonucleotide conjugate comprises at least about 1 modified nucleotide (e.g., at least 1, at least 5, at least 10, at least 15, at least 20, or more).

In some embodiments, all the nucleotides of the sense strand of the RNAi oligonucleotide conjugate are modified. In some embodiments, all the nucleotides of the antisense strand of the RNAi oligonucleotide conjugate are modified. In some embodiments, all the nucleotides of the RNAi oligonucleotide conjugate (i.e., both the sense strand and the antisense strand) are modified. In some embodiments, the modified nucleotide comprises a 2′-modification (e.g., a 2′-F or 2′-OMe, 2′-MOE, and 2′-deoxy-2′-fluoro-β-d-arabinonucleic acid).

In some embodiments, the disclosure provides RNAi oligonucleotide conjugates having different modification patterns. In some embodiments, the modified RNAi oligonucleotide conjugates comprise a sense strand sequence having a modification pattern as set forth in the Examples and Sequence Listing and an antisense strand having a modification pattern as set forth in the Examples and Sequence Listing.

In some embodiments, an RNAi oligonucleotide conjugate disclosed herein comprises an antisense strand having nucleotides that are modified with 2′-F. In some embodiments, an RNAi oligonucleotide conjugate disclosed herein comprises an antisense strand comprises nucleotides that are modified with 2′-F and 2′-OMe. In some embodiments, an RNAi oligonucleotide conjugate disclosed herein comprises a sense strand having nucleotides that are modified with 2′-F. In some embodiments, an RNAi oligonucleotide conjugate disclosed herein comprises a sense strand comprises nucleotides that are modified with 2′-F and 2′-OMe.

In some embodiments, an oligonucleotide described herein comprises a sense strand with about 10-25%, 10%, 11%, 12%, 13%, 14% 15%, 16%, 17%, 18%, 19% or 20% of the nucleotides of the sense strand comprising a 2′-fluoro modification. In some embodiments, about 11% of the nucleotides of the sense strand comprise a 2-fluoro modification. In some embodiments, about 20% of the nucleotides of the sense strand comprise a 2-fluoro modification. In some embodiments, an oligonucleotide described herein comprises an antisense strand with about 25-35%, 25%, 26%, 27%, 28%, 29%, 30%, 31%, 32%, 33%, 34% or 35% of the nucleotides of the antisense strand comprising a 2′-fluoro modification. In some embodiments, about 32% of the nucleotides of the antisense strand comprise a 2′-fluoro modification. In some embodiments, the oligonucleotide has about 15-25%, 15%, 16%, 17%, 18%, 19%, 20%, 21%, 22%, 23%, 24%, or 25% of its nucleotides comprising a 2′-fluoro modification. In some embodiments, about 19% of the nucleotides in the oligonucleotide comprise a 2′-fluoro modification. In some embodiments, about 26% of the nucleotides in the oligonucleotide comprise a 2′-fluoro modification.

In some embodiments, for these oligonucleotides, one or more of positions 8, 9, 10 or 11 of the sense strand is modified with a 2′-F group. In some embodiments, for these oligonucleotides, the sugar moiety at each of nucleotides at positions 1-7 and 12-20 in the sense strand is modified with a 2′-OMe. In some embodiments, for these oligonucleotides, the sugar moiety at each of nucleotides at positions 1-7, 12-27, and 29-36 in the sense strand is modified with a 2′-OMe.

In some embodiments, for these oligonucleotides, one or more of positions 3, 5, 8, 10, 12, 13, 15, and 17 of the sense strand is modified with a 2′-F group. In some embodiments, for these oligonucleotides, the sugar moiety at each of nucleotides at positions 1, 2, 4, 6, 7, 9, 11, 14, 16, 18-27 and 29-36 in the sense strand is modified with a 2′-OMe.

In some embodiments, the antisense strand has 3 nucleotides that are modified at the 2′-position of the sugar moiety with a 2′-F. In some embodiments, the sugar moiety at nucleotides at positions 2, 5 and 14 and optionally up to 3 of the nucleotides at positions 1, 3, 7 and 10 of the antisense strand are modified with a 2′-F. In other embodiments, the sugar moiety at each of the nucleotides at positions 2, 5 and 14 of the antisense strand is modified with the 2′-F. In other embodiments, the sugar moiety at each of the nucleotides at positions 1, 2, 5 and 14 of the antisense strand is modified with the 2′-F. In still other embodiments, the sugar moiety at each of the nucleotides at positions 1, 2, 3, 5, 7 and 14 of the antisense strand is modified with the 2′-F. In yet another embodiment, the sugar moiety at each of the nucleotides at positions 1, 2, 3, 5, 10 and 14 of the antisense strand is modified with the 2′-F. In another embodiment, the sugar moiety at each of the nucleotides at positions 2, 3, 5, 7, 10 and 14 of the antisense strand is modified with the 2′-F. In some embodiments, the antisense strand has 9 nucleotides that are modified at the 2′-position of the sugar moiety with a 2′-F. In certain additional embodiments, the sugar moiety at each of the nucleotides at positions 2, 3, 4, 5, 7, 10, 14, 16 and 19 of the antisense strand is modified with the 2′-F.

In some embodiments, an RNAi oligonucleotide conjugate provided herein comprises an antisense strand having the sugar moiety at positions 2 and 14 modified with 2′-F. In some embodiments, an RNAi oligonucleotide conjugate provided herein comprises an antisense strand having the sugar moiety at positions 2, 5, and 14 modified with 2′-F. In some embodiments, an RNAi oligonucleotide conjugate provided herein comprises an antisense strand having the sugar moiety at positions 1, 2, 5, and 14 modified with 2′-F. In some embodiments, an RNAi oligonucleotide conjugate provided herein comprises an antisense strand having the sugar moiety at positions 1, 2, 3, 5, 7, and 14 modified with 2′-F. In some embodiments, an RNAi oligonucleotide conjugate provided herein comprises an antisense strand having the sugar moiety at positions 1, 2, 3, 5, 10, and 14 modified with 2′-F. In some embodiments, an RNAi oligonucleotide conjugate provided herein comprises an antisense strand having the sugar moiety at positions 2, 3, 4, 5, 7, 10, 14, 16 and 19 modified with 2′-F.

In some embodiments, an RNAi oligonucleotide conjugate provided herein comprises an antisense strand having the sugar moiety of each of the nucleotides at positions 2, 5, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an RNAi oligonucleotide conjugate provided herein comprises an antisense strand having the sugar moiety of each of the nucleotides at positions 2, 3, 4, 5, 7, 10, 14, 16 and 19 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an RNAi oligonucleotide conjugate provided herein comprises an antisense strand having the sugar moiety of each of the nucleotides at positions 1, 2, 5, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an RNAi oligonucleotide conjugate provided herein comprises an antisense strand having the sugar moiety of each of the nucleotides at positions 1, 2, 3, 5, 7, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an RNAi oligonucleotide conjugate provided herein comprises an antisense strand having the sugar moiety of each of the nucleotides at positions 1, 2, 3, 5, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an RNAi oligonucleotide conjugate provided herein comprises an antisense strand having the sugar moiety of each of the nucleotides at positions 2, 3, 5, 7, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an RNAi oligonucleotide conjugate provided herein comprises an antisense strand having the sugar moiety of each of the nucleotides at positions 2, 3, 4, 5, 7, 10, 14, 16 and 19 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an RNAi oligonucleotide conjugate provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with 2′-F.

In some embodiments, an RNAi oligonucleotide conjugate provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with 2′-OMe.

In some embodiments, an RNAi oligonucleotide conjugate provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an RNAi oligonucleotide conjugate provided herein comprises a sense strand having the sugar moiety at positions 8-11 modified with 2′-F. In some embodiments, an RNAi oligonucleotide conjugate provided herein comprises a sense strand having the sugar moiety at positions 3, 5, 8, 10, 12, 13, 15 and 17 modified with 2′-F. In some embodiments, an RNAi oligonucleotide conjugate provided herein comprises a sense strand having the sugar moiety at positions 1-7 and 12-17 or 12-20 modified with 2′OMe. In some embodiments, an RNAi oligonucleotide conjugate provided herein comprises a sense strand having the sugar moiety at positions 1, 2, 4, 6, 7, 9, 11, 14, 16 and 18-20 modified with 2′OMe. In some embodiments, an RNAi oligonucleotide conjugate provided herein comprises a sense strand having the sugar moiety of each of the nucleotides at positions 1-7 and 12-17 or 12-20 of the sense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA). In some embodiments, an RNAi oligonucleotide conjugate provided herein comprises a sense strand having the sugar moiety at positions 1, 2, 4, 6, 7, 9, 11, 14, 16 and 18-20 of the sense strand modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

In some embodiments, an RNAi oligonucleotide conjugate provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 modified with 2′-F.

In some embodiments, an RNAi oligonucleotide conjugate provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 modified with 2′-OMe.

In some embodiments, an RNAi oligonucleotide conjugate provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 modified with a modification selected from the group consisting of 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA).

### 5′-Terminal Phosphate

In some embodiments, an RNAi oligonucleotide conjugate described herein comprises a 5′-terminal phosphate. In some embodiments, the 5′-terminal phosphate groups of the RNAi oligonucleotide conjugate enhance the interaction with Ago2. However, oligonucleotides comprising a 5′-phosphate group may be susceptible to degradation via phosphatases or other enzymes, which can limit their bioavailability in vivo. In some embodiments, an RNAi oligonucleotide conjugate herein comprises analogs of 5′ phosphates that are resistant to such degradation. In some embodiments, the phosphate analog is oxymethylphosphonate, vinylphosphonate or malonylphosphonate, or a combination thereof. In certain embodiments, the 5′ end of an RNAi oligonucleotide conjugate strand is attached to chemical moiety that mimics the electrostatic and steric properties of a natural 5′-phosphate group (“phosphate mimic”).

In some embodiments, an RNAi oligonucleotide conjugate herein has a phosphate analog at a 4′-carbon position of the sugar (referred to as a “4′-phosphate analog”). See, e.g., Intl. Patent Application Publication No. WO 2018/045317. In some embodiments, an RNAi oligonucleotide conjugate herein comprises a 4′-phosphate analog at a 5′-terminal nucleotide. In some embodiments, a phosphate analog is an oxymethylphosphonate, in which the oxygen atom of the oxymethyl group is bound to the sugar moiety (e.g., at its 4′-carbon) or analog thereof. In other embodiments, a 4′-phosphate analog is a thiomethylphosphonate or an aminomethylphosphonate, in which the sulfur atom of the thiomethyl group or the nitrogen atom of the amino methyl group is bound to the 4′-carbon of the sugar moiety or analog thereof. In certain embodiments, a 4′-phosphate analog is an oxymethylphosphonate. In some embodiments, an oxymethylphosphonate is represented by the formula —O—CH₂—PO(OH)₂, —O—CH₂—PO(OR)₂, or —O—CH₂—POOH(R), in which R is independently selected from H, CH₃, an alkyl group, CH₂CH₂CN, CH₂OCOC(CH₃)₃, CH₂OCH₂CH₂Si (CH₃)₃ or a protecting group. In certain embodiments, the alkyl group is CH₂CH₃. More typically, R is independently selected from H, CH₃ or CH₂CH₃. In some embodiment, R is CH₃. In some embodiments, the 4′-phosphate analog is 5′-methoxyphosphonate-4′-oxy. In some embodiments, the 4′-phosphate analog is 4′-oxymethylphosphonate.

In some embodiments, an RNAi oligonucleotide conjugate provided herein comprises an antisense strand comprising a 4′-phosphate analog at the 5′-terminal nucleotide, wherein 5′-terminal nucleotide comprises the following structure:

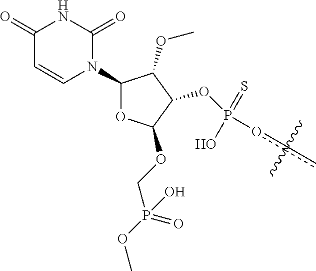

4′-O-monomethylphosphonate-2′-O-methyluridine phosphorothioate

### [MePhosphonate-4O-mUs]

### Modified Internucleoside Linkage

In some embodiments, an RNAi oligonucleotide conjugate herein comprises a modified internucleoside linkage. In some embodiments, phosphate modifications or substitutions result in an oligonucleotide that comprises at least about 1 (e.g., at least 1, at least 2, at least 3 or at least 5) modified internucleotide linkage. In some embodiments, any one of the oligonucleotides disclosed herein comprises about 1 to about 10 (e.g., 1 to 10, 2 to 8, 4 to 6, 3 to 10, 5 to 10, 1 to 5, 1 to 3 or 1 to 2) modified internucleotide linkages. In some embodiments, any one of the oligonucleotides disclosed herein comprises 1, 2, 3, 4, 5, 6, 7, 8, 9 or 10 modified internucleotide linkages.

A modified internucleotide linkage may be a phosphorodithioate linkage, a phosphorothioate linkage, a phosphotriester linkage, a thionoalkylphosphonate linkage, a thionalkylphosphotriester linkage, a phosphoramidite linkage, a phosphonate linkage or a boranophosphate linkage. In some embodiments, at least one modified internucleotide linkage of any one of the oligonucleotides as disclosed herein is a phosphorothioate linkage.

In some embodiments, an RNAi oligonucleotide conjugate provided herein has a phosphorothioate linkage between one or more of positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand. In some embodiments, the oligonucleotide described herein has a phosphorothioate linkage between each of positions 1 and 2 of the sense strand, positions 1 and 2 of the antisense strand, positions 2 and 3 of the antisense strand, positions 20 and 21 of the antisense strand, and positions 21 and 22 of the antisense strand.

In some embodiments, an oligonucleotide conjugate described herein comprises a peptide nucleic acid (PNA). PNAs are oligonucleotide mimics in which the sugar-phosphate backbone has been replaced by a pseudopeptide skeleton, composed of N-(2-aminoethyl)glycine units. Nucleobases are linked to this skeleton through a two-atom carboxymethyl spacer. In some embodiments, an oligonucleotide conjugate described herein comprises a morpholino oligomer (PMO) comprising an internucleotide linkage backbone of methylene morpholine rings linked through phosphorodiamidate groups.

### Base Modifications

In some embodiments, an RNAi oligonucleotide conjugate herein comprises one or more modified nucleobases. In some embodiments, modified nucleobases (also referred to herein as base analogs) are linked at the 1′ position of a nucleotide sugar moiety. In certain embodiments, a modified nucleobase is a nitrogenous base. In certain embodiments, a modified nucleobase does not contain nitrogen atom. See, e.g., US Patent Application Publication No. 2008/0274462. In some embodiments, a modified nucleotide comprises a universal base. In some embodiments, a modified nucleotide does not contain a nucleobase (abasic).

In some embodiments, a universal base is a heterocyclic moiety located at the 1′ position of a nucleotide sugar moiety in a modified nucleotide, or the equivalent position in a nucleotide sugar moiety substitution, that, when present in a duplex, can be positioned opposite more than one type of base without substantially altering structure of the duplex. In some embodiments, compared to a reference single-stranded nucleic acid (e.g., oligonucleotide) that is fully complementary to a target nucleic acid, a single-stranded nucleic acid containing a universal base forms a duplex with the target nucleic acid that has a lower Tₘ than a duplex formed with the complementary nucleic acid. In some embodiments, when compared to a reference single-stranded nucleic acid in which the universal base has been replaced with a base to generate a single mismatch, the single-stranded nucleic acid containing the universal base forms a duplex with the target nucleic acid that has a higher Tₘ than a duplex formed with the nucleic acid comprising the mismatched base.

Non-limiting examples of universal-binding nucleotides include, but are not limited to, inosine, 1-β-D-ribofuranosyl-5-nitroindole and/or 1-β-D-ribofuranosyl-3-nitropyrrole (see, US Patent Application Publication No. 2007/0254362; Van Aerschot et al. (1995) NUCLEIC ACIDS RES. 23:4363-4370; Loakes et al. (1995) NUCLEIC ACIDS RES. 23:2361-66; and Loakes & Brown (1994) NUCLEIC ACIDS RES. 22:4039-43).

### Targeting Ligands

In some embodiments, it is desirable to target the oligonucleotides of the disclosure (e.g., RNAi oligonucleotide conjugates) to one or more cells or one or more organs. Such a strategy can help to avoid undesirable effects in other organs or avoid undue loss of the oligonucleotide to cells, tissue or organs that would not benefit from the oligonucleotide. Accordingly, in some embodiments, an RNAi oligonucleotide conjugate disclosed herein is modified to facilitate targeting and/or delivery to a particular tissue, cell, or organ (e.g., to facilitate delivery of the conjugate to the CNS). In some embodiments, an RNAi oligonucleotide conjugate comprises at least one nucleotide (e.g., 1, 2, 3, 4, 5, 6 or more nucleotides) conjugated to one or more targeting ligand(s).

In some embodiments, the targeting ligand comprises a carbohydrate, amino sugar, cholesterol, peptide, polypeptide, protein, or part of a protein (e.g., an antibody or antibody fragment), or lipid. In some embodiments, the targeting ligand is an aptamer. For example, a targeting ligand may be an RGD peptide that is used to target tumor vasculature or glioma cells, CREKA peptide to target tumor vasculature or stoma, transferring, lactoferrin, or an aptamer to target transferrin receptors expressed on CNS vasculature, or an anti-EGFR antibody to target EGFR on glioma cells. In certain embodiments, the targeting ligand is one or more GalNAc moieties.

In some embodiments, 1 or more (e.g., 1, 2, 3, 4, 5 or 6) nucleotides of an RNAi oligonucleotide conjugate disclosed herein are each conjugated to a separate targeting ligand. In some embodiments, 2 to 4 nucleotides of an RNAi oligonucleotide conjugate herein are each conjugated to a separate targeting ligand. In some embodiments, targeting ligands are conjugated to 2 to 4 nucleotides at either ends of the sense or antisense strand (e.g., targeting ligands are conjugated to a 2 to 4 nucleotide overhang or extension on the 5′ or 3′ end of the sense or antisense strand) such that the targeting ligands resemble bristles of a toothbrush and the RNAi oligonucleotide conjugate resembles a toothbrush. For example, an RNAi oligonucleotide conjugate may comprise a stem-loop at either the 5′ or 3′ end of the sense strand and 1, 2, 3 or 4 nucleotides of the loop of the stem may be individually conjugated to a targeting ligand. In some embodiments, an RNAi oligonucleotide conjugate provided by the disclosure comprises a stem-loop at the 3′ end of the sense strand, wherein the loop of the stem-loop comprises a triloop or a tetraloop, and wherein the 3 or 4 nucleotides comprising the triloop or tetraloop, respectfully, are individually conjugated to a targeting ligand.

GalNAc is a high affinity ligand for the ASGPR, which is primarily expressed on the sinusoidal surface of hepatocyte cells and has a major role in binding, internalizing and subsequent clearing circulating glycoproteins that contain terminal galactose or GalNAc residues (asialoglycoproteins). Conjugation (either indirect or direct) of GalNAc moieties to oligonucleotide of the instant disclosure can be used to target these oligonucleotides to the ASGPR expressed on cells. In some embodiments, an oligonucleotide of the instant disclosure is conjugated to at least one or more GalNAc moieties, wherein the GalNAc moieties target the oligonucleotide to an ASGPR expressed on human liver cells (e.g., human hepatocytes). In some embodiments, the GalNAc moiety target the oligonucleotide to the liver.

In some embodiments, an oligonucleotide of the instant disclosure is conjugated directly or indirectly to a monovalent GalNAc. In some embodiments, the oligonucleotide is conjugated directly or indirectly to more than one monovalent GalNAc (i.e., is conjugated to 2, 3 or 4 monovalent GalNAc moieties, and is typically conjugated to 3 or 4 monovalent GalNAc moieties). In some embodiments, an oligonucleotide is conjugated to one or more bivalent GalNAc, trivalent GalNAc or tetravalent GalNAc moieties.

In some embodiments, 1 or more (e.g., 1, 2, 3, 4, 5 or 6) nucleotides of an oligonucleotide are each conjugated to a GalNAc moiety. In some embodiments, 2 to 4 nucleotides of a tetraloop are each conjugated to a separate GalNAc. In some embodiments, 1 to 3 nucleotides of a triloop are each conjugated to a separate GalNAc. In some embodiments, targeting ligands are conjugated to 2 to 4 nucleotides at either ends of the sense or antisense strand (e.g., ligands are conjugated to a 2 to 4 nucleotide overhang or extension on the 5′ or 3′ end of the sense or antisense strand) such that the GalNAc moieties resemble bristles of a toothbrush and the oligonucleotide resembles a toothbrush. In some embodiments, GalNAc moieties are conjugated to a nucleotide of the sense strand. For example, four (4) GalNAc moieties can be conjugated to nucleotides in the tetraloop of the sense strand where each GalNAc moiety is conjugated to 1 nucleotide.

In some embodiments, the tetraloop is any combination of adenine and guanine nucleotides.

In some embodiments, the tetraloop (L) has a monovalent GalNAc moiety attached to any one or more guanine nucleotides of the tetraloop via any linker described herein, as depicted below (X=heteroatom):

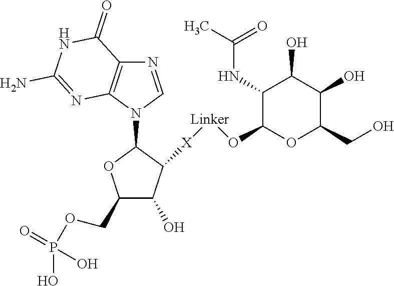

In some embodiments, the tetraloop (L) has a monovalent GalNAc attached to any one or more adenine nucleotides of the tetraloop via any linker described herein, as depicted below (X=heteroatom):

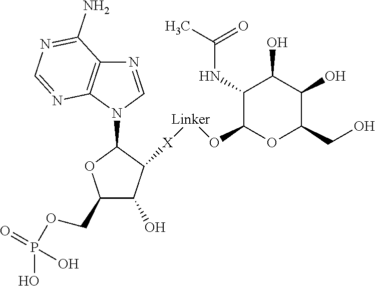

In some embodiments, an RNAi oligonucleotide conjugate herein comprises a monovalent GalNAc attached to a guanine nucleotide referred to as [ademG-GalNAc] or 2′-aminodiethoxymethanol-Guanine-GalNAc, as depicted below:

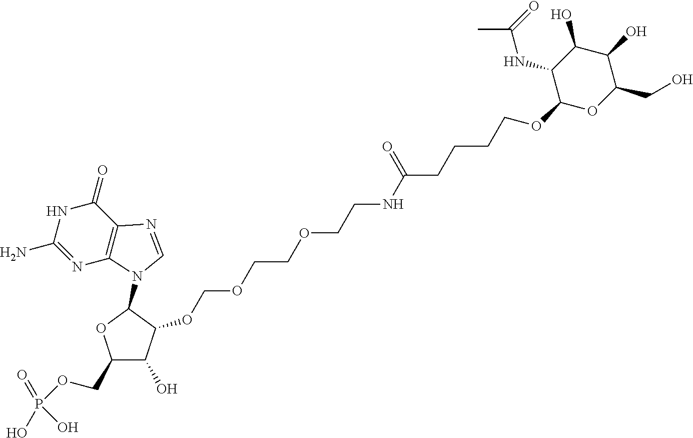 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(=O)[nH]c(N)nc32)O[C@H](CO)[C@H](O)[C@@H]1O]

In some embodiments, an RNAi oligonucleotide conjugate herein comprises a monovalent GalNAc attached to an adenine nucleotide, referred to as [ademA-GalNAc] or 2′-aminodiethoxymethanol-Adenine-GalNAc, as depicted below:

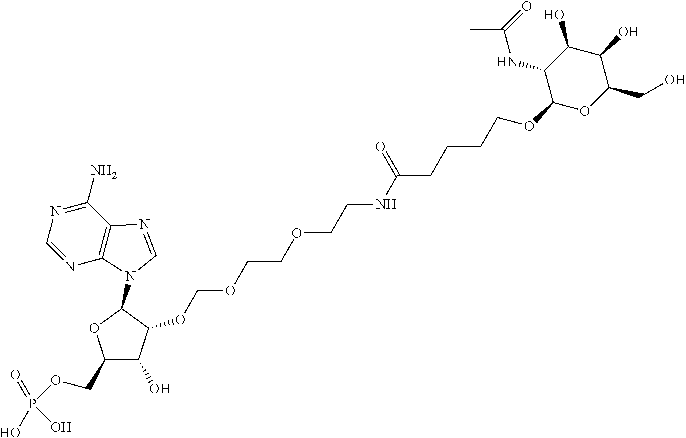 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

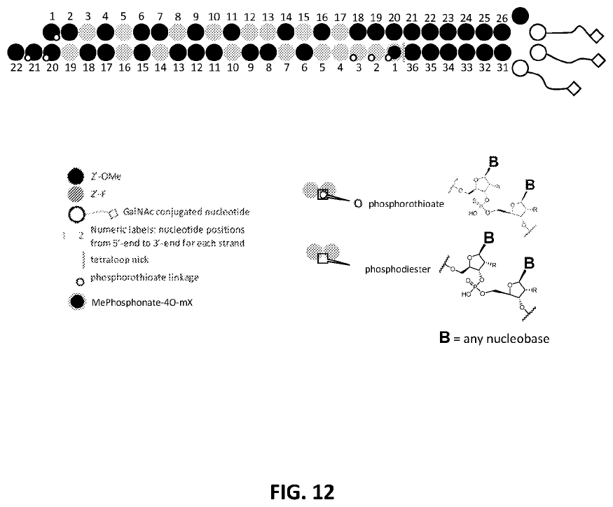

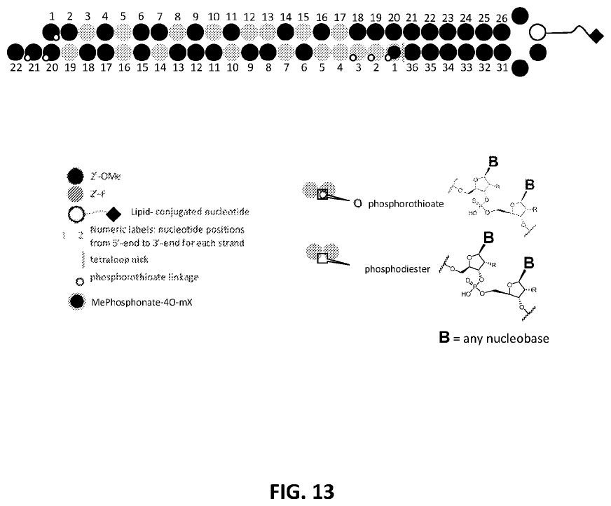

An example of such conjugation is shown below for a loop comprising from 5′ to 3′ the nucleotide sequence GAAA (L=linker, X=heteroatom). Such a loop may be present, for example, at positions 27-30 of the sense strand listed in Tables 3 or 5 and as shown in FIGS. 12-13. In the chemical formula, [symbol] is used to describe an attachment point to the oligonucleotide strand.

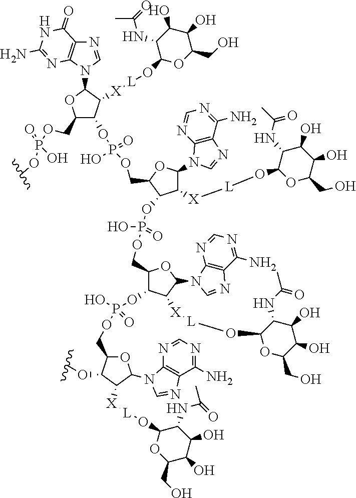

Appropriate methods or chemistry (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in Intl. Patent Application Publication No. WO 2016/100401. In some embodiments, the linker is a labile linker. However, in other embodiments, the linker is stable. An example is shown below for a loop comprising from 5′ to 3′ the nucleotides GAAA, in which GalNAc moieties are attached to nucleotides of the loop using an acetal linker. Such a loop may be present, for example, at positions 27-30 of the any one of the sense strand listed in Tables 3 or 5 and as shown in FIGS. 12-13. In the chemical formula, [symbol] is an attachment point to the oligonucleotide strand.

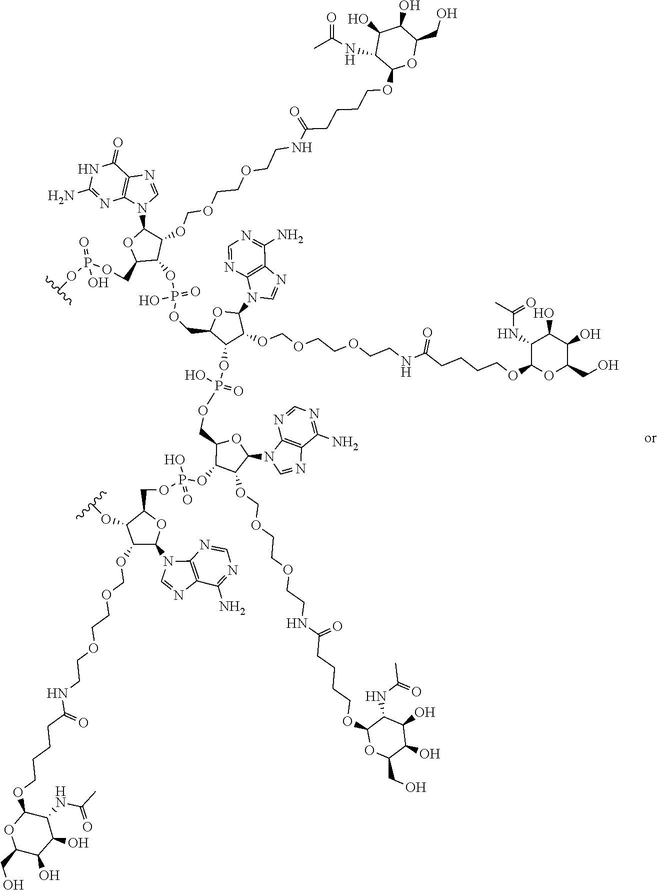 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]

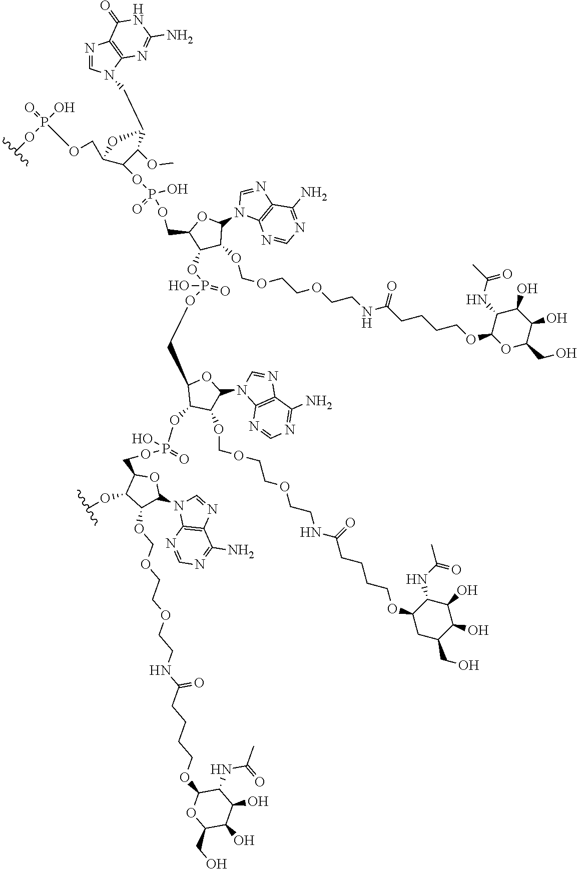

As mentioned, various appropriate methods or chemistry synthetic techniques (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in Intl. Patent Application Publication No. WO 2016/100401. In some embodiments, the linker is a labile linker. However, in other embodiments, the linker is a stable linker.

In some embodiments, a duplex extension (e.g., of up to 3, 4, 5 or 6 bp in length) is provided between a targeting ligand (e.g., a GalNAc moiety) and an RNAi oligonucleotide conjugate. In some embodiments, an RNAi oligonucleotide conjugate herein does not have a GalNAc conjugated thereto.

### Exemplary RNAi Oligonucleotide Conjugates

In some embodiments, the RNAi oligonucleotide conjugate comprises an oligonucleotide conjugated to one or more lipid moieties. In some embodiments, the one or more lipid moieties comprises a fatty acid. In some embodiments, the fatty acid is a saturated fatty acid. In some embodiments, the fatty acid is an unsaturated fatty acid. In some embodiments, the one or more lipid moieties comprises a hydrocarbon chain. In some embodiments, the hydrocarbon chain is saturated. In some embodiments, the hydrocarbon chain is unsaturated. In some embodiments, the one or more lipid moieties comprises a hydrocarbon chain of 50-carbons (C50) in length or shorter (e.g., about 50-carbons (C50), 40-carbons (C40), 30-carbons (C30), 25-carbons (C25), 22-carbons (C22), 20-carbons (C20), 18-carbons (C18), 16-carbons (C16), 14-carbons (C14), 12-carbons (C12), 10-carbons (C10), 8-carbons (C8), or 6-carbons (C6). In some embodiments, the one or more lipid moieties comprises a hydrocarbon chain of 16-carbons to 50-carbons (e.g., C16-C50, C18-C50, C20-C50, C22-C50, C16, C18, C20, or C22). In some embodiments, the one or more lipid moieties comprises a hydrocarbon chain of 15-carbons or fewer (e.g., C6-C15, C6-C14, C6-C12, C6-C10, C8-C14, C8-C12, C8-C10, C10-C14, C10-C12, C15, C14, C13, C12, C11, C10, C9, C8, C7, or C6).

In some embodiments, the one or more lipid moieties comprises a C8 hydrocarbon chain. In some embodiments, the C8 hydrocarbon chain comprises at least one double bond. In some embodiments, the one or more lipid moieties comprises a C10 hydrocarbon chain. In some embodiments, the C10 hydrocarbon chain is unsaturated (i.e., C10:0). In some embodiments, the C10 hydrocarbon chain comprises at least one double bond. In some embodiments, the one or more lipid moieties comprises a C12 hydrocarbon chain. In some embodiments, the C12 hydrocarbon chain is unsaturated (i.e., C12:0). In some embodiments, the C12 hydrocarbon chain comprises at least one double bond. In some embodiments, the one or more lipid moieties comprises a C14 hydrocarbon chain. In some embodiments, the C14 hydrocarbon chain is unsaturated (i.e., C14:0). In some embodiments, the C14 hydrocarbon chain comprises at least one double bond. In some embodiments, the one or more lipid moieties comprises a C16 hydrocarbon chain. In some embodiments, the C16 hydrocarbon chain is unsaturated (i.e., C16:0). In some embodiments, the C16 hydrocarbon chain comprises at least one double bond. In some embodiments, the one or more lipid moieties comprises a C18 hydrocarbon chain. In some embodiments, the C18 hydrocarbon chain is unsaturated (i.e., C18:0). In some embodiments, the C18 hydrocarbon chain comprises at least one double bond (e.g., C18:1, C18:2, C18:3, C18:4, C18:5, or C18:6). In some embodiments, the C18 hydrocarbon chain comprises two double bonds (e.g., C18:2). In some embodiments, the one or more lipid moieties comprises a C22 hydrocarbon chain. In some embodiments, the C22 hydrocarbon chain comprises at least one double bond (e.g., C22:1, C22:2, C22:3, C22:4, C22:5, or C22:6). In some embodiments, the one or more lipid moieties comprises a C24 hydrocarbon chain. In some embodiments, the C24 hydrocarbon chain comprises at least one double bond (e.g., C24:1, C24:2, C24:3, C24:4, C24:5, or C24:6).

In some embodiments, the oligonucleotide of the RNAi oligonucleotide conjugate comprises a tetraloop wherein at least one nucleotide of the tetraloop is conjugated with a C8 lipid. In some embodiments, the second nucleotide of the tetraloop is conjugated with a C8 lipid. In some embodiments, the oligonucleotide of the RNAi oligonucleotide conjugate comprises a tetraloop wherein at least one nucleotide of the tetraloop is conjugated with a C10 lipid. In some embodiments, the second nucleotide of the tetraloop is conjugated with a C10 lipid. In some embodiments, the oligonucleotide of the RNAi oligonucleotide conjugate comprises a tetraloop wherein at least one nucleotide of the tetraloop is conjugated with a C12 lipid. In some embodiments, the second nucleotide of the tetraloop is conjugated with a C12 lipid. In some embodiments, the oligonucleotide of the RNAi oligonucleotide conjugate comprises a tetraloop wherein at least one nucleotide of the tetraloop is conjugated with a C14 lipid. In some embodiments, the second nucleotide of the tetraloop is conjugated with a C14 lipid. In some embodiments, the oligonucleotide of the RNAi oligonucleotide conjugate comprises a tetraloop wherein at least one nucleotide of the tetraloop is conjugated with a C16 lipid. In some embodiments, the second nucleotide of the tetraloop is conjugated with a C16 lipid. In some embodiments, the oligonucleotide of the RNAi oligonucleotide conjugate comprises a tetraloop wherein at least one nucleotide of the tetraloop is conjugated with a C18 lipid (e.g., C18:0, C18:1, or C18:2). In some embodiments, the second nucleotide of the tetraloop is conjugated with a C18 lipid (e.g., C18:0, C18:1, or C18:2). In some embodiments, the oligonucleotide of the RNAi oligonucleotide conjugate comprises a tetraloop wherein at least one nucleotide of the tetraloop is conjugated with a C22 lipid (e.g., C22:0, C22:1, C22:2, C22:3, C22:4, C22:5, or C22:6). In some embodiments, the second nucleotide of the tetraloop is conjugated with a C22 lipid (e.g., C22:0, C22:1, C22:2, C22:3, C22:4, C22:5, or C22:6). In some embodiments, the oligonucleotide of the RNAi oligonucleotide conjugate comprises a tetraloop wherein at least one nucleotide of the tetraloop is conjugated with a C24 lipid (e.g., C24:0, C24:1, C24:2, C24:3, C24:4, C24:5, or C24:6). In some embodiments, the second nucleotide of the tetraloop is conjugated with a C24 lipid (e.g., C24:0, C24:1, C24:2, C24:3, C24:4, C24:5, or C24:6).

In some embodiments, an RNAi oligonucleotide conjugate comprises a nucleotide sequence having at least one modified nucleoside. In some embodiments, an oligonucleotide-ligand conjugate comprises an antisense strand and a sense strand, wherein each strand comprises at least one modified nucleoside.

In some embodiments, the RNAi oligonucleotide conjugate is represented by the following formula, with modifications as shown in Table 1:
Sense Strand:
[mXs] [mX] [fX] [mX] [fX] [mX] [mX] [fX] [mX] [fX] [mX] [fX] [fX] [mX] [fX] [mX] [fX] [mX] [m X] [mX] [mX] [mX] [mX] [mX] [mX] [mX] [mX] [ademX-TL] [mX] [mX] [mX] [mX] [mX][mX] [mX] [mX]
Hybridized to
Antisense Strand: [MePhosphonate-4O-mXs] [fXs] [fXs] [fX] [fX] [mX] [fX] [mX] [mX][fX] [mX] [mX] [mX] [fX] [mX] [fX] [mX] [mX] [fX] [mXs] [mXs] [mX]
Or
Sense Strand:
[mXs] [mX] [fX] [mX] [fX] [mX] [mX] [fX] [mX] [fX] [mX] [fX] [fX] [mX] [fX] [mX] [fX] [mX] [m X] [mX] [mX] [mX] [mX] [mX] [mX] [mX] [mX] [ademX-C#] [mX] [mX] [mX] [mX][mX] [mX] [mX] [mX]
Hybridized to
Antisense Strand: [MePhosphonate-4O-mXs] [fXs] [fXs] [fX] [fX] [mX] [fX] [mX] [mX][fX] [mX] [mX] [mX] [fX] [mX] [fX] [mX] [mX] [fX] [mXs] [mXs] [mX]

TABLE 1
Modification Key

| | |
|---|---|
| [MePhosphonate- | 5′-methoxyphosphonate-4-oxy modified nucleotide |
| 4O-mX] | |
| TL | targeting ligand attached to a nucleotide |
| ademX-C# | Lipid conjugate attached to a nucleotide (e.g. C8-C22) |
| [mXs] | 2′-O-methyl modified nucleotide with a |
| | phosphorothioate linkage to the neighboring nucleotide |
| [fXs] | 2′-fluoro modified nucleotide with a phosphorothioate |
| | linkage to the neighboring nucleotide |
| [mX] | 2′-O-methyl modified nucleotide with phosphodiester |
| | linkages to neighboring nucleotides |
| [fX] | 2′-fluoro modified nucleotide with phosphodiester |
| | linkages to neighboring nucleotides |

In some embodiments, the oligonucleotide of the RNAi oligonucleotide conjugate is conjugated to a C8 lipid as shown in:

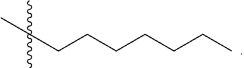 [SMILES: *CCCCCCC]

In some embodiments, the oligonucleotide of the RNAi oligonucleotide conjugate is conjugated to a C10 lipid as shown in:

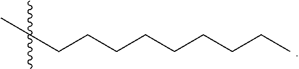 [SMILES: *CCCCCCCCC]

In some embodiments, the oligonucleotide of the RNAi oligonucleotide conjugate is conjugated to a C12 lipid as shown in:

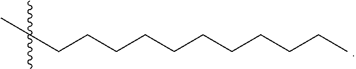 [SMILES: *CCCCCCCCCCC]

In some embodiments, the oligonucleotide of the RNAi oligonucleotide conjugate is conjugated to a C14 lipid as shown in:

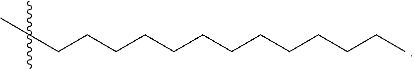 [SMILES: *CCCCCCCCCCCCC]

In some embodiments, the oligonucleotide of the RNAi oligonucleotide conjugate is conjugated to a C16 lipid as shown in:

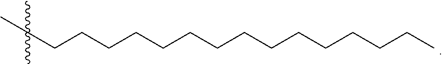 [SMILES: *CCCCCCCCCCCCCCC]

In some embodiments, the oligonucleotide of the RNAi oligonucleotide conjugate is conjugated to a C18:0 lipid as shown in:

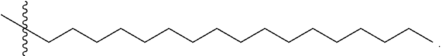 [SMILES: *CCCCCCCCCCCCCCCCC]

In some embodiments, the oligonucleotide of the RNAi oligonucleotide conjugate is conjugated to a C18:1 lipid as shown in:

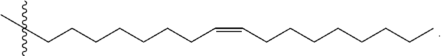 [SMILES: *CCCCCCC/C=C\CCCCCCCC]

In some embodiments, the oligonucleotide of the RNAi oligonucleotide conjugate is conjugated to a C18:2 lipid as shown in:

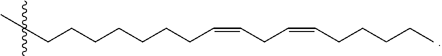 [SMILES: *CCCCCCC/C=C\C/C=C\CCCCC]

In some embodiments, the oligonucleotide of the RNAi oligonucleotide conjugate is conjugated to a C22:0 lipid as shown in:

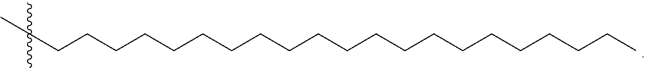 [SMILES: *CCCCCCCCCCCCCCCCCCCCC]

In some embodiments, the oligonucleotide of the RNAi oligonucleotide conjugate is conjugated to a C22:6 lipid as shown in:

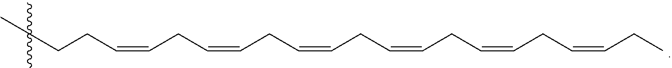 [SMILES: *CC/C=C\C/C=C\C/C=C\C/C=C\C/C=C\C/C=C\CC]

In some embodiments, the RNAi oligonucleotide conjugate reduces target mRNA in in the CNS (e.g., in the somatosensory cortex (SS cortex), hippocampus (HP), striatum, frontal cortex, cerebellum, hypothalamus (HY), cervical spinal cord (CSC), thoracic spinal cord (TSC), and/or lumbar spinal cord (LSC)).

In some embodiments, the RNAi oligonucleotide conjugate reduces target mRNA in in the CNS (e.g., in the SS cortex, HP, HY, CSC, TSC, and/or LSC) without reducing expression of the target mRNA outside the CNS. In some embodiments, the RNAi oligonucleotide conjugate reduces target mRNA in in the CNS (e.g., in the SS cortex, HP, HY, CSC, TSC, and/or LSC) without reducing expression of the target mRNA outside the CNS without reducing expression of the target mRNA in the liver.

In some embodiments, the RNAi oligonucleotide conjugate comprises a tetraloop wherein at least one nucleotide of the tetraloop is conjugated to a C8 lipid, wherein the RNAi oligonucleotide conjugate reduces target mRNA in in the CNS (e.g., in the SS cortex, HP, HY, CSC, TSC, and/or LSC) without reducing expression of the target mRNA outside the CNS. In some embodiments, the RNAi oligonucleotide conjugate reduces target mRNA in in the CNS (e.g., in the SS cortex, HP, HY, CSC, TSC, and/or LSC) without reducing expression of the target mRNA outside the CNS without reducing expression of the target mRNA in the liver. In some embodiments, the C8 lipid is conjugated to the second nucleotide of the tetraloop. In some embodiments, the tetraloop consists of 5′-GAAA-3′.

In some embodiments, the RNAi oligonucleotide conjugate comprises a tetraloop wherein at least one nucleotide of the tetraloop is conjugated to a C10 lipid, wherein the RNAi oligonucleotide conjugate reduces target mRNA in in the CNS (e.g., in the SS cortex, HP, HY, CSC, TSC, and/or LSC) without reducing expression of the target mRNA outside the CNS. In some embodiments, the RNAi oligonucleotide conjugate reduces target mRNA in in the CNS (e.g., in the SS cortex, HP, HY, CSC, TSC, and/or LSC) without reducing expression of the target mRNA outside the CNS without reducing expression of the target mRNA in the liver. In some embodiments, the C10 lipid is conjugated to the second nucleotide of the tetraloop. In some embodiments, the tetraloop consists of 5′-GAAA-3′.

In some embodiments, the RNAi oligonucleotide conjugate comprises a tetraloop wherein at least one nucleotide of the tetraloop is conjugated to a C12 lipid, wherein the RNAi oligonucleotide conjugate reduces target mRNA in in the CNS (e.g., in the SS cortex, HP, HY, CSC, TSC, and/or LSC) without reducing expression of the target mRNA outside the CNS. In some embodiments, the RNAi oligonucleotide conjugate reduces target mRNA in in the CNS (e.g., in the SS cortex, HP, HY, CSC, TSC, and/or LSC) without reducing expression of the target mRNA outside the CNS without reducing expression of the target mRNA in the liver. In some embodiments, the C12 lipid is conjugated to the second nucleotide of the tetraloop. In some embodiments, the tetraloop consists of 5′-GAAA-3′.

In some embodiments, the RNAi oligonucleotide conjugate comprises a tetraloop wherein at least one nucleotide of the tetraloop is conjugated to a C14 lipid, wherein the RNAi oligonucleotide conjugate reduces target mRNA in in the CNS (e.g., in the SS cortex, HP, HY, CSC, TSC, and/or LSC) without reducing expression of the target mRNA outside the CNS. In some embodiments, the RNAi oligonucleotide conjugate reduces target mRNA in in the CNS (e.g., in the SS cortex, HP, HY, CSC, TSC, and/or LSC) without reducing expression of the target mRNA outside the CNS without reducing expression of the target mRNA in the liver. In some embodiments, the C14 lipid is conjugated to the second nucleotide of the tetraloop. In some embodiments, the tetraloop consists of 5′-GAAA-3′.

### General Methods of Providing the Nucleic Acids and Analogues Thereof

The nucleic acids and analogues thereof comprising lipid conjugate described herein can be made using a variety of synthetic methods known in the art, including standard phosphoramidite methods. Any phosphoramidite synthesis method can be used to synthesize the provided nucleic acids of this disclosure. In certain embodiments, phosphoramidites are used in a solid phase synthesis method to yield reactive intermediate phosphite compounds, which are subsequently oxidized using known methods to produce phosphonate-modified oligonucleotides, typically with a phosphodiester or phosphorothioate internucleotide linkages. The oligonucleotide synthesis of the present disclosure can be performed in either direction: from 5′ to 3′ or from 3′ to 5′ using art known methods.

In certain embodiments, the method for synthesizing a provided nucleic acid comprises (a) attaching a nucleoside or analogue thereof to a solid support via a covalent linkage; (b) coupling a nucleoside phosphoramidite or analogue thereof to a reactive hydroxyl group on the nucleoside or analogue thereof of step (a) to form an internucleotide bond there between, wherein any uncoupled nucleoside or analogue thereof on the solid support is capped with a capping reagent; (c) oxidizing said internucleotide bond with an oxidizing agent; and (d) repeating steps (b) to (c) iteratively with subsequent nucleoside phosphoramidites or analogue thereof to form a nucleic acid or analogue thereof, wherein at least the nucleoside or analogue thereof of step (a), the nucleoside phosphoramidite or analogue thereof of step (b) or at least one of the subsequent nucleoside phosphoramidites or analogues thereof of step (d) comprises a lipid conjugate moiety as described herein. Typically, the coupling, capping/oxidizing steps and optionally, the deprotecting steps, are repeated until the oligonucleotide reaches the desired length and/or sequence, after which it is cleaved from the solid support. In certain embodiments, an oligonucleotide is prepared comprising 1-3 nucleic acid or analogues thereof comprising lipid conjugates units on a tetraloop.

In Scheme A below, where a particular protecting group, leaving group, or transformation condition is depicted, one of ordinary skill in the art will appreciate that other protecting groups, leaving groups, and transformation conditions are also suitable and are contemplated. Certain reactive functional groups (e.g., —N(H)—, —OH, etc.) envisioned in the genera in Scheme A requiring additional protection group strategies are also contemplated and is appreciated by those having ordinary skill in the art. Such groups and transformations are described in detail in March's Advanced Organic Chemistry: Reactions, Mechanisms, and Structure, M. B. Smith and J. March, 5^{th} Edition, John Wiley & Sons, 2001, COMPREHENSIVE ORGANIC TRANSFORMATIONS, (R. C. Larock, 2^{nd} Edition, John Wiley & Sons, 1999), and PROTECTING GROUPS IN ORGANIC SYNTHESIS, (T. W. Greene and P. G. M. Wuts, 3^{rd} edition, John Wiley & Sons, 1999), the entirety of each of which is hereby incorporated herein by reference.

In certain embodiments, nucleic acids, and analogues thereof of the present disclosure are generally prepared according to Scheme A, Scheme A1 and Scheme B set forth below:

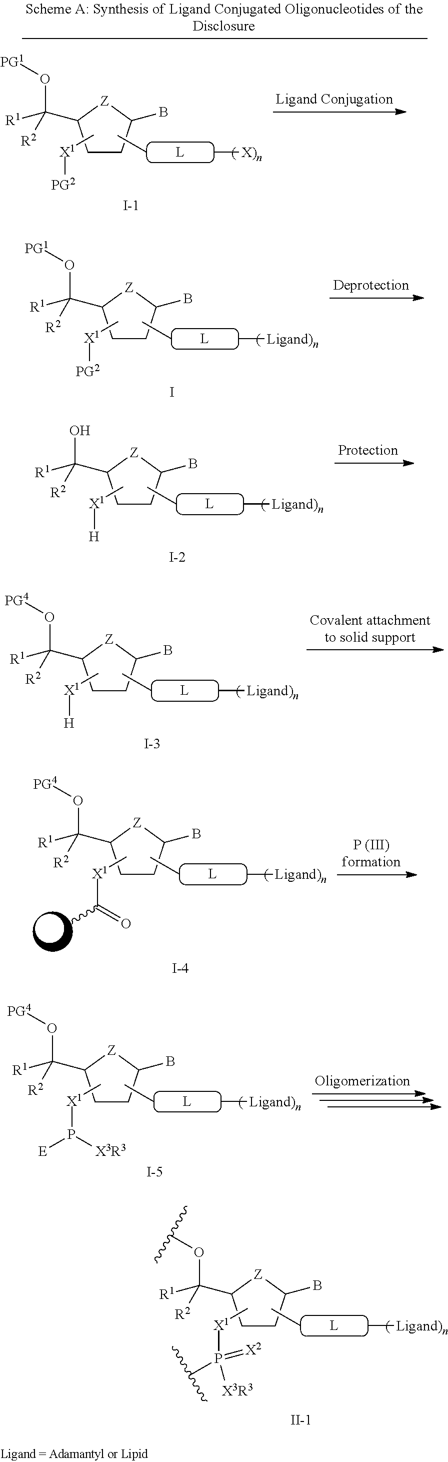

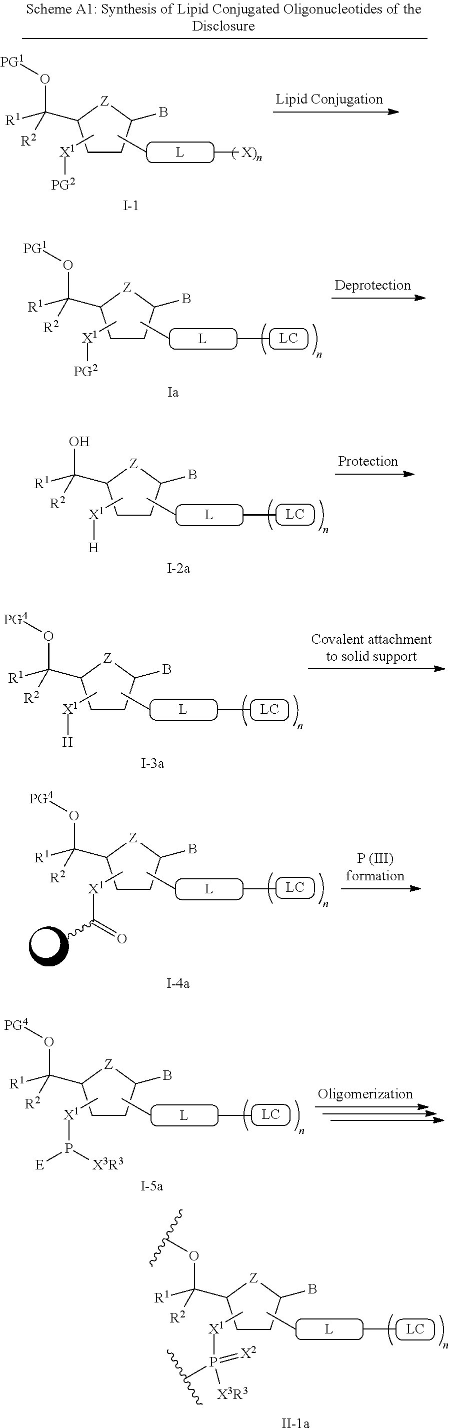

As depicted in Scheme A and Scheme A1 above, a nucleic acid or analogue thereof of formula I-1 is conjugated with one or more ligand/lipophilic compound to form a compound of formula I or Ia comprising one more ligand/lipid conjugates. Typically, conjugation is performed through an esterification or amidation reaction between a nucleic acid or analogue thereof of formula I-1 or I-1a and one or more adamantyl and/or lipophilic compound (e.g., fatty acid) in series or in parallel by known techniques in the art. Nucleic acid or analogue thereof of formula I or Ia can then be deprotected to form a compound of formula I-2 or I-2a and protected with a suitable hydroxyl protecting group (e.g., DMTr) to form a compound of formula 1-3 or I-3a. In one aspect, nucleic acid-ligand conjugates of formula I-3 or I-3a can be covalently attached to a solid support (e.g., through a succinic acid linking group) to form a solid support nucleic acid-ligand conjugate or analogue thereof of formula I-4 or I-4a comprising one or more adamantyl and/or lipid conjugate. In another aspect, a nucleic acid-ligand conjugates of formula 1-3 or I-3a can react with a P(III) forming reagent (e.g., 2-cyanoethyl N,N-di-isopropylchlorophosphoramidite) to form a nucleic acid or analogue thereof of formula I-5 or I-5a comprising a P(III) group. A nucleic acid-ligand conjugate or analogue thereof of formula I-5 or I-5a can then be subjected to oligomerization forming conditions preformed using known and commonly applied processes to prepare oligonucleotides in the art. For example, the compound of formula I-5 or I-5a is coupled to a solid supported nucleic acid-ligand conjugate or analogue thereof bearing a 5′-hydroxyl group. Further steps can comprise one or more deprotections, couplings, phosphite oxidation, and/or cleavage from the solid support to provide an oligonucleotide of various nucleotide lengths, including one or more lipid conjugate nucleotide units represented by a compound of formula II-1 or II-Ia. Each of B, E, L, ligand, LC, n, PG¹, PG², PG⁴, R¹, R², R³, X, X¹, X², X³, and Z is as defined above and described herein.

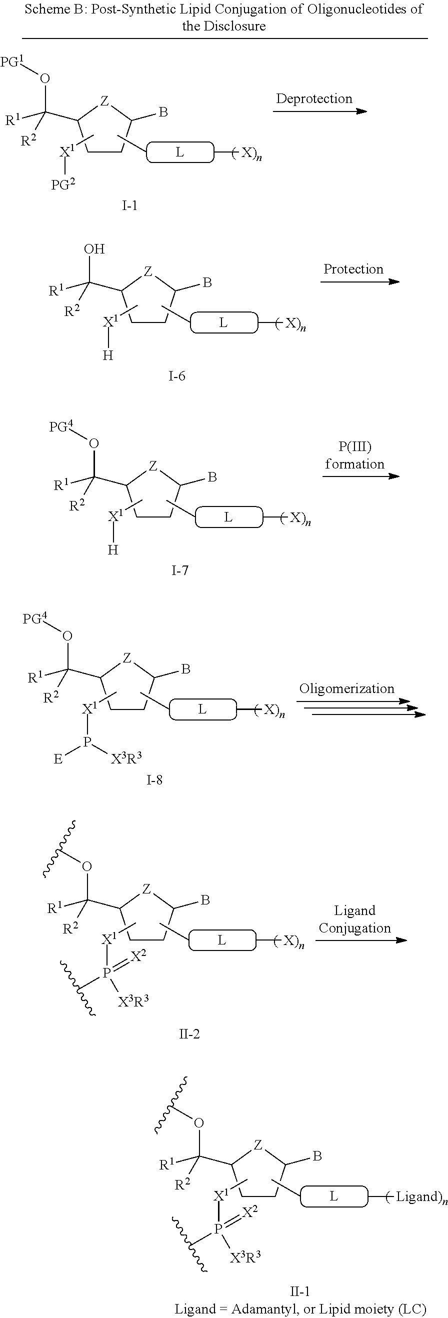

As depicted in Scheme B above, a nucleic acid or analogue thereof of formula I-1 can be deprotected to form a compound of formula I-6, protected with a suitable hydroxyl protecting group (e.g., DMTr) to form a compound of formula I-7, and reacted with a P(III) forming reagent (e.g., 2-cyanoethyl N,N-di-isopropylchlorophosphoramidite) to form a nucleic acid or analogue thereof of formula I-8 comprising a P(III) group. Next, a nucleic acid or analogue thereof of formula I-8 is subjected to oligomerization forming conditions preformed using known and commonly applied processes to prepare oligonucleotides in the art. For example, the compound of formula I-8 is coupled to a solid supported nucleic acid or analogue thereof bearing a 5′-hydroxyl group. Further steps can comprise one or more deprotections, couplings, phosphite oxidation, and/or cleavage from the solid support to provide an oligonucleotide of various nucleotide lengths represented by a compound of formula II-2. An oligonucleotide of formula II-2 can then be conjugated with one or more ligands e.g., adamantyl, or lipophilic compound (e.g., fatty acid) to form a compound of formula II-1 comprising one or more ligand conjugates. Typically, conjugation is performed through an esterification or amidation reaction between a nucleic acid or analogue thereof of formula II-2 and one or more adamantyl or fatty acid in series or in parallel by known techniques in the art. Each of B, E, L, ligand, LC, n, PG¹, PG², PG⁴, R¹, R², R³, X, X¹, X², X³, and Z is as defined above and described herein.

In certain embodiments, nucleic acids, and analogues thereof of the present disclosure are prepared according to Scheme C and Scheme D set forth below:

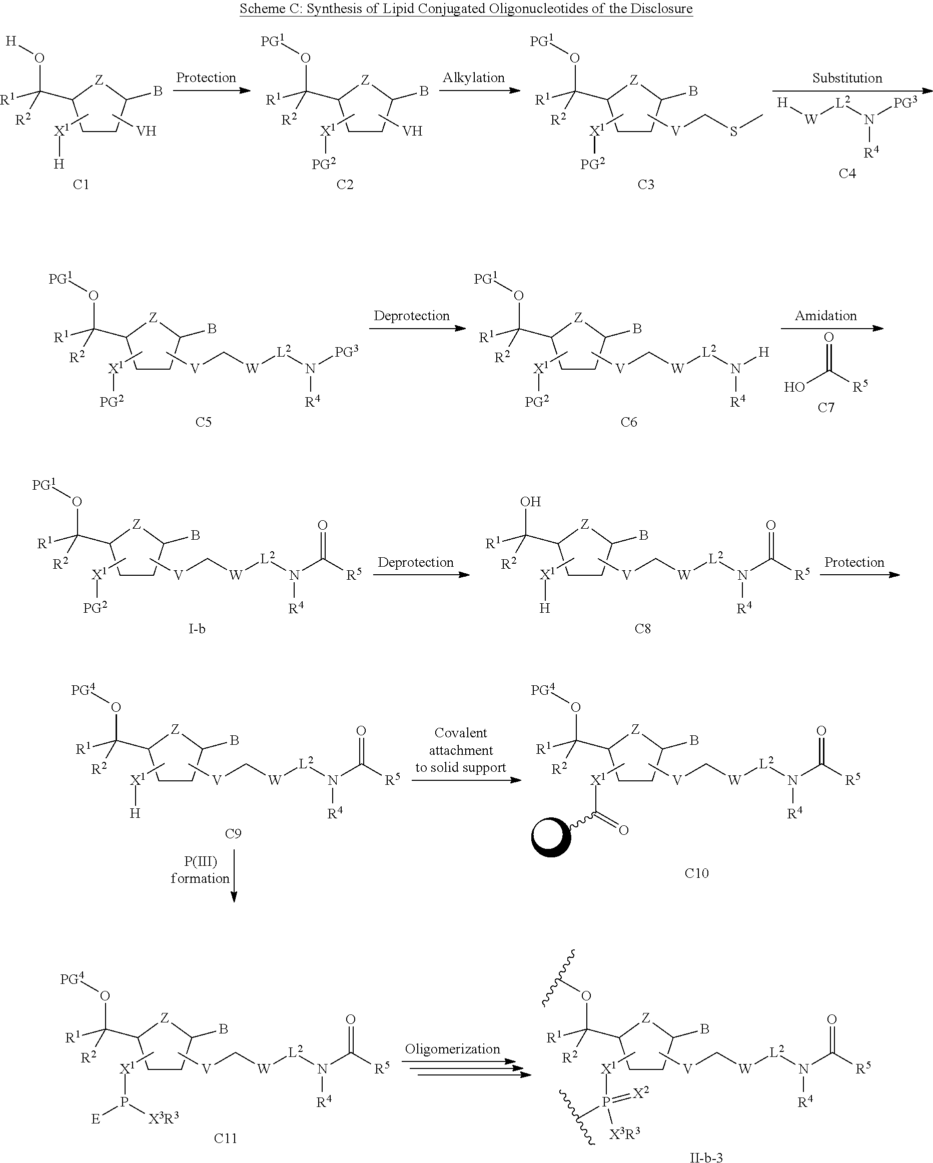

As depicted in Scheme C above, a nucleic acid or analogue thereof of formula C1 is protected to form a compound of formula C2. Nucleic acid or analogue thereof of formula C2 is then alkylated (e.g., using DMSO and acetic acid via the Pummerer rearrangement) to form a monothioacetal compound of formula C3. Next, nucleic acid or analogue thereof of formula C3 is coupled with C4 under appropriate conditions (e.g., mild oxidizing conditions) to form a nucleic acid or analogue thereof of formula C5. Nucleic acid or analogue thereof of formula C5 can then be deprotected to form a compound of formula C6 and coupled with a ligand (adamantyl or lipophilic compound (e.g., a fatty acid)) of formula C7 under appropriate amide forming conditions (e.g., HATU, DIPEA), to form a nucleic acid-ligand conjugate or analogue thereof of formula I-b comprising a lipid conjugate of the disclosure. Nucleic acid-ligand conjugate or analogue thereof of formula I-b can then be deprotected to form a compound of formula C8 and protected with a suitable hydroxyl protecting group (e.g., DMTr) to form a compound of formula C9. In one aspect, nucleic acid, or analogue thereof of formula C9 can be covalently attached to a solid support (e.g., through a succinic acid linking group) to form a solid support nucleic acid-ligand conjugate or analogue thereof of formula C10 comprising a ligand conjugate (adamantyl or lipid moiety) of the disclosure. In another aspect, a nucleic acid-ligand conjugate or analogue thereof of formula C9 can reacted with a P(III) forming reagent (e.g., 2-cyanoethyl N,N-di-isopropylchlorophosphoramidite) to form a nucleic acid-ligand conjugate or analogue thereof of formula C11 comprising a P(III) group. A nucleic acid-ligand conjugate or analogue thereof of formula C11 can then be subjected to oligomerization forming conditions preformed using known and commonly applied processes to prepare oligonucleotides in the art. For example, the compound of formula C11 is coupled to a solid supported nucleic acid-ligand conjugate or analogue thereof bearing a 5′-hydroxyl group. Further steps can comprise one or more deprotections, couplings, phosphite oxidation, and/or cleavage from the solid support to provide an oligonucleotide of various nucleotide lengths, including one or more adamantyl and/or lipid conjugate nucleotide units represented by a compound of formula II-b-3. Each of B, E, L², PG¹, PG², PG³, PG⁴, R¹, R², R³, R⁴, R⁵, X¹, X², X³, V, W, and Z is as defined above and described herein.

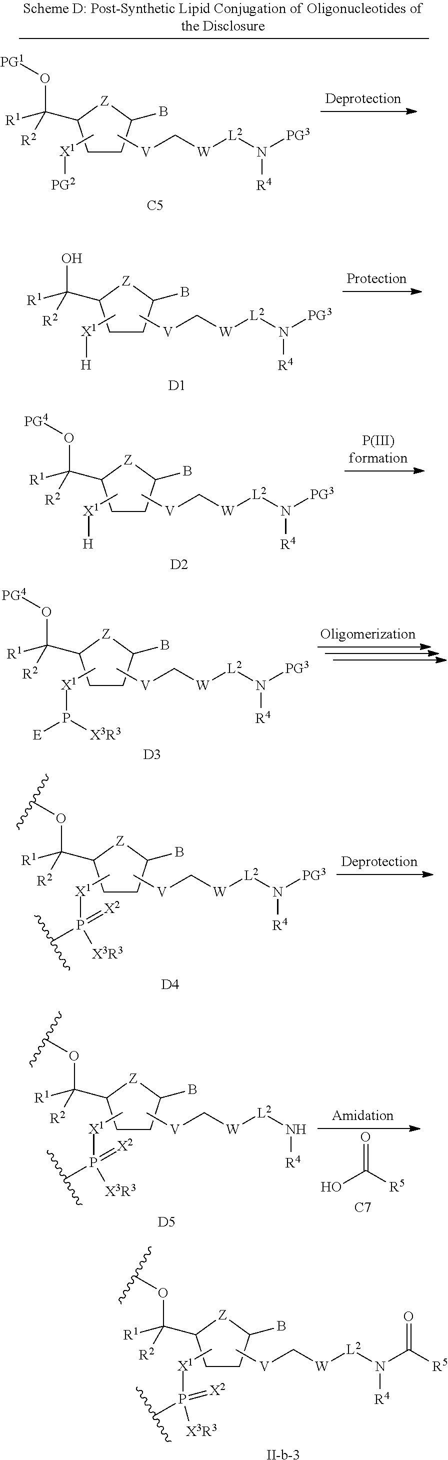

Each of B, E, L², PG¹, PG², PG³, PG⁴, R¹, R², R³, R⁴, R⁵, X¹, X², X³, V, W, and Z is as defined above and described herein. As depicted in Scheme D above, a nucleic acid or analogue thereof of formula C5 can be selectively deprotected to form a compound of formula D1, protected with a suitable hydroxyl protecting group (e.g., DMTr) to form a compound of formula D2, and reacted with a P(III) forming reagent (e.g., 2-cyanoethyl N,N-di-isopropylchlorophosphoramidite) to form a nucleic acid or analogue thereof of formula D3. Next, a nucleic acid or analogue thereof of formula D3 is subjected to oligomerization forming conditions preformed using known and commonly applied processes to prepare oligonucleotides in the art. For example, the compound of formula D3 is coupled to a solid supported nucleic acid or analogue thereof bearing a 5′-hydroxyl group. Further steps can comprise one or more deprotections, couplings, phosphite oxidation, and/or cleavage from the solid support to provide an oligonucleotide of various nucleotide lengths, represented by a compound of formula D4. An oligonucleotide of formula D4 can then be deprotected to form a compound of formula D5 and coupled with a hydrophobic ligand (e.g., adamantyl or a lipophilic moiety) to form a compound of formula C7 (e.g., adamantyl or a fatty acid) under appropriate amide forming conditions (e.g., HATU, DIPEA), to form an oligonucleotide of formula II-b-3 comprising a ligand (e.g., adamantyl or a fatty acid) conjugate of the disclosure.

One of skill in the art will appreciate that various functional groups present in the nucleic acid or analogues thereof of the disclosure such as aliphatic groups, alcohols, carboxylic acids, esters, amides, aldehydes, halogens, and nitriles can be interconverted by techniques well known in the art including, but not limited to reduction, oxidation, esterification, hydrolysis, partial oxidation, partial reduction, halogenation, dehydration, partial hydration, and hydration. See for example, “MARCH'S ADVANCED ORGANIC CHEMISTRY”, (5^{th} Ed., Ed.: Smith, M. B. and March, J., John Wiley & Sons, New York: 2001), the entirety of each of which is herein incorporated by reference. Such interconversions may require one or more of the aforementioned techniques, and certain methods for synthesizing the provided nucleic acids of the disclosure are described below in the Exemplification.

In some embodiments, the present disclosure provides a method for preparing an oligonucleotide comprising one or more lipid conjugate, said lipid conjugate unit represent by formula II-a-1:

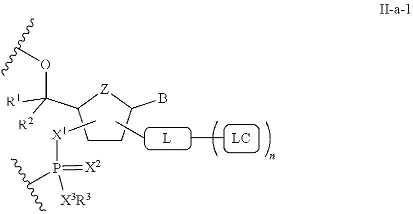

or a pharmaceutically acceptable salt thereof, comprising the steps of:
(a) providing a nucleic acid or analogue thereof of formula I-5a:

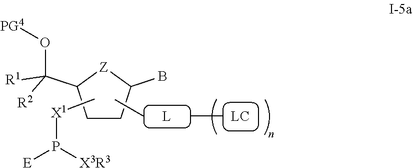

or salt thereof, and
(b) oligomerizing said compound of formula I-5a to form a compound of formula II-1a, wherein each of B, E, L, LC, n, PG⁴, R¹, R², R³, X, X¹, X², X³, E, and Z is as defined above and described herein.

In step (b) above, oligomerizing refers to preforming oligomerization forming conditions using known and commonly applied processes to prepare oligonucleotides in the art. For example, the compound of formula I-5a is coupled to a solid supported nucleic acid or analogue thereof bearing a 5′-hydroxyl group. Further steps can comprise one or more deprotections, couplings, phosphite oxidation, and cleavage from the solid support to provide an oligonucleotide of various nucleotide lengths, represented by a compound of formula II-1a comprising a lipid conjugate of the disclosure.

In some embodiments, the present disclosure provides a method for preparing an oligonucleotide comprising one or more lipid conjugate, further comprising preparing a nucleic acid or analogue thereof of formula I-5a:

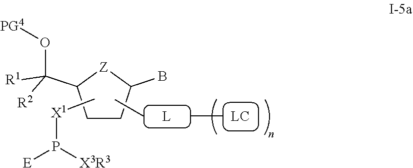

or a salt thereof, comprising the steps of:
(a) providing a nucleic acid or analogue thereof of formula Ia:

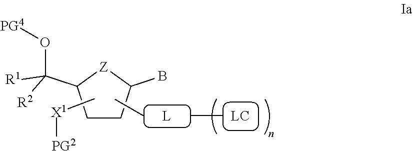

or salt thereof,
(b) deprotecting said nucleic acid or analogue thereof of formula Ia to form a compound of formula I-2a:

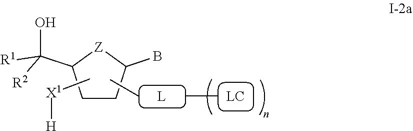

or salt thereof,
(c) protecting said nucleic acid or analogue thereof of formula I-2 to form a compound of formula I-3a:

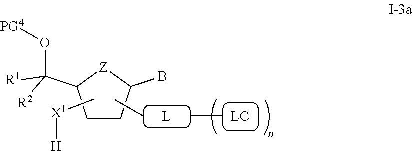

or salt thereof, and
(d) treating said nucleic acid or analogue thereof of formula I-3a with a P(III) forming reagent to form a nucleic acid or analogue thereof of formula I-5a, wherein each of B, E, L, LC, n, PG⁴, R¹, R², R³, X, X¹, X², X³, E, and Z is as defined above and described herein.

In step (b) above, PG¹ and PG² of a compound of formula Ia comprise silyl ethers or cyclic silylene derivatives that can be removed under acidic conditions or with fluoride anion. Examples of reagents providing fluoride anion for the removal of silicon-based protecting groups include hydrofluoric acid, hydrogen fluoride pyridine, triethylamine trihydrofluoride, tetra-N-butylammonium fluoride, and the like.

In step (c) above, a compound of formula I-2a is protected with a suitable hydroxyl protecting group. In certain embodiments, the protecting group PG⁴ used for protection of the 5′-hydroxyl group of a compound of formula I-2a includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

In step (d) above, a compound of formula I-3a is treated with a P(III) forming reagent to afford a compound of formula I-5a. In the context of the present disclosure, a P(III) forming reagent is a phosphorus reagent that is reacted to for a phosphorus (III) compound. In some embodiments, the P(III) forming reagent is 2-cyanoethyl N,N-diisopropylchlorophosphoramidite or 2-cyanoethyl phosphorodichloridate. In certain embodiments, the P(III) forming reagent is 2-cyanoethyl N,N-diisopropylchlorophosphoramidite. One of ordinary skill would recognize that the displacement of a leaving group in a P(III) forming reagent by X¹ of a compound of formula I-3a is achieved either with or without the presence of a suitable base. Such suitable bases are well known in the art and include organic and inorganic bases. In certain embodiments, the base is a tertiary amine such as triethylamine or diisopropylethylamine. In other embodiments, step (d) above is preformed using N,N-dimethylphosphoramic dichloride as a P(V) forming reagent.

In some embodiments, the present disclosure provides a method for preparing an oligonucleotide comprising one or more lipid conjugates, further comprising preparing a nucleic acid-lipid conjugate or analogue thereof of formula Ia:

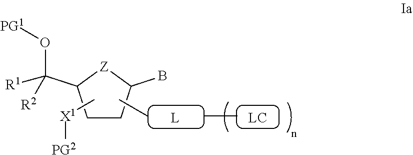

or a salt thereof, comprising the steps of:
(a) providing a nucleic acid or analogue thereof of formula I-1:

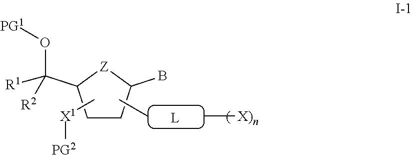

or salt thereof, and,
(b) conjugating one or more lipophilic compounds to a nucleic acid or analogue thereof of formula I-1 to form a nucleic acid or analogue thereof of formula Ia comprising one or more lipid conjugates, wherein: each of B, E, L, LC, n, PG¹, PG², R1, R², X, X¹, and Z is as defined above and described herein.

In step (b) above, a nucleic acid or analogue thereof of formula I-1a is conjugated with one or more lipophilic compounds to form a compound of formula Ia comprising one more lipid conjugates of the disclosure. Typically, conjugation is performed through an esterification or amidation reaction between a nucleic acid or analogue thereof of formula I-1a and one or more fatty acids in series or in parallel by known techniques in the art. In certain embodiments, conjugation is performed under suitable amide forming conditions to afford a compound of formula I comprising one more lipid conjugates. Suitable amide forming conditions can include the use of an amide coupling reagent known in the art such as, but not limited to HATU, PyBOP, DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU. Alternatively, conjugation of a lipophilic compound can be accomplished by any one of the cross-coupling technologies described in Table A herein.

In some embodiments, the present disclosure provides a method for preparing an oligonucleotide comprising one or more lipid conjugate, said lipid conjugate unit represent by formula II-1:

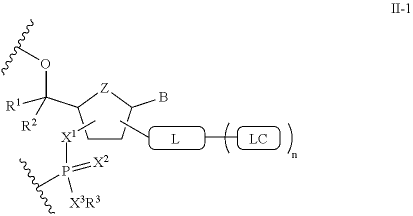

or a pharmaceutically acceptable salt thereof, comprising the steps of:
(a) providing an oligonucleotide of formula II-2:

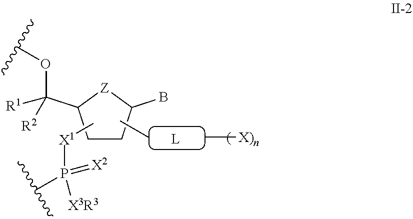

or salt thereof, and,
(b) conjugating one or more lipophilic compounds to an oligonucleotide of formula II-2 to form an oligonucleotide of formula II-1 comprising one or more lipid conjugates. In step (b) above, an oligonucleotide of formula II-2 is conjugated with one or more lipophilic compounds to form an oligonucleotide of formula II-1 comprising one more lipid conjugates of the disclosure.

Typically, conjugation is performed through an esterification or amidation reaction between an oligonucleotide of formula II-2 and one or more fatty acids in series or in parallel by known techniques in the art. In certain embodiments, conjugation is performed under suitable amide forming conditions to afford an oligonucleotide of formula II-1 comprising one more lipid conjugates. Suitable amide forming conditions can include the use of an amide coupling reagent known in the art such as, but not limited to HATU, PyBOP, DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU. Alternatively, conjugation of a lipophilic compound can be accomplished by any one of the cross-coupling technologies described in Table A herein.

In some embodiments, the present disclosure provides a method for preparing an oligonucleotide comprising a unit represent by formula II-2:

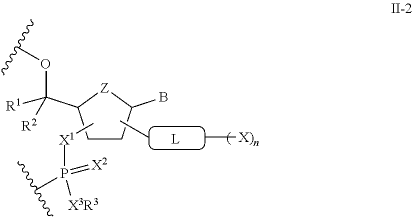

or a pharmaceutically acceptable salt thereof, comprising the steps of:
(a) providing a nucleic acid or analogue thereof of formula I-8:

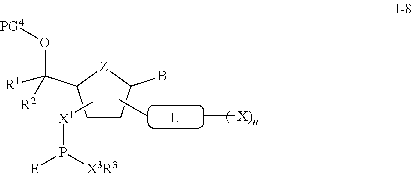

or salt thereof, and
(b) oligomerizing said compound of formula I-8 to form a compound of formula II-2.

In step (b) above, oligomerizing refers to preforming oligomerization forming conditions using known and commonly applied processes to prepare oligonucleotides in the art. For example, the compound of formula I-8 is coupled to a solid supported nucleic acid or analogue thereof bearing a 5′-hydroxyl group. Further steps can comprise one or more deprotections, couplings, phosphite oxidation, and cleavage from the solid support to provide an oligonucleotide of various nucleotide lengths, represented by a compound of formula II-2.

In some embodiments, the present disclosure provides a method for preparing a nucleic acid or analogue thereof comprising one or more lipid conjugate, further comprising preparing a nucleic acid or analogue thereof of formula I-8:

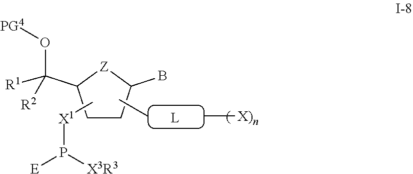

or a salt thereof, comprising the steps of:
(a) providing a nucleic acid or analogue thereof of formula I-1:

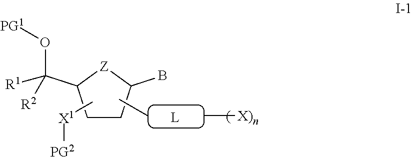

or salt thereof,
(b) deprotecting said nucleic acid or analogue thereof of formula I-1 to form a compound of formula I-6:

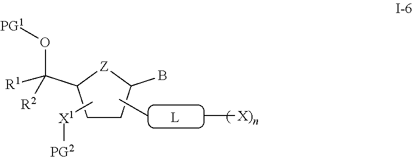

or salt thereof,
(c) protecting said nucleic acid or analogue thereof of formula I-6 to form a compound of formula I-7:

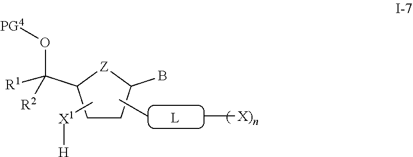

or salt thereof, and
(d) treating said nucleic acid or analogue thereof of formula I-7 with a P(III) forming reagent to form a nucleic acid or analogue thereof of formula I-8, In step (b) above, PG¹ and PG² of a compound of formula I-1 comprise silyl ethers or cyclic silylene derivatives that can be removed under acidic conditions or with fluoride anion. Examples of reagents providing fluoride anion for the removal of silicon-based protecting groups include hydrofluoric acid, hydrogen fluoride pyridine, triethylamine trihydrofluoride, tetra-N-butylammonium fluoride, and the like.

In step (c) above, a compound of formula I-6 is protected with a suitable hydroxyl protecting group. In certain embodiments, the protecting group PG⁴ used for protection of the 5′-hydroxyl group of a compound of formula I-6 includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

In step (d) above, a compound of formula I-7 is treated with a P(III) forming reagent to afford a compound of formula I-8. In the context of the present disclosure, a P(III) forming reagent is a phosphorus reagent that is reacted to for a phosphorus (III) compound. In some embodiments, the P(III) forming reagent is 2-cyanoethyl N,N-diisopropylchlorophosphoramidite or 2-cyanoethyl phosphorodichloridate. In certain embodiments, the P(III) forming reagent is 2-cyanoethyl N,N-diisopropylchlorophosphoramidite. One of ordinary skill would recognize that the displacement of a leaving group in a P(III) forming reagent by X¹ of a compound of formula I-7 is achieved either with or without the presence of a suitable base. Such suitable bases are well known in the art and include organic and inorganic bases. In certain embodiments, the base is a tertiary amine such as triethylamine or diisopropylethylamine. In other embodiments, step (d) above is preformed using N,N-dimethylphosphoramic dichloride as a P(V) forming reagent.

In some embodiments, the present disclosure provides a method for preparing an oligonucleotide-ligand conjugate comprising one or more adamantyl and/or lipid moieties, said conjugate unit represented by formula II-b-3:

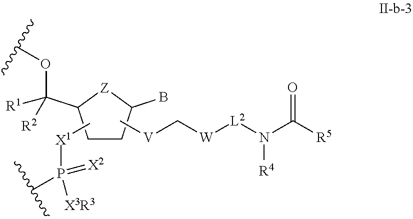

or a pharmaceutically acceptable salt thereof, comprising the steps of:
(a) providing a nucleic acid-ligand conjugate or analogue thereof of formula C11:

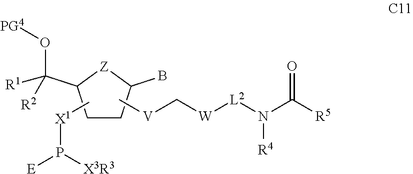

or salt thereof, and
(b) oligomerizing said compound of formula C11 to form a compound of formula II-b-3, In step (b) above, oligomerizing refers to preforming oligomerization forming conditions using known and commonly applied processes to prepare oligonucleotides in the art. For example, the compound of formula C11 is coupled to a solid supported nucleic acid or analogue thereof bearing a 5′-hydroxyl group. Further steps can comprise one or more deprotections, couplings, phosphite oxidation, and cleavage from the solid support to provide an oligonucleotide-ligand conjugate of various nucleotide lengths, with one or more nucleic acid-ligand conjugate units, wherein each unit is represented by a compound of formula II-b-3 comprising an adamantyl or lipid moiety of the disclosure.

In some embodiments, the method for preparing an oligonucleotide of formula II-b-3 comprising one or more lipid conjugate, further comprises preparing a nucleic acid-ligand conjugate or analogue thereof of formula C11:

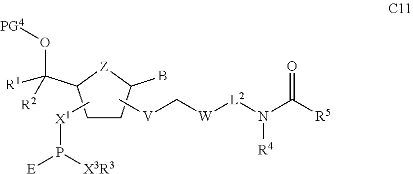

or a salt thereof, comprising the steps of:
(a) providing a nucleic acid-ligand conjugate or analogue thereof of formula I-b:

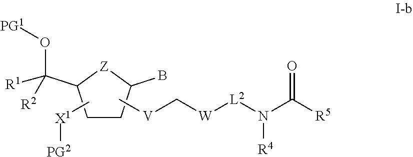

or salt thereof,
(b) deprotecting said nucleic acid-ligand conjugate or analogue thereof of formula I-b to form a compound of formula C8:

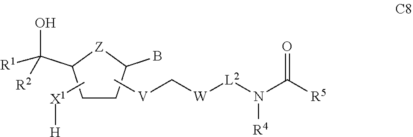

or salt thereof,
(c) protecting said nucleic acid-ligand conjugate or analogue thereof of formula C8 to form a compound of formula C9:

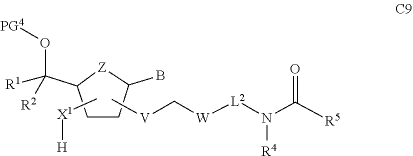

or salt thereof, and
(d) treating said nucleic acid-ligand conjugate or analogue thereof of formula C9 with a P(III) forming reagent to form a nucleic acid or analogue thereof of formula C11. In step (b) above, PG¹ and PG² of a compound of formula I-b comprise silyl ethers or cyclic silylene derivatives that can be removed under acidic conditions or with fluoride anion. Examples of reagents providing fluoride anion for the removal of silicon-based protecting groups include hydrofluoric acid, hydrogen fluoride pyridine, triethylamine trihydrofluoride, tetra-N-butylammonium fluoride, and the like.

In step (c) above, a compound of formula C8 is protected with a suitable hydroxyl protecting group. In certain embodiments, the protecting group PG⁴ used for protection of the 5′-hydroxyl group of a compound of formula C8 includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

In step (d) above, a compound of formula C9 is treated with a P(III) forming reagent to afford a compound of formula C11. In the context of the present disclosure, a P(III) forming reagent is a phosphorus reagent that is reacted to for a phosphorus (III) compound. In some embodiments, the P(III) forming reagent is 2-cyanoethyl N,N-diisopropylchlorophosphoramidite or 2-cyanoethyl phosphorodichloridate. In certain embodiments, the P(III) forming reagent is 2-cyanoethyl N,N-diisopropylchlorophosphoramidite. One of ordinary skill would recognize that the displacement of a leaving group in a P(III) forming reagent by X¹ of a compound of formula C9 is achieved either with or without the presence of a suitable base. Such suitable bases are well known in the art and include organic and inorganic bases. In certain embodiments, the base is a tertiary amine such as triethylamine or diisopropylethylamine. In other embodiments, step (d) above is preformed using N,N-dimethylphosphoramic dichloride as a P(V) forming reagent.

In some embodiments, the present disclosure provides a method for preparing an oligonucleotide-ligand conjugate of formula II-b-3 comprising one or more nucleic acid-ligand conjugate units each comprising one or more adamantyl or lipid moieties, further comprising preparing a nucleic acid-ligand conjugate or analogue thereof of formula I-b:

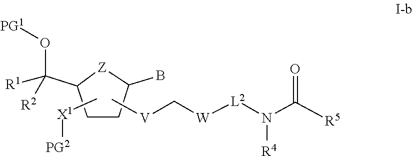

or a salt thereof, comprising the steps of: (a) providing a nucleic acid-ligand conjugate or analogue thereof of formula C6:

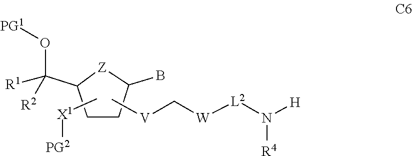

or salt thereof, and,
(b) conjugating a lipophilic compound to a nucleic acid or analogue thereof of formula C6 to form a nucleic acid-ligand conjugate or analogue thereof of formula I-b comprising one or more adamantyl and/or lipid conjugates. In step (b) above, conjugation is performed under suitable amide forming conditions to afford a compound of formula I-b comprising an adamantyl and/or lipid conjugate. Suitable amide forming conditions can include the use of an amide coupling reagent known in the art such as, but not limited to HATU, PyBOP, DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU. In certain embodiments, the amide forming conditions comprise HATU and DIPEA or TEA.

In certain embodiments, a nucleic acid-ligand conjugate or analogue thereof of formula C6 is provided in salt form (e.g., a fumarate salt) and is first converted to the free base (e.g., using sodium bicarbonate) before preforming the conjugation step.

In some embodiments, the present disclosure provides a method for preparing an oligonucleotide-ligand conjugate of formula II-b-3 comprising one or more nucleic acid-ligand conjugate units, further comprises preparing a nucleic acid-ligand conjugate or analogue thereof of formula C6:

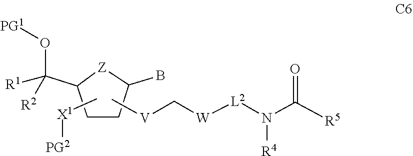

or a salt thereof, comprising the steps of:
(a) providing a nucleic acid or analogue thereof of formula C1:

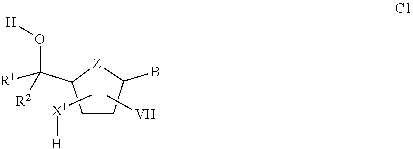

or salt thereof, and,
(b) protecting said nucleic acid or analogue thereof of formula C1 to form a compound of formula C2:

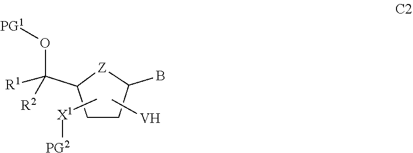

or salt thereof,
(c) alkylating said nucleic acid or analogue thereof of formula C2 to form a compound of formula C3:

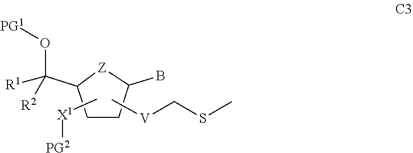

or salt thereof,
(d) substituting said nucleic acid or analogue thereof of formula C3 with a compound of formula C4:

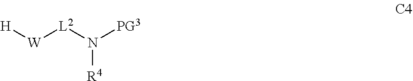

or salt thereof, to form a compound of formula C5:

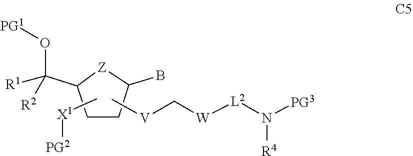

or salt thereof,
(e) deprotecting said nucleic acid or analogue thereof of formula C5 to form a nucleic acid-ligand conjugate or analogue thereof of formula C6. In step (b) above, PG¹ and PG² groups of formula C2 are taken together with their intervening atoms to form a cyclic diol protecting group, such as a cyclic acetal or ketal. Such groups include methylene, ethylidene, benzylidene, isopropylidene, cyclohexylidene, and cyclopentylidene, silylene derivatives such as di-t-butylsilylene and 1,1,3,3-tetraisopropylidisiloxanylidene, a cyclic carbonate, a cyclic boronate, and cyclic monophosphate derivatives based on cyclic adenosine monophosphate (i.e., cAMP). In certain embodiments, the cyclic diol protection group is 1,1,3,3-tetraisopropylidisiloxanylidene prepared from the reaction of a diol of formula C1 and 1,3-dichloro-1,1,3,3-tetraisopropyldisiloxane under basic conditions.

In step (c) above, a nucleic acid or analogue thereof of formula C2 is alkylated with a mixture of DMSO and acetic anhydride under acidic conditions. In certain embodiments, when —V—H is a hydroxyl group, the mixture of DMSO and acetic anhydride in the presence of acetic acid forms (methylthio)methyl acetate in situ via the Pummerer rearrangement which then reacts with the hydroxyl group of the nucleic acid or analogue thereof of formula C2 to provide a monothioacetal functionalized fragment nucleic acid or analogue thereof of formula C3.

In step (d) above, substitution of the thiomethyl group of a nucleic acid or analogue thereof of formula C3 using a nucleic acid or analogue thereof of formula C4 affords a nucleic acid or analogue thereof of formula C4. In certain embodiments, substitution occurs under mild oxidizing and/or acidic conditions. In some embodiments, V is oxygen. In some embodiments, the mild oxidation reagent includes a mixture of elemental iodine and hydrogen peroxide, urea hydrogen peroxide complex, silver nitrate/silver sulfate, sodium bromate, ammonium peroxodisulfate, tetrabutylammonium peroxydisulfate, Oxone®, Chloramine T, Selectfluor®, Selectfluor® II, sodium hypochlorite, or potassium iodate/sodium periodiate. In certain embodiments, the mild oxidizing agent includes N-iodosuccinimide, N-bromosuccinimide, N-chlorosuccinimide, 1,3-diiodo-5,5-dimethylhydantion, pyridinium tribromide, iodine monochloride or complexes thereof, etc. Acids that are typically used under mild oxidizing condition include sulfuric acid, p-toluenesulfonic acid, trifluoromethanesulfonic acid, methanesulfonic acid, and trifluoroacetic acid. In certain embodiments, the mild oxidation reagent includes a mixture of N-iodosuccinimide and trifluoromethanesulfonic acid.

In step (e) above, removal of PG³ and optionally R⁴ (when R⁴ is a suitable amine protecting group) of a nucleic acid-ligand conjugate or analogue thereof of formula C5 affords a nucleic acid-ligand conjugate or analogue thereof of formula C6 or a salt thereof. In some embodiments, PG³ and/or R⁴ comprise carbamate derivatives that can be removed under acidic or basic conditions. In certain embodiments, the protecting groups (e.g., both PG³ and R⁴ or either of PG³ or R⁴ independently) of a nucleic acid-ligand conjugate or analogue thereof of formula C5 are removed by acid hydrolysis. It will be appreciated that upon acid hydrolysis of the protecting groups of a nucleic acid-ligand conjugate or analogue thereof of formula C5, a salt of formula C6 thereof is formed. For example, when an acid-labile protecting group of a nucleic acid-ligand conjugate or analogue thereof of formula C5 is removed by treatment with an acid such as hydrochloric acid, then the resulting amine compound would be formed as its hydrochloride salt. One of ordinary skill in the art would recognize that a wide variety of acids are useful for removing amino protecting groups that are acid-labile and therefore a wide variety of salt forms of a nucleic acid or analogue thereof of formula C6 are contemplated.

In other embodiments, the protecting groups (e.g., both PG³ and R⁴ or either of PG³ or R⁴ independently) of a nucleic acid or analogue thereof of formula C5 are removed by base hydrolysis. For example, Fmoc and trifluoroacetyl protecting groups can be removed by treatment with base. One of ordinary skill in the art would recognize that a wide variety of bases are useful for removing amino protecting groups that are base-labile. In some embodiments, a base is piperidine. In some embodiments, a base is 1,8-diazabicyclo[5.4.0]undec-7-ene (DBU). In certain embodiments, a nucleic acid-ligand conjugate or analogue thereof of formula C5 is deprotected under basic conditions followed by treating with an acid to form a salt of formula C6. In certain embodiments, the acid is fumaric acid the salt of formula C6 is the fumarate.

In some embodiments, the present disclosure provides a method for preparing an oligonucleotide-ligand conjugate comprising one or more nucleic acid-ligand conjugate, said nucleic acid-ligand conjugate unit represented by formula II-b-3:

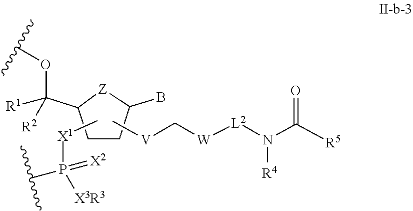

or a pharmaceutically acceptable salt thereof, comprising the steps of:
(a) providing an oligonucleotide of formula D5:

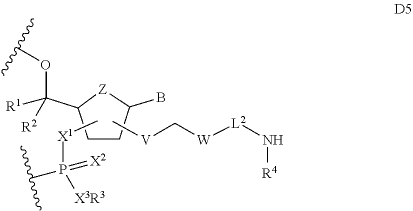

or salt thereof, and,
(b) conjugating one or more adamantyl or lipophilic compounds to an oligonucleotide of formula D5 to form an oligonucleotide-ligand conjugate of formula II-b-3 comprising one or more nucleic acid-ligand conjugate units. In step (b) above, conjugation is performed under suitable amide forming conditions to afford a compound of formula D5 comprising an adamantyl or lipid conjugate. Suitable amide forming conditions can include the use of an amide coupling reagent known in the art such as, but not limited to HATU, PyBOP, DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU. In certain embodiments, the amide forming conditions comprise HATU and DIPEA or TEA.

In some embodiments, the present disclosure provides a method for preparing an oligonucleotide-ligand conjugate comprising a unit represent by formula D5:

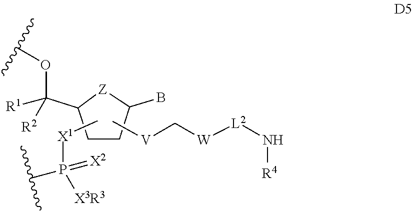

or a salt thereof, comprising the steps of:
(a) providing a nucleic acid-ligand conjugate or analogue thereof of formula D4:

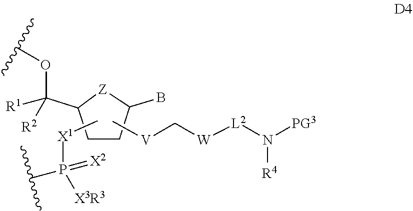

or salt thereof, and
(b) deprotecting said compound of formula D4 to form a compound of formula D5. In step (b) above, removal of PG³ and optionally R⁴ (when R⁴ is a suitable amine protecting group) of an oligonucleotide of formula D4 affords an oligonucleotide-ligand conjugate of formula D5 or a salt thereof. In some embodiments, PG³ and/or R⁴ comprise carbamate derivatives that can be removed under acidic or basic conditions. In certain embodiments, the protecting groups (e.g., both PG³ and R⁴ or either of PG³ or R⁴ independently) of an oligonucleotide-ligand conjugate of formula D4 are removed by acid hydrolysis. It will be appreciated that upon acid hydrolysis of the protecting groups of an oligonucleotide-ligand conjugate of formula D4, a salt of formula D5 thereof is formed. For example, when an acid-labile protecting group of an oligonucleotide of formula D4 is removed by treatment with an acid such as hydrochloric acid, then the resulting amine compound would be formed as its hydrochloride salt. One of ordinary skill in the art would recognize that a wide variety of acids are useful for removing amino protecting groups that are acid-labile and therefore a wide variety of salt forms of a nucleic acid-ligand conjugate unit or analogue thereof of formula D5 are contemplated.

In other embodiments, the protecting groups (e.g., both PG³ and R⁴ or either of PG³ or R⁴ independently) of an oligonucleotide-ligand conjugate of formula D4 are removed by base hydrolysis. For example, Fmoc and trifluoroacetyl protecting groups can be removed by treatment with base. One of ordinary skill in the art would recognize that a wide variety of bases are useful for removing amino protecting groups that are base-labile. In some embodiments, a base is piperidine. In some embodiments, a base is 1,8-diazabicyclo[5.4.0]undec-7-ene (DBU).

In some embodiments, the present disclosure provides a method for preparing an oligonucleotide-ligand conjugate comprising one or more nucleic acid-ligand conjugate unit with one or more adamantyl and/or lipid moiety, said conjugate unit represented by formula D4:

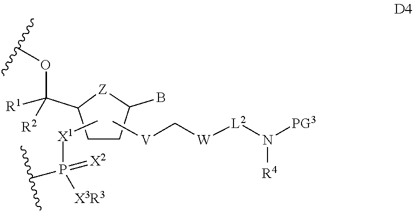

or a pharmaceutically acceptable salt thereof, comprising the steps of:
(a) providing a nucleic acid or analogue thereof of formula D3:

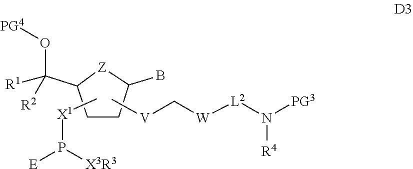

or salt thereof, and
(b) oligomerizing said compound of formula D3 to form a compound of formula D4,

In step (b) above, oligomerizing refers to preforming oligomerization forming conditions using known and commonly applied processes to prepare oligonucleotides in the art. For example, the nucleic acid or analogue thereof of formula D3 is coupled to a solid supported nucleic acid or analogue thereof bearing a 5′-hydroxyl group. Further steps can comprise one or more deprotections, couplings, phosphite oxidation, and cleavage from the solid support to provide an oligonucleotide of various nucleotide lengths, represented by a compound of formula D4 comprising an adamantyl or lipid conjugate of the disclosure.

In some embodiments, the present disclosure provides a method for preparing a nucleic acid or analogue thereof comprising one or more lipid conjugate, further comprising preparing a nucleic acid or analogue thereof of formula D3:

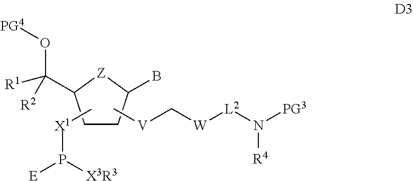

or a salt thereof, comprising the steps of:
(a) providing a nucleic acid or analogue thereof of formula C5:

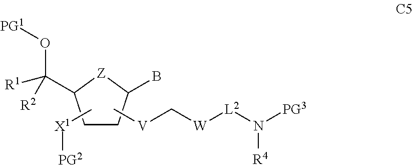

or salt thereof,
(b) deprotecting said nucleic acid or analogue thereof of formula C5 to form a compound of formula D1:

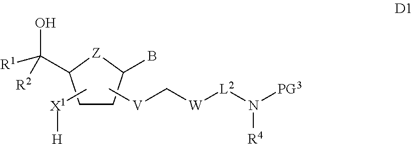

or salt thereof,
(c) protecting said nucleic acid or analogue thereof of formula D1 to form a nucleic acid or analogue thereof of formula D2:

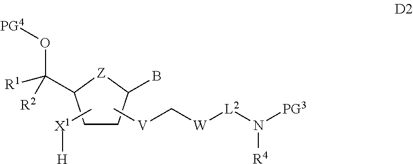

or salt thereof, and
(d) treating said nucleic acid or analogue thereof of formula D2 with a P(III) forming reagent to form a nucleic acid or analogue thereof of formula D3. In step (b) above, PG¹ and PG² of a nucleic acid or analogue thereof of formula C5 comprise silyl ethers or cyclic silylene derivatives that can be removed under acidic conditions or with fluoride anion. Examples of reagents providing fluoride anion for the removal of silicon-based protecting groups include hydrofluoric acid, hydrogen fluoride pyridine, triethylamine trihydrofluoride, tetra-N-butylammonium fluoride, and the like.

In step (c) above, a nucleic acid or analogue thereof of formula D1 is protected with a suitable hydroxyl protecting group. In certain embodiments, the protecting group PG⁴ used for protection of the 5′-hydroxyl group of a compound of formula D1 includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

In step (d) above, a nucleic acid or analogue thereof of formula D2 is treated with a P(III) forming reagent to afford a compound of formula D3. In the context of the present disclosure, a P(III) forming reagent is a phosphorus reagent that is reacted to for a phosphorus (III) compound. In some embodiments, the P(III) forming reagent is 2-cyanoethyl N,N-diisopropylchlorophosphoramidite or 2-cyanoethyl phosphorodichloridate. In certain embodiments, the P(III) forming reagent is 2-cyanoethyl N,N-diisopropylchlorophosphoramidite. One of ordinary skill would recognize that the displacement of a leaving group in a P(III) forming reagent by X¹ of a compound of formula D2 is achieved either with or without the presence of a suitable base. Such suitable bases are well known in the art and include organic and inorganic bases. In certain embodiments, the base is a tertiary amine such as triethylamine or diisopropylethylamine. In other embodiments, step (d) above is preformed using N,N-dimethylphosphoramic dichloride as a P(V) forming reagent.

### Formulations

Various formulations have been developed to facilitate oligonucleotide use. For example, oligonucleotides (e.g., RNAi oligonucleotide conjugates) can be delivered to a subject or a cellular environment using a formulation that minimizes degradation, facilitates delivery and/or uptake, or provides another beneficial property to the oligonucleotides in the formulation. In some embodiments, provided herein are compositions comprising oligonucleotides (e.g., RNAi oligonucleotide conjugates) reduce the expression of a target mRNA (e.g., a target mRNA expressed in the CNS). Such compositions can be suitably formulated such that when administered to a subject, either into the immediate environment of a target cell or systemically, a sufficient portion of the oligonucleotides enter the cell to reduce target gene expression. Any variety of suitable oligonucleotide formulations can be used to deliver oligonucleotides for the reduction of target gene expression as disclosed herein. In some embodiments, an oligonucleotide is formulated in buffer solutions such as phosphate buffered saline solutions, liposomes, micellar structures, and capsids.

Formulations of oligonucleotides with cationic lipids can be used to facilitate transfection of the oligonucleotides into cells. For example, cationic lipids, such as lipofectin, cationic glycerol derivatives, and polycationic molecules (e.g., polylysine, can be used. Suitable lipids include Oligofectamine, Lipofectamine (Life Technologies), NC388 (Ribozyme Pharmaceuticals, Inc., Boulder, Colo.), or FuGene 6 (Roche) all of which can be used according to the manufacturer's instructions.

Accordingly, in some embodiments, a formulation comprises a lipid nanoparticle. In some embodiments, an excipient comprises a liposome, a lipid, a lipid complex, a microsphere, a microparticle, a nanosphere or a nanoparticle, or may be otherwise formulated for administration to the cells, tissues, organs, or body of a subject in need thereof (see, e.g., Remington: THE SCIENCE AND PRACTICE OF PHARMACY, 22^{nd} edition, Pharmaceutical Press, 2013).

In some embodiments, the formulations herein comprise an excipient. In some embodiments, an excipient confers to a composition improved stability, improved absorption, improved solubility and/or therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide, or mineral oil). In some embodiments, an oligonucleotide is lyophilized for extending its shelf-life and then made into a solution before use (e.g., administration to a subject). Accordingly, an excipient in a composition comprising any one of the oligonucleotides described herein may be a lyoprotectant (e.g., mannitol, lactose, polyethylene glycol or polyvinylpyrrolidone) or a collapse temperature modifier (e.g., dextran, Ficoll™ or gelatin). Likewise, the oligonucleotides herein may be provided in the form of their free acids.

In some embodiments, a pharmaceutical composition is formulated to be compatible with its intended route of administration. Examples of routes of administration include parenteral (e.g., intravenous, intramuscular, intraperitoneal, intradermal, subcutaneous), oral (e.g., inhalation), transdermal (e.g., topical), transmucosal and rectal administration. In some embodiments, a pharmaceutical composition is formulated for delivery to the central nervous system (e.g., intrathecal, epidural). In some embodiments, a pharmaceutical composition is formulated for delivery to the eye (e.g., ophthalmic, intraocular, subconjunctival, intravitreal, retrobulbar, intracameral).

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (e.g., glycerol, propylene glycol, and liquid polyethylene glycol, and the like), and suitable mixtures thereof. In many cases, it will be preferable to include isotonic agents, for example, sugars, polyalcohols such as mannitol, sorbitol, sodium chloride in the composition. Sterile injectable solutions can be prepared by incorporating the oligonucleotides in a required amount in a selected solvent with one or a combination of ingredients enumerated above, as required, followed by filtered sterilization.

In some embodiments, a composition may contain at least about 0.1% of the therapeutic agent (e.g., an RNAi oligonucleotide conjugate herein) or more, although the percentage of the active ingredient(s) may be between about 1% to about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

### Methods of Use

### Reducing Target Gene Expression

In some embodiments, the disclosure provides methods for contacting or delivering to a cell or population of cells an effective amount of any of the RNAi oligonucleotide conjugates herein to reduce target gene expression (e.g., reduce expression of a target gene in the CNS). In some embodiments, a reduction of target gene expression is determined by measuring a reduction in the amount or level of target mRNA, protein encoded by the target mRNA, or target gene (mRNA or protein) activity in a cell. The methods include those described herein and known to one of ordinary skill in the art. In some embodiments, the disclosure provides methods for contacting or delivering to a cell or population of cells in one or more regions of the CNS an effective amount of any of the RNAi oligonucleotide conjugates herein to reduce target gene expression in the CNS. In some embodiments, regions of the CNS include, but are not limited to, cerebrum, prefrontal cortex, frontal cortex, motor cortex, temporal cortex, parietal cortex, occipital cortex, somatosensory cortex, hippocampus, caudate, striatum, globus pallidus, thalamus, midbrain, tegmentum, substantia nigra, pons, brainstem, cerebellar white matter, cerebellum, dentate nucleus, medulla, cervical spinal cord, thoracic spinal cord, lumbar spinal cord, cervical dorsal root ganglion, thoracic dorsal root ganglion, lumbar dorsal root ganglion, sacral dorsal root ganglion, nodose ganglia, femoral nerve, sciatic nerve, sural nerve, amygdala, hypothalamus, putamen, corpus callosum, and cranial nerve.

In some embodiments, the disclosure provides methods for reducing expression of a target mRNA in the CNS in a subject, comprising administering one or more RNAi oligonucleotide conjugates described herein. In some embodiments, the method comprises a reduced expression of a target mRNA in the somatosensory cortex (SS cortex). In some embodiments, the method comprises a reduced expression of a target mRNA in the hippocampus (HP). In some embodiments, the method comprises a reduced expression of a target mRNA in the striatum. In some embodiments, the method comprises a reduced expression of a target mRNA in the frontal cortex. In some embodiments, the method comprises a reduced expression of a target mRNA in the cerebellum. In some embodiments, the method comprises a reduced expression of a target mRNA in the hypothalamus (HY). In some embodiments, the method comprises a reduced expression of a target mRNA in the cervical spinal cord (CSC). In some embodiments, the method comprises a reduced expression of a target mRNA in the thoracic spinal cord (TSC). In some embodiments, the method comprises a reduced expression of a target mRNA in the lumbar spinal cord (LSC).

Methods provided herein are useful in any appropriate cell type. In some embodiments, a cell is any cell that expresses the target mRNA. In some embodiments, the cell is a primary cell obtained from a subject. In some embodiments, the primary cell has undergone a limited number of passages such that the cell substantially maintains is natural phenotypic properties. In some embodiments, a cell to which the oligonucleotide is delivered is ex vivo or in vitro (i.e., can be delivered to a cell in culture or to an organism in which the cell resides).

In some embodiments, the RNAi oligonucleotide conjugates disclosed herein are delivered to a cell or population of cells using a nucleic acid delivery method known in the art including, but not limited to, injection of a solution or pharmaceutical composition containing the RNAi oligonucleotide conjugate, bombardment by particles covered by the RNAi oligonucleotide conjugate, exposing the cell or population of cells to a solution containing the RNAi oligonucleotide conjugate, or electroporation of cell membranes in the presence of the RNAi oligonucleotide conjugate. Other methods known in the art for delivering oligonucleotides to cells may be used, such as lipid-mediated carrier transport, chemical-mediated transport, and cationic liposome transfection such as calcium phosphate, and others.

In some embodiments, reduction of target gene expression is determined by an assay or technique that evaluates one or more molecules, properties or characteristics of a cell or population of cells associated with target gene expression, or by an assay or technique that evaluates molecules that are directly indicative of target gene expression in a cell or population of cells (e.g., target mRNA or protein). In some embodiments, the extent to which an RNAi oligonucleotide conjugate provided herein reduces target gene expression is evaluated by comparing target gene expression in a cell or population of cells contacted with the RNAi oligonucleotide conjugate to a control cell or population of cells (e.g., a cell or population of cells not contacted with the RNAi oligonucleotide conjugate or contacted with a control RNAi oligonucleotide conjugate). In some embodiments, a control amount or level of target gene expression in a control cell or population of cells is predetermined, such that the control amount or level need not be measured in every instance the assay or technique is performed. The predetermined level or value can take a variety of forms. In some embodiments, a predetermined level or value can be single cut-off value, such as a median or mean.

In some embodiments, contacting or delivering an RNAi oligonucleotide conjugate described herein to a cell or a population of cells results in a reduction in target gene expression. In some embodiments, the reduction in target gene expression is relative to a control amount or level of target gene expression in cell or population of cells not contacted with the RNAi oligonucleotide conjugate or contacted with a control RNAi oligonucleotide conjugate. In some embodiments, the reduction in target gene expression is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a control amount or level of target gene expression. In some embodiments, the control amount or level of target gene expression is an amount or level of target mRNA and/or protein in a cell or population of cells that has not been contacted with an RNAi oligonucleotide conjugate herein. In some embodiments, the effect of delivery of an RNAi oligonucleotide conjugate to a cell or population of cells according to a method herein is assessed after any finite period or amount of time (e.g., minutes, hours, days, weeks, months). For example, in some embodiments, target gene expression is determined in a cell or population of cells at least about 4 hours, about 8 hours, about 12 hours, about 18 hours, about 24 hours; or at least about 1 day, about 2 days, about 3 days, about 4 days, about 5 days, about 6 days, about 7 days, about 8 days, about 9 days, about 10 days, about 11 days, about 12 days, about 13 days, about 14 days, about 21 days, about 28 days, about 35 days, about 42 days, about 49 days, about 56 days, about 63 days, about 70 days, about 77 days, or about 84 days or more after contacting or delivering the RNAi oligonucleotide conjugate to the cell or population of cells. In some embodiments, target gene expression is determined in a cell or population of cells at least about 1 month, about 2 months, about 3 months, about 4 months, about 5 months, or about 6 months or more after contacting or delivering the RNAi oligonucleotide conjugate to the cell or population of cells.

### Tissue-Specific Regulation of Gene Expression

In some embodiments, the disclosure provides a method for contacting or delivering to a cell or a population of cells an RNAi oligonucleotide conjugate described herein, wherein the cell or the population of cells is present in one or more target tissues in a subject. In some embodiments, the method comprises administering an RNAi oligonucleotide conjugate described herein to the subject, wherein the conjugate is distributed to one or more target tissues of the subject, and wherein the conjugate is contacted or delivered a cell or a population of cells within the one or more target tissues.

As used herein, a “target tissue” refers to a tissue of a subject wherein reduced expression of a target gene by a cell or a population of cells within the tissue provides one or more desirable physiological outcomes. In some embodiments, the target gene has abnormal expression in a cell or a population of cells within the one or more target tissues, wherein the abnormal expression contributes to the pathology of a disease or disorder in the subject. In some embodiments, reduced expression of the target gene by a cell or a population of cells within the target tissue functions to treat, mitigate, prevent, or alleviate the disease or disorder in the subject.

Although the distribution and/or function of an RNAi oligonucleotide conjugate within a target tissue is desirable for reducing target gene expression within a cell or population of cells that reside within the target tissue, the distribution and/or function of the conjugate to a non-target tissue has the potential to cause deleterious effects. For example, distribution of the conjugate to a non-target tissue may limit its availability for distribution to a target tissue, which in turn limits the potency and/or activity of the conjugate for reducing target gene expression within a cell or population of cells that resides within the target tissue. As another example, while the target tissue may have aberrant expression of the target gene and would benefit from reduced target gene expression to restore normal physiology, the non-target tissue may require expression of the target gene for normal physiological function. Under such circumstances, the distribution and/or function of the conjugate within the non-target tissue will impair the expression of the target gene in a manner that results in an undesirable or deleterious pathology. Accordingly, for numerous in vivo therapeutic contexts, it is beneficial to distribute the RNAi oligonucleotide to a target tissues in the subject, while limiting its distribution and/or function within one or more non-target tissues (e.g., the liver) in the subject.

In some embodiments, the disclosure provides a method for reducing or inhibiting expression of a target gene in a population of cells associated with one or more target tissues in a subject, comprising administering an RNAi oligonucleotide conjugate described herein, or a pharmaceutical composition thereof. In some embodiments, the method comprises distribution of the RNAi oligonucleotide conjugate to one or more target tissues of a subject, with minimal distribution to one or more non-target tissues of the subject. In some embodiments, the RNAi oligonucleotide conjugate is contacted or delivered to a cell or population of cells present in the one or more target tissues of the subject, with minimal contacting or delivery to a cell or population of cells present in one or more non-target tissues of the subject. In some embodiments, the expression of the target gene is reduced in the one or more target tissues without being reduced to the same or similar level in one or more non-target tissues.

In some embodiments, the method results in (i) a reduced expression of the target gene by a cell or population of cells in one or more target tissues relative to a control expression of the target gene; and (ii) a substantially equivalent expression of the target gene by a cell or population of cells in one or more non-target tissues relative to a control expression of the target gene. In some embodiments, the control expression of the target gene corresponds to the amount or level of expression of the target gene in a cell or population of cells from an equivalent tissue that is not contacted with the RNAi oligonucleotide conjugate or contacted with a control RNAi oligonucleotide conjugate. In some embodiments, the reduction of target gene expression is measured as a reduction in the amount or level of a target mRNA transcribed from the target gene or protein encoded by the target gene. In some embodiments, the method results in (i) a reduced expression of target mRNA in one or more target tissues relative to a control; and (ii) a substantially equivalent expression of target mRNA in one or more non-target tissues relative to a control. In some embodiments, the method results in (i) a reduced level of target protein in one or more target tissues relative to a control; and (ii) a substantially equivalent level of target protein in one or more non-target tissues relative to a control.

In some embodiments, the disclosure provides a method for reducing or inhibiting expression of a target gene in a population of cells associated with the CNS in a subject, comprising administering an RNAi oligonucleotide conjugate described herein, or a pharmaceutical composition thereof. In some embodiments, the method comprises distribution of the RNAi oligonucleotide conjugate to the CNS in the subject, with minimal distribution to one or more non-target tissues of the subject (e.g., the liver). In some embodiments, the RNAi oligonucleotide conjugate is contacted or delivered to a cell or population of cells present in the CNS of the subject, with minimal contacting or delivery to a cell or population of cells present in one or more non-target tissues of the subject (e.g., the liver). In some embodiments, the expression of the target gene is reduced in the CNS of the subject without being reduced to the same level in one or more non-target tissues (e.g., the liver).

In some embodiments, expression of the target gene is reduced in the CNS without being reduced to the same level in one or more non-target tissues. In some embodiments, the one or more non-target tissues comprises liver tissue. In some embodiments, the method results in (i) a reduced expression of a target gene in a cell or population of cells of the CNS relative to a control expression of the target gene; and (ii) substantially equivalent expression of the target gene in a cell or population of cells of one or more non-target tissues relative to a control expression of the target gene. In some embodiments, the control expression of the target gene corresponds to the amount or level of expression of the target gene in a cell or population of cells from an equivalent tissue that is not contacted with the RNAi oligonucleotide conjugate or contacted with a control RNAi oligonucleotide conjugate. In some embodiments, the method results in (i) a reduced expression of target gene in a cell or population of cells of the CNS relative to a control expression of the target gene (e.g., expression of the target gene in a cell or population of cells of the CNS not contacted with the RNAi oligonucleotide conjugate or contacted with a control RNAi oligonucleotide conjugate); and (ii) substantially equivalent expression of the target gene in a cell or population of cells of the liver relative to a control expression of the target gene (e.g., expression of the target gene in a cell or population of cells of the liver not contacted with the RNAi oligonucleotide conjugate or contacted with a control RNAi oligonucleotide conjugate). In some embodiments, the method results in expression of the target gene in a cell or population of cells of the CNS that is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a control expression of the target gene. In some embodiments, expression of the target gene in the liver is comparable to a control expression of the target gene (e.g., having a difference not more than about ±30%, about ±25%, about ±20%, about ±15%, about ±10%, about ±5%, about ±4%, about ±3%, about ±2%, or about ±1%). In some embodiments, the reduction of target gene expression in the CNS is measured as a reduction in the amount or level of a target mRNA transcribed from the target gene or protein encoded by the target gene.

### Treatment Methods

The disclosure provides oligonucleotides for use as a medicament, in particular for use in a method for the treatment of diseases, disorders, and conditions associated with the CNS. The disclosure also provides RNAi oligonucleotide conjugates for use, or adaptable for use, to treat a subject (e.g., a human having a disease, disorder or condition associated with target gene expression) that would benefit from reducing target gene expression (e.g., in the CNS). In some aspects, the disclosure provides RNAi oligonucleotide conjugates for use, or adapted for use, to treat a subject having a disease, disorder or condition associated with target gene expression. The disclosure also provides RNAi oligonucleotide conjugates for use, or adaptable for use, in the manufacture of a medicament or pharmaceutical composition for treating a disease, disorder or condition associated with target gene expression. In some embodiments, the RNAi oligonucleotide conjugates for use, or adaptable for use, target mRNA and reduce target gene expression (e.g., via the RNAi pathway). In some embodiments, the RNAi oligonucleotide conjugates for use, or adaptable for use, target mRNA and reduce the amount or level of target mRNA, protein and/or activity.

In addition, in some embodiments of the methods herein, a subject having a disease, disorder or condition associated with target gene expression or is predisposed to the same is selected for treatment with an RNAi oligonucleotide conjugate herein. In some embodiments, the method comprises selecting an individual having a marker (e.g., a biomarker) for a disease, disorder or condition associated with target gene expression, or predisposed to the same, such as, but not limited to, target mRNA, protein, or a combination thereof. Likewise, and as detailed below, some embodiments of the methods provided by the disclosure include steps such as measuring or obtaining a baseline value for a marker of target gene expression, and then comparing such obtained value to one or more other baseline values or values obtained after the subject is administered the RNAi oligonucleotide conjugate to assess the effectiveness of treatment.

The disclosure also provides methods of treating a subject having, suspected of having, or at risk of developing a disease, disorder or condition associated with target gene expression with an RNAi oligonucleotide conjugate provided herein. In some aspects, the disclosure provides methods of treating or attenuating the onset or progression of a disease, disorder or condition associated with target gene expression using the RNAi oligonucleotide conjugates provided herein. In other aspects, the disclosure provides methods to achieve one or more therapeutic benefits in a subject having a disease, disorder or condition associated with target gene expression using the RNAi oligonucleotide conjugates provided herein. In some embodiments of the methods herein, the subject is treated by administering a therapeutically effective amount of any one or more of the RNAi oligonucleotide conjugates provided herein. In some embodiments, treatment comprises reducing target gene expression (e.g., in the CNS). In some embodiments, the subject is treated therapeutically. In some embodiments, the subject is treated prophylactically.

In some embodiments of the methods herein, an RNAi oligonucleotide conjugate provided herein, or a pharmaceutical composition comprising the RNAi oligonucleotide conjugate, is administered to a subject having a disease, disorder or condition associated with target gene expression such that target gene expression is reduced in the subject, thereby treating the subject. In some embodiments, an amount or level of target mRNA is reduced in the subject. In some embodiments, an amount or level of protein encoded by the target mRNA is reduced in the subject.

In some embodiments of the methods herein, an RNAi oligonucleotide conjugate provided herein, or a pharmaceutical composition comprising the RNAi oligonucleotide conjugate, is administered to a subject having a disease, disorder or condition associated with target gene expression such that target gene expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression prior to administration of the RNAi oligonucleotide conjugate or pharmaceutical composition. In some embodiments, target gene expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression in a subject (e.g., a reference or control subject) not receiving the RNAi oligonucleotide conjugate or pharmaceutical composition or receiving a control RNAi oligonucleotide conjugate, pharmaceutical composition or treatment.

In some embodiments of the methods herein, an RNAi oligonucleotide conjugate herein, or a pharmaceutical composition comprising the RNAi oligonucleotide conjugate, is administered to a subject having a disease, disorder or condition associated with target gene expression such that an amount or level of target mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of target mRNA prior to administration of the RNAi oligonucleotide conjugate or pharmaceutical composition. In some embodiments, an amount or level of target mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of target mRNA in a subject (e.g., a reference or control subject) not receiving the RNAi oligonucleotide conjugate or pharmaceutical composition or receiving a control RNAi oligonucleotide conjugate, pharmaceutical composition or treatment.

In some embodiments of the methods herein, an RNAi oligonucleotide conjugate herein, or a pharmaceutical composition comprising the RNAi oligonucleotide conjugate, is administered to a subject having a disease, disorder or condition associated with target gene expression such that an amount or level of protein encoded by a target gene is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of protein encoded by the target gene prior to administration of the RNAi oligonucleotide conjugate or pharmaceutical composition. In some embodiments, an amount or level of protein encoded by a target gene is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of protein encoded by the target gene in a subject (e.g., a reference or control subject) not receiving the RNAi oligonucleotide conjugate or pharmaceutical composition or receiving a control RNAi oligonucleotide conjugate, pharmaceutical composition or treatment.

In some embodiments of the methods herein, an RNAi oligonucleotide conjugate herein, or a pharmaceutical composition comprising the RNAi oligonucleotide conjugate, is administered to a subject having a disease, disorder or condition associated with target gene expression such that an amount or level of target gene activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of target gene activity prior to administration of the RNAi oligonucleotide conjugate or pharmaceutical composition. In some embodiments, an amount or level of target gene activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of target gene activity in a subject (e.g., a reference or control subject) not receiving the RNAi oligonucleotide conjugate or pharmaceutical composition or receiving a control RNAi oligonucleotide conjugate, pharmaceutical composition or treatment.

Suitable methods for determining target gene expression, an amount or level of target mRNA, an amount or level of protein encoded by the target gene, and/or an amount or level of target gene activity, in the subject, or in a sample from the subject, are known in the art. Further, the Examples set forth herein illustrate exemplary methods for determining target gene expression.

In some embodiments, target gene expression, an amount or level of target gene mRNA, an amount or level of protein encoded by a target gene, an amount or level of target gene activity, or any combination thereof, is reduced in a cell, a population or a group of cells (e.g., an organoid), an organ (e.g., CNS), blood or a fraction thereof (e.g., plasma), a tissue (e.g., brain tissue), a sample (e.g., CSF sample or a brain biopsy sample), or any other biological material obtained or isolated from the subject. In some embodiments, target gene expression, an amount or level of target gene mRNA, an amount or level of protein encoded by a target gene, an amount or level of target gene activity, or any combination thereof, is reduced in more than one type of cell, more than one groups of cells, more than one organ (e.g., brain and one or more other organ(s)), more than one fraction of blood (e.g., plasma and one or more other blood fraction(s)), more than one type of tissue (e.g., brain tissue and one or more other type(s) of tissue), more than one type of sample (e.g., a brain biopsy sample and one or more other type(s) of biopsy sample) obtained or isolated from the subject.

In some embodiments, the method provides for a reduction in target gene expression in a cell or population of cells in one or more target tissues (e.g., the CNS), while maintaining target gene expression in a cell or population of cells of one or more non-target tissues (e.g., the liver). In some embodiments, target gene expression within the one or more non-target tissues contributes to normal physiological function in the subject. In some embodiments, the method provides for differential target gene expression, such that irregular target gene expression in the target tissue is reduced to treat, mitigate, or alleviate a disease or disorder associated with the target gene, without inducing dysregulation of the target gene in a non-target tissue that would result in disrupted physiological function.

In some embodiments, the disclosure provides a method of treating a disease, disorder or condition associated with target gene comprising administering an RNAi oligonucleotide conjugate described herein, or a pharmaceutical composition thereof, to a subject in need thereof, wherein (i) the amount or level of target gene expression is reduced in a target tissue of the subject (e.g., the CNS) by at least about 30%, about 35%, about 40%, about 45%, about 50%, about 55%, about 60%, about 65%, about 70%, about 75%, about 80%, about 85%, about 90%, about 95%, about 99% or greater than 99% compared to a control amount or level of a target gene expression; and (ii) the amount or level of target gene expression in one or more non-target tissues (e.g., the liver) is comparable to a control expression level of the target gene (e.g., having a difference not more than about ±30%, about ±25%, about ±20%, about ±15%, about ±10%, about ±5%, about ±4%, about ±3%, about ±2%, or about ±1%).

In some embodiments, an amount or level of target mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of target mRNA in a subject (e.g., a reference or control subject) not receiving the RNAi oligonucleotide conjugate or pharmaceutical composition or receiving a control RNAi oligonucleotide conjugate, pharmaceutical composition or treatment.

Examples of a disease, disorder or condition associated with target gene expression include, but are not limited to, Progressive Supranuclear Palsy (PSP), Corticobasal degeneration (CBD), Argyrophilic grain disease (AGD), Globular glial tauopathy (GGT), Aging-related tau astrogliopathy (ARTAG), Familial Frontotemporal Dementia 17 (FTD-17), Tauopathy with Respiratory Failure, Dementia with Seizures, Pick's disease, Myotonic dystrophy 1 or 2 (MD1 or MD2), Down's syndrome, Spastic Paraplegia (SP), Niemann-Pick disease type C, Dementia with Lewy bodies (DLB), Lewy body dysphagia, Lewy body disease, Olivopontocerebellar atrophy, Striatonigral degeneration, Shy-Drager syndrome, Spinal muscular atrophy V (SMAV), Huntington's Disease (HD), Alzheimer's Disease, SCA1, SCA2, SCA3, SCA7, SCA10 (spinocerebellar ataxia type 1, 2, 3, 7 or 10), Multiple System Atrophy (MSA), Spinal and Bulbar Muscular Atrophy (SBMA, Kennedy disease), Friedrich Ataxia, Fragile X-associated tremor/ataxia syndrome (FXTAS), Fragile X syndrome (FRAXA), X-Linked Mental Retardation (XLMR), Parkinson's Disease, Dystonia, SBMA (spinobulbar muscular atrophy), neuropathic pain disorders, spinal cord injury, Dentatorubral-pallidoluysian atrophy (DRPLA), recessive CNS disorders, ALS (amyotrophic lateral sclerosis), M2DS (MECP2 duplication syndrome), FTD (frontotemporal dementia), Prion disease, Adult Onset Leukodystrophy, Alexander's Disease, Krabbe Disease, Chronic Traumatic Encephalopathy, Pelizaeus-Merzbacher disease (PMD), Lafora disease, stroke, Cerebral Amyloid Angiopathy (CAA), and Metachromatic Leukodystrophy (MLD).

In some embodiments, the target gene may be a target gene from any mammal, such as a human. Any gene may be silenced according to the method described herein.

Methods described herein are typically involve administering to a subject a therapeutically effective amount of an RNAi oligonucleotide conjugate herein, that is, an amount capable of producing a desirable therapeutic result. A therapeutically acceptable amount may be an amount that can therapeutically treat a disease or disorder. The appropriate dosage for any one subject will depend on certain factors, including the subject's size, body surface area, age, the composition to be administered, the active ingredient(s) in the composition, time and route of administration, general health, and other drugs being administered concurrently.

In some embodiments, a subject is administered any one of the compositions herein either enterally (e.g., orally, by gastric feeding tube, by duodenal feeding tube, via gastrostomy or rectally), parenterally (e.g., subcutaneous injection, intravenous injection or infusion, intra-arterial injection or infusion, intraosseous infusion, intramuscular injection, intracerebral injection, intracerebroventricular injection, intrathecal), topically (e.g., epicutaneous, inhalational, via eye drops, or through a mucous membrane), or by direct injection into a target organ (e.g., the brain of a subject).

In some embodiments, an RNAi oligonucleotide conjugate herein, or a composition thereof, is administered intrathecally into cerebrospinal fluid (CSF) (e.g., injection or infusion into the fluid within the subarachnoid space). In some embodiments, intrathecal administration of an RNAi oligonucleotide conjugate herein, or a composition thereof, is performed as a bolus injection into the subarachnoid space. In some embodiments, intrathecal administration of an RNAi oligonucleotide conjugate herein, or a composition thereof, is performed as an infusion into the subarachnoid space. In some embodiments, intrathecal administration of an RNAi oligonucleotide conjugate herein, or a composition thereof, is performed via a catheter into the subarachnoid space. In some embodiments, intrathecal administration of an RNAi oligonucleotide conjugate herein, or a composition thereof, is performed via a pump. In some embodiments, intrathecal administration of an RNAi oligonucleotide conjugate herein, or a composition thereof, is performed via an implantable pump. In some embodiments, administration is performed via an implantable device that operates or functions a reservoir.

In some embodiments, an RNAi oligonucleotide conjugate herein, or a composition thereof, is administered intrathecally into the cerebellomedullary cistern (also referred to as the cisterna magna). Intrathecal administration into the cisterna magna is referred to as “intracisternal administration” or “intracisternal magna (i.c.m.) administration. In some embodiments, an RNAi oligonucleotide conjugate herein, or composition thereof, is administered intrathecally into the subarachnoid space of the lumbar spinal cord. Intrathecal administration into the subarachnoid space of the lumbar spinal cord is referred to as “lumbar intrathecal (i.t.) administration”. In some embodiments, an RNAi oligonucleotide conjugate herein, or composition thereof, is administered intrathecally into the subarachnoid space of the cervical spinal cord. Intrathecal administration into the subarachnoid space of the cervical spinal cord is referred to as “cervical intrathecal (i.t.) administration”. In some embodiments, an RNAi oligonucleotide conjugate herein, or composition thereof, is administered intrathecally into the subarachnoid space of the thoracic spinal cord. Intrathecal administration into the subarachnoid space of the thoracic spinal cord is referred to as “thoracic intrathecal (i.t.) administration”. In some embodiments, an RNAi oligonucleotide conjugate herein, or composition thereof, is administered by intracerebroventricular injection or infusion into the cerebral ventricles. Intracerebroventricular administration into the ventricular space is referred to as “intracerebroventricular (i.c.v.) administration”. In some embodiments, an Ommaya reservoir is used to administer an RNAi oligonucleotide conjugate herein, or composition thereof, by intracerebroventricular injection or infusion.

In some embodiments, an RNAi oligonucleotide conjugate herein, or a composition thereof, is administered once every year, once every 6 months, once every 4 months, quarterly (once every three months), bi-monthly (once every two months), monthly or weekly. In some embodiments, an RNAi oligonucleotide conjugate herein, or a composition thereof, is administered every week or at intervals of two, or three weeks. In some embodiments, an RNAi oligonucleotide conjugate herein, or a composition thereof, is administered daily. In some embodiments, a subject is administered one or more loading doses of an RNAi oligonucleotide conjugate herein, or a composition thereof, followed by one or more maintenance doses of the RNAi oligonucleotide conjugate, or a composition thereof.

In some embodiments, the subject to be treated is a human or non-human primate or other mammalian subject. Other exemplary subjects include domesticated animals such as dogs and cats; livestock such as horses, cattle, pigs, sheep, goats, and chickens; and animals such as mice, rats, guinea pigs, and hamsters.

### Kits

In some embodiments, the disclosure provides a kit comprising an RNAi oligonucleotide conjugate herein, or a composition thereof, described herein, and instructions for use. In some embodiments, the kit comprises an RNAi oligonucleotide conjugate herein, or a composition thereof, described herein, and a package insert containing instructions for use of the kit and/or any component thereof. In some embodiments, the kit comprises, in a suitable container, an RNAi oligonucleotide conjugate herein, or a composition thereof, described herein, one or more controls, and various buffers, reagents, enzymes and other standard ingredients well known in the art. In some embodiments, the container comprises at least one vial, well, test tube, flask, bottle, syringe, or other container means, into which the RNAi oligonucleotide conjugate herein, or a composition thereof, is placed, and in some instances, suitably aliquoted. In some embodiments where an additional component is provided, the kit contains additional containers into which this component is placed. The kits can also include a means for containing an RNAi oligonucleotide conjugate herein, or a composition thereof, and any other reagent in close confinement for commercial sale. Such containers may include injection or blow-molded plastic containers into which the desired vials are retained. Containers and/or kits can include labeling with instructions for use and/or warnings.

In some embodiments, a kit comprises an RNAi oligonucleotide conjugate herein, or a composition thereof, described herein, and a pharmaceutically acceptable carrier, or a pharmaceutical composition comprising the RNAi oligonucleotide conjugate and instructions for treating or delaying progression of a disease, disorder or condition associated with target gene expression in a subject in need thereof.

In some embodiments, the disclosure provides a kit comprising an RNAi oligonucleotide conjugate herein, or a composition thereof, described herein, and a pharmaceutically acceptable carrier, or a pharmaceutical composition comprising the RNAi oligonucleotide conjugate, and instructions for reducing or inhibiting expression of a target mRNA in a subject expressed by a population of cells associated with the CNS.

In some embodiments, the disclosure provides a kit comprising an RNAi oligonucleotide conjugate herein, or a composition thereof, described herein, and a pharmaceutically acceptable carrier, or a pharmaceutical composition comprising the RNAi oligonucleotide conjugate, and instructions for reducing or inhibiting expression of a target mRNA in a subject expressed by a population of cells associated with the CNS without reducing expression of the target mRNA to the same level outside the CNS.

In some embodiments, the disclosure provides a kit comprising an RNAi oligonucleotide conjugate herein, or a composition thereof, described herein, and a pharmaceutically acceptable carrier, or a pharmaceutical composition comprising the RNAi oligonucleotide conjugate, and instructions for reducing or inhibiting expression of a target mRNA in a subject expressed by a population of cells associated with the CNS without reducing expression of the target mRNA to the same level in cells of the liver.

In some embodiments, the disclosure provides a kit comprising an RNAi oligonucleotide conjugate herein, or a composition thereof, described herein, and a pharmaceutically acceptable carrier, or a pharmaceutical composition comprising the RNAi oligonucleotide conjugate, and instructions for reducing or inhibiting expression of a target mRNA in a subject in the CNS, wherein the target mRNA is associated with a disease or disorder, optionally a neurological disease or disorder.

In some embodiments, the disclosure provides a kit comprising an RNAi oligonucleotide conjugate herein, or a composition thereof, described herein, and a pharmaceutically acceptable carrier, or a pharmaceutical composition comprising the RNAi oligonucleotide conjugate, and instructions for reducing or inhibiting expression of a target mRNA in a subject in the CNS without reducing expression of the target mRNA to the same level outside the CNS, wherein the target mRNA is associated with a disease or disorder, optionally a neurological disease or disorder.

In some embodiments, the disclosure provides a kit comprising an RNAi oligonucleotide conjugate herein, or a composition thereof, described herein, and a pharmaceutically acceptable carrier, or a pharmaceutical composition comprising the RNAi oligonucleotide conjugate, and instructions for reducing or inhibiting expression of a target mRNA in a subject in the CNS without reducing expression of the target mRNA to the same level in the liver, wherein the target mRNA is associated with a disease or disorder, optionally a neurological disease or disorder.

### OTHER EMBODIMENTS

The disclosure relates to the following embodiments. Throughout this section, the term embodiment is abbreviated as “E” followed by an ordinal. For example, E1 is equivalent to Embodiment 1.

E1. A nucleic acid-ligand conjugate represented by formula I-a:

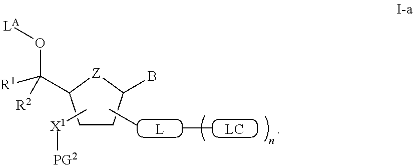

or a pharmaceutically acceptable salt thereof, wherein:
B is a nucleobase or hydrogen;
R¹ and R² are independently hydrogen, halogen, R^{A}, —CN, —S(O)R, —S(O)₂R, —Si(OR)₂R, —Si(OR)R2, or —SiR₃, or
   R¹ and R² on the same carbon are taken together with their intervening atoms to form a 3-membered saturated or partially unsaturated ring having 0-3 heteroatoms, independently selected from nitrogen, oxygen, and sulfur;
each R^{A} is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur;
   L^{A} is independently PG¹, or -L-ligand;
   PG¹ is hydrogen or a suitable hydroxyl protecting group;
   each ligand is independently -(LC)n, and/or an adamantyl group;
each LC is independently a lipid conjugate moiety comprising a saturated or unsaturated, straight, or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, or —P(S)OR—;
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, adamantanenyl, a 4-7 membered saturated or partially unsaturated heterocyclylenyl having 1-3 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
n is 1-10;
L is a covalent bond or a bivalent saturated or unsaturated, straight or branched C₁₋₅₀ hydrocarbon chain, wherein 0-10 methylene units of the hydrocarbon chain are independently replaced by -Cy-, —O—, —NR—, —N(R)—C(O)—, —S—, —C(O)—, —S(O)—, —S(O)₂—, —P(O)OR—, —P(S)OR—, —V¹CR²W¹— or

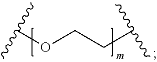

m is 1-50;
X¹, V¹ and W¹ are independently —C(R)₂—, —OR, —O—, —S—, —Se—, or —NR—;
Z is —O—, —S—, —NR—, or —CR₂—; and
PG² is hydrogen, a phosphoramidite analogue, or a suitable protecting group.

E2. The nucleic acid-ligand conjugate of E1, wherein the conjugate is represented by formula I-b or I-c:

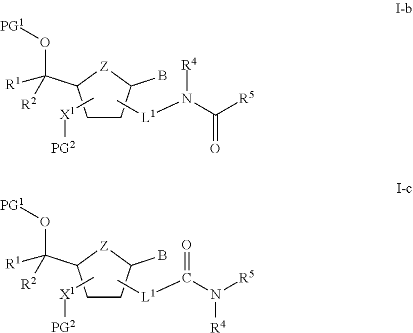

or a pharmaceutically acceptable salt thereof; wherein
L¹ is a covalent bond or a bivalent saturated or unsaturated, straight or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, —P(S)OR—, or

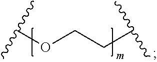

R⁴ is hydrogen, R^{A}, or a suitable amine protection group; and
R⁵ is adamantyl, or a saturated or unsaturated, straight, or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, or —P(S)OR—.

E3. A nucleic acid-ligand conjugate represented by formula I-Ib or I-Ic:

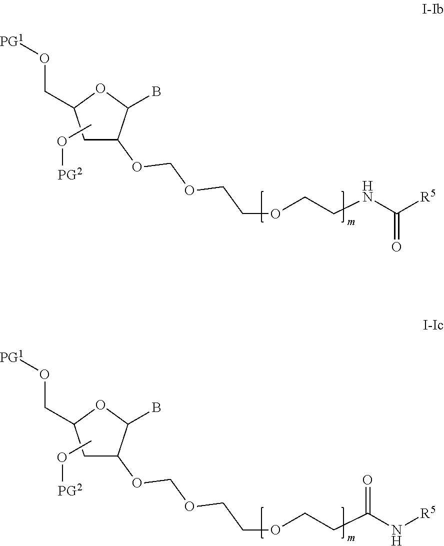

or a pharmaceutically acceptable salt thereof; wherein
B is a nucleobase or hydrogen;
m is 1-50;
PG¹ and PG² are independently a hydrogen, a phosphoramidite analogue, or a suitable protecting group; and
R⁵ is adamantyl, or a saturated or unsaturated, straight, or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, or —P(S)OR—.

E4. The nucleic acid-ligand conjugate of E3, wherein:
R⁵ is selected from

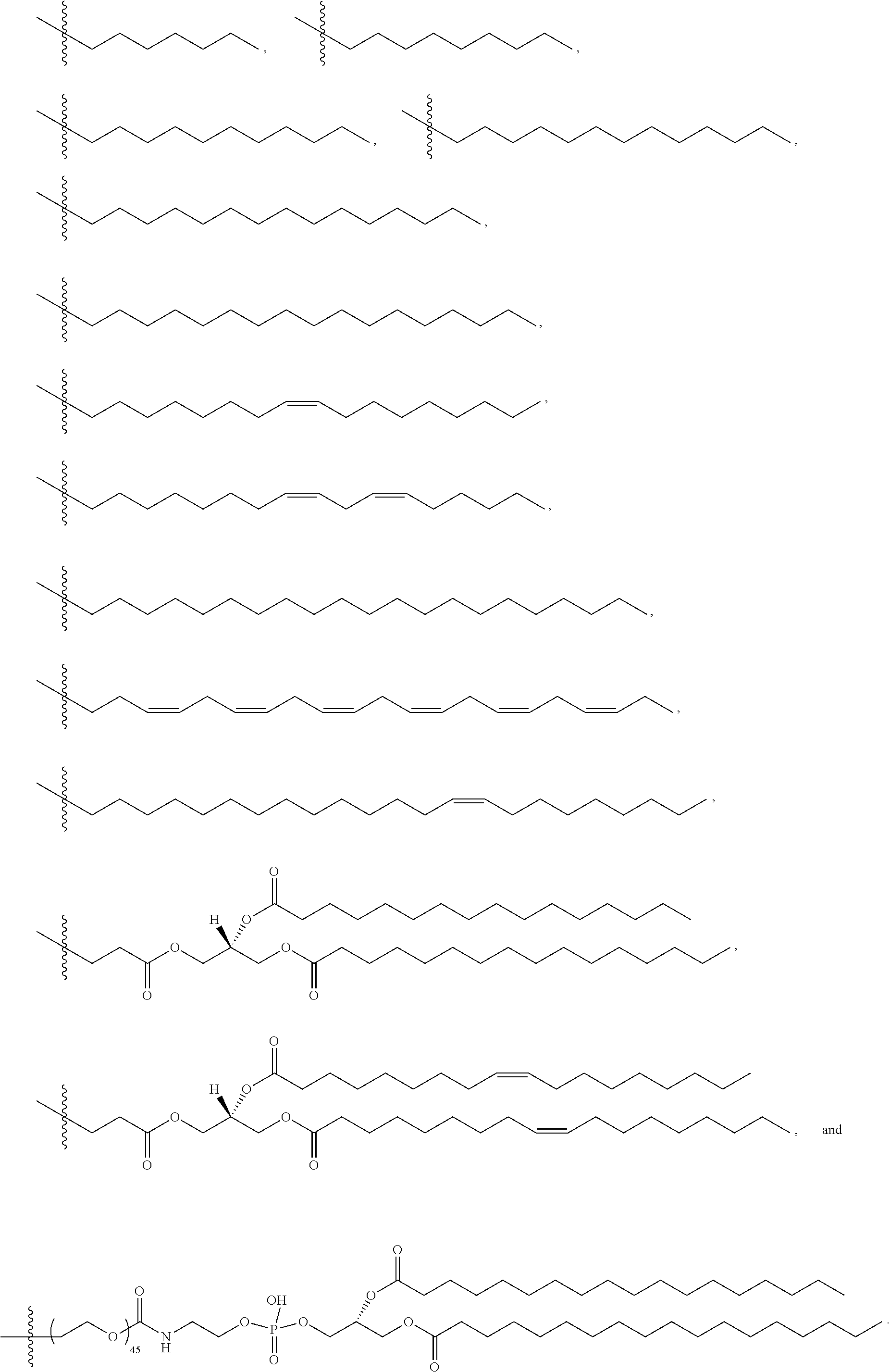

E5. The nucleic acid-ligand conjugate of E4, wherein:
R⁵ is selected from

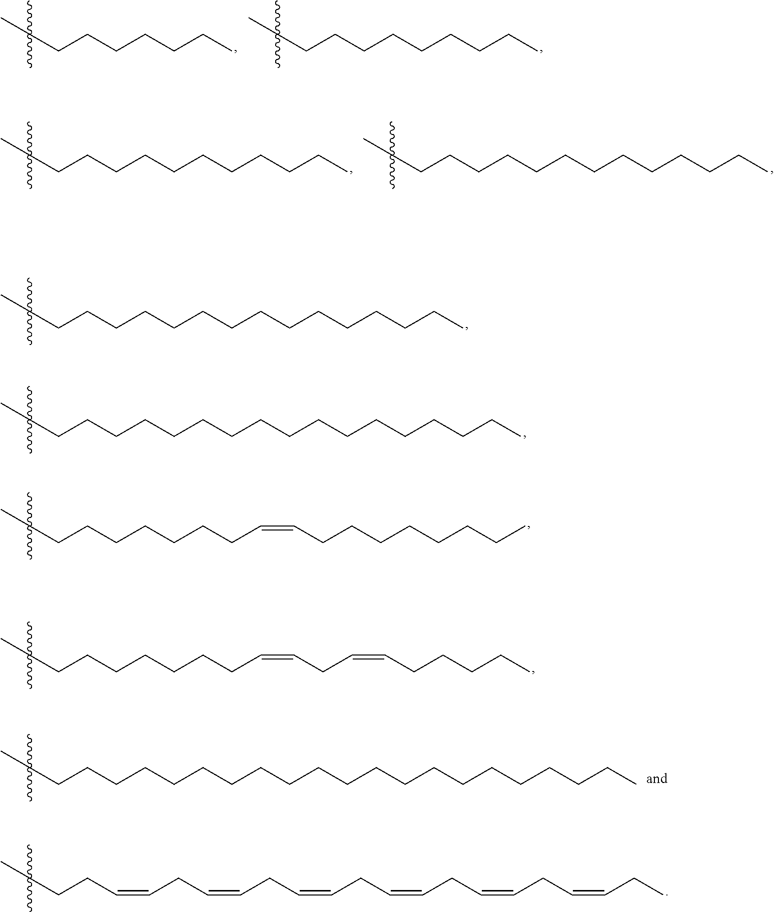

E6. An oligonucleotide ligand conjugate comprising one or more nucleic acid-ligand conjugate of any one of E1 to E5.

E7. The oligonucleotide-ligand conjugate of E6, wherein the conjugate comprises 1, 2, 3, 4, 5, 6, 7, 8, 9 or 10 nucleic acid-ligand conjugates.

E8. An oligonucleotide-ligand conjugate comprising one or more nucleic acid-ligand conjugates represented by formula II-a:

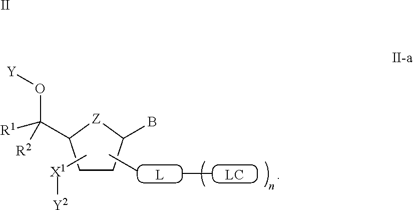

or a pharmaceutically acceptable salt thereof, wherein:
B is a nucleobase or hydrogen;
R¹ and R² are independently hydrogen, halogen, R^{A}, —CN, —S(O)R, —S(O)₂R, —Si(OR)₂R, —Si(OR)R2, or —SiR₃; or
   R¹ and R² on the same carbon are taken together with their intervening atoms to form a 3-7 membered saturated or partially unsaturated ring having 0-3 heteroatoms, independently selected from nitrogen, oxygen, and sulfur;
each R^{A} is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, a suitable protecting group, or an optionally substituted group selected from C1-6 aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur; or
   two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur;
each LC is independently a lipid conjugate moiety comprising a saturated or unsaturated, straight, or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, —P(S)OR—;
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-3 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
n is 1-10;
L is a covalent bond or a bivalent saturated or unsaturated, straight or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, —P(S)OR—, —V¹CR²W¹—, or

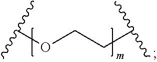

m is 1-50;
X¹, V¹ and W¹ are independently —C(R)₂—, —OR, —O—, —S—, —Se—, or —NR—;
Y is hydrogen, a suitable hydroxyl protecting group,

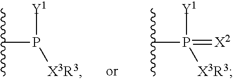

R³ is hydrogen, a suitable protecting group, a suitable prodrug, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur;
X² is O, S, or NR;
X³ is —O—, —S—, —BH₂—, or a covalent bond;
Y¹ is a linking group attaching to the 2′- or 3′-terminal of a nucleoside, a nucleotide, or an oligonucleotide;
Y² is hydrogen, a suitable protecting group, a phosphoramidite analogue, an internucleotide linking group attaching to the 5′-terminal of a nucleoside, a nucleotide, or an oligonucleotide, or a linking group attaching to a solid support; and
Z is —O—, —S—, —NR—, or —CR₂—.

E9. The oligonucleotide-ligand conjugate of E8, wherein the conjugate is represented by formula II-b or II-c:

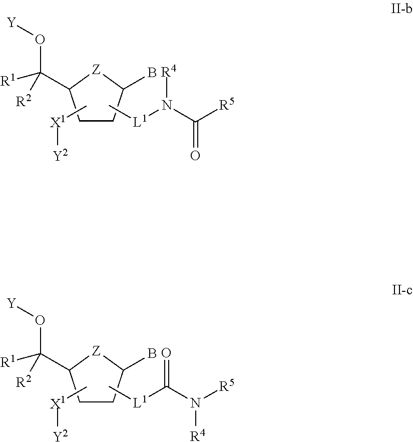

or a pharmaceutically acceptable salt thereof, wherein:
L¹ is a covalent bond, a monovalent or a bivalent saturated or unsaturated, straight or branched C₁₋₅₀ hydrocarbon chain, wherein 0-10 methylene units of the hydrocarbon chain are independently replaced by —Cy-, —O—, —C(O)NR—, —NR—, —S—, —C(O)—, —C(O)O—, —S(O)—, —S(O)₂—, —P(O)OR—, —P(S)OR—, or

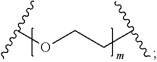

R⁴ is hydrogen, R^{A}, or a suitable amine protection group; and
R⁵ is adamantyl, or a saturated or unsaturated, straight, or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, or —P(S)OR.

E10. The oligonucleotide-ligand conjugate of E9, wherein:
R⁵ is selected from

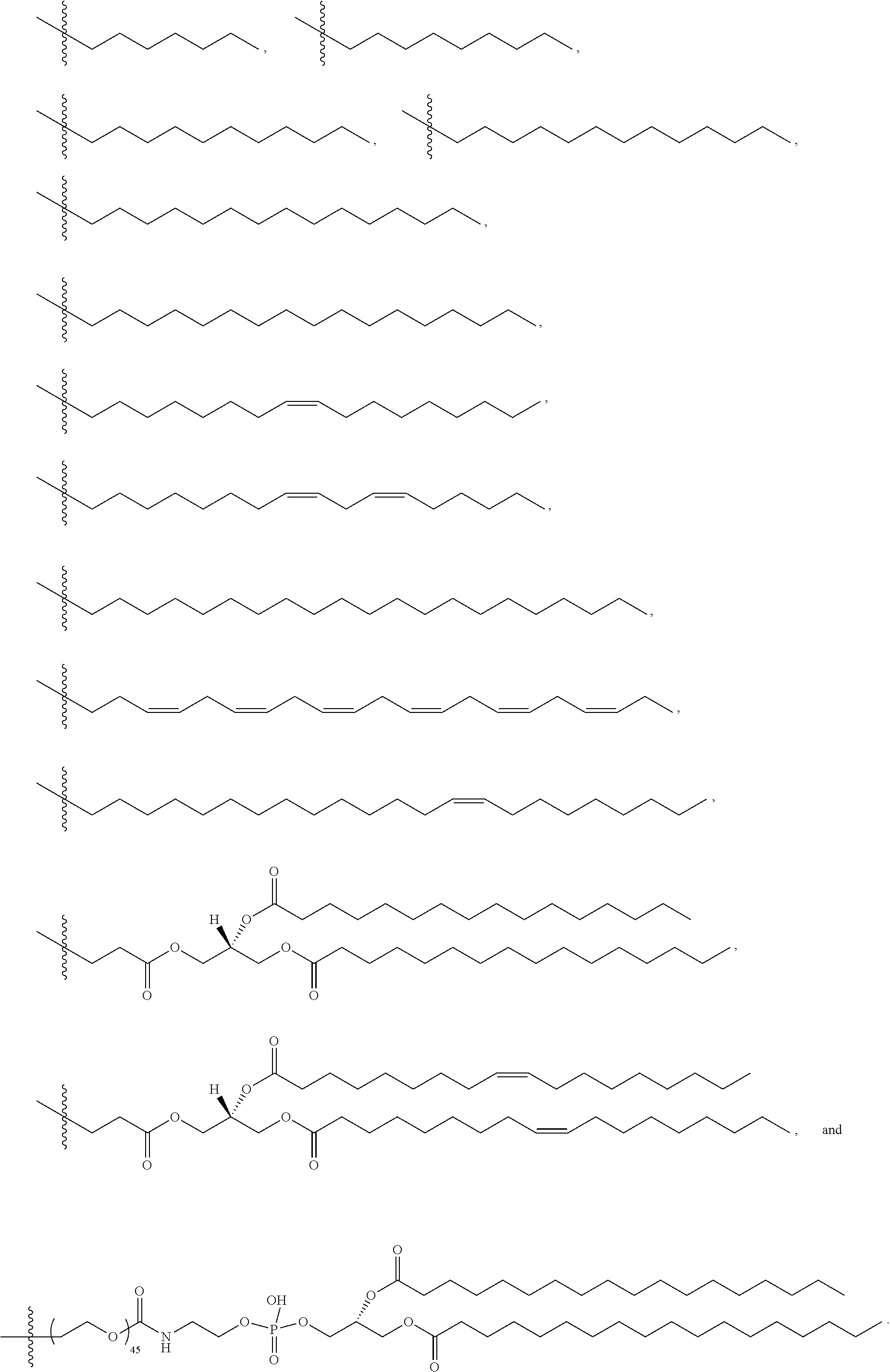

E11. The oligonucleotide-ligand conjugate of E9, wherein:
R⁵ is selected from

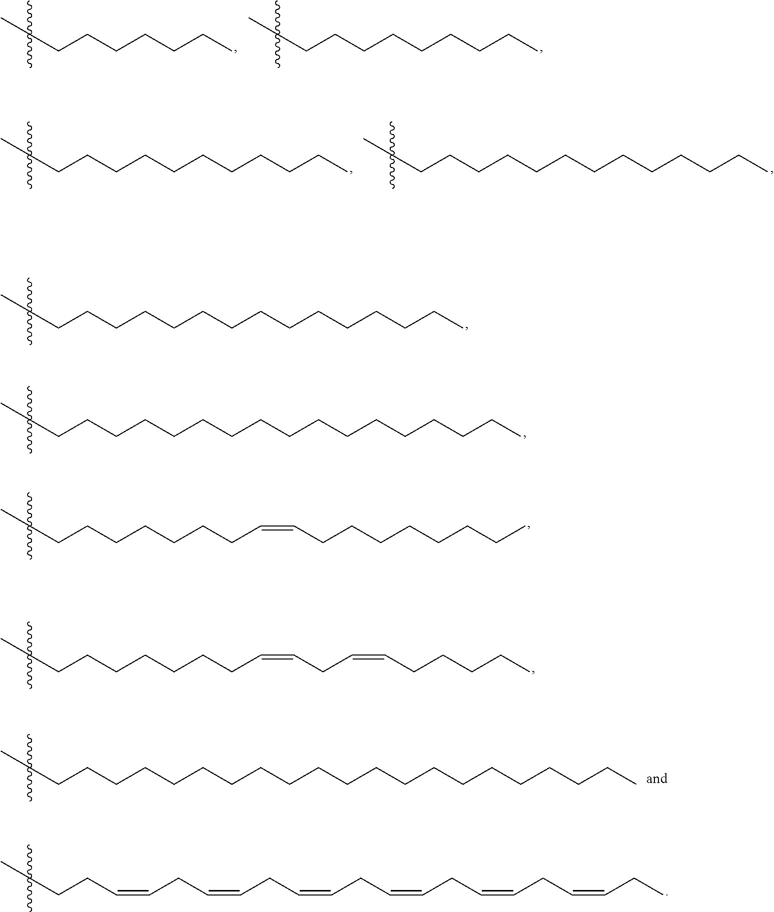

E12. An oligonucleotide-ligand conjugate represented by formula II-Ib or II-Ic:

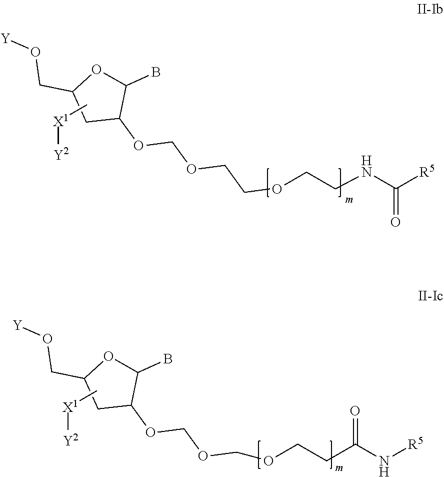

or a pharmaceutically acceptable salt thereof; wherein
B is a nucleobase or hydrogen;
m is 1-50;
X¹ is —O—, or —S—;
Y is hydrogen,

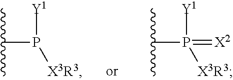

R³ is hydrogen, or a suitable protecting group;
X² is O, or S;
X³ is —O—, —S—, or a covalent bond;
Y¹ is a linking group attaching to the 2′- or 3′-terminal of a nucleoside, a nucleotide, or an oligonucleotide;
Y² is hydrogen, a phosphoramidite analogue, an internucleotide linking group attaching to the 5′-terminal of a nucleoside, a nucleotide, or an oligonucleotide, or a linking group attaching to a solid support;
R⁵ is adamantyl, or a saturated or unsaturated, straight, or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, or —P(S)OR—; and
R is hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur.

E13. The oligonucleotide-ligand conjugate of E12, wherein:
R⁵ is selected from

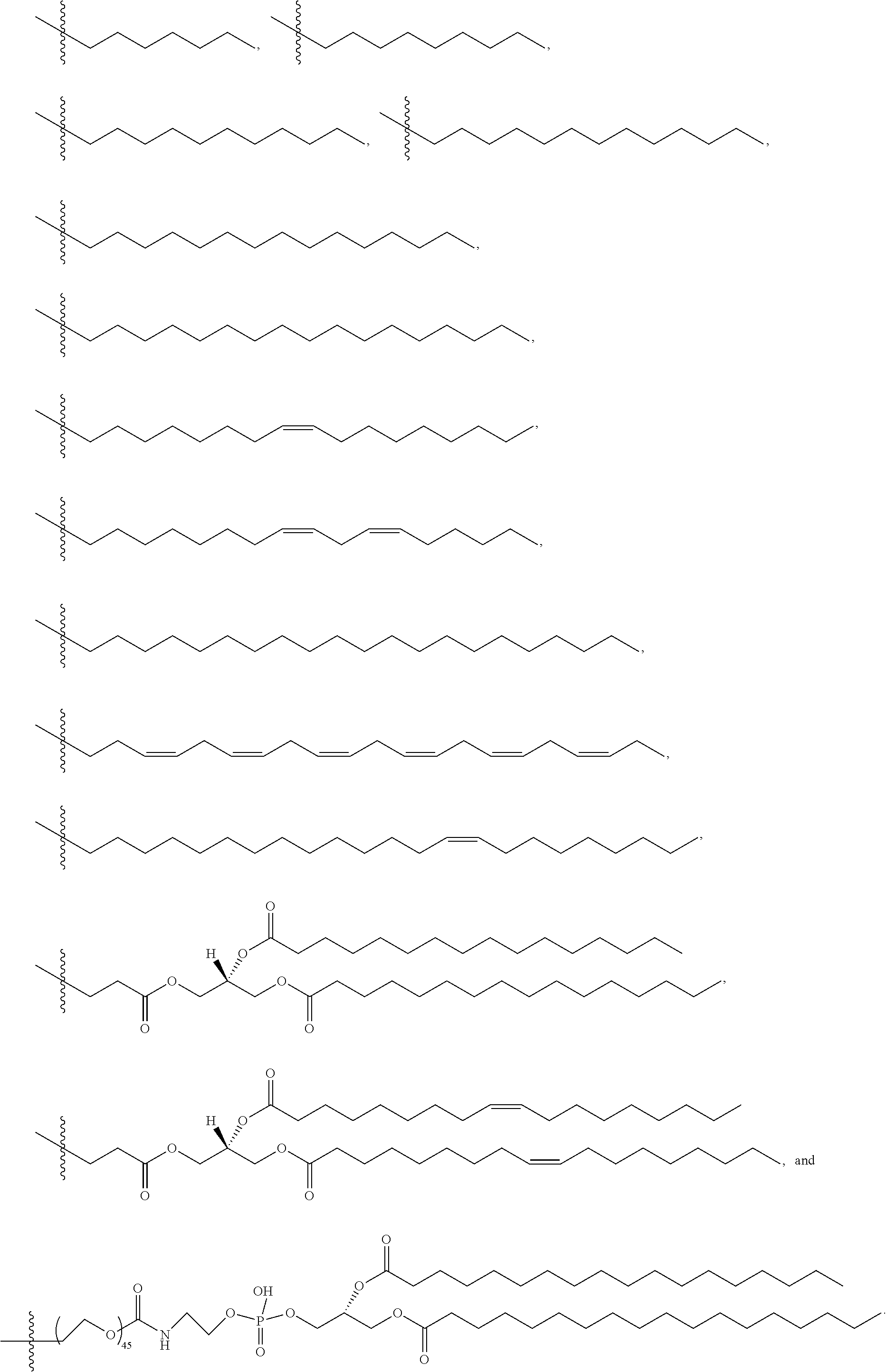

E14. The oligonucleotide-ligand conjugate of any one of E8-E13, wherein the conjugate comprises 1-10 nucleic acid-ligand conjugate units.

E15. The oligonucleotide-ligand conjugate of any one of E8-E13, wherein the conjugate comprises 1, 2 or 3 nucleic acid-ligand conjugate units.

E16. The oligonucleotide-ligand conjugate of any one of E6-E15, wherein the oligonucleotide comprises a sense strand of 10-53 nucleotides in length and an antisense strand of 15-53 nucleotides in length, wherein the antisense oligonucleotide strand has sequence complementary to at least 15 consecutive nucleotides of a target gene sequence and reduces the gene expression when the oligonucleotide-conjugate is introduced into a mammalian cell.

E17. The oligonucleotide-ligand conjugate of E16, wherein the nucleic acid-ligand conjugate units are present in the sense strand.

E18. The oligonucleotide-ligand conjugate of E16, wherein the antisense strand is 19 to 27 nucleotides in length.

E19. The oligonucleotide-ligand conjugate of E16, wherein the sense strand is 12 to 40 nucleotides in length.

E20. The oligonucleotide-ligand conjugate of any one of E16-E19, wherein the sense strand forms a duplex region with the antisense strand.

E21. The oligonucleotide-ligand conjugate of E16, wherein the region of complementarity is fully complementary to the target sequence.

E22. The oligonucleotide-ligand conjugate of any one of E16 to E21, wherein the sense strand comprises at its 3′-end a stem-loop set forth as: S₁-L-S2, wherein S₁ is complementary to S₂, and wherein L forms a loop between S₁ and S2 of 3 to 5 nucleotides in length.

E23. The oligonucleotide-ligand conjugate of E22, wherein L is a tetraloop.

E24. The oligonucleotide-ligand conjugate of E22, wherein L comprises a sequence set forth as GAAA.

E25. The oligonucleotide-ligand conjugate of any one of E16 to E24, further comprising a 3′-overhang sequence on the antisense strand of two nucleotides in length.

E26. The oligonucleotide-ligand conjugate of any one of E16 to E24, wherein the oligonucleotide further comprises a 3′-overhang sequence of one or more nucleotides in length, wherein the 3′-overhang sequence is present on the antisense strand, the sense strand, or the antisense strand and sense strand.

E27. The oligonucleotide-ligand conjugate of any one of E16 to E26, wherein the oligonucleotide comprises at least one modified nucleotide.

E28. The oligonucleotide-ligand conjugate of E27, wherein the modified nucleotide comprises a 2′-modification.

E29. The oligonucleotide-ligand conjugate of E28, wherein the 2′-modification is a modification selected from: 2′-aminoethyl, 2′-fluoro, 2′-O-methyl, 2′-O-methoxyethyl, 2′-deoxy-2′-fluoro, and 2′-deoxy-2′-fluoro-β-d-arabino.

E30. The oligonucleotide-ligand conjugate of any one of E16 to E26, wherein all the nucleotides of the oligonucleotide are modified.

E31. The oligonucleotide-ligand conjugate of any one of E16 to E30, wherein the oligonucleotide comprises at least one modified internucleotide linkage.

E32. The oligonucleotide-ligand conjugate of E31, wherein the at least one modified internucleotide linkage is a phosphorothioate linkage.

E33. The oligonucleotide-ligand conjugate of any one of E16 to E30, wherein the 4′-carbon of the sugar of the 5′-nucleotide of the antisense strand comprises a phosphate analog.

E34. The oligonucleotide-ligand conjugate of E33, wherein the phosphate analog is oxymethylphosphonate, vinylphosphonate, or malonylphosphonate.

E35. A composition comprising an oligonucleotide-ligand conjugate of any one of E16-E34 and an excipient.

E36. A method of delivering an oligonucleotide-ligand conjugate to a subject, the method comprising administering the composition of E35 to the subject.

E37. An oligonucleotide-ligand conjugate of any one of E16-E34 for reducing expression of a target gene.

E38. The oligonucleotide-ligand conjugate of E37, wherein the target gene is expressed in the CNS.

E39. The oligonucleotide-ligand conjugate of E38, wherein the target gene is associated with a disease or disorder, optionally a neurological disease or disorder.

E40. The oligonucleotide-ligand conjugate of E39, wherein the disease or disorder is selected from Progressive Supranuclear Palsy (PSP), Corticobasal degeneration (CBD), Argyrophilic grain disease (AGD), Globular glial tauopathy (GGT), Aging-related tau astrogliopathy (ARTAG), Familial Frontotemporal Dementia 17 (FTD-17), Tauopathy with Respiratory Failure, Dementia with Seizures, Pick's disease, Myotonic dystrophy 1 or 2 (MD1 or MD2), Down's syndrome, Spastic Paraplegia (SP), Niemann-Pick disease type C, Dementia with Lewy bodies (DLB), Lewy body dysphagia, Lewy body disease, Olivopontocerebellar atrophy, Striatonigral degeneration, Shy-Drager syndrome, Spinal muscular atrophy V (SMAV), Huntington's Disease (HD), Alzheimer's Disease, SCA1, SCA2, SCA3, SCA7, SCA10 (spinocerebellar ataxia type 1, 2, 3, 7 or 103), Multiple System Atrophy (MSA), Spinal and Bulbar Muscular Atrophy (SBMA, Kennedy disease), Friedrich Ataxia, Fragile X-associated tremor/ataxia syndrome (FXTAS), Fragile X syndrome (FRAXA), X-Linked Mental Retardation (XLMR), Parkinson's Disease, Dystonia, SBMA (spinobulbar muscular atrophy), neuropathic pain disorders, spinal cord injury, Dentatorubral-pallidoluysian atrophy (DRPLA), recessive CNS disorders, and ALS (amyotrophic lateral sclerosis), M2DS (MECP2 duplication syndrome), FTD (frontotemporal dementia), Prion disease, Adult Onset Leukodystrophy, Alexander's Disease, Krabbe Disease, Chronic Traumatic Encephalopathy, Pelizaeus-Merzbacher disease (PMD), Lafora disease, stroke, Cerebral Amyloid Angiopathy (CAA), and Metachromatic Leukodystrophy (MLD).

### Definitions

As used herein, “approximately” or “about,” as applied to one or more values of interest, refers to a value that is similar to a stated reference value. In certain embodiments, “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1% or less in either direction (greater than or less than) of the stated reference value unless otherwise stated or otherwise evident from the context (except where such number would exceed 100% of a possible value).

As used herein, “administer,” “administering,” “administration” and the like refers to providing a substance (e.g., an RNAi oligonucleotide conjugate) to a subject in a manner that is pharmacologically useful (e.g., to treat a condition in the subject).

As used herein, “asialoglycoprotein receptor” or “ASGPR” refers to a bipartite C-type lectin formed by a major 48 kDa subunit (ASGPR-1) and minor 40 kDa subunit (ASGPR-2). ASGPR is primarily expressed on the sinusoidal surface of hepatocyte cells and has a major role in binding, internalizing and subsequent clearing of circulating glycoproteins that contain terminal galactose or GalNAc residues (asialoglycoproteins).

As used herein, “attenuate,” “attenuating,” “attenuation” and the like refers to reducing or effectively halting. As a non-limiting example, one or more of the treatments herein may reduce or effectively halt the onset or progression of a disease associated with target gene expression (e.g., a neurological disease or disorder) in a subject. This attenuation may be exemplified by, for example, a decrease in one or more aspects (e.g., symptoms, tissue characteristics, and cellular, inflammatory, or immunological activity, etc.) of a disease associated with target gene expression, no detectable progression (worsening) of one or more aspects of the disease, or no detectable aspects of the disease in a subject when they might otherwise be expected.

As used herein, “complementary” refers to a structural relationship between two nucleotides (e.g., on two opposing nucleic acids or on opposing regions of a single nucleic acid strand) that permits the two nucleotides to form base pairs with one another. For example, a purine nucleotide of one nucleic acid that is complementary to a pyrimidine nucleotide of an opposing nucleic acid may base pair together by forming hydrogen bonds with one another. In some embodiments, complementary nucleotides can base pair in the Watson-Crick manner or in any other manner that allows for the formation of stable duplexes. In some embodiments, two nucleic acids may have regions of multiple nucleotides that are complementary with each other to form regions of complementarity, as described herein.

As used herein, “CNS mRNA” and “CNS gene” refers to any gene, mRNA and/or protein encoded/expressed by a gene in a cell or tissue of the central nervous system.

As used herein, “deoxyribonucleotide” refers to a nucleotide having a hydrogen in place of a hydroxyl at the 2′ position of its pentose sugar when compared with a ribonucleotide. A modified deoxyribonucleotide is a deoxyribonucleotide having one or more modifications or substitutions of atoms other than at the 2′ position, including modifications or substitutions in or of the sugar, phosphate group or base.

As used herein, “double-stranded oligonucleotide” or “ds oligonucleotide” refers to an oligonucleotide that is substantially in a duplex form. In some embodiments, the complementary base-pairing of duplex region(s) of a double-stranded oligonucleotide is formed between antiparallel sequences of nucleotides of covalently separate nucleic acid strands. In some embodiments, complementary base-pairing of duplex region(s) of a double-stranded oligonucleotide is formed between antiparallel sequences of nucleotides of nucleic acid strands that are covalently linked. In some embodiments, complementary base-pairing of duplex region(s) of a double-stranded oligonucleotide is formed from single nucleic acid strand that is folded (e.g., via a hairpin) to provide complementary antiparallel sequences of nucleotides that base pair together. In some embodiments, a double-stranded oligonucleotide comprises two covalently separate nucleic acid strands that are fully duplexed with one another. However, in some embodiments, a double-stranded oligonucleotide comprises two covalently separate nucleic acid strands that are partially duplexed (e.g., having overhangs at one or both ends). In some embodiments, a double-stranded oligonucleotide comprises antiparallel sequence of nucleotides that are partially complementary, and thus, may have one or more mismatches, which may include internal mismatches or end mismatches.

As used herein, “duplex,” in reference to nucleic acids (e.g., oligonucleotides), refers to a structure formed through complementary base pairing of two antiparallel sequences of nucleotides.

As used herein, “excipient” refers to a non-therapeutic agent that may be included in a composition, for example, to provide or contribute to a desired consistency or stabilizing effect.

As used herein, “labile linker” refers to a linker that can be cleaved (e.g., by acidic pH). A “fairly stable linker” refers to a linker that cannot be cleaved.

As used herein, “loop” refers to an unpaired region of a nucleic acid (e.g., oligonucleotide) that is flanked by two antiparallel regions of the nucleic acid that are sufficiently complementary to one another, such that under appropriate hybridization conditions (e.g., in a phosphate buffer, in a cells), the two antiparallel regions, which flank the unpaired region, hybridize to form a duplex (referred to as a “stem”).

As used herein, “modified internucleotide linkage” refers to an internucleotide linkage having one or more chemical modifications when compared with a reference internucleotide linkage comprising a phosphodiester bond. In some embodiments, a modified nucleotide is a non-naturally occurring linkage. Typically, a modified internucleotide linkage confers one or more desirable properties to a nucleic acid in which the modified internucleotide linkage is present. For example, a modified nucleotide may improve thermal stability, resistance to degradation, nuclease resistance, solubility, bioavailability, bioactivity, reduced immunogenicity, etc.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide and thymidine deoxyribonucleotide. In some embodiments, a modified nucleotide is a non-naturally occurring nucleotide. In some embodiments, a modified nucleotide has one or more chemical modification in its sugar, nucleobase and/or phosphate group. In some embodiments, a modified nucleotide has one or more chemical moieties conjugated to a corresponding reference nucleotide. Typically, a modified nucleotide confers one or more desirable properties to a nucleic acid in which the modified nucleotide is present. For example, a modified nucleotide may improve thermal stability, resistance to degradation, nuclease resistance, solubility, bioavailability, bioactivity, reduced immunogenicity, etc.

As used herein, “nicked tetraloop structure” refers to a structure of an RNAi oligonucleotide (e.g., comprising an RNAi oligonucleotide conjugate) that is characterized by separate sense (passenger) and antisense (guide) strands, in which the sense strand has a region of complementarity with the antisense strand, and in which at least one of the strands, generally the sense strand, has a tetraloop configured to stabilize an adjacent stem region formed within the at least one strand.

As used herein, “oligonucleotide” refers to a short nucleic acid (e.g., less than about 100 nucleotides in length). An oligonucleotide may be single stranded (ss) or ds. An oligonucleotide may or may not have duplex regions. As a set of non-limiting examples, an oligonucleotide may be, but is not limited to, a small interfering RNA (siRNA), microRNA (miRNA), short hairpin RNA (shRNA), dicer substrate interfering RNA (dsiRNA), antisense oligonucleotide, short siRNA, or single-stranded siRNA. In some embodiments, a double-stranded oligonucleotide is an RNAi oligonucleotide.

As used herein, “overhang” refers to terminal non-base pairing nucleotide(s) resulting from one strand or region extending beyond the terminus of a complementary strand with which the one strand or region forms a duplex. In some embodiments, an overhang comprises one or more unpaired nucleotides extending from a duplex region at the 5′ terminus or 3′ terminus of a double-stranded oligonucleotide. In certain embodiments, the overhang is a 3′ or 5′ overhang on the antisense strand or sense strand of a double-stranded oligonucleotides.

As used herein, “phosphate analog” refers to a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. In some embodiments, a 5′ phosphate analog contains a phosphatase-resistant linkage. Examples of phosphate analogs include, but are not limited to, 5′ phosphonates, such as 5′ methylenephosphonate (5′-MP) and 5′-(E)-vinylphosphonate (5′-VP). In some embodiments, an oligonucleotide has a phosphate analog at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof. See, e.g., US Provisional Patent Application Nos. 62/383,207 (filed on 2 Sep. 2016) and 62/393,401 (filed on 12 Sep. 2016). Other modifications have been developed for the 5′ end of oligonucleotides (see, e.g., Intl. Patent Application No. WO 2011/133871; U.S. Pat. No. 8,927,513; and Prakash et al. (2015) NUCLEIC ACIDS RES. 43:2993-3011).

As used herein, “reduced expression” of a target gene refers to a decrease in the amount or level of RNA transcript (e.g., target mRNA) or protein encoded by the target gene and/or a decrease in the amount or level of activity of the gene in a cell, a population of cells, a sample, or a subject, when compared to an appropriate reference (e.g., a reference cell, population of cells, sample, or subject). For example, the act of contacting a cell with an oligonucleotide or conjugate herein (e.g., an RNAi oligonucleotide conjugate comprising an antisense strand having a nucleotide sequence that is complementary to a nucleotide sequence comprising a target mRNA) may result in a decrease in the amount or level of target mRNA, protein encoded by a target gene, and/or target gene activity (e.g., via inactivation and/or degradation of target mRNA by the RNAi pathway) when compared to a cell that is not treated with the double-stranded oligonucleotide. Similarly, and as used herein, “reducing expression” refers to an act that results in reduced expression of a target gene.

As used herein, “reduction of target gene expression” refers to a decrease in the amount or level of target mRNA, protein encoded by the target gene, and/or target gene activity in a cell, a population of cells, a sample or a subject when compared to an appropriate reference (e.g., a reference cell, population of cells, sample, or subject).

As used herein, “region of complementarity” refers to a sequence of nucleotides of a nucleic acid (e.g., a double-stranded oligonucleotide or an RNAi oligonucleotide conjugate as described herein) that is sufficiently complementary to an antiparallel sequence of nucleotides to permit hybridization between the two sequences of nucleotides under appropriate hybridization conditions (e.g., in a phosphate buffer, in a cell, etc.). In some embodiments, an oligonucleotide herein comprises a targeting sequence having a region of complementary to a mRNA target sequence.

As used herein, “ribonucleotide” refers to a nucleotide having a ribose as its pentose sugar, which contains a hydroxyl group at its 2′ position. A modified ribonucleotide is a ribonucleotide having one or more modifications or substitutions of atoms other than at the 2′ position, including modifications or substitutions in or of the ribose, phosphate group or base.

As used herein, “RNAi oligonucleotide” refers to either (a) a double-stranded oligonucleotide having a sense strand (passenger) and antisense strand (guide), in which the antisense strand or part of the antisense strand is used by the Argonaute 2 (Ago2) endonuclease in the cleavage of a target mRNA (e.g., a target mRNA expressed in the CNS) or (b) a single-stranded oligonucleotide having a single antisense strand, where that antisense strand (or part of that antisense strand) is used by the Ago2 endonuclease in the cleavage of a target mRNA (e.g., a target mRNA expressed in the CNS).

As used herein, “strand” refers to a single, contiguous sequence of nucleotides linked together through internucleotide linkages (e.g., phosphodiester linkages or phosphorothioate linkages). In some embodiments, a strand has two free ends (e.g., a 5′ end and a 3′ end).

As used herein, “subject” means any mammal, including mice, rabbits, and humans. In one embodiment, the subject is a human or NHP. Moreover, “individual” or “patient” may be used interchangeably with “subject.”

As used herein, “synthetic” refers to a nucleic acid or other molecule that is artificially synthesized (e.g., using a machine (e.g., a solid-state nucleic acid synthesizer)) or that is otherwise not derived from a natural source (e.g., a cell or organism) that normally produces the molecule.

As used herein, “targeting ligand” refers to a molecule (e.g., a carbohydrate, amino sugar, cholesterol, polypeptide, or lipid) that selectively binds to a cognate molecule (e.g., a receptor) of a tissue or cell of interest and that is conjugatable to another substance for purposes of targeting the other substance to the tissue or cell of interest. For example, in some embodiments, a targeting ligand may be conjugated to an oligonucleotide for purposes of targeting the oligonucleotide to a specific tissue or cell of interest. In some embodiments, a targeting ligand selectively binds to a cell surface receptor. Accordingly, in some embodiments, a targeting ligand when conjugated to an oligonucleotide facilitates delivery of the oligonucleotide into a particular cell through selective binding to a receptor expressed on the surface of the cell and endosomal internalization by the cell of the complex comprising the oligonucleotide, targeting ligand and receptor. In some embodiments, a targeting ligand is conjugated to an oligonucleotide via a linker that is cleaved following or during cellular internalization such that the oligonucleotide is released from the targeting ligand in the cell.

As used herein, “tetraloop” refers to a loop that increases stability of an adjacent duplex formed by hybridization of flanking sequences of nucleotides. The increase in stability is detectable as an increase in melting temperature (Tₘ) of an adjacent stem duplex that is higher than the Tₘ of the adjacent stem duplex expected, on average, from a set of loops of comparable length consisting of randomly selected sequences of nucleotides. For example, a tetraloop can confer a Tₘ of at least about 50° C., at least about 55° C., at least about 56° C., at least about 58° C., at least about 60° C., at least about 65° C. or at least about 75° C. in 10 mM NaHPO₄ to a hairpin comprising a duplex of at least 2 base pairs (bp) in length. In some embodiments, a tetraloop may stabilize a bp in an adjacent stem duplex by stacking interactions. In addition, interactions among the nucleotides in a tetraloop include, but are not limited to, non-Watson-Crick base pairing, stacking interactions, hydrogen bonding and contact interactions (Cheong et al. (1990) Nature 346:680-682; Heus & Pardi (1991) SCIENCE 253:191-94). In some embodiments, a tetraloop comprises or consists of 3 to 6 nucleotides and is typically 4 to 5 nucleotides. In certain embodiments, a tetraloop comprises or consists of 3, 4, 5 or 6 nucleotides, which may or may not be modified (e.g., which may or may not be conjugated to a targeting moiety). In one embodiment, a tetraloop consists of 4 nucleotides. Any nucleotide may be used in the tetraloop and standard IUPAC-IUB symbols for such nucleotides may be used as described in Cornish-Bowden (1985) NUCLEIC ACIDS RES. 13:3021-30. For example, the letter “N” may be used to mean that any base may be in that position, the letter “R” may be used to show that A (adenine) or G (guanine) may be in that position, and “B” may be used to show that C (cytosine), G (guanine), T (thymine) or U (uracil) may be in that position. Examples of tetraloops include the UNCG family of tetraloops (e.g., UUCG), the GNRA family of tetraloops (e.g., GAAA), and the CUUG tetraloop (Woese et al. (1990) PROC. NATL. ACAD. SCI. USA 87:8467-71; Antao et al. (1991) NUCLEIC ACIDS RES. 19:5901-05). Examples of DNA tetraloops include the d(GNNA) family of tetraloops (e.g., d(GTTA), the d(GNRA)) family of tetraloops, the d(GNAB) family of tetraloops, the d(CNNG) family of tetraloops, and the d(TNCG) family of tetraloops (e.g., d(TTCG)). See, e.g., Nakano et al. (2002) BIOCHEM. 41:4281-92; Shinji et al. (2000) NIPPON KAGAKKAI KOEN YOKOSHU 78:731. In some embodiments, the tetraloop is contained within a nicked tetraloop structure.

As used herein, “treat” or “treating” refers to the act of providing care to a subject in need thereof, for example, by administering a therapeutic agent (e.g., an RNAi oligonucleotide conjugate herein) to the subject, for purposes of improving the health and/or well-being of the subject with respect to an existing condition (e.g., a disease, disorder) or to prevent or decrease the likelihood of the occurrence of a condition. In some embodiments, treatment involves reducing the frequency or severity of at least one sign, symptom or contributing factor of a condition (e.g., disease, disorder) experienced by a subject.

### EXAMPLES

In order that the invention described herein may be more fully understood, the following examples are set forth. The examples described in this application are offered to illustrate the methods, compositions, and systems provided herein and are not to be construed in any way as limiting their scope.

### Abbreviations

Ac: acetyl
AcOH: acetic acid
ACN: acetonitrile
Ad: adamantly
AIBN: 2,2′-azo bisisobutyronitrile
Anhyd: anhydrous
Aq: aqueous
B₂Pin₂: bis (pinacolato)diboron-4,4,4′,4′,5,5,5′,5′-octamethyl-2,2′-bi(1,3,2-dioxaborolane)
BINAP: 2,2′-bis(diphenylphosphino)-1,1′-binaphthyl
BH₃: Borane
Bn: benzyl
Boc: tert-butoxycarbonyl
Boc₂O: di-tert-butyl dicarbonate
BPO: benzoyl peroxide
ⁿBuOH: n-butanol
CDI: carbonyldiimidazole
COD: cyclooctadiene
d: days
DABCO: 1,4-diazobicyclo[2.2.2]octane
DAST: diethylaminosulfur trifluoride
dba: dibenzylideneacetone
DBU: 1,8-diazobicyclo[5.4.0]undec-7-ene
DCE: 1,2-dichloroethane
DCM: dichloromethane
DEA: diethylamine
DHP: dihydropyran
DIBAL-H: diisobutylaluminum hydride
DIPA: diisopropylamine
DIPEA or DIEA: N,N-diisopropylethylamine
DMA: N,N-dimethylacetamide
DME: 1,2-dimethoxyethane
DMAP: 4-dimethylaminopyridine
DMF: N,N-dimethylformamide
DMP: Dess-Martin periodinane
DMSO-dimethyl sulfoxide
DMTr: 4,4′-dimethyoxytrityl
DPPA: diphenylphosphoryl azide
dppf: 1,1′-bis(diphenylphosphino)ferrocene
EDC or EDCI: 1-(3-dimethylaminopropyl)-3-ethylcarbodiimide hydrochloride
ee: enantiomeric excess
ESI: electrospray ionization
EA: ethyl acetate
EtOAc: ethyl acetate
EtOH: ethanol
FA: formic acid
h or hrs: hours
HATU: N,N,N′,N′-tetramethyl-O-(7-azabenzotriazol-1-yl)uronium hexafluorophosphate
HCl: hydrochloric acid
HPLC: high performance liquid chromatography
HOAc: acetic acid
IBX: 2-iodoxybenzoic acid
IPA: isopropyl alcohol
KHMDS: potassium hexamethyldisilazide
K₂CO₃: potassium carbonate
LAH: lithium aluminum hydride
LDA: lithium diisopropylamide
L-DBTA: dibenzoyl-L-tartaric acid
m-CPBA: meta-chloroperbenzoic acid
M: molar
MeCN: acetonitrile
MeOH: methanol
Me₂S: dimethyl sulfide
MeONa: sodium methylate
MeI: iodomethane
min: minutes
mL: milliliters
mM: millimolar
mmol: millimoles
MPa: mega pascal
MOMCl: methyl chloromethyl ether
MsCl: methanesulfonyl chloride
MTBE: methyl tert-butyl ether
nBuLi: n-butyllithium
NaNO₂: sodium nitrite
NaOH: sodium hydroxide
Na₂SO₄: sodium sulfate
NBS: N-bromosuccinimide
NCS: N-chlorosuccinimide
NFSI: N-Fluorobenzenesulfonimide
NMO: N-methylmorpholine N-oxide
NMP: N-methylpyrrolidine
NMR: Nuclear Magnetic Resonance
° C.: degrees Celsius
Pd/C: Palladium on Carbon
Pd(OAc)₂: Palladium Acetate
PBS: phosphate buffered saline
PE: petroleum ether
POCl₃: phosphorus oxychloride
PPh₃: triphenylphosphine
PyBOP: (Benzotriazol-1-yloxy)tripyrrolidinophosphonium hexafluorophosphate
Rel: relative
R.T. or rt: room temperature
s or sec: second
sat: saturated
SEMCl: chloromethyl-2-trimethylsilylethyl ether
SFC: supercritical fluid chromatography
SOCl₂: sulfur dichloride
tBuOK: potassium tert-butoxide
TBAB: tetrabutylammonium bromide
TBAF: tetrabutylammmonium fluoride
TBAI: tetrabutylammonium iodide
TEA: triethylamine
Tf: trifluoromethanesulfonate
TfAA, TFMSA or Tf₂O: trifluoromethanesulfonic anhydride
TFA: trifluoroacetic acid
TIBSCl: 2,4,6-triisopropylbenzenesulfonyl chloride
TIPS: triisopropylsilyl
THF: tetrahydrofuran
THP: tetrahydropyran
TLC: thin layer chromatography
TMEDA: tetramethylethylenediamine
pTSA: para-toluene sulfonic acid
UPLC: Ultra Performance Liquid Chromatography
wt: weight
Xantphos: 4,5-bis(diphenylphosphino)-9,9-dimethylxanthene

### General Synthetic Methods

The following examples are intended to illustrate the disclosure and are not to be construed as being limitations thereon. Temperatures are given in degrees centigrade (C). If not mentioned otherwise, all evaporations are performed under reduced pressure, preferably between about 15 mm Hg and 100 mm Hg (=20-133 mbar). The structure of final products, intermediates and starting materials was confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized for synthesis of the nucleic acid or analogues thereof of the present disclosure are either commercially available or can be produced by organic synthesis methods known to one of ordinary skill in the art (METHODS OF ORGANIC SYNTHESIS, Thieme, Volume 21 (Houben-Weyl 4th Ed. 1952)). Further, the nucleic acid or analogues thereof of the present disclosure can be produced by organic synthesis methods known to one of ordinary skill in the art as shown in the following examples.

All reactions are carried out under nitrogen or argon unless otherwise stated.

Proton NMR (¹H NMR) was conducted in deuterated solvent. In certain nucleic acid or analogues thereof disclosed herein, one or more ¹H shifts overlap with residual proteo solvent signals; these signals have not been reported in the experimental provided hereinafter.

As depicted in the Examples below, in certain exemplary embodiments, the nucleic acid or analogues thereof were prepared according to the following general procedures. It will be appreciated that, although the general methods depict the synthesis of certain nucleic acid or analogues thereof of the present disclosure, the following general methods, and other methods known to one of ordinary skill in the art, can be applied to all nucleic acid or analogues thereof and subclasses and species of each of these nucleic acid or analogues thereof, as described herein.

### General Methods of Preparation of Double-Stranded RNAi Oligonucleotides

### Oligonucleotide Synthesis and Purification

The double-stranded RNAi (dsRNA) oligonucleotides described in the following Examples are chemically synthesized using methods described herein. Generally, dsRNAi oligonucleotides are synthesized using solid phase oligonucleotide synthesis methods as described for 19-23mer siRNAs (see, e.g., Scaringe et al. (1990) NUCLEIC ACIDS RES. 18:5433-5441 and Usman et al. (1987) J. AM. CHEM. Soc. 109:7845-7845; see also, U.S. Pat. Nos. 5,804,683; 5,831,071; 5,998,203; 6,008,400; 6,111,086; 6,117,657; 6,353,098; 6,362,323; 6,437,117 and 6,469,158) in addition to using known phosphoramidite synthesis (see, e.g. Hughes and Ellington (2017) COLD SPRING HARB PERSPECT BIOL. 9 (1):a023812; Beaucage S. L., Caruthers M. H. STUDIES ON NUCLEOTIDE CHEMISTRY V: Deoxynucleoside Phosphoramidites—A New Class of Key Intermediates for Deoxypolynucleotide Synthesis. TETRAHEDRON LETT. 1981; 22:1859-1862. doi: 10.1016/S0040-4039 (01)90461-7).

Individual RNA strands were synthesized and HPLC purified according to standard methods (Integrated DNA Technologies; Coralville, IA). For example, RNA oligonucleotides were synthesized using solid phase phosphoramidite chemistry, deprotected and desalted on NAP-5 columns (Amersham Pharmacia Biotech; Piscataway, NJ) using standard techniques (Damha & Olgivie (1993) METHODS MOL. BIOL. 20:81-114; Wincott et al. (1995) NUCLEIC ACIDS RES. 23:2677-2684). The oligomers were purified using ion-exchange high performance liquid chromatography (IE-HPLC) on an Amersham Source 15Q column (1.0 cm×25 cm; Amersham Pharmacia Biotech) using a 15 min step-linear gradient. The gradient varied from 90:10 Buffers A:B to 52:48 Buffers A:B, where Buffer A is 100 mM Tris pH 8.5 and Buffer B is 100 mM Tris pH 8.5, 1 M NaCl. Samples were monitored at 260 nm and peaks corresponding to the full-length oligonucleotide species were collected, pooled, desalted on NAP-5 columns, and lyophilized.

The purity of each oligomer was determined by capillary electrophoresis (CE) on a Beckman PACE 5000 (Beckman Coulter, Inc.; Fullerton, CA). The CE capillaries have a 100 μm inner diameter and contain ssDNA 100R Gel (Beckman-Coulter). Typically, about 0.6 nmole of oligonucleotide was injected into a capillary, run in an electric field of 444 V/cm and was detected by UV absorbance at 260 nm. Denaturing Tris-Borate-7 M-urea running buffer was purchased from Beckman-Coulter. Oligoribonucleotides were obtained that were at least 90% pure as assessed by CE for use in experiments described below. Compound identity was verified by matrix-assisted laser desorption ionization time-of-flight (MALDI-TOF) mass spectroscopy on a Voyager DE™ Biospectometry Work Station (Applied Biosystems; Foster City, CA) following the manufacturer's recommended protocol. Relative molecular masses of all oligomers were obtained, often within 0.2% of expected molecular mass.

### Preparation of Duplexes

Single strand RNA oligomers were resuspended (e.g., at 100 μM concentration) in duplex buffer consisting of 100 mM potassium acetate, 30 mM HEPES, pH 7.5. Complementary sense and antisense strands were mixed in equal molar amounts to yield a final solution of, for example, 50 μM duplex. Samples were heated to 100° C. for 5′ in RNA buffer (IDT) and were allowed to cool to room temperature before use. The dsRNA oligonucleotides were stored at −20° C. Single strand RNA oligomers were stored lyophilized or in nuclease-free water at −80° C.

### Example 1: Synthesis of 2-(2-((((6aR,8R,9R,9aR)-8-(6-benzamido-9H-purin-9-yl)-2,2,4,4-tetraisopropyltetrahydro-6H-furo[3,2-f][1,3,5,2,4]trioxadisilocin-9-yl)oxy)methoxy)ethoxy) ethan-1-ammonium formate (1-6)

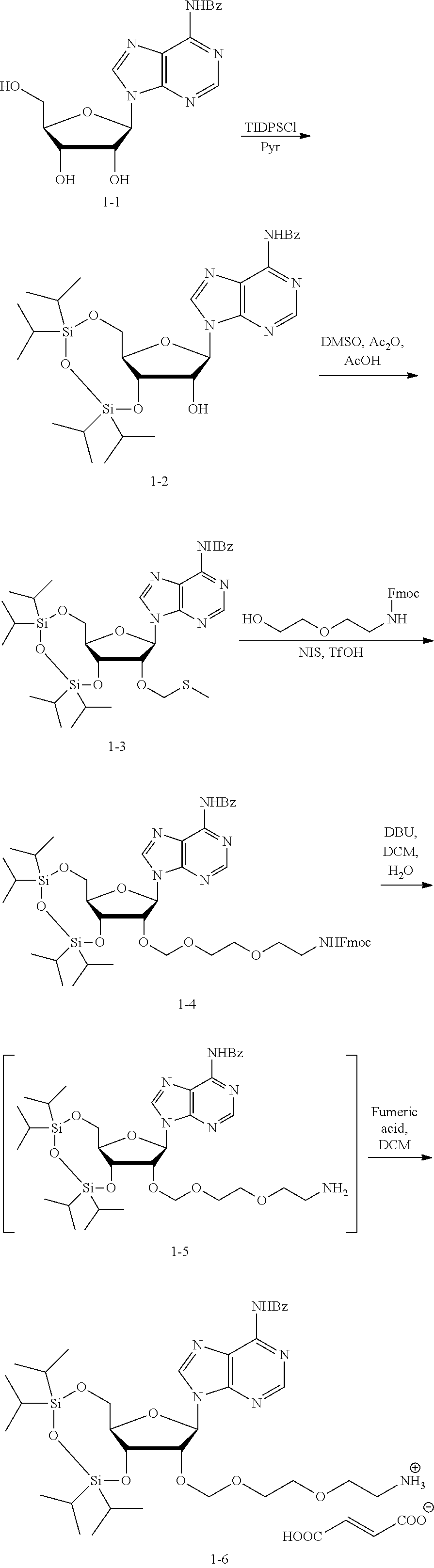

A solution of compound 1-1 (25.00 g, 67.38 mmol) in 20 mL of DMF was treated with pyridine (11 mL, 134.67 mmol) and tetraisopropyldisiloxane dichloride (22.63 mL, 70.75 mmol) at 10° C. The resulting mixture was stirred at 25° C. for 3 h and quenched with 20% citric acid (50 mL). The aqueous layer was extracted with EtOAc (3×50 mL) and the combined organic layers were concentrated in vacuo. The crude residue was recrystallized from a mixture of MTBE and n-heptane (1:15, 320 mL) to afford compound 1-2 (37.20 g, 90%) as a white oily solid.

A solution of compound 1-2 (37.00 g, 60.33 mmol) in 20 mL of DMSO was treated with AcOH (20 mL, 317.20 mmol) and Ac₂O (15 mL, 156.68 mmol). The mixture was stirred at 25° C. for 15 h. The reaction was diluted with EtOAc (100 mL) and quenched with sat. K₂CO₃ (50 mL). The aqueous layer was extracted with EtOAc (3×50 mL). The combined organic layers were concentrated and recrystallized with ACN (30 mL) to afford compound 1-3 (15.65 g, 38.4%) as a white solid.

A solution of compound 1-3 (20.00 g, 29.72 mmol) in 120 mL of DCM was treated with Fmoc-amino-ethoxy ethanol (11.67 g, 35.66 mmol) at 25° C. The mixture was stirred to afford a clear solution and then treated with 4 Å molecular sieves (20.0 g), N-iodosuccinimide (8.02 g, 35.66 mmol), and TfOH (5.25 mL, 59.44 mmol). The mixture was stirred at 30° C. until the HPLC analysis indicated >95% consumption of compound 1-3. The reaction was quenched with TEA (6 mL) and filtered. The filtrate was diluted with EtOAc, washed with sat. NaHCO₃ (2×100 mL), sat. Na₂SO₃ (2×100 mL), and water (2×100 mL) and concentrated in vacuo to afford crude compound 1-4 (26.34 g, 93.9%) as a yellow solid, which was used directly for the next step without further purification.

A solution of compound 1-4 (26.34 g, 27.62 mmol) in a mixture of DCM/water (10:7, 170 mL) was treated with DBU (7.00 mL, 45.08 mmol) at 5° C. The mixture was stirred at 5-25° C. for 1 h. The organic layer was then separated, washed with water (100 mL), and diluted with DCM (130 mL). The solution was treated with fumaric acid (7.05 g, 60.76 mmol) and 4 Å molecular sieves (26.34 g) in four portions. The mixture was stirred for 1 h, concentrated, and recrystallized from a mixture of MTBE and DCM (5:1) to afford compound 1-6 (14.74 g, 62.9%) as a white solid: ¹H NMR (400 MHz, d6-DMSO) 8.73 (s, 1H), 8.58 (s, 1H), 8.15-8.02 (m, 2H), 7.65-7.60 (m, 1H), 7.59-7.51 (m, 2H), 6.52 (s, 2H), 6.15 (s, 1H), 5.08-4.90 (m, 3H), 4.83-4.78 (m, 1H), 4.15-3.90 (m, 3H), 3.79-3.65 (m, 2H), 2.98-2.85 (m, 6H), 1.20-0.95 (m, 28H).

### Example 2: Synthesis of (2R,3R,4R,5R)-5-(6-benzamido-9H-purin-9-yl)-2-((bis(4-methoxyphenyl)(phenyl)methoxy)methyl)-4-((2-(2-[lipid]-amidoethoxy)ethoxy)methoxy) tetrahydrofuran-3-yl (2-cyanoethyl) diisopropylphosphoramidite (2-4a to 2-4e)

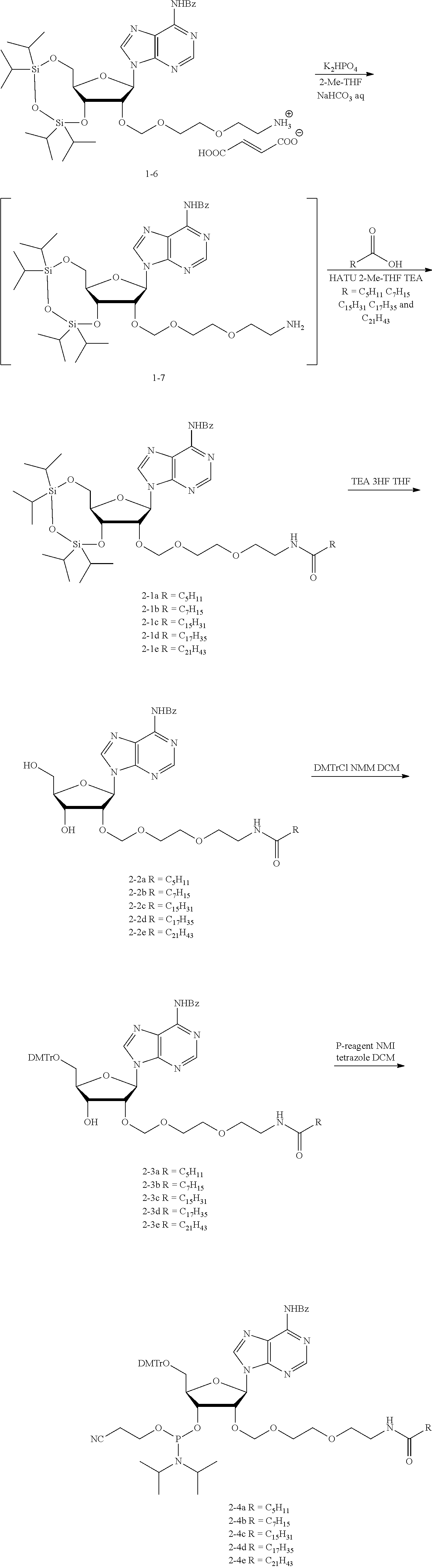

A solution of compound 1-6 (50.00 g, 59.01 mmol) in 150 mL of 2-methyltetrahydrofuran was washed with ice cold aqueous K₂HPO₄ (6%, 100 mL) and brine (20%, 2×100 mL). The organic layer was separated and treated with hexanoic acid (10.33 mL, 82.61 mmol), HATU (33.66 g, 88.52 mmol), and DMAP (10.81 g, 147.52 mmol) at 0° C. The resulting mixture was warmed to 25° C. and stirred for 1 h. The solution was washed with water (2×100 mL), brine (100 mL), and concentrated in vacuo to afford a crude residue. Flash chromatography on silica gel (1:1 hexanes/acetone) gave compound 2-1a (34.95 g, 71.5%) as a white solid.

A mixture of compound 2-1a (34.95 g, 42.19 mmol) and TEA (9.28 mL, 126.58 mmol) in 80 mL of THF was treated with triethylamine trihydrofluoride (20.61 mL, 126.58 mmol) dropwise at 10° C. The mixture was warmed to 25° C. and stirred for 2 h. The reaction was concentrated, dissolved in DCM (100 mL), and washed with sat. NaHCO₃ (5×20 mL) and brine (50 mL). The organic layer was concentrated in vacuo to afford crude compound 2-2a (24.72 g, 99%), which was used directly for the next step without further purification.

A solution of compound 2-2a (24.72 g, 42.18 mmol) in 50 mL of DCM was treated with N-methylmorpholine (18.54 mL, 168.67 mmol) and DMTr-Cl (15.69 g, 46.38 mmol). The mixture was stirred at 25° C. for 2 h and quenched with sat. NaHCO₃ (50 mL). The organic layer was separated, washed with water, concentrated to afford a slurry crude. Flash chromatography on silica gel (1:1 hexanes/acetone) gave compound 2-3a (30.05 g, 33.8 mmol, 79.9%) as a white solid.

A solution of compound 2-3a (25.00 g, 28.17 mmol) in 50 mL of DCM was treated with N-methylmorpholine (3.10 mL, 28.17 mmol) and tetrazole (0.67 mL, 14.09 mmol) under nitrogen atmosphere. Bis(diisopropylamino) chlorophosphine (9.02 g, 33.80 mmol) was added to the solution dropwise and the resulting mixture was stirred at 25° C. for 4 h. The reaction was quenched with water (15 mL), and the aqueous layer was extracted with DCM (3×50 mL). The combined organic layers were washed with sat. NaHCO₃ (50 mL), concentrated to afford a crude solid that was recrystallized from a mixture of DCM/MTBE/n-hexane (1:4:40) to afford compound 2-4a (25.52 g, 83.4%) as a white solid: ¹H NMR (400 MHz, d6-DMSO) 11.25 (s, 1H), 8.65-8.60 (m, 2H), 8.09-8.02 (m, 2H), 7.71 (s, 1H), 7.67-7.60 (m, 1H), 7.59-7.51 (m, 2H), 7.38-7.34 (m, 2H), 7.30-7.25 (m, 7H), 6.85-6.79 (m, 4H), 6.23-6.20 (m, 1H), 5.23-5.14 (m, 1H), 4.80-4.69 (m, 3H), 4.33-4.23 (m, 2H), 3.90-3.78 (m, 1H), 3.75 (s, 6H), 3.74-3.52 (m, 3H), 3.50-3.20 (m, 6H), 3.14-3.09 (m, 2H), 3.09 (s, 1H), 2.82-2.80 (m, 1H), 2.65-2.60 (m, 1H), 2.05-1.96 (m, 2H), 1.50-1.39 (m, 2H), 1.31-1.10 (m, 14H), 1.08-1.05 (m, 2H), 0.85-0.79 (m, 3H); ³¹P NMR (162 MHz, d6-DMSO) 149.43, 149.18.

Compound 2-4b, 2-4c, 2-4d, and 2-4e were prepared using similar procedures described above for compound 2-4a. Compound 2-4b was obtained (25.50 g, 85.4%) as a white solid: ¹H NMR (400 MHz, d6-DMSO) 11.23 (s, 1H), 8.65-8.60 (m, 2H), 8.05-8.02 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.23-5.17 (m, 1H), 4.80-4.69 (m, 3H), 4.40-4.21 (m, 2H), 3.91-3.80 (m, 1H), 3.74 (s, 6H), 3.74-3.52 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.97 (m, 2H), 1.50-1.38 (m, 2H), 1.31-1.10 (m, 18H), 1.08-1.05 (m, 2H), 0.85-0.78 (m, 3H); ³¹P NMR (162 MHz, d6-DMSO) 149.43, 149.19.

Compound 2-4c was obtained (36.60 g, 66.3%) as an off-white solid: ¹H NMR (400 MHz, d6-DMSO) 11.22 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.25-5.17 (m, 1H), 4.80-4.69 (m, 3H), 4.40-4.21 (m, 2H), 3.91-3.80 (m, 1H), 3.74 (s, 6H), 3.74-3.50 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.33-1.12 (m, 38H), 1.08-1.05 (m, 2H), 0.86-0.80 (m, 3H); ³¹P NMR (162 MHz, d6-DMSO) 149.42, 149.17.

Compound 2-4d was obtained (26.60 g, 72.9%) as an off-white solid: ¹H NMR (400 MHz, d6-DMSO) 11.22 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.33 (m, 2H), 7.30-7.25 (m, 7H), 6.89-6.80 (m, 4H), 6.21-6.15 (m, 1H), 5.22-5.17 (m, 1H), 4.80-4.69 (m, 3H), 4.40-4.21 (m, 2H), 3.91-3.80 (m, 1H), 3.74 (s, 6H), 3.74-3.52 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.35-1.08 (m, 38H), 1.08-1.05 (m, 2H), 0.85-0.79 (m, 3H); ³¹P NMR (162 MHz, d6-DMSO) 149.47, 149.22.

Compound 2-4e was obtained (38.10 g, 54.0%) as a white solid: ¹H NMR (400 MHz, d6-DMSO) 11.21 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.23-5.17 (m, 1H), 4.80-4.69 (m, 3H), 4.40-4.21 (m, 2H), 3.91-3.80 (m, 1H), 3.73 (s, 6H), 3.74-3.52 (m, 3H), 3.47-3.22 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.35-1.06 (m, 46H), 1.08-1.06 (m, 2H), 0.85-0.77 (m, 3H); ³¹P NMR (162 MHz, d6-DMSO) 149.41, 149.15.

### Example 3. Synthesis of GalXC RNAi Oligonucleotide-Lipid Conjugates

Scheme 1. Synthesis of GalXC RNAi oligonucleotide-lipid conjugates with mono-lipid (linear and branched) conjugated to the tetraloop. Post-synthetic conjugation was realized through amide coupling reactions.

R₁COOH group represents fatty acid C8:0, C10:0, C11:0, C12:0, C14:0, C16:0, C17:0, C18:0, C18:1, C18:2, C22:5, C22:0, C24:0, C26:0, C22:6, C24:1, diacyl C16:0 or diacyl C18:1

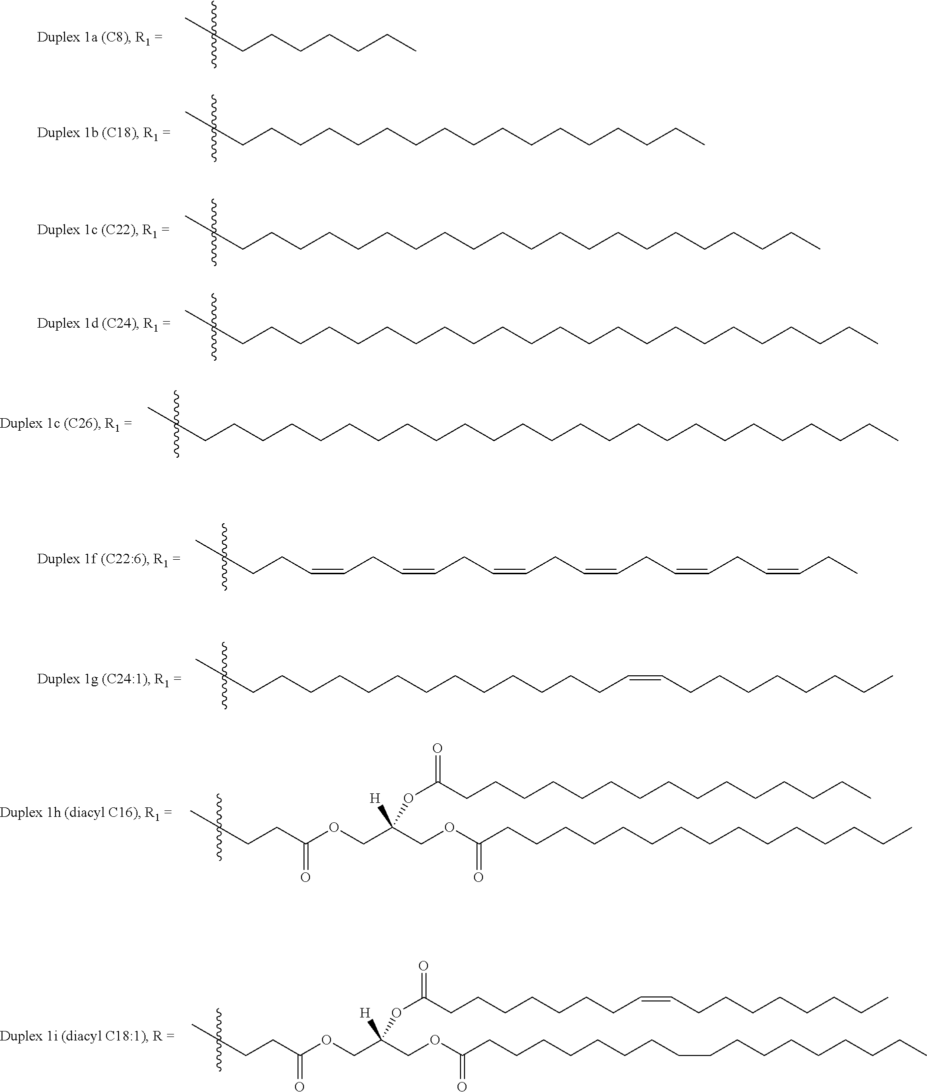

Synthesis Sense 1 and Antisense 1 were prepared by solid-phase synthesis.

Synthesis of Conjugated Sense 1a-1i.

Conjugated Sense 1a was synthesized through post-syntenic conjugation approach. In Eppendorf tube 1, a solution of octanoic acid (0.58 mg, 4 umol) in DMA (0.75 mL) was treated with HATU (1.52 mg, 4 umol) at rt. In Eppendorf tube 2, a solution of oligo Sense 1 (10.00 mg, 0.8 umol) in H₂O (0.25 mL) was treated with DIPEA (1.39 uL, 8 umol). The solution in Eppendorf tube 1 was added to the Eppendorf tube 2 and mixed using ThermoMixer at rt. After the reaction was completed indicated by LC-MS analysis, the reaction mixture was diluted with 5 mL of water and purified by revers phase XBridge C18 column using a 5-95% gradient of 100 mM TEAA in ACN and H₂O. The product fractions were concentrated under reduced pressure using Genevac. The combined residual solvent was dialyzed against water (1×), saline (1×), and water (3×) using Amicon® Ultra-15 Centrifugal (3K). The Amicon membrane was washed with water (3×2 mL) and the combined solvents were then lyophilized to afford an amorphous white solid of Conjugated Sense 1a (6.43 mg, 64% yield).

Conjugated Sense 1b-1i were prepared using similar procedures as described for the synthesis of Conjugated Sense 1a and obtained in 42%-69% yields.

Annealing of Duplex 1a-1j.

Conjugated Sense 1a (10 mg, measured by weight) was dissolved in 0.5 mL deionized water to prepare a 20 mg/mL solution. Antisense 1 (10 mg, measured by OD) was dissolved in 0.5 mL deionized water to prepare a 20 mg/mL solution, which was used for the titration of the conjugated sense and quantification of the duplex amount. Based on the calculation of molar amounts of both conjugated sense and antisense, a proportion of required Antisense 1 was added to the Conjugated Sense 1a solution. The resulting mixture was stirred at 95° C. for 5 min and allowed to cool down to rt. The annealing progress was monitored by ion-exchange HPLC. Based on the annealing progress, several proportions of Antisense 1 were further added to complete the annealing with >95% purity. The solution was lyophilized to afford Duplex 1a (C8) and its amount was calculated based on the molar amount of the antisense consumed in the annealing.

Duplex 1b-1i were prepared using the same procedures as described for the annealing of Duplex 1a (C8).

The following Scheme 1-2 depicts the synthesis of Nicked tetraloop GalXC conjugates with mono-lipid on the loop. Post-synthetic conjugation was realized through Cu-catalyzed alkyne-azide cycloaddition reaction.

Sense 1B and Antisense 1B were prepared by solid-phase synthesis.

### Synthesis of Conjugated Sense 1j.

In Eppendorf tube 1, a solution of oligo (10.00 mg, 0.8 umol) in a 3:1 mixture of DMA/H₂O (0.5 mL) was treated with the lipid linker azide (11.26 mg, 4 umol). In Eppendorf tube 2, CuBr dimethyl sulfide (1.64 mg, 8 umol) was dissolved in ACN (0.5 mL). Both solutions were degassed for 10 min by bubbling N2 through them. The ACN solution of CuBrSMe₂ was then added into tube 1 and the resulting mixture was stirred at 40° C. After the reaction was completed indicated by LC-MS analysis, the reaction mixture was diluted with 0.5 M EDTA (2 mL) and dialyzed against water (2×) using a Amicon® Ultra-15 Centrifugal (3K). The reaction crude was purified by revers phase XBridge C18 column using a 5-95% gradient of 100 mM TEAA in ACN (with 30% IPA spiked in) and H₂O. The product fractions were concentrated under reduced pressure using Genevac. The combined residual solvent was dialyzed against water (1×), saline (1×), and water (3×) using Amicon® Ultra-15 Centrifugal (3K). The Amicon membrane was washed with water (3×2 mL) and the combined solvents were lyophilized to afford an amorphous white solid of Conjugated Sense 1j (6.90 mg, 57% yield).

Duplex 1j (PEG2K-diacyl C18) was prepared using the same procedures as described for the annealing of Duplex 1a (C8).

The following Scheme 1-3 depicts the synthesis of Nicked tetraloop GalXC conjugates with di-lipid on the loop using post-synthetic conjugation approach.

Sense 2 and Antisense 2 were prepared by solid-phase synthesis.

Conjugated Sense 2a and 2b were prepared using similar procedures as described for the synthesis of Conjugated Sense 1a but with 10 eq of lipid, 10 eq of HATU, and 20 eq of DIPEA.

Duplex 2a (2XC11) and 2b (2XC22) were prepared using the same procedures as described for the annealing of Duplex 1a (C8).

The following Scheme 1-4 depicts the synthesis of GalXC of fully phosphorothioated stem-loop conjugated with mono-lipid using post-synthetic conjugation approach.

Sense 3 and Antisense 3 were prepared by solid-phase synthesis.

Conjugated Sense 3a was prepared using similar procedures as described for the synthesis of Conjugated Sense 1a and obtained in a 65% yield.

Duplex 3a (PS-C22) was prepared using the same procedures as described for the annealing of Duplex 1a (C8).

The following Scheme 1-5 depicts the synthesis of GalXC of short sense conjugated with mono-lipid using post-synthetic conjugation approach.

Sense 4 and Antisense 4 were prepared by solid-phase synthesis.

Conjugated Sense 4a was prepared using similar procedures as described for the synthesis of Conjugated Sense 1a and obtained in a 74% yield.

Duplex 4a (SS-C22) was prepared using the same procedures as described for the annealing of Duplex 1a (C8).

The following Scheme 1-6 depicts the synthesis of Nicked tetraloop GalXC conjugated with tri-adamantane moiety on the loop using post-synthetic conjugation approach.

Sense 5 and Antisense 5 were prepared by solid-phase synthesis.

Conjugated Sense 5a and 5b were prepared using similar procedures as described for the synthesis of Conjugated Sense 1a and obtained in 42%-73% yields.

Duplex 5a (3Xadamantane) and Duplex 5b (3Xacetyladamantane) were prepared using the same procedures as described for the annealing of Duplex 1a (C8).

The following scheme 1-7 depicts an example of solid phase synthesis of Nicked tetraloop GalXC conjugated with lipid(s) on the loop.

Synthesis of Conjugated Sense 6.

Conjugated Sense 6 was prepared by solid-phase synthesis using a commercial oligo synthesizer. The oligonucleotides were synthesized using 2′-modified nucleoside phosphoramidites, such as 2′-F or 2′-OMe, and 2′-diethoxymethanol linked fatty acid amide nucleoside phosphoramidites. Oligonucleotide synthesis was conducted on a solid support in the 3′ to 5′direction using a standard oligonucleotide synthesis protocol. 5-ethylthio-1H-tetrazole (ETT) was used as an activator for the coupling reaction. Iodine solution was used for phosphite triester oxidation. 3-(Dimethylaminomethylidene)amino-3H-1,2,4-dithiazole-3-thione (DDTT) was used for the formation of phosphorothioate linkages. Synthesized oligonucleotides were treated with concentrated aqueous ammonium for 10 h. The ammonia was removed from the suspension and the solid support residues were removed by filtration. The crude oligonucleotide was treated with TEAA, analyzed, and purified by strong anion exchange high performance liquid chromatography (SAX-HPLC). The fractions were combined and dialyzed against water (3×), saline (1×), and water (3×) using Amicon® Ultra-15 Centrifugal (3K). The remaining solvent was then lyophilized to afford the desired Conjugated Sense 6.

Duplex 6 was prepared using the same procedures as described for the annealing of Duplex 1a (C8).

Scheme 8. Synthesis of Nicked tetraloop GalXC conjugated with one adamantane unit on the loop via a post-synthetic conjugation approach.

### Synthesis of Conjugated Sense 7a and 7b

Conjugated Sense 7a and Sense 7b were obtained using the same method or a substantially similar method to the synthesis of Conjugated Sense 5.

### Synthesis Example of Duplex 7a and 7b

Duplex 7a and Duplex 7b were obtained using the same method or a substantially similar method to the synthesis of Duplex 5.

Scheme 9. Synthesis of nicked tetraloop GalXC conjugated with two adamantane units on the loop via a post-synthetic conjugation approach.

### Synthesis of Conjugated Sense 8a and 8b

Conjugated Sense 8a and Sense 8b were obtained using the same method or a substantially similar method to the synthesis of Conjugated Sense 5.

### Synthesis Example of Duplex 8a and 8b

Duplex 8a and Duplex 8b were obtained using the same method or a substantially similar method to the synthesis of Duplex 5.

The following Scheme 1-10 depicts the synthesis of GalXC of short sense and short stem loop conjugated with mono-lipid using post-synthetic conjugation approach.

### Synthesis of Sense 9a

Conjugated Sense 9a was obtained using the same method or a substantially similar method to the synthesis of Conjugated Sense 5.

### Synthesis Example of Duplex 9a

Duplex 9a was obtained using the same method or a substantially similar method to the synthesis of Duplex 5.

The following Scheme 1-11 depicts the synthesis of GalXC conjugated with mono-lipid at 5′-end using post-synthetic conjugation approach.

### Synthesis of Conjugated Sense 10a

Conjugated Sense 10a was obtained using the same method or a substantially similar method to the synthesis of Conjugated Sense 5.

### Synthesis Example of Duplex 10a

Duplex 10a was obtained using the same method or a substantially similar method to the synthesis of Duplex 5.

The following Scheme 1-12a and 1-12b depict the synthesis of GalXC with blunt end conjugated with mono-lipid at 3′-end or 5′-end using post-synthetic conjugation approach.

### Synthesis of Conjugated Sense 11a and 12a

Conjugated Sense 11a and 12a were obtained using the same method or a substantially similar method to the synthesis of Conjugated Sense 5.

### Synthesis Example of Duplex 11a and 12a

Duplex 11a and 12a were obtained using the same method or a substantially similar method to the synthesis of Duplex 5.

Conjugates Duplex 8D and Duplex 9D were obtained using the same method or a substantially similar method to the synthesis of Duplex 5.

### Example 4: GalNAc-Conjugated RNAi Oligonucleotides Reduce Gene Expression in the Central Nervous System of Rodents

To evaluate the ability of GalNAc-conjugated RNAi oligonucleotides generated by general methods described herein and/or in Example 1-3 to reduce target gene expression in the central nervous system (CNS), mice or rats were treated with GalNAc-conjugated RNAi oligonucleotides that target murine or rat Aldh2 (alternatively “mALDH2” or “rALDH2” herein) mRNA via either intracerebroventricular (i.c.v.) or intrathecal (i.t.) administration into cerebrospinal fluid (CSF) and the subsequent effect(s) on Aldh2 expression in rodent (mouse and rat) CNS was determined.

Briefly, an RNAi oligonucleotide conjugate comprising a GalNAc-conjugated nicked tetraloop structure having a 36-mer passenger strand and a 22-mer guide strand, the nucleotide sequences of which are set forth in Table 3, was generated (henceforth the “GalXC-ALDH2 RNAi oligonucleotide”). The nucleotide sequences comprising the passenger strand and guide strand of the GalXC-ALDH2 RNAi oligonucleotide each comprise a distinct pattern of modified nucleotides and phosphorothioate linkages, as depicted in FIG. 12 and shown below. Three of the nucleotides comprising the tetraloop of the GalXC-ALDH2 RNAi oligonucleotide are each conjugated to a GalNAc moiety (CAS #14131-60-3; e.g., see FIG. 12 for a depiction of the generic structure and chemical modification pattern of the GalNAc-conjugated RNAi oligonucleotides).

Sense (Passenger) Strand: 5′-mX-S-mX-fX-mX-fX-mX-mX-fX-mX-fX-mX-fX-fX-mX-fX-mX-fX[-mX-]₁₀-[ademX-GalNAc]-[ademX-GalNAc]-[ademX-GalNAc]-mX-mX-mX-mX-mX-mX-3′.

### Hybridized to:

### Antisense (Guide) Strand: 5′-[MePhosphonate-4O-mX]—S-fX-S-fX-fX-fX-mX-fX-mX-mX-fX-mX-mX-mX-fX-mX-fX-mX-mX-fX-mX-S-mX-S-mX-3′ (Modification key: Table 2)

TABLE 2
Modification Key

| Symbol | Modification/linkage |
|---|---|
| mX | 2′-O-methyl modified nucleotide |
| fX | 2′-fluoro modified nucleotide |
| -S- | phosphorothioate linkage |
| - | phosphodiester linkage |
| [MePhosphonate- | 5′-methoxyphosphonate-4-oxy modified nucleotide |
| 4O-mX] | |
| ademX-GalNAc | GalNAc-conjugated nucleotide |

The nucleotide sequences the sense and antisense strands comprising the GalXC-ALDH2 RNAi oligonucleotide is provided in Table 3.

TABLE 3
GalNAc-Conjugated GalXC-ALDH2 RNAi Oligonucleotide
Sense and Antisense Strand Nucleotide Sequences

| | | SEQ ID NO | SEQ ID NO | SEQ ID NO | SEQ ID NO |
|---|---|---|---|---|---|
| RNAi | | (Sense) | (Antisense) | (Sense) | (Antisense) |
| Oligonucleotide | DP# | Unmodified | | Modified (GalNAc) | |
| GalXC-ALDH2 | DP11518P:DP11674G | 2 | 1 | 4 | 3 |

### Mouse Intracerebroventricular (i.c.v.) Administration

The GalXC-ALDH2 RNAi oligonucleotide provided in Table 3 was administered as a single dose of 100 μg (˜4 mg/kg) of oligonucleotide formulated in phosphate buffered saline (PBS) (n=4) via intracerebroventricular (i.c.v.) injection (10 μl) into the brain of female CD-1 mice age 6-8 weeks old. A control group of mice (n=4) was administered only PBS. Five (5) days post-injection, mice were sacrificed. Whole brain, lumbar spinal cord, and liver were dissected and preserved for RT-qPCR analysis. RNA was extracted from tissue samples from the somatosensory cortex, striatum, hippocampus, cerebellum, and liver to determine murine Aldh2mRNA levels by qPCR (normalized to endogenous housekeeping genes Hprt, as indicated). The levels of murine Aldh2 mRNA were determined using PrimeTime™ qPCR Probe Assays (IDT). The qPCR was performed using PrimeTime™ qPCR Probe Assays, which consisted of a primer pair and fluorescently labeled 5′ nuclease probe specific to murine Aldh2mRNA. The percentage of murine Aldh2mRNA remaining in the samples from treated mice was determined using the 2^{-ΔΔCt} (“delta-delta Ct”) method (Livak and Schmittgen (2001) METHODS 25:402-408).

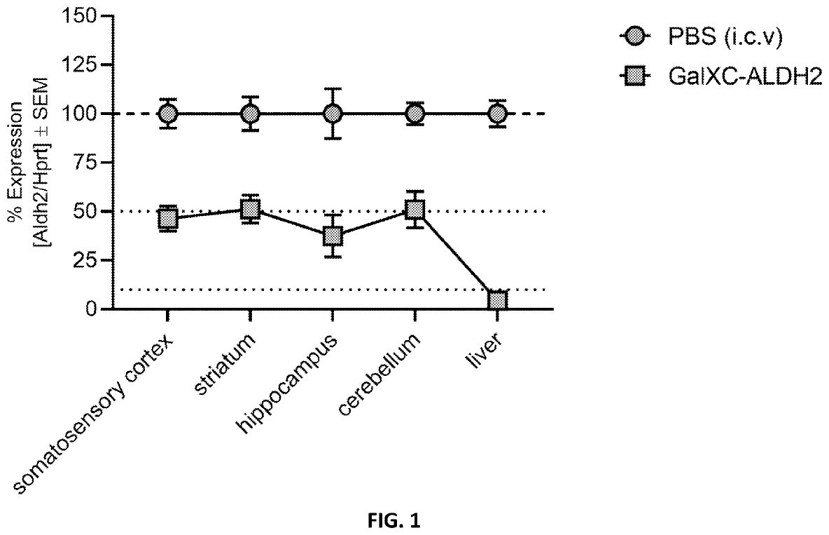

As shown in FIG. 1, i.c.v. administration of a GalNAc-conjugated RNAi oligonucleotide reduced target gene expression in the CNS, as determined by comparison of the percentage of murine Aldh2 mRNA remaining in samples from mice treated with the GalXC-ALDH2 RNAi oligonucleotide provided in Table 3 relative to the percentage of murine Aldh2 mRNA remaining in samples from control mice treated with PBS. This comparison affirms that the reduction in the level of murine Aldh2 mRNA in the somatosensory cortex, striatum, hippocampus, and cerebellum from mice treated with the GalXC-ALDH2 RNAi oligonucleotide provided in Table 3 relative to control mice treated with PBS is a result of the effect of GalXC-ALDH2 RNAi oligonucleotide. Inhibition of murine Aldh2 expression was also observed in the liver of mice treated with the GalXC-ALDH2 RNAi oligonucleotide (FIG. 1). These results demonstrate that GalNAc-conjugated RNAi oligonucleotides exhibit the ability to reduce target gene expression in multiple different anatomical regions of the CNS and in the liver following i.c.v. injection.

### Mouse Dose Range and Durability

To further demonstrate the ability of GalNAc-conjugated RNAi oligonucleotides generated by general methods described herein and/or in Example 1-3 to reduce target gene expression in the CNS, the GalXC-ALDH2 RNAi oligonucleotide provided in Table 3 was administered as a single dose of either 250 μg (10 mg/kg) or 500 μg (20 mg/kg) of oligonucleotide formulated in phosphate buffered saline (PBS) (n=4) via intracerebroventricular (i.c.v.) injection (10 μl) into the brain of female CD-1 mice age 6-8 weeks old. A control group of mice (n=5) was administered only PBS. Twenty-one (21), fifty-six (56) or eighty-four (84) days post-injection, mice were sacrificed. Whole brain and lumbar spinal cord were dissected and preserved for RT-qPCR analysis. RNA was extracted from tissue samples from the frontal cortex, hippocampus, hypothalamus, striatum, somatosensory cortex, cerebellum, cervical spinal cord, thoracic spinal cord, and lumbar spinal cord to determine murine Aldh2 mRNA levels by qPCR (normalized to endogenous housekeeping genes Hprt, as indicated). The levels of murine Aldh2 mRNA were determined using PrimeTime™ qPCR Probe Assays (IDT). The qPCR was performed using PrimeTime™ qPCR Probe Assays, which consisted of a primer pair and fluorescently labeled 5′ nuclease probe specific to murine Aldh2 mRNA. The percentage of murine Aldh2 mRNA remaining in the samples from treated mice was determined using the 2^{-ΔΔCt} (“delta-delta Ct”) method (Livak and Schmittgen (2001) METHODS 25:402-408).

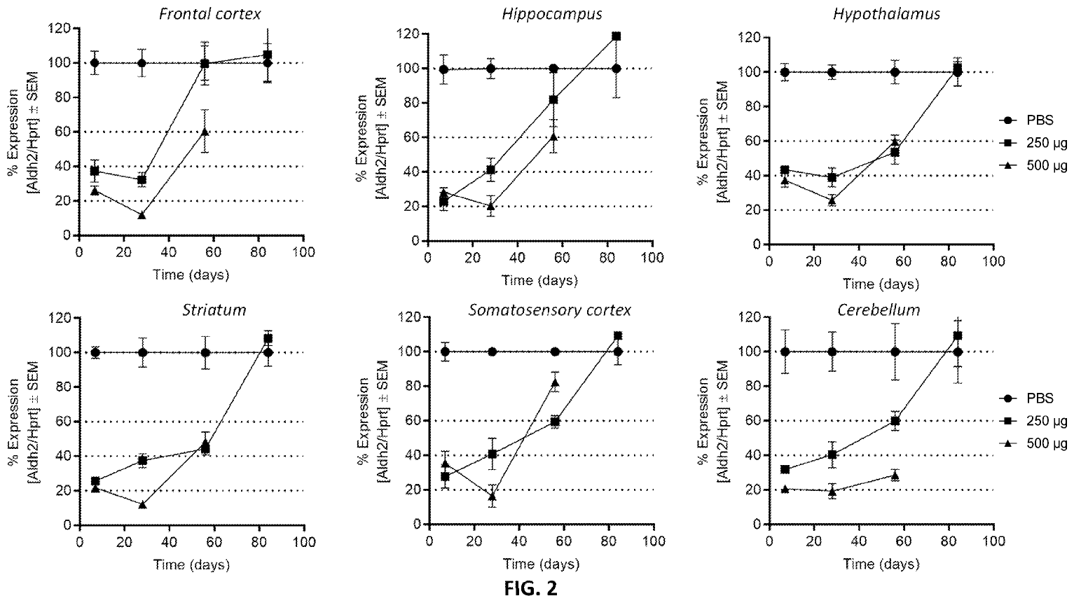

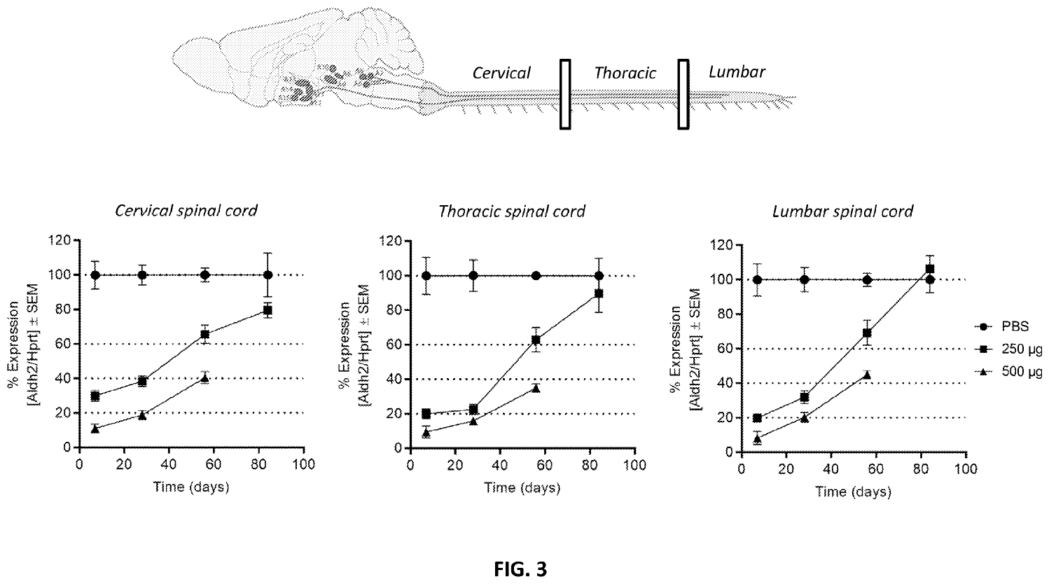

As shown in FIG. 2 and FIG. 3, i.c.v. administration of a GalNAc-conjugated RNAi oligonucleotide reduced target gene expression in the CNS in a dose-dependent manner, as determined by comparison of the percentage of murine Aldh2 mRNA remaining in the tissue samples from mice treated with the GalXC-ALDH2 RNAi oligonucleotide provided in Table 3 relative to the percentage of murine Aldh2mRNA remaining in samples from control mice treated with PBS. A reduction of murine Aldh2mRNA expression in the tissue samples from mice treated with the GalXC-ALDH2 RNAi oligonucleotide was measurable for at least 60 days (2 months) following a single dose (FIG. 2 and FIG. 3). These further results demonstrate that GalNAc-conjugated RNAi oligonucleotides exhibit the ability to reduce target gene expression in multiple different anatomical regions of the CNS following i.c.v. injection and in a dose-dependent manner. Further, these results demonstrate that the reduction of target gene expression in the CNS is measurable for at least two (2) months following a single administration, indicating the ability of GalNAc-conjugated RNAi oligonucleotides to provide an extended pharmacodynamic durability in the CNS.

### Mouse Lumbar Intrathecal (i.t.) Administration

To further demonstrate the ability of GalNAc-conjugated RNAi oligonucleotides generated by general methods described herein and/or in Example 1-3 to inhibit gene expression in the CNS, the GalXC-ALDH2 RNAi oligonucleotide provided in Table 3 was administered as a single dose of 500 ug (20 mg/kg) of oligonucleotide formulated in phosphate buffered saline (PBS) (n=10) via lumbar intrathecal (i.t.) injection (10 μl) into the spinal cord of female CD-1 mice age 6-8 weeks old. A control group of mice (n=10) was administered only PBS. Seven (7) days post-injection, mice were sacrificed. Whole brain and lumbar spinal cord were dissected and preserved for RT-qPCR analysis. RNA was extracted from tissue samples from the frontal cortex, somatosensory cortex, striatum, hippocampus, hypothalamus, cerebellum, cervical spinal cord, thoracic spinal cord, lumbar spinal cord, dorsal root ganglion, liver, and kidney to determine murine Aldh2 mRNA levels by qPCR (normalized to endogenous housekeeping genes Hprt, as indicated). The levels of murine Aldh2mRNA were determined using PrimeTime™ qPCR Probe Assays (IDT). The qPCR was performed using PrimeTime™ qPCR Probe Assays, which consisted of a primer pair and fluorescently labeled 5′ nuclease probe specific to murine Aldh2mRNA. The percentage of murine Aldh2 mRNA remaining in the samples from treated mice was determined using the 2^{-ΔΔCt} (“delta-delta Ct”) method (Livak and Schmittgen (2001) METHODS 25:402-408).

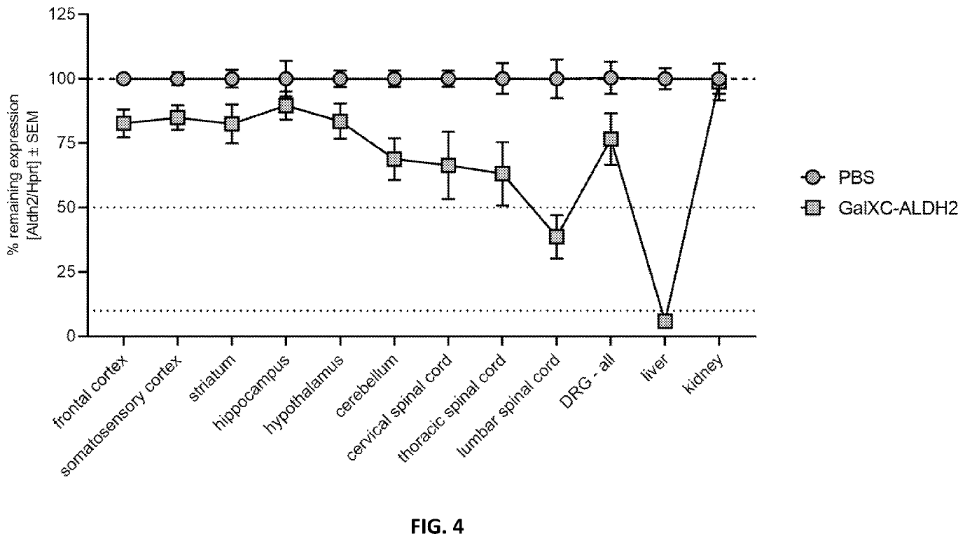

As shown in FIG. 4, lumbar i.t. administration of a GalNAc-conjugated RNAi oligonucleotide reduced target gene expression in the CNS, as determined by comparison of the percentage of murine Aldh2 mRNA remaining in the tissue samples from mice treated with the GalXC-ALDH2 RNAi oligonucleotide provided in Table 3 relative to the percentage of murine Aldh2mRNA remaining in samples from control mice treated with PBS. These results demonstrate that GalNAc-conjugated RNAi oligonucleotides exhibit the ability to reduce target gene expression in multiple different anatomical regions of the CNS following lumber i.t. injection.

### Rat Dose and Durability

To further demonstrate the ability of GalNAc-conjugated RNAi oligonucleotides generated by general methods described herein and/or in Example 1-3 to reduce target gene expression in the CNS, the GalXC-ALDH2 RNAi oligonucleotide provided in Table 3 was administered as a single dose of either 1000 μg (4 mg/kg) or 6000 μg (24 mg/kg) of oligonucleotide formulated in phosphate buffered saline (PBS) (n=8) via lumbar intrathecal (i.t.) injection (30 μl) into the lumbar spinal cord of female Sprague-Dawley rats.

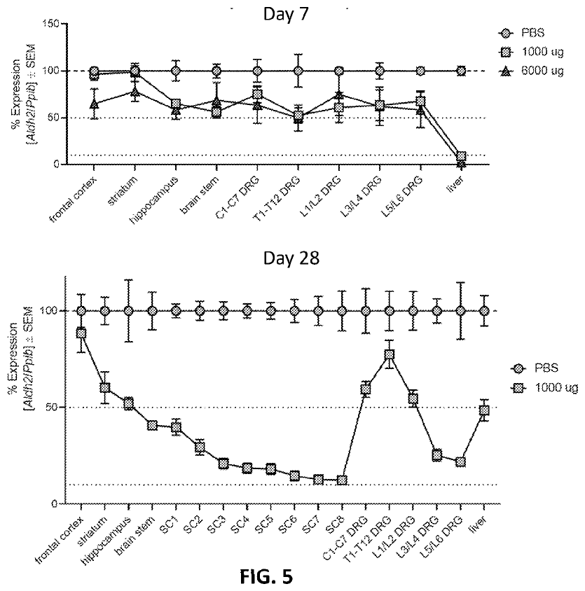

As shown in FIG. 5, lumbar i.t. administration of a GalNAc-conjugated RNAi oligonucleotide reduced target gene expression in the CNS, as determined by comparison of the percentage of rat Aldh2mRNA remaining in the tissue samples from rats treated with the GalXC-ALDH2 RNAi oligonucleotide provided in Table 3 relative to the percentage of rat Aldh2mRNA remaining in samples from control rats treated with PBS. These results demonstrate that GalNAc-conjugated RNAi oligonucleotides exhibit the ability to reduce target gene expression in multiple different anatomical regions of the CNS following lumber i.t. injection in rats.

### Example 5: GalNAc-Conjugated RNAi Oligonucleotides Inhibit Gene Expression in the Central Nervous System of Non-Human Primates (NHPs)

To further evaluate the ability of GalNAc-conjugated RNAi oligonucleotides generated by general methods described herein and/or in Example 1-3 to reduce target gene expression in the central nervous system (CNS), NHPs (cynomolgus (Macaca fascicularis) and Rhesus (Macaca mulatta) macaques) were treated with GalNAc-conjugated RNAi oligonucleotides that target ALDH2 mRNA via either intracerebroventricular (i.c.v.) or intrathecal (i.t.) administration into cerebrospinal fluid (CSF) and the subsequent effect(s) on ALDH2 expression in NHP CNS was determined.

### NHP Lumbar Intrathecal (i.t.) Administration

The GalXC-ALDH2 RNAi oligonucleotide provided in Table 3 was administered as a single dose of 75 mg of oligonucleotide formulated in phosphate buffered saline (PBS) via lumbar intrathecal (i.t.) injection (3 mL) into the lumbar spine of male cynomolgus NHPs (M. fascicularis; n=3; average weight 3-3.5 kg). A control group of cynomolgus NHPs (n=3) was administered only PBS. Twenty-eight (28) days post-injection, cynomolgus NHPs were sacrificed. Whole brain and lumbar spinal cord were dissected and preserved for RT-qPCR analysis. RNA was extracted from tissue samples from the frontal cortex, caudate nucleus, hippocampus, cerebellum, cervical spinal cord, thoracic spinal cord, lumbar spinal cord, dorsal root ganglion, and liver to determine ALDH2 mRNA levels by qPCR (normalized to endogenous housekeeping genes HPRT, as indicated). The levels of ALDH2mRNA were determined using PrimeTime™ qPCR Probe Assays (IDT). The qPCR was performed using PrimeTime™ qPCR Probe Assays, which consisted of a primer pair and fluorescently labeled 5′ nuclease probe specific to ALDH2mRNA. The percentage of ALDH2 mRNA remaining in the samples from treated NHPs was determined using the 2^{-ΔΔCt}(“delta-delta Ct”) method (Livak and Schmittgen (2001) METHODS 25:402-08).

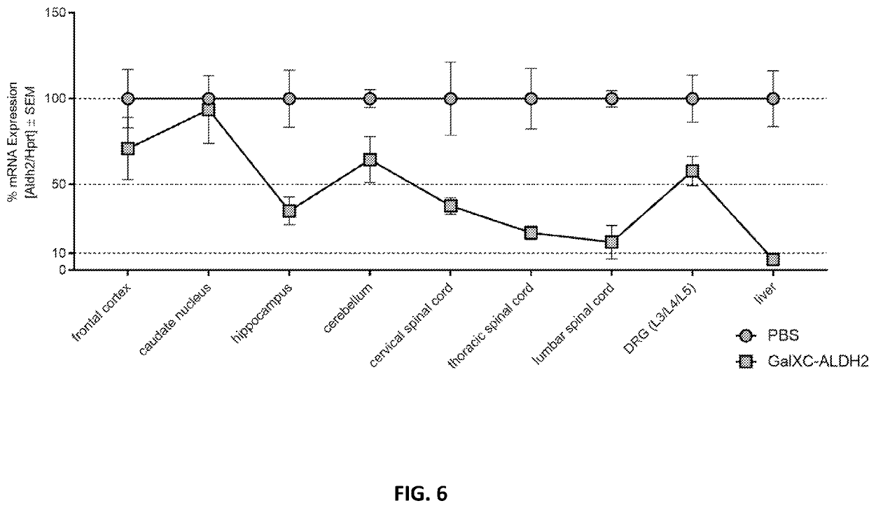

As shown in FIG. 6, lumbar i.t. administration of a GalNAc-conjugated RNAi oligonucleotide reduced target gene expression in the CNS of cynomolgus NHPs, as determined by comparison of the percentage of ALDH2 mRNA remaining in the tissue samples from cynomolgus NHPs treated with the GalXC-ALDH2 RNAi oligonucleotide provided in Table 3 relative to the percentage of ALDH2mRNA remaining in samples from control cynomolgus NHPs treated with PBS. These results demonstrate that GalNAc-conjugated RNAi oligonucleotides exhibit the ability to reduce target gene expression in multiple different anatomical regions of the NHP CNS following lumber i.t. injection.

To further evaluate the ability of the GalXC-ALDH2 RNAi oligonucleotide provided in Table 3 to reduce target gene expression in the central nervous system (CNS) of NHPs, Rhesus macaques (M. mulatta; n=3) were administered a dose of 75 mg, 150 mg, or 300 mg of the oligonucleotide formulated in phosphate buffered saline (PBS) via lumbar intrathecal (i.t.) injection (3 mL) into the lumbar spine. A control group of Rhesus NHPs (n=3) was administered only PBS. Twenty-eight (28) days post-injection, Rhesus NHPs were sacrificed. Whole brain and lumbar spinal cord were dissected and preserved for RT-qPCR analysis. RNA was extracted from tissue samples from the frontal cortex, hippocampus, temporal cortex, cerebellum, brainstem, cervical spinal cord, thoracic spinal cord, lumbar spinal cord, dorsal root ganglion (DRGs L1-L6), and liver to determine ALDH2 mRNA levels by qPCR (normalized to endogenous housekeeping genes HPRT and PPIB, as indicated). The levels of ALDH2 mRNA were determined using PrimeTime™ qPCR Probe Assays (IDT). The qPCR was performed using PrimeTime™ qPCR Probe Assays, which consisted of a primer pair and fluorescently labeled 5′ nuclease probe specific to ALDH2mRNA. The percentage of ALDH2mRNA remaining in the samples from treated NHPs was determined using the 2^{-ΔΔCt} (“delta-delta Ct”) method (Livak and Schmittgen (2001) METHODS 25:402-408).

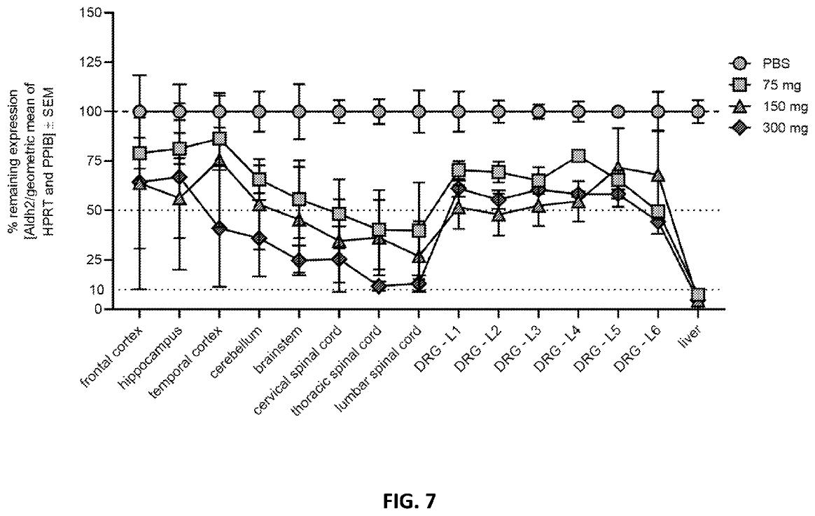

As shown in FIG. 7, lumbar i.t. administration of the GalXC-ALDH2 RNAi oligonucleotide reduced target gene expression in the CNS of rhesus NHPs in a dose-related manner, as determined by comparison of the percentage of ALDH2mRNA remaining in the tissue samples from Rhesus NHPs treated with the GalXC-ALDH2 RNAi oligonucleotide provided in Table 3 relative to the percentage of ALDH2 mRNA remaining in samples from control Rhesus NHPs treated with PBS. These results demonstrate that GalNAc-conjugated RNAi oligonucleotides exhibit the ability to reduce target gene expression in a dose-related manner in multiple different anatomical regions of the NHP CNS following lumber i.t. injection.

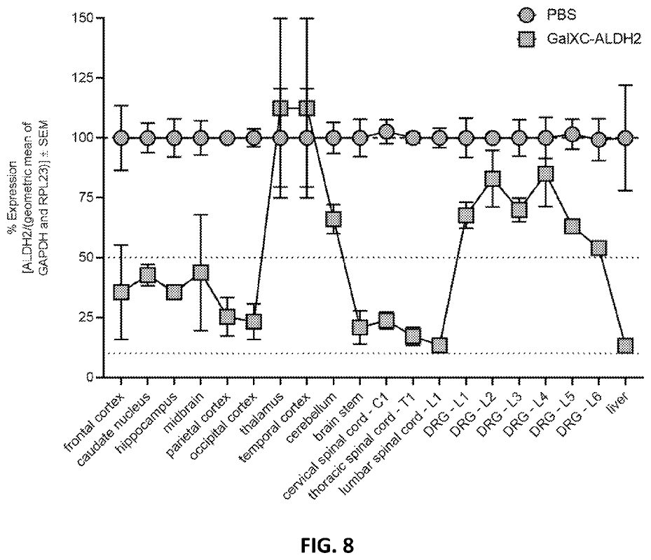

To further evaluate the ability of the GalXC-ALDH2 RNAi oligonucleotide provided in Table 3 to reduce target gene expression in the central nervous system (CNS) of NHPs, cynomolgus NHPs (M. fascicularis; n=3) were administered a dose of 75 mg of the oligonucleotide formulated in phosphate buffered saline (PBS) via lumbar intrathecal (i.t.) injection (3 mL) into the lumbar spine. A control group of cynomolgus NHPs (n=3) was administered only PBS. Prior to treatment, a catheter and port were surgically implanted into each NHP to allow for slow (˜15 min.) infusion of the formulated oligonucleotide or PBS control solution into the lumbar intrathecal space. Twenty-eight (28) days post-injection, cynomolgus NHPs were sacrificed. Whole brain and lumbar spinal cord were dissected and preserved for RT-qPCR analysis. RNA was extracted from tissue samples from the frontal cortex, caudate nucleus, hippocampus, midbrain, parietal cortex, occipital cortex, thalamus, temporal cortex, cerebellum, brainstem, cervical spinal cord, thoracic spinal cord, lumbar spinal cord, dorsal root ganglion (DRGs L1-L6), and liver to determine ALDH2mRNA levels by qPCR (normalized to endogenous housekeeping genes GAPDH and RPL23, as indicated). The levels of ALDH2 mRNA were determined using PrimeTime™ qPCR Probe Assays (IDT). The qPCR was performed using PrimeTime™ qPCR Probe Assays, which consisted of a primer pair and fluorescently labeled 5′ nuclease probe specific to ALDH2 mRNA. The percentage of ALDH2 mRNA remaining in the samples from treated NHPs was determined using the 2^{-ΔΔCt} (“delta-delta Ct”) method (Livak and Schmittgen (2001) METHODS 25:402-408). As shown in FIG. 8, lumbar i.t. administration of the GalXC-ALDH2 RNAi oligonucleotide reduced target gene expression in the CNS of cynomolgus NHPs in a dose-dependent manner, as determined by comparison of the percentage of ALDH2 mRNA remaining in the tissue samples from cynomolgus NHPs treated with the GalXC-ALDH2 RNAi oligonucleotide provided in Table 3 relative to the percentage of ALDH2mRNA remaining in samples from control cynomolgus NHPs treated with PBS. These results demonstrate that GalNAc-conjugated RNAi oligonucleotides exhibit the ability to reduce target gene expression in a dose-dependent matter in multiple different anatomical regions of the NHP CNS following lumber i.t. injection.

### NHP Intra-Cisterna Magna (i.c.m.) Administration

The GalXC-ALDH2 RNAi oligonucleotide provided in Table 3 was administered as a single dose of 75 mg of oligonucleotide formulated in phosphate buffered saline (PBS) via lumbar intrathecal (i.t.) injection (3 mL) into the lumbar spine of male cynomolgus monkeys (n=3; average weight 3-3.5 kg). A control group of NHPs (n=3) was administered only PBS. Twenty-eight (28) days post-injection, NHPs were sacrificed. Whole brain and lumbar spinal cord were dissected and preserved for RT-qPCR analysis. RNA was extracted from tissue samples from the frontal cortex, caudate nucleus, hippocampus, cerebellum, cervical spinal cord, thoracic spinal cord, lumbar spinal cord, dorsal root ganglion, and liver to determine ALDH2 mRNA levels by qPCR (normalized to endogenous housekeeping genes Hprt, as indicated). The levels of ALDH2 mRNA were determined using PrimeTime™ qPCR Probe Assays (IDT). The qPCR was performed using PrimeTime™ qPCR Probe Assays, which consisted of a primer pair and fluorescently labeled 5′ nuclease probe specific to ALDH2 mRNA. The percentage of ALDH2 mRNA remaining in the samples from treated NHPs was determined using the 2^{-ΔΔCt} (“delta-delta Ct”) method (Livak and Schmittgen (2001) METHODS 25:402-08).

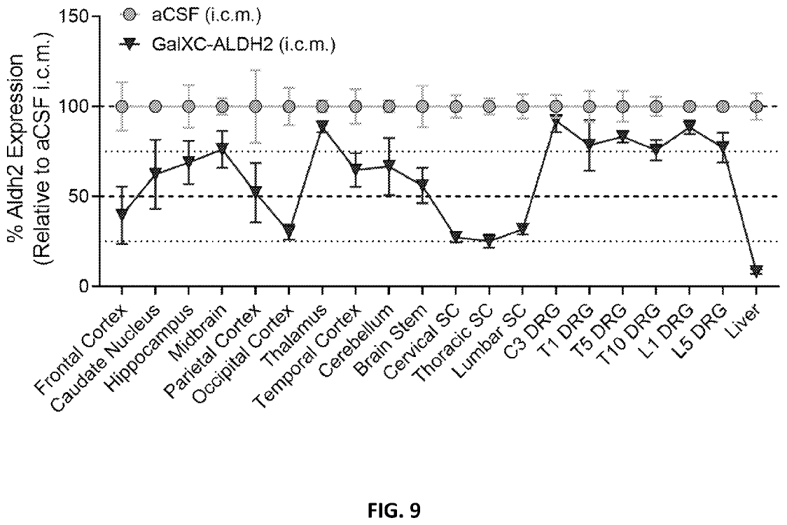

As shown in FIG. 9, lumbar i.t. administration of a GalNAc-conjugated RNAi oligonucleotide inhibited gene expression in the CNS, as determined by comparison of the percentage of Aldh2mRNA remaining in the tissue samples from cynomolgus NHPs treated with the GalXC-ALDH2 RNAi oligonucleotide provided in Table 3 relative to the percentage of Aldh2 mRNA remaining in samples from control cynomolgus NHPs treated with PBS. These results demonstrate that GalNAc-conjugated RNAi oligonucleotides exhibit the ability to inhibit gene expression in multiple different anatomical regions of the CNS following lumbar i.t. injection.

Taken together, the results provided in Examples 4-5 indicate that the reduction of the level of Aldh2 or ALDH2mRNA in the CNS of animals (rodents and NHPs) upon treatment with the GalXC-ALDH2 RNAi oligonucleotide provided in Table 3 relative to control animals treated with PBS is attributable to the RNAi-mediated silencing (RNA interference) of Aldh2 or ALDH2 mRNA by the GalXC-ALDH2 RNAi oligonucleotide in the CNS.

### Example 6: Lipid-Conjugated RNAi Oligonucleotides Inhibit Gene Expression in the Central Nervous System of Rodents

To evaluate the ability of lipid-conjugated RNAi oligonucleotides generated by general methods described herein and/or in Example 1-3 to reduce target gene expression in the central nervous system (CNS), mice were treated with lipid-conjugated RNAi oligonucleotides that targeted murine Aldh2 (alternatively “mALDH2” herein) mRNA via either intracerebroventricular (i.c.v.) or intrathecal (i.t.) administration into cerebrospinal fluid (CSF) and the subsequent effect(s) on Aldh2 expression in rodent (mouse) CNS was determined.

Briefly, RNAi oligonucleotide-lipid conjugates that target murine Aldh2 and comprising a lipid-conjugated nicked tetraloop structure having a 36-mer passenger strand and a 22-mer guide strand, the nucleotide sequences of which are set forth in Table 3, were generated (henceforth the “GalXC-ALDH2 RNAi oligonucleotide-lipid conjugate(s)”). The nucleotide sequences comprising the passenger strand and guide strand of the RNAi oligonucleotide-lipid conjugates each comprise a distinct pattern of modified nucleotides and phosphorothioate linkages, as depicted in FIG. 13 and shown below. Various lipid moieties were conjugated to one of the nucleotides comprising the tetraloop of the RNAi oligonucleotide-lipid conjugates, as described in Examples 1-3, specifically Scheme 1 shown in Example 3. The structures of the lipid moieties are provided in Table 4. The number of double bonds is referred to after the colon if present, e.g., a C18 hydrocarbon chain with one double bond is C18:1, a C22 hydrocarbon chain with 6 double bonds is C22:6.

TABLE 4
Structures of Exemplary Lipid Moieties

| HC | |
|---|---|
| Chain | HC Chain Structure |
| C8 | 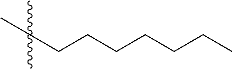 [SMILES: *CCCCCCC] |
| C10 | 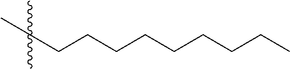 [SMILES: *CCCCCCCCC] |
| C12 | 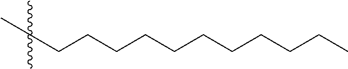 [SMILES: *CCCCCCCCCCC] |
| C14 | 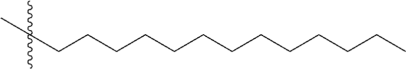 [SMILES: *CCCCCCCCCCCCC] |
| C16 | 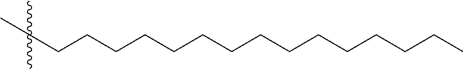 [SMILES: *CCCCCCCCCCCCCCC] |
| C18 | 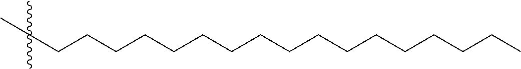 [SMILES: *CCCCCCCCCCCCCCCCC] |
| C18: 1 | 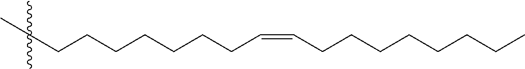 [SMILES: *CCCCCCC/C=C\CCCCCCCC] |
| C18: 2 | 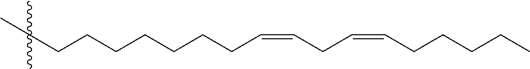 [SMILES: *CCCCCCC/C=C\C/C=C\CCCCC] |
| C22 | 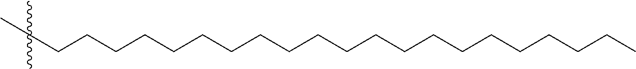 [SMILES: *CCCCCCCCCCCCCCCCCCCCC] |
| C22: 6 | 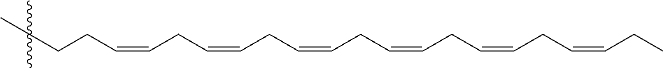 [SMILES: *CC/C=C\C/C=C\C/C=C\C/C=C\C/C=C\C/C=C\CC] |

*HC = hydrocarbon

The GalXC-ALDH2 RNAi oligonucleotide-lipid conjugates provided in Table 5 were individually administered as a single dose of 250 μg (10 μL; 25 mg/mL; 4 mg/kg) of oligonucleotide-lipid conjugate formulated in phosphate buffered saline (PBS) via intracerebroventricular (i.c.v.) injection into the brain of 6- to 8-week-old female CD-1 mice (n=4) or via lumbar intrathecal (i.t.) injection into the spine of 8- to 10-week-old female C57BL/6 mice (n=4). A control group of mice (n=4) was administered only PBS. Seven (7) days post-injection, mice were sacrificed. Whole brain and lumbar spinal cord were dissected and preserved for RT-qPCR analysis. RNA was extracted from tissue samples from the somatosensory cortex (SS cortex), hippocampus (HP), striatum, frontal cortex, cerebellum, hypothalamus (HY), cervical spinal cord (CSC), thoracic spinal cord (TSC), and lumbar spinal cord (LSC) to determine Aldh2 mRNA levels by qPCR (normalized to endogenous housekeeping gene Hprt, as indicated). The liver was also harvested from mice that were administered the GalXC-ALDH2 RNAi oligonucleotide-lipid conjugates or PBS by i.c.v injection, and RNA from the liver was extracted for RT-qPCR analysis. The levels of Aldh2 mRNA were determined using PrimeTime™ qPCR Probe Assays (IDT). The qPCR was performed using PrimeTime™ qPCR Probe Assays, which consisted of a primer pair and fluorescently labeled 5′ nuclease probe specific to Aldh2 mRNA. The percentage of Aldh2mRNA remaining in the samples from treated mice was determined using the 2^{-ΔΔCt} (“delta-delta Ct”) method (Livak and Schmittgen (2001) METHODS 25:402-08).

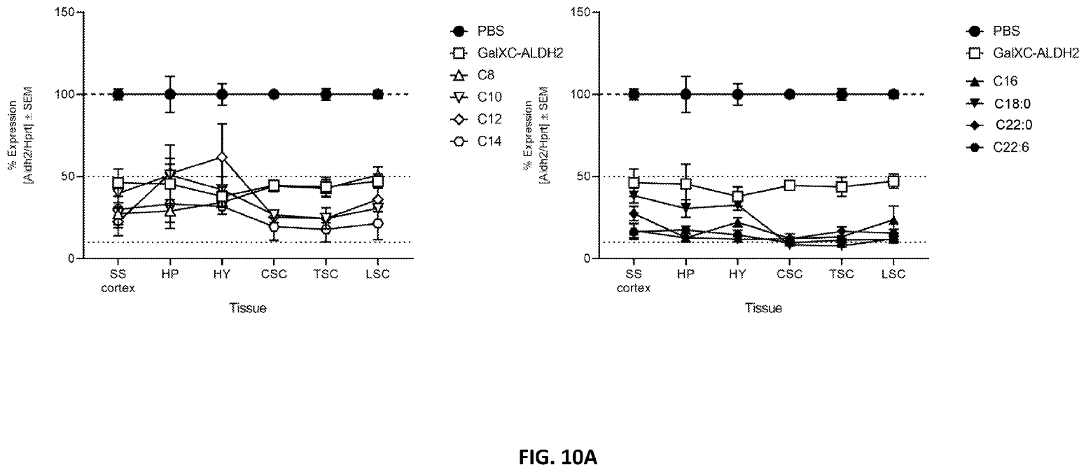

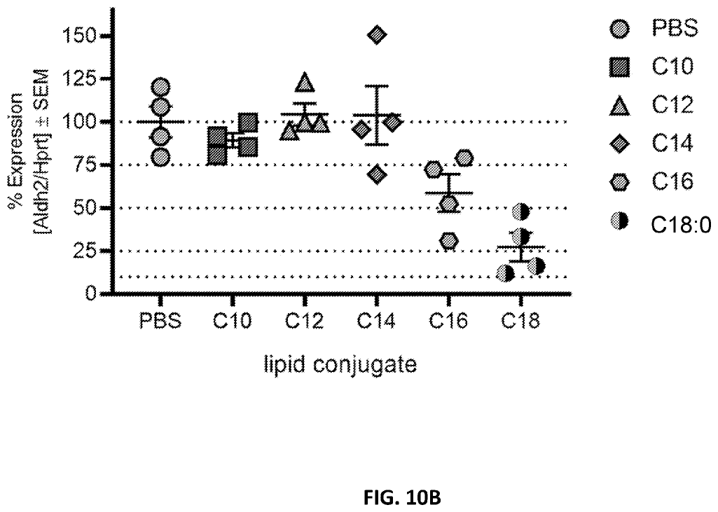

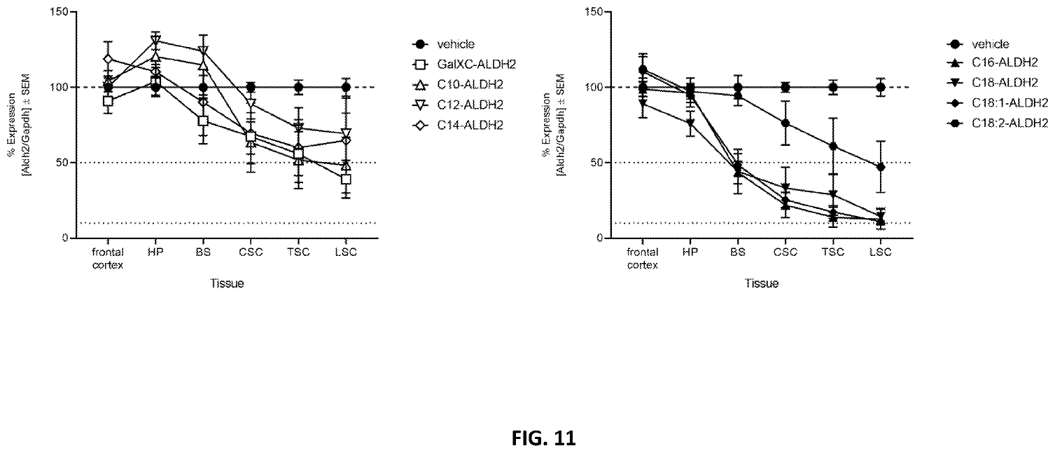

As shown in FIG. 10A and FIG. 11, i.c.v. administration (FIG. 10A) or i.t. administration (FIG. 11) of GalXC-ALDH2 RNAi oligonucleotide-lipid conjugates reduced target gene expression in the CNS, as determined by comparison of the percentage of murine Aldh2 mRNA remaining in the tissue samples from mice treated with the indicated GalXC-ALDH2 RNAi oligonucleotide-lipid conjugates relative to the percentage of murine Aldh2 mRNA remaining in samples from control mice treated with PBS. Further, as shown in FIG. 10A, all of the GalXC-ALDH2 RNAi oligonucleotide-lipid conjugates tested in mice via i.c.v. administration reduced Aldh2 target gene expression in the CNS to similar or greater extent than the control GalNAc-conjugated GalXC-ALDH2 RNAi oligonucleotide (GalXC-ALDH2). These results demonstrate that GalXC-ALDH2 RNAi oligonucleotide-lipid conjugates exhibit the ability to reduce target gene expression in multiple different anatomical regions of the CNS following i.c.v. or i.t. administration. Further, taken together, these results demonstrate that conjugation of a hydrophobic moiety (e.g., a lipid) to an RNAi oligonucleotide improves the ability of the RNAi oligonucleotide-lipid conjugate to reduce target gene expression in the CNS relative to a GalNAc-conjugated RNAi oligonucleotide making it useful in the therapeutic treatment of neurological diseases or disorders.

Target gene expression in the liver, as measured by the percentage of murine Aldh2 mRNA in the liver samples from mice administered a given GalXC-ALDH2 RNAi oligonucleotide-lipid conjugate relative to the percentage of murine Aldh2mRNA in liver samples from control mice, was evaluated for mice administered GalXC-ALDH2 RNAi oligonucleotide-lipid conjugates by i.c.v. injection. As shown in FIG. 10B, reduction of Aldh2 target gene expression in the liver was only observed in mice that were administered a GalXC-ALDH2 RNAi oligonucleotide-lipid conjugate having a longer lipid tail (i.e., C16 or C18). In contrast, mice that were administered GalXC-ALDH2 RNAi oligonucleotide-lipid conjugate with a shorter lipid tail (i.e., C10, C12, or C14) had no reduction in Aldh2 target gene expression in the liver relative to control mice. Together these data demonstrate that while GalXC-ALDH2 RNAi oligonucleotide-lipid conjugates with longer lipid tails reduce Aldh2 target gene expression in both the CNS and the liver, GalXC-ALDH2 RNAi oligonucleotide-lipid conjugates with shorter lipid tails are sufficiently versatile for reducing Aldh2 target gene expression in the CNS without affecting target gene expression in the liver. Such an effect provides for therapeutic manipulation of target gene expression in a tissue-specific manner, which is useful in disease contexts wherein the target gene is abnormally expressed in the CNS, but has expression in the liver that is required for normal physiological function. These data demonstrate that GalXC-ALDH2 RNAi oligonucleotide-lipid conjugates with shorter lipid tails can be used to reduce abnormal expression of the target gene in the CNS, without introducing deleterious reduction to the physiologically normal expression of the target gene in the liver.

TABLE 5
GalNAc- and Lipid-Conjugated GalXC-ALDH2 RNAi Oligonucleotides: Unmodified
and Modified Sense and Antisense Strand Nucleotide Sequences

| | | | SEQ ID NO | SEQ ID NO | SEQ ID NO | SEQ ID NO |
|---|---|---|---|---|---|---|
| RNAi | | | (Sense) | (Antisense) | (Sense) | (Antisense) |
| Oligonucleotide | Conjugate | DP# | Unmodified | | Modified | |
| GalXC- | 3xGalNAc | DP11518P: | 2 | 1 | 4 | 3 |
| ALDH2 | | DP11674G | | | | |
| GalXC- | Lipid | DP15541P: | 2 | 1 | 5 | 3 |
| ALDH2 | C8:0 | DP11674G | | | | |
| GalXC- | Lipid | DP15536P: | 2 | 1 | 6 | 3 |
| ALDH2 | C10:0 | DP11674G | | | | |
| GalXC- | Lipid | DP15537P: | 2 | 1 | 7 | 3 |
| ALDH2 | C12:0 | DP11674G | | | | |
| GalXC- | Lipid | DP15538P: | 2 | 1 | 8 | 3 |
| ALDH2 | C14:0 | DP11674G | | | | |
| GalXC- | Lipid | DP15542P: | 2 | 1 | 9 | 3 |
| ALDH2 | C16:0 | DP11674G | | | | |
| GalXC- | Lipid | DP15543P: | 2 | 1 | 10 | 3 |
| ALDH2 | C18:0 | DP11674G | | | | |
| GalXC- | Lipid | DP17180P: | 2 | 1 | 11 | 3 |
| ALDH2 | C18:1 | DP11674G | | | | |
| GalXC- | Lipid | DP17179P: | 2 | 1 | 12 | 3 |
| ALDH2 | C18:2 | DP11674G | | | | |
| GalXC- | Lipid | DP15545P: | 2 | 1 | 13 | 3 |
| ALDH2 | C22:0 | DP11674G | | | | |
| GalXC- | Lipid | DP15539P: | 2 | 1 | 14 | 3 |
| ALDH2 | C22:6 | DP11674G | | | | |

GalNAc-conjugated GalXC-ALDH2 RNAi oligonucleotide:

### Sense (Passenger) Strand:

5′-mX-S-mX-fX-mX-fX-mX-mX-fX-mX-fX-mX-fX-fX-mX-fX-mX-fX-[mX]₁₀-[ademX-GalNAc]-[ademX-GalNAc]-[ademX-GalNAc]-mX-mX-mX-mX-mX-mX-3′

### Hybridized to:

### Antisense (Guide) Strand:

### 5′-[MePhosphonate-4O-mX]—S-fX-S-fX-fX-fX-mX-fX-mX-mX-fX-mX-mX-mX-fX-mX-fX-mX-mX-fX-mX-S-mX-S-mX-3′

Lipid-conjugated (Caprylic acid C8:0) GalXC-ALDH2 RNAi oligonucleotide:

### Sense (Passenger) Strand:

5′-mX-S-mX-fX-mX-fX-mX-mX-fX-mX-fX-mX-fX-fX-mX-fX-mX-fX-[mX]₁₀-[ademX-C8]-mX-mX-mX-mX-mX-mX-mX-mX-3′.

### Hybridized to:

### Antisense (Guide) Strand:

### 5′-[MePhosphonate-4O-mX]—S-fX-S-fX-fX-fX-mX-fX-mX-mX-fX-mX-mX-mX-fX-mX-fX-mX-mX-fX-mX-S-mX-S-mX-3′

Lipid-conjugated (Decanoic acid C10:0) GalXC-ALDH2 RNAi oligonucleotide:

### Sense (Passenger) Strand:

5′-mX-S-mX-fX-mX-fX-mX-mX-fX-mX-fX-mX-fX-fX-mX-fX-mX-fX-[mX]₁₀-[ademX-C10]-mX-mX-mX-mX-mX-mX-mX-mX-3′.

### Hybridized to:

### Antisense (Guide) Strand:

### 5′-[MePhosphonate-4O-mX]—S-fX-S-fX-fX-fX-mX-fX-mX-mX-fX-mX-mX-mX-fX-mX-fX-mX-mX-fX-mX-S-mX-S-mX-3′

Lipid-conjugated (Capric acid C12:0) GalXC-ALDH2 RNAi oligonucleotide:

### Sense (Passenger) Strand:

5′-mX-S-mX-fX-mX-fX-mX-mX-fX-mX-fX-mX-fX-fX-mX-fX-mX-fX-[mX]₁₀-[ademX-C12]-mX-mX-mX-mX-mX-mX-mX-mX-3′.

### Hybridized to:

### Antisense (Guide) Strand:

### 5′-[MePhosphonate-4O-mX]—S-fX-S-fX-fX-fX-mX-fX-mX-mX-fX-mX-mX-mX-fX-mX-fX-mX-mX-fX-mX-S-mX-S-mX-3′

Lipid-conjugated (Myristic acid C14:0) GalXC-ALDH2 RNAi oligonucleotide:

### Sense (Passenger) Strand:

5′-mX-S-mX-fX-mX-fX-mX-mX-fX-mX-fX-mX-fX-fX-mX-fX-mX-fX-[mX]₁₀-[ademX-C14]-mX-mX-mX-mX-mX-mX-mX-mX-3′.

### Hybridized to:

### Antisense (Guide) Strand:

### 5′-[MePhosphonate-4O-mX]—S-fX-S-fX-fX-fX-mX-fX-mX-mX-fX-mX-mX-mX-fX-mX-fX-mX-mX-fX-mX-S-mX-S-mX-3′

Lipid-conjugated (Palmitic acid C16:0) GalXC-ALDH2 RNAi oligonucleotide:

### Sense (Passenger) Strand:

5′-mX-S-mX-fX-mX-fX-mX-mX-fX-mX-fX-mX-fX-fX-mX-fX-mX-fX-[mX]₁₀-[ademX-C16]-mX-mX-mX-mX-mX-mX-mX-mX-3′.

### Hybridized to:

### Antisense (Guide) Strand:

### 5′-[MePhosphonate-4O-mX]—S-fX-S-fX-fX-fX-mX-fX-mX-mX-fX-mX-mX-mX-fX-mX-fX-mX-mX-fX-mX-S-mX-S-mX-3′

Lipid-conjugated (Stearic acid C18:0) GalXC-ALDH2 RNAi oligonucleotide:

### Sense (Passenger) Strand:

5′-mX-S-mX-fX-mX-fX-mX-mX-fX-mX-fX-mX-fX-fX-mX-fX-mX-fX-[mX]₁₀-[ademX-C18]-mX-mX-mX-mX-mX-mX-mX-mX-3′.

### Hybridized to:

### Antisense (Guide) Strand:

### 5′-[MePhosphonate-4O-mX]—S-fX-S-fX-fX-fX-mX-fX-mX-mX-fX-mX-mX-mX-fX-mX-fX-mX-mX-fX-mX-S-mX-S-mX-3′

Lipid-conjugated (Oleic acid C18:1) GalXC-ALDH2 RNAi oligonucleotide:

### Sense (Passenger) Strand:

5′-mX-S-mX-fX-mX-fX-mX-mX-fX-mX-fX-mX-fX-fX-mX-fX-mX-fX-[mX]₁₀-[ademX-C18-OLE]-mX-mX-mX-mX-mX-mX-mX-mX-3′.

### Hybridized to:

### Antisense (Guide) Strand:

### 5′-[MePhosphonate-4O-mX]—S-fX-S-fX-fX-fX-mX-fX-mX-mX-fX-mX-mX-mX-fX-mX-fX-mX-mX-fX-mX-S-mX-S-mX-3′

Lipid-conjugated (Linoleic acid C18:2) GalXC-ALDH2 RNAi oligonucleotide:

### Sense (Passenger) Strand:

5′-mX-S-mX-fX-mX-fX-mX-mX-fX-mX-fX-mX-fX-fX-mX-fX-mX-fX-[mX]₁₀-[ademX-C18-LIN]-mX-mX-mX-mX-mX-mX-mX-mX-3′.

### Hybridized to:

### Antisense (Guide) Strand:

### 5′-[MePhosphonate-4O-mX]—S-fX-S-fX-fX-fX-mX-fX-mX-mX-fX-mX-mX-mX-fX-mX-fX-mX-mX-fX-mX-S-mX-S-mX-3′

Lipid-conjugated (Docosanoic acid C22:0) GalXC-ALDH2 RNAi oligonucleotide:

### Sense (Passenger) Strand:

5′-mX-S-mX-fX-mX-fX-mX-mX-fX-mX-fX-mX-fX-fX-mX-fX-mX-fX-[mX]₁₀-[ademX-C22]-mX-mX-mX-mX-mX-mX-mX-mX-3′.

### Hybridized to:

### Antisense (Guide) Strand:

### 5′-[MePhosphonate-4O-mX]—S-fX-S-fX-fX-fX-mX-fX-mX-mX-fX-mX-mX-mX-fX-mX-fX-mX-mX-fX-mX-S-mX-S-mX-3′

Lipid-conjugated (Docosahexaenoic acid C22:6) GalXC-ALDH2 RNAi oligonucleotide:

### Sense (Passenger) Strand:

5′-mX-S-mX-fX-mX-fX-mX-mX-fX-mX-fX-mX-fX-fX-mX-fX-mX-fX-[mX]₁₀-[ademX-C22-DHA]-mX-mX-mX-mX-mX-mX-mX-mX-3′.

### Hybridized to:

### Antisense (Guide) Strand:

### 5′-[MePhosphonate-4O-mX]—S-fX-S-fX-fX-fX-mX-fX-mX-mX-fX-mX-mX-mX-fX-mX-fX-mX-mX-fX-mX-S-mX-S-mX-3′

### (Modification key: Table 6)

TABLE 6
Modification Key

| Symbol | Modification/linkage |
|---|---|
| mX | 2′-O-methyl modified nucleotide |
| fX | 2′-fluoro modified nucleotide |
| -S- | phosphorothioate linkage |
| - | phosphodiester linkage |
| [MePhosphonate- | 5′-methoxyphosphonate-4-oxy modified nucleotide |
| 4O-mX] | |
| ademX-GalNAc | GalNAc-conjugated nucleotide |
| ademX-C8 | Lipid (C8:0)-conjugated nucleotide |
| ademX-C10 | Lipid (C10:0)-conjugated nucleotide |
| ademX-C12 | Lipid (C12:0)-conjugated nucleotide |
| ademX-C14 | Lipid (C14:0)-conjugated nucleotide |
| ademX-C16 | Lipid (C16:0)-conjugated nucleotide |
| ademX-C18 | Lipid (C18:0)-conjugated nucleotide |
| ademX-C18-OLE | Lipid (C18:1)-conjugated nucleotide |
| ademX-C18-LIN | Lipid (C18:2)-conjugated nucleotide |
| ademX-C22 | Lipid (C22:0)-conjugated nucleotide |
| ademX-C22-DHA | Lipid (C22:6)-conjugated nucleotide |

SEQUENCE LISTING

| | | | | | SEQ |
|---|---|---|---|---|---|
| | Descrip- | | | | ID |
| Name | tion | Conjugate | Strand | Sequence | NO |
| GalXC- | Unmodified | | 22-mer | UAAACUGAGUUUCAUCCAC | 1 |
| ALDH | | | antisense | CGG | |
| 2 | | | strand | | |
| GalXC- | Unmodified | | 36-mer | GGUGGAUGAAACUCAGUUU | 2 |
| ALDH | | | sense | AGCAGCCGAAAGGCUGC | |
| 2 | | | strand | | |
| GalXC- | Modified | | 22-mer | [MePhosphonate-40- | 3 |
| ALDH | | | antisense | mUs][fAs][fA][fA][fC][mU][fG] | |
| 2 | | | strand | [mA][mG][fU][mU][mU][mC][fA | |
| | | | | ][mU][fC][mC][mA][fC][mCs][m | |
| | | | | Gs][mG] | |
| GalXC- | Modified | GalNAc | 36-mer | [mGs][mG][fU][mG][fG][mA][m | 4 |
| ALDH | | | sense | U][fG][mA][fA][mA][fC][fU][m | |
| 2 | | | strand | C][fA][mG][fU][mU][mU][mA][ | |
| | | | | mG][mC][mA][mG][mC][mC][m | |
| | | | | G][ademA-GalNAc][ademA- | |
| | | | | GalNAc][ademA- | |
| | | | | GalNAc][mG][mG][mC][mU][m | |
| | | | | G][mC] | |
| GalXC- | Modified | Lipid | 36-mer | [mGs][mG][fU][mG][fG][mA][m | 5 |
| ALDH | | (C8:0) | sense | U][fG][mA][fA][mA][fC][fU] | |
| 2 | | | strand | [mC][fA][mG][fU][mU][mU][mA] | |
| | | | | [mG][mC][mA][mG][mC][mC][m | |
| | | | | G][ademA- | |
| | | | | C8][mA][mA][mG][mG][mC][m | |
| | | | | U][mG][mC] | |
| GalXC- | Modified | Lipid | 36-mer | [mGs][mG][fU][mG][fG][mA][m | 6 |
| ALDH | | (C10:0) | sense | U][fG][mA][fA][mA][fC][fU][m | |
| 2 | | | strand | C][fA][mG][fU][mU][mU][mA][ | |
| | | | | mG][mC][mA][mG][mC][mC][m | |
| | | | | G][ademA- | |
| | | | | C10][mA][mA][mG][mG][mC][ | |
| | | | | mU][mG][mC] | |
| GalXC- | Modified | Lipid | 36-mer | [mGs][mG][fU][mG][fG][mA][m | 7 |
| ALDH | | (C12:0) | sense | U][fG][mA][fA][mA][fC][fU][m | |
| 2 | | | strand | C][fA][mG][fU][mU][mU][mA] | |
| | | | | [mG][mC][mA][mG][mC][mC][mG] | |
| | | | | [ademA- | |
| | | | | C12][mA][mA][mG][mG][mC][ | |
| | | | | mU][mG][mC] | |
| GalXC- | Modified | Lipid | 36-mer | [mGs][mG][fU][mG][fG][mA] | 8 |
| ALDH | | (C14:0) | sense | [mU][fG][mA][fA][mA][fC][fU] | |
| 2 | | | strand | [mC][fA][mG][fU][mU][mU][mA] | |
| | | | | [mG][mC][mA][mG][mC][mC] | |
| | | | | [mG][ademA- | |
| | | | | C14][mA][mA][mG][mG][mC][ | |
| | | | | mU][mG][mC] | |
| GalXC- | Modified | Lipid | 36-mer | [mGs][mG][fU][mG][fG][mA][m | 9 |
| ALDH | | (C16:0) | sense | U][fG][mA][fA][mA][fC][fU][m | |
| 2 | | | strand | C][fA][mG][fU][mU][mU][mA][ | |
| | | | | mG][mC][mA][mG][mC][mC][m | |
| | | | | G][ademA- | |
| | | | | C16][mA][mA][mG][mG][mC] | |
| | | | | [mU][mG][mC] | |
| GalXC- | Modified | Lipid | 36-mer | [mGs][mG][fU][mG][fG][mA][m | 10 |
| ALDH | | (C18:0) | sense | U][fG][mA][fA][mA][fC][fU][m | |
| 2 | | | strand | C][fA][mG][fU][mU][mU][mA] | |
| | | | | [mG][mC][mA][mG][mC][mC] | |
| | | | | [mG][ademA- | |
| | | | | C18][mA][mA][mG][mG][mC][ | |
| | | | | mU][mG][mC] | |
| GalXC- | Modified | Lipid | 36-mer | [mGs][mG][fU][mG][fG][mA][m | 11 |
| ALDH | | (C18:1) | sense | U][fG][mA][fA][mA][fC][fU][m | |
| 2 | | | strand | C][fA][mG][fU][mU][mU][mA][ | |
| | | | | mG][mC][mA][mG][mC][mC] | |
| | | | | [mG][ademA-C18-OLE] | |
| | | | | [mA][mA][mG][mG][mC] | |
| | | | | [mU][mG][mC] | |
| GalXC- | Modified | Lipid | 36-mer | [mGs][mG][fU][mG][fG][mA][m | 12 |
| ALDH | | (C18:2) | sense | U][fG][mA][fA][mA][fC][fU][m | |
| 2 | | | strand | C][fA][mG][fU][mU][mU][mA] | |
| | | | | [mG][mC][mA][mG][mC][mC] | |
| | | | | [mG][ademA-C18- | |
| | | | | LIN][mA][mA][mG][mG][mC][ | |
| | | | | mU][mG][mC] | |
| GalXC- | Modified | Lipid | 36-mer | [mGs][mG][fU][mG][fG][mA][m | 13 |
| ALDH | | (C22:0) | sense | U][fG][mA][fA][mA][fC][fU][m | |
| 2 | | | strand | C][fA][mG][fU][mU][mU][mA] | |
| | | | | [mG][mC][mA][mG][mC][mC][m | |
| | | | | G][ademA-C22] | |
| | | | | [mA][mA][mG][mG][mC] | |
| | | | | [mU][mG][mC] | |
| GalXC- | Modified | Lipid | 36-mer | [mGs][mG][fU][mG][fG][mA][m | 14 |
| ALDH | | (C22:6) | sense | U][fG][mA][fA][mA][fC][fU][m | |
| 2 | | | strand | C][fA][mG][fU][mU][mU][mA] | |
| | | | | [mG][mC][mA][mG][mC][mC] | |
| | | | | [mG][ademA-C22- | |
| | | | | DHA][mA][mA][mG][mG][mC] | |
| | | | | [mU][mG][mC] | |
| Stem | | | | GCAGCCGAAAGGCUGC | 15 |
| loop | | | | | |

We claim:

1. An oligonucleotide-ligand conjugate for reducing expression of a target mRNA in the central nervous system (CNS) comprising
(i) a double-stranded oligonucleotide comprising an antisense strand of 15 to 30 nucleotides in length and a sense strand of 15 to 40 nucleotides in length, wherein the antisense strand and the sense strand each comprise a 5′ end and a 3′ end, wherein the antisense strand and the sense strand form a duplex region, wherein the antisense strand has a region of complementarity to a target sequence in the target mRNA in the CNS, wherein the region of complementarity is at least 15 contiguous nucleotides in length, wherein the oligonucleotide comprises a stem-loop; and
(ii) one or more lipid moieties conjugated to the stem-loop.

2. The oligonucleotide-ligand conjugate of claim 1, wherein the sense strand comprises the stem-loop at its 3′ end.

3. The oligonucleotide-ligand conjugate of claim 1 or 2, wherein the stem-loop comprises a nucleotide sequence represented by the formula: 5′-S1-L-S2-3′, wherein S1 is complementary to S2, and wherein L forms a loop between S1 and S2.

4. The oligonucleotide-ligand conjugate of claim 3, wherein S1 and S2 are 1 to 8 nucleotides in length.

5. The oligonucleotide-ligand conjugate of claim 3 or 4, wherein the one or more lipid moieties is conjugated to a nucleotide of S1.

6. The oligonucleotide-ligand conjugate of claim 3 or 4, wherein the one or more lipid moieties is conjugated to a nucleotide of S2.

7. The oligonucleotide-ligand conjugate of any one of claims 3-6, wherein L is 3 to 5 nucleotides in length.

8. The oligonucleotide-ligand conjugate of any one of claims 3-7, wherein L is a tetraloop, optionally wherein L is 4 nucleotides in length.

9. The oligonucleotide-ligand conjugate of any one of claims 3-8, wherein L comprises the nucleotide sequence set forth as 5′-GAAA-3′.

10. The oligonucleotide-ligand conjugate of any one of claims 3-9, wherein the one or more lipid moieties is conjugated to a nucleotide of L.

11. The oligonucleotide-ligand conjugate of claim 3 or 4, wherein L is 3 nucleotides in length and has 5′ to 3′ a first, second, and third nucleotide, wherein the oligonucleotide-ligand conjugate comprises one lipid moiety, and wherein the lipid moiety is conjugated to the first, second, or third nucleotide of L.

12. The oligonucleotide-ligand conjugate of claim 3 or 4, wherein L is 4 nucleotides in length and has 5′ to 3′ a first, second, third, and fourth nucleotide, wherein the oligonucleotide-ligand conjugate comprises one lipid moiety, and wherein the lipid moiety is conjugated to the first, second, third, or fourth nucleotide of L.

13. The oligonucleotide-ligand conjugate of claim 12, wherein L consists of 5′-GAAA-3′.

14. The oligonucleotide-ligand conjugate of any one of claims 11-13, wherein the lipid moiety is conjugated to the second nucleotide of L.

15. The oligonucleotide-ligand conjugate of claim 3 or 4, wherein L is 5 nucleotides in length and has 5′ to 3′ a first, second, third, fourth, and fifth nucleotide, wherein the oligonucleotide-ligand conjugate comprises one lipid moiety, and wherein the lipid moiety is conjugated to the first, second, third, fourth, or fifth nucleotide of L.

16. The oligonucleotide-ligand conjugate of any one of claims 1-15, wherein the antisense strand is 19 to 27 nucleotides in length.

17. The oligonucleotide-ligand conjugate of claims 1-16, wherein the antisense strand is 21 to 27 nucleotides in length, optionally wherein the antisense strand is 22 nucleotides in length.

18. The oligonucleotide-ligand conjugate of any one of claims 1-17, wherein the sense strand is 19 to 40 nucleotides in length, optionally wherein the sense strand is 36 nucleotides in length.

19. The oligonucleotide-ligand conjugate of any one of claims 1-18, wherein the duplex region is at least 19 nucleotides in length.

20. The oligonucleotide-ligand conjugate of any one of claims 1-19, wherein the duplex region is at least 20 nucleotides in length, optionally wherein the duplex region is 21 nucleotides in length.

21. The oligonucleotide-ligand conjugate of any one of claims 1-20, wherein the region of complementarity is at least 19 contiguous nucleotides in length.

22. The oligonucleotide-ligand conjugate of any one of claims 1-21, wherein the oligonucleotide comprises a 3′-overhang sequence of one or more nucleotides in length, wherein the 3′-overhang sequence is present on the antisense strand, the sense strand, or the antisense strand and sense strand.

23. The oligonucleotide-ligand conjugate of claim 22, wherein the 3′-overhang sequence is on the antisense strand, and wherein the 3′-overhang sequence is two nucleotides in length.

24. The oligonucleotide-ligand conjugate of any one of claims 1-23, wherein the oligonucleotide comprises at least one modified nucleotide.

25. The oligonucleotide-ligand conjugate of claim 24, wherein the modified nucleotide comprises a 2′-modification.

26. The oligonucleotide-ligand conjugate of claim 25, wherein the 2′-modification is a modification selected from: 2′-aminoethyl, 2′-fluoro, 2′-O-methyl, 2′-O-methoxyethyl, 2′-deoxy-2′-fluoro, and 2′-deoxy-2′-fluoro-β-d-arabino.

27. The oligonucleotide-ligand conjugate of any one of claims 24-26, wherein all the nucleotides of the oligonucleotide are modified.

28. The oligonucleotide-ligand conjugate of any one of claims 1-27, wherein the oligonucleotide comprises at least one modified internucleotide linkage.

29. The oligonucleotide-ligand conjugate of claim 28, wherein the at least one modified internucleotide linkage is a phosphorothioate linkage.

30. The oligonucleotide-ligand conjugate of any one of claims 1-29, wherein the 4′-carbon of the sugar of the 5′-nucleotide of the antisense strand comprises a phosphate analog.

31. The oligonucleotide-ligand conjugate of claim 30, wherein the phosphate analog is oxymethylphosphonate, vinylphosphonate, or malonylphosphonate.

32. The oligonucleotide-ligand conjugate of any one of claims 1-31, wherein the one or more lipid moieties comprise a saturated or unsaturated C1-C50 hydrocarbon chain.

33. The oligonucleotide-ligand conjugate of any one of claims 1-32, wherein the one or more lipid moieties comprise a saturated or unsaturated C5-C25 hydrocarbon chain.

34. The oligonucleotide-ligand conjugate of claim 33, wherein the one or more lipid moieties comprise a saturated C8, C9, C10, C11, C12, C13, or C14 hydrocarbon chain.

35. The oligonucleotide-ligand conjugate of claim 34, wherein the oligonucleotide-ligand conjugate reduces expression of the target mRNA in the CNS without reducing expression of the target mRNA outside the CNS, optionally without reducing expression of the target mRNA in the liver.

36. The oligonucleotide-ligand conjugate of claim 34 or 35, wherein the target mRNA is expressed in liver cells, and wherein the oligonucleotide-ligand conjugate does not reduce expression of the target mRNA in liver cells to the same or similar level as in the cells of the CNS.

37. The oligonucleotide-ligand conjugate of claim 33, wherein the lipid moiety comprises a saturated C16, C17, C18, C19, C20, C21, or C22 hydrocarbon chain.

38. The oligonucleotide-ligand conjugate of claim 33, wherein the lipid moiety comprises an unsaturated C16, C17, C18, C19, C20, C21, or C22 hydrocarbon chain, optionally wherein the lipid moiety comprises C22:6.

39. The oligonucleotide-ligand conjugate of any one of claims 1-38, wherein the target gene is associated with a disease or disorder, optionally a neurological disease or disorder.

40. The oligonucleotide-ligand conjugate of claim 39, wherein the disease or disorder is selected from Progressive Supranuclear Palsy (PSP), Corticobasal degeneration (CBD), Argyrophilic grain disease (AGD), Globular glial tauopathy (GGT), Aging-related tau astrogliopathy (ARTAG), Familial Frontotemporal Dementia 17 (FTD-17), Tauopathy with Respiratory Failure, Dementia with Seizures, Pick's disease, Myotonic dystrophy 1 or 2 (MD1 or MD2), Down's syndrome, Spastic Paraplegia (SP), Niemann-Pick disease type C, Dementia with Lewy bodies (DLB), Lewy body dysphagia, Lewy body disease, Olivopontocerebellar atrophy, Striatonigral degeneration, Shy-Drager syndrome, Spinal muscular atrophy V (SMAV), Huntington's Disease (HD), Alzheimer's Disease, SCA1, SCA2, SCA3, SCA7, SCA10 (spinocerebellar ataxia type 1, 2, 3, 7 or 103), Multiple System Atrophy (MSA), Spinal and Bulbar Muscular Atrophy (SBMA, Kennedy disease), Friedrich Ataxia, Fragile X-associated tremor/ataxia syndrome (FXTAS), Fragile X syndrome (FRAXA), X-Linked Mental Retardation (XLMR), Parkinson's Disease, Dystonia, SBMA (spinobulbar muscular atrophy), neuropathic pain disorders, spinal cord injury, Dentatorubral-pallidoluysian atrophy (DRPLA), recessive CNS disorders, and ALS (amyotrophic lateral sclerosis), M2DS (MECP2 duplication syndrome), FTD (frontotemporal dementia), Prion disease, Adult Onset Leukodystrophy, Alexander's Disease, Krabbe Disease, Chronic Traumatic Encephalopathy, Pelizaeus-Merzbacher disease (PMD), Lafora disease, stroke, Cerebral Amyloid Angiopathy (CAA), and Metachromatic Leukodystrophy (MLD).

41. An oligonucleotide-ligand conjugate for reducing expression of a target mRNA in the CNS comprising
(i) a double-stranded oligonucleotide comprising an antisense strand of 15 to 30 nucleotides in length and a sense strand of 15 to 40 nucleotides in length, wherein the antisense strand and the sense strand each comprise a 5′ end and a 3′ end, wherein the antisense strand and the sense strand form a duplex region, wherein the antisense strand has a region of complementarity to a target sequence in the target mRNA in the CNS, wherein the region of complementarity is at least 15 contiguous nucleotides in length, wherein the oligonucleotide comprises a stem-loop; and
(ii) one or more lipid moieties conjugated to the stem-loop, wherein the one or more lipid moieties are selected from saturated or unsaturated C8-14 hydrocarbon chains,
wherein expression of the target mRNA in the liver is not reduced to the same or similar level as in the CNS.

42. A pharmaceutical composition comprising the oligonucleotide-ligand conjugate of any one of claims 1-41, and a pharmaceutically acceptable carrier.

43. A method of reducing or inhibiting expression of a target mRNA in a subject expressed by a population of cells associated with the CNS in a subject, comprising administering the oligonucleotide-ligand conjugate of any one of claims 1-41 or the pharmaceutical composition of claim 42 to the subject.

44. The method of claim 43, wherein the level of expression of the target mRNA is reduced in the population of cells associated with the CNS compared to a control population of cells.

45. The method of claim 44, wherein the level of expression of the target mRNA is not reduced in population of cells residing outside the CNS compared to a control population of cells.

46. The method of claim 45, wherein the population of cells residing outside the CNS are in the liver.


SEQUENCE LISTING (15 sequences)

SEQ ID NO: 1 - RNA, 22 nt, Artificial Sequence
  synthetic construct
uaaacugaguuucauccaccgg

SEQ ID NO: 2 - RNA, 36 nt, Artificial Sequence
  synthetic construct
gguggaugaaacucaguuuagcagccgaaaggcugc

SEQ ID NO: 3 - RNA, 22 nt, Artificial Sequence
  synthetic construct
  misc_feature (1)..(3): linked via phosphorothioate
  modified_base (1)..(1): 5′-methoxyphosphonate-4-oxy modified
  modified_base (2)..(2): 2′- fluoro modified
  modified_base (3)..(3): 2′- fluoro modified
  modified_base (4)..(4): 2′- fluoro modified
  modified_base (5)..(5): 2′- fluoro modified
  modified_base (6)..(6): 2′-O-methyl modified
  modified_base (7)..(7): 2′- fluoro modified
  modified_base (8)..(8): 2′-O-methyl modified
  modified_base (9)..(9): 2′-O-methyl modified
  modified_base (10)..(10): 2′- fluoro modified
  modified_base (11)..(11): 2′-O-methyl modified
  modified_base (12)..(12): 2′-O-methyl modified
  modified_base (13)..(13): 2′-O-methyl modified
  modified_base (14)..(14): 2′- fluoro modified
  modified_base (15)..(15): 2′-O-methyl modified
  modified_base (16)..(16): 2′- fluoro modified
  modified_base (17)..(17): 2′-O-methyl modified
  modified_base (18)..(18): 2′-O-methyl modified
  modified_base (19)..(19): 2′- fluoro modified
  misc_feature (20)..(22): linked via phosphorothioate
  modified_base (20)..(20): 2′-O-methyl modified
  modified_base (21)..(21): 2′-O-methyl modified
  modified_base (22)..(22): 2′-O-methyl modified
uaaacugaguuucauccaccgg

SEQ ID NO: 4 - RNA, 36 nt, Artificial Sequence; identical to SEQ ID NO: 2
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′- fluoro modified
  modified_base (4)..(4): 2′-O-methyl modified
  modified_base (5)..(5): 2′- fluoro modified
  modified_base (6)..(6): 2′-O-methyl modified
  modified_base (7)..(7): 2′-O-methyl modified
  modified_base (8)..(8): 2′- fluoro modified
  modified_base (9)..(9): 2′-O-methyl modified
  modified_base (10)..(10): 2′- fluoro modified
  modified_base (11)..(11): 2′-O-methyl modified
  modified_base (12)..(12): 2′- fluoro modified
  modified_base (13)..(13): 2′- fluoro modified
  modified_base (14)..(14): 2′-O-methyl modified
  modified_base (15)..(15): 2′- fluoro modified
  modified_base (16)..(16): 2′-O-methyl modified
  modified_base (17)..(17): 2′- fluoro modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 5 - RNA, 36 nt, Artificial Sequence; identical to SEQ ID NO: 2
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′- fluoro modified
  modified_base (4)..(4): 2′-O-methyl modified
  modified_base (5)..(5): 2′- fluoro modified
  modified_base (6)..(6): 2′-O-methyl modified
  modified_base (7)..(7): 2′-O-methyl modified
  modified_base (8)..(8): 2′- fluoro modified
  modified_base (9)..(9): 2′-O-methyl modified
  modified_base (10)..(10): 2′- fluoro modified
  modified_base (11)..(11): 2′-O-methyl modified
  modified_base (12)..(12): 2′- fluoro modified
  modified_base (13)..(13): 2′- fluoro modified
  modified_base (14)..(14): 2′-O-methyl modified
  modified_base (15)..(15): 2′- fluoro modified
  modified_base (16)..(16): 2′-O-methyl modified
  modified_base (17)..(17): 2′- fluoro modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): Lipid (C80)-conjugated
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 6 - RNA, 36 nt, Artificial Sequence; identical to SEQ ID NO: 2
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′- fluoro modified
  modified_base (4)..(4): 2′-O-methyl modified
  modified_base (5)..(5): 2′- fluoro modified
  modified_base (6)..(6): 2′-O-methyl modified
  modified_base (7)..(7): 2′-O-methyl modified
  modified_base (8)..(8): 2′- fluoro modified
  modified_base (9)..(9): 2′-O-methyl modified
  modified_base (10)..(10): 2′- fluoro modified
  modified_base (11)..(11): 2′-O-methyl modified
  modified_base (12)..(12): 2′- fluoro modified
  modified_base (13)..(13): 2′- fluoro modified
  modified_base (14)..(14): 2′-O-methyl modified
  modified_base (15)..(15): 2′- fluoro modified
  modified_base (16)..(16): 2′-O-methyl modified
  modified_base (17)..(17): 2′- fluoro modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): Lipid (C100)-conjugated
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 7 - RNA, 36 nt, Artificial Sequence; identical to SEQ ID NO: 2
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′- fluoro modified
  modified_base (4)..(4): 2′-O-methyl modified
  modified_base (5)..(5): 2′- fluoro modified
  modified_base (6)..(6): 2′-O-methyl modified
  modified_base (7)..(7): 2′-O-methyl modified
  modified_base (8)..(8): 2′- fluoro modified
  modified_base (9)..(9): 2′-O-methyl modified
  modified_base (10)..(10): 2′- fluoro modified
  modified_base (11)..(11): 2′-O-methyl modified
  modified_base (12)..(12): 2′- fluoro modified
  modified_base (13)..(13): 2′- fluoro modified
  modified_base (14)..(14): 2′-O-methyl modified
  modified_base (15)..(15): 2′- fluoro modified
  modified_base (16)..(16): 2′-O-methyl modified
  modified_base (17)..(17): 2′- fluoro modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): Lipid (C120)-conjugated
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 8 - RNA, 36 nt, Artificial Sequence; identical to SEQ ID NO: 2
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′- fluoro modified
  modified_base (4)..(4): 2′-O-methyl modified
  modified_base (5)..(5): 2′- fluoro modified
  modified_base (6)..(6): 2′-O-methyl modified
  modified_base (7)..(7): 2′-O-methyl modified
  modified_base (8)..(8): 2′- fluoro modified
  modified_base (9)..(9): 2′-O-methyl modified
  modified_base (10)..(10): 2′- fluoro modified
  modified_base (11)..(11): 2′-O-methyl modified
  modified_base (12)..(12): 2′- fluoro modified
  modified_base (13)..(13): 2′- fluoro modified
  modified_base (14)..(14): 2′-O-methyl modified
  modified_base (15)..(15): 2′- fluoro modified
  modified_base (16)..(16): 2′-O-methyl modified
  modified_base (17)..(17): 2′- fluoro modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): Lipid (C140)-conjugated
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 9 - RNA, 36 nt, Artificial Sequence; identical to SEQ ID NO: 2
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′- fluoro modified
  modified_base (4)..(4): 2′-O-methyl modified
  modified_base (5)..(5): 2′- fluoro modified
  modified_base (6)..(6): 2′-O-methyl modified
  modified_base (7)..(7): 2′-O-methyl modified
  modified_base (8)..(8): 2′- fluoro modified
  modified_base (9)..(9): 2′-O-methyl modified
  modified_base (10)..(10): 2′- fluoro modified
  modified_base (11)..(11): 2′-O-methyl modified
  modified_base (12)..(12): 2′- fluoro modified
  modified_base (13)..(13): 2′- fluoro modified
  modified_base (14)..(14): 2′-O-methyl modified
  modified_base (15)..(15): 2′- fluoro modified
  modified_base (16)..(16): 2′-O-methyl modified
  modified_base (17)..(17): 2′- fluoro modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): Lipid (C160)-conjugated
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 10 - RNA, 36 nt, Artificial Sequence; identical to SEQ ID NO: 2
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′- fluoro modified
  modified_base (4)..(4): 2′-O-methyl modified
  modified_base (5)..(5): 2′- fluoro modified
  modified_base (6)..(6): 2′-O-methyl modified
  modified_base (7)..(7): 2′-O-methyl modified
  modified_base (8)..(8): 2′- fluoro modified
  modified_base (9)..(9): 2′-O-methyl modified
  modified_base (10)..(10): 2′- fluoro modified
  modified_base (11)..(11): 2′-O-methyl modified
  modified_base (12)..(12): 2′- fluoro modified
  modified_base (13)..(13): 2′- fluoro modified
  modified_base (14)..(14): 2′-O-methyl modified
  modified_base (15)..(15): 2′- fluoro modified
  modified_base (16)..(16): 2′-O-methyl modified
  modified_base (17)..(17): 2′- fluoro modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): Lipid (C180)-conjugated
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 11 - RNA, 36 nt, Artificial Sequence; identical to SEQ ID NO: 2
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′- fluoro modified
  modified_base (4)..(4): 2′-O-methyl modified
  modified_base (5)..(5): 2′- fluoro modified
  modified_base (6)..(6): 2′-O-methyl modified
  modified_base (7)..(7): 2′-O-methyl modified
  modified_base (8)..(8): 2′- fluoro modified
  modified_base (9)..(9): 2′-O-methyl modified
  modified_base (10)..(10): 2′- fluoro modified
  modified_base (11)..(11): 2′-O-methyl modified
  modified_base (12)..(12): 2′- fluoro modified
  modified_base (13)..(13): 2′- fluoro modified
  modified_base (14)..(14): 2′-O-methyl modified
  modified_base (15)..(15): 2′- fluoro modified
  modified_base (16)..(16): 2′-O-methyl modified
  modified_base (17)..(17): 2′- fluoro modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): Lipid (C181)-conjugated
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 12 - RNA, 36 nt, Artificial Sequence; identical to SEQ ID NO: 2
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′- fluoro modified
  modified_base (4)..(4): 2′-O-methyl modified
  modified_base (5)..(5): 2′- fluoro modified
  modified_base (6)..(6): 2′-O-methyl modified
  modified_base (7)..(7): 2′-O-methyl modified
  modified_base (8)..(8): 2′- fluoro modified
  modified_base (9)..(9): 2′-O-methyl modified
  modified_base (10)..(10): 2′- fluoro modified
  modified_base (11)..(11): 2′-O-methyl modified
  modified_base (12)..(12): 2′- fluoro modified
  modified_base (13)..(13): 2′- fluoro modified
  modified_base (14)..(14): 2′-O-methyl modified
  modified_base (15)..(15): 2′- fluoro modified
  modified_base (16)..(16): 2′-O-methyl modified
  modified_base (17)..(17): 2′- fluoro modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): Lipid (C182)-conjugated
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 13 - RNA, 36 nt, Artificial Sequence; identical to SEQ ID NO: 2
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′- fluoro modified
  modified_base (4)..(4): 2′-O-methyl modified
  modified_base (5)..(5): 2′- fluoro modified
  modified_base (6)..(6): 2′-O-methyl modified
  modified_base (7)..(7): 2′-O-methyl modified
  modified_base (8)..(8): 2′- fluoro modified
  modified_base (9)..(9): 2′-O-methyl modified
  modified_base (10)..(10): 2′- fluoro modified
  modified_base (11)..(11): 2′-O-methyl modified
  modified_base (12)..(12): 2′- fluoro modified
  modified_base (13)..(13): 2′- fluoro modified
  modified_base (14)..(14): 2′-O-methyl modified
  modified_base (15)..(15): 2′- fluoro modified
  modified_base (16)..(16): 2′-O-methyl modified
  modified_base (17)..(17): 2′- fluoro modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): Lipid (C220)-conjugated
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 14 - RNA, 36 nt, Artificial Sequence; identical to SEQ ID NO: 2
  synthetic construct
  misc_feature (1)..(2): linked via phosphorothioate
  modified_base (1)..(1): 2′-O-methyl modified
  modified_base (2)..(2): 2′-O-methyl modified
  modified_base (3)..(3): 2′- fluoro modified
  modified_base (4)..(4): 2′-O-methyl modified
  modified_base (5)..(5): 2′- fluoro modified
  modified_base (6)..(6): 2′-O-methyl modified
  modified_base (7)..(7): 2′-O-methyl modified
  modified_base (8)..(8): 2′- fluoro modified
  modified_base (9)..(9): 2′-O-methyl modified
  modified_base (10)..(10): 2′- fluoro modified
  modified_base (11)..(11): 2′-O-methyl modified
  modified_base (12)..(12): 2′- fluoro modified
  modified_base (13)..(13): 2′- fluoro modified
  modified_base (14)..(14): 2′-O-methyl modified
  modified_base (15)..(15): 2′- fluoro modified
  modified_base (16)..(16): 2′-O-methyl modified
  modified_base (17)..(17): 2′- fluoro modified
  modified_base (18)..(18): 2′-O-methyl modified
  modified_base (19)..(19): 2′-O-methyl modified
  modified_base (20)..(20): 2′-O-methyl modified
  modified_base (21)..(21): 2′-O-methyl modified
  modified_base (22)..(22): 2′-O-methyl modified
  modified_base (23)..(23): 2′-O-methyl modified
  modified_base (24)..(24): 2′-O-methyl modified
  modified_base (25)..(25): 2′-O-methyl modified
  modified_base (26)..(26): 2′-O-methyl modified
  modified_base (27)..(27): 2′-O-methyl modified
  modified_base (28)..(28): Lipid (C226)-conjugated
  modified_base (29)..(29): 2′-O-methyl modified
  modified_base (30)..(30): 2′-O-methyl modified
  modified_base (31)..(31): 2′-O-methyl modified
  modified_base (32)..(32): 2′-O-methyl modified
  modified_base (33)..(33): 2′-O-methyl modified
  modified_base (34)..(34): 2′-O-methyl modified
  modified_base (35)..(35): 2′-O-methyl modified
  modified_base (36)..(36): 2′-O-methyl modified

SEQ ID NO: 15 - RNA, 16 nt, Artificial Sequence
  synthetic construct
gcagccgaaaggcugc